SEQUENCE LISTING (13 sequences)

SEQ ID NO: 1 - DNA, 17 bp, Human
tgcccgccatcatccta

SEQ ID NO: 2 - DNA, 21 bp, Human
tcctcatcgccctcccatccc

SEQ ID NO: 3 - DNA, 23 bp, Human
cgtctgttatgtaaaggatgcgt

SEQ ID NO: 4 - DNA, 17 bp, Artificial Sequence
  Sense primer for exon 3 of the ?-actin gene (GenBank accession number E01094)
gcgcggctacagcttca

SEQ ID NO: 5 - DNA, 18 bp, Artificial Sequence
  Probe for exon 3 of the ?-actin gene (GenBank accession number E01094)
caccacggccgagcggga

SEQ ID NO: 6 - DNA, 22 bp, Artificial Sequence
  Antisense primer for exon 3 of the ?-actin gene (GenBank accession number E01094)
tctccttaatgtcacgcacgat

SEQ ID NO: 7 - RNA, 12 nt, Human
uuuugccgcgcc

SEQ ID NO: 8 - DNA, 18 bp, Human
  misc_feature (10)..(10): n is a, c, g, or t
gggaatgcanggcgcggc

SEQ ID NO: 9 - DNA, 24 bp, Artificial Sequence
  DNA primer
tcaggtccctgttcgggcgccact

SEQ ID NO: 10 - DNA, 48 bp, Artificial Sequence
  DNA Template
cagtgtggaaaatctctagcagtggcgcccgaacagggacctgaaagc

SEQ ID NO: 11 - DNA, 25 bp, Artificial Sequence
  Forward primer against SARS-CoV2 non-structural protein 3 (nsp3)
agaagattggttagatgatgatagt

SEQ ID NO: 12 - DNA, 25 bp, Artificial Sequence
  Reverse primer against SARS-CoV2 non-structural protein 3 (nsp3)
ttccatctctaattgaggttgaacc

SEQ ID NO: 13 - DNA, 24 bp, Artificial Sequence
  Probe against SARS-CoV2 non-structural protein 3 (nsp3)
tcctcactgccgtcttgttgacca

(19) United States
(12) Patent Application Publication
(10) Pub. No.: US 2025/0114389 A1
(43) Pub. Date: Apr. 10, 2025

(54) NUCLEOSIDES AND NUCLEOTIDES ANALOGS AS ANTIVIRAL AGENTS

(71) Applicant: Emory University, Atlanta, GA (US)

(72) Inventors: Raymond SCHINAZI, Miami, FL (US); Franck AMBLARD, Atlanta, GA (US); Mahesh KASTHURI, Atlanta, GA (US); Nicolas BITEAU, Atlanta, GA (US); Junxing SHI, Atlanta, GA (US); Longhu ZHOU, Atlanta, GA (US); Keivan ZANDI, Atlanta, GA (US)

(21) Appl. No.: 18/285,778

(22) PCT Filed: Apr. 11, 2022
(86) PCT No.: PCT/US2022/024286

§ 371 Date: Oct. 5, 2023

Related U.S. Application Data
(60) Provisional application No. 63/173,354, filed on Apr. 9, 2021.
(60) Provisional application No. 63/175,673, filed on Apr. 16, 2021.
(60) Provisional application No. 63/210,246, filed on Jun. 14, 2021.
(60) Provisional application No. 63/288,163, filed on Dec. 10, 2021.
(60) Provisional application No. 63/298,836, filed on Jan. 12, 2022.

(51) Int. Cl. A61K 31/7068; A61K 45/06; A61P 31/14

(52) U.S. Cl. CPC A61K 31/7068; A61K 45/06; A61P 31/14

(57) ABSTRACT

Compounds, compositions and methods for preventing, treating or curing a coronavirus infection in human subjects or other animal hosts. In one embodiment, the compounds can be used to treat an infection with a severe acute respiratory syndrome virus, such as human coronavirus 229E, SARS, MERS, SARS-CoV-1, OC43, and SARS-CoV-2. In another embodiment, the methods are used to treat a patient infected with a Flavivirus, Picornavus, Togavirus, or Bunyavirus.

### FIELD

Compounds, methods and compositions for treating or preventing coronavirus infections are disclosed. More specifically, certain nucleoside and nucleotide analogs, pharmaceutically acceptable salts, or other derivatives thereof, and the use thereof in the treatment of coronaviruses, especially SARS-CoV2, are disclosed.

### BACKGROUND

Coronaviruses are a species of virus belonging to the subfamily Coronavirinae in the family Coronaviridae, and are enveloped viruses with a positive-sense single-stranded RNA genome and with a nucleocapsid of helical symmetry.

Coronaviruses primarily infect the upper respiratory and gastrointestinal tract of mammals and birds, though several known strains infect humans as well. Coronaviruses are believed to cause a significant percentage of all common colds in human adults and children.

Coronaviruses cause colds in humans, primarily in the winter and early spring seasons. Coronaviruses can also cause pneumonia, either direct viral pneumonia or a secondary bacterial pneumonia, bronchitis, either direct viral bronchitis or a secondary bacterial bronchitis, and severe acute respiratory syndrome (SARS).

Coronaviruses also cause a range of diseases in farm animals and domesticated pets, some of which can be serious and are a threat to the farming industry. In chickens, the infectious bronchitis virus (IBV), a coronavirus, targets not only the respiratory tract, but also the uro-genital tract. The virus can spread to different organs throughout the chicken.

Economically significant coronaviruses of farm animals include porcine coronavirus (transmissible gastroenteritis coronavirus, TGE) and bovine coronavirus, which both result in diarrhea in young animals. Feline Coronavirus: two forms, Feline enteric coronavirus is a pathogen of minor clinical significance, but spontaneous mutation of this virus can result in feline infectious peritonitis (FIP), a disease associated with high mortality. There are two types of canine coronavirus (CCoV), one that causes mild gastrointestinal disease and one that has been found to cause respiratory disease. Mouse hepatitis virus (MHV) is a coronavirus that causes an epidemic murine illness with high mortality, especially among colonies of laboratory mice.

Some strains of MHV cause a progressive demyelinating encephalitis in mice which has been used as a murine model for multiple sclerosis.

More recently a coronavirus pandemic has caused a dual threat to the health and the economy of the U.S. and the world. COVID-19 was first identified in December 2019 in Wuhan, Hubei province, China, resulting in the ongoing 2019-2020 pandemic. COVID-19 is caused by the severe acute respiratory syndrome coronavirus 2 (SARS-CoV-2). Common symptoms of the disease include fever (88%), dry cough (68%), shortness of breath (19%), and loss of smell (15 to 30%). Complications may include pneumonia, viral sepsis, acute respiratory distress syndrome, diarrhea, stomach aches, renal disease, cardiac issues and encephalitis. As of June, 2020, the total number of infected worldwide stood at over 4 million and at least 102,753 had died, and, according to the Johns Hopkins University Coronavirus Resource Center, almost two million people had tested positive for coronavirus in the U.S. and over one hundred thousand people had died of the disease. Local transmission of the disease has been recorded in over 200 countries. Risk factors include travel and viral exposure, and prevention is assisted by social distancing and quarantine.

Current treatments for these infections are mainly supportive, minimizing the symptoms rather than treating the underlying viral infection. For example, patients may be treated with analgesics to relieve pain, and patients with enteroviral carditis can be treated for complications such as arrhythmias, pericardial effusion, and cardiac failure.

It would be advantageous to provide new antiviral agents, compositions including these agents, and methods of treatment using these agents to treat coronaviruses. The present disclosure provides such agents, compositions and methods.

### SUMMARY

Compounds, methods and compositions for treating or preventing coronaviruses and/or other viral infections in a host are disclosed. The methods involve administering a therapeutically or prophylactically-effective amount of at least one compound described herein to treat or prevent an infection by, or an amount sufficient to reduce the biological activity of, coronaviruses or other viral infections including, but not limited to, SARS-CoV2, MERS, SARS, and OC-43. In other embodiments, the compounds described herein can be used for treating or preventing infections by Flaviviruses, Picornaviridae, Togavirodae, Bunyaviridae and other RNA viruses.

In one embodiment, methods of using potent, selective antiviral agents to target coronaviruses and other viral infections and thus help eliminate and/or treat infection in patients infected by these viruses are disclosed.

In one aspect of this embodiment, the compounds used include one or more of the specific nucleoside inhibitors described herein.

In another embodiment, pharmaceutical compositions including one or more of the compounds described herein are disclosed, which in one embodiment comprises a combination of a cytidine and a uridine analog, in combination with a pharmaceutically acceptable carrier or excipient. These compositions can be used to treat a host infected with a coronavirus or other viral infections, to prevent one of these infections, and/or to reduce the biological activity of one of these viruses. The compositions can include a combination of one or more of the compounds described herein, optionally with other antiviral compounds or biological agents, including anti-SARS-CoV2 compounds and biological agents, fusion inhibitors, entry inhibitors, protease inhibitors, polymerase inhibitors, antiviral nucleosides, such as remdesivir, GS-441524, N⁴-hydroxycytidine, and other compounds disclosed in U.S. Pat. No. 9,809,616, and their prodrugs, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

In yet another embodiment, processes for preparing the specific nucleoside compounds described herein are disclosed.

In some embodiments, the compounds described herein are deuterated at one or more positions. Where the compounds are nucleosides, deuteration can be present in one or more positions on the sugar moiety of the compounds, the base portion of the compounds, and/or the prodrug portion of the compounds, at any position.

In some embodiments, ester prodrugs were prepared to allow more drug, when given orally, to reach the plasma and not be trapped in the gut as a triphosphate.

In another embodiment, ester prodrugs were prepared to improve the oral bioavailability of drugs.

The present invention will be better understood with reference to the following Detailed Description.

### BRIEF DESCRIPTION OF THE DRAWINGS

FIG. 1A is a schematic illustration of the model of SARS-CoV-2 infection of human lung epithelium and monocytes system.

FIG. 1B is a chart showing the effect of Compound A at different concentrations on the number of copies of SARS-CoV-2 in infected monocytes in the model of SARS-CoV-2 infection of human lung epithelium and monocytes system.

FIG. 1C is a chart showing the effect of Compound A at different concentrations on the number of copies of SARS-CoV-2 in infected epithelial cells in the model of SARS-CoV-2 infection of human lung epithelium and monocytes system.

FIG. 1D is a chart showing the effect of Compound A at different concentrations on the number of copies of SARS-CoV-2 (extracellular) in the model of SARS-CoV-2 infection of human lung epithelium and monocytes system.

FIG. 1E is a chart showing the effect of Compound A at different concentrations on the number of copies of SARS-CoV-2 (total virus; cell-associated and supernatant) in the model of SARS-CoV-2 infection of human lung epithelium and monocytes system.

FIG. 2 is a chart showing the stability of Compound A in human plasma (log concentration over time (minutes).

FIG. 3 is a chart showing the stability of Compound A in mouse plasma (log concentration over time (minutes).

FIG. 4 is a chart showing the stability of Compound A in hamster plasma (log concentration over time (minutes).

FIG. 5 is a chart showing the amount of intracellular Compound A-triphosphate (pmol/million cells) after a four hour incubation in a variety of different cell types.

FIG. 6 is a chart showing the egress of Compound A-triphosphate in Vero cells (pmol/million cells versus time in hours).

FIG. 7 is a chart showing the egress of Compound A-triphosphate in Calu-3 cells (pmol/million cells versus time in hours).

FIG. 8A is a chart showing the concentration (g/ml) over time (hours) where Compound A is administered orally at a concentration of 30 mg/kg.

FIG. 8B is a chart showing the concentration (g/ml) over time (hours) where Compound A is administered intravenously at a concentration of 15 mg/kg.

FIG. 9 is a chart showing the body weight of individual Syrian hamsters, in grams, during nucleoside compound A (in days).

FIG. 10 is a chart showing the temperature of individual Syrian hamsters, in degrees c., during compound A treatment (in days).

### DETAILED DESCRIPTION

The compounds described herein show inhibitory activity against Coronaviridae in cell-based assays. Therefore, the compounds can be used to treat or prevent a Coronaviridae infection in a host, or reduce the biological activity of the virus. The host can be a mammal, and in particular, a human, infected with Coronaviridae virus. The compounds are also effective against Flaviviridae, Picornaviridae, Togavirodae, Bunyaviridae viruses and other RNA viruses. The methods involve administering an effective amount of one or more of the compounds described herein.

Pharmaceutical formulations including one or more compounds described herein, in combination with a pharmaceutically acceptable carrier or excipient, are also disclosed. In one embodiment, the formulations include at least one compound described herein and at least one further therapeutic agent.

The present invention will be better understood with reference to the following definitions:

### I. Definitions

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

As used herein, the term “enantiomerically pure” refers to a compound composition that comprises at least approximately 95%, and, preferably, approximately 97%, 98%, 99% or 100% of a single enantiomer of that compound.

As used herein, the term “substantially free of” or “substantially in the absence of” refers to a compound composition that includes at least 85 to 90% by weight, preferably 95% to 98% by weight, and, even more preferably, 99% to 100% by weight, of the designated enantiomer of that compound. In a preferred embodiment, the compounds described herein are substantially free of enantiomers.

Similarly, the term “isolated” refers to a compound composition that includes at least 85 to 90% by weight, preferably 95% to 98% by weight, and, even more preferably, 99% to 100% by weight, of the compound, the remainder comprising other chemical species or enantiomers.

The term “alkyl,” as used herein, unless otherwise specified, refers to a saturated straight, branched, or cyclic, primary, secondary, or tertiary hydrocarbons, including both substituted and unsubstituted alkyl groups. The alkyl group can be optionally substituted with any moiety that does not otherwise interfere with the reaction or that provides an improvement in the process, including but not limited to but limited to halo, C₁₋₆ haloalkyl, hydroxyl, carboxyl, C₁₋₆ acyl, aryl, C₁₋₆ acyloxy, amino, amido, carboxyl derivatives, alkylamino, di-C₁₋₆-alkylamino, arylamino, C₁₋₆ alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included are CF₃ and CH₂CF₃.

In the text, whenever the term C(alkyl range) is used, the term independently includes each member of that class as if specifically and separately set out. The term “alkyl” includes C₁₋₂₂ alkyl moieties, and the term “lower alkyl” includes C₁₋₆ alkyl moieties. It is understood to those of ordinary skill in the art that the relevant alkyl radical is named by replacing the suffix “-ane” with the suffix “-yl”.

As used herein, a “bridged alkyl” refers to a bicyclo- or tricyclo alkane, for example, a 2:1:1 bicyclohexane.

As used herein, a “spiro alkyl” refers to two rings that are attached at a single (quaternary) carbon atom.

The term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to but not limited to those described for substituents on alkyl moieties.

Non-limiting examples of alkenyl groups include ethylene, methylethylene, isopropylidene, 1,2-ethane-diyl, 1,1-ethane-diyl, 1,3-propane-diyl, 1,2-propane-diyl, 1,3-butane-diyl, and 1,4-butane-diyl.

The term “alkynyl” refers to an unsaturated, acyclic hydrocarbon radical, linear or branched, in so much as it contains one or more triple bonds. The alkynyl group can be optionally substituted with any moiety that does not adversely affect the reaction process, including but not limited to those described above for alkyl moeities. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

The term “alkylamino” or “arylamino” refers to an amino group that has one or two alkyl or aryl substituents, respectively.

The term “fatty alcohol” as used herein refers to straight-chain primary alcohols with between 4 and 26 carbons in the chain, preferably between 8 and 26 carbons in the chain, and most preferably, between 10 and 22 carbons in the chain. The precise chain length varies with the source. Representative fatty alcohols include lauryl, stearyl, and oleyl alcohols. They are colourless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. They are widely used in industry. As with fatty acids, they are often referred to generically by the number of carbon atoms in the molecule, such as “a C₁₂ alcohol”, that is an alcohol having 12 carbons, for example dodecanol.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis, and are described, for example, in Greene et al., Protective Groups in Organic Synthesis, supra.

The term “aryl”, alone or in combination, means a carbocyclic aromatic system containing one, two or three rings wherein such rings can be attached together in a pendent manner or can be fused. Non-limiting examples of aryl include phenyl, biphenyl, or naphthyl, or other aromatic groups that remain after the removal of a hydrogen from an aromatic ring. The term aryl includes both substituted and unsubstituted moieties. The aryl group can be optionally substituted with any moiety that does not adversely affect the process, including but not limited to but not limited to those described above for alkyl moieties. Non-limiting examples of substituted aryl include heteroarylamino, N-aryl-N-alkylamino, N-heteroarylamino-N-alkylamino, heteroaralkoxy, arylamino, aralkylamino, arylthio, monoarylamidosulfonyl, arylsulfonamido, diarylamidosulfonyl, monoaryl amidosulfonyl, arylsulfinyl, arylsulfonyl, heteroarylthio, heteroarylsulfinyl, heteroarylsulfonyl, aroyl, heteroaroyl, aralkanoyl, heteroaralkanoyl, hydroxyaralkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, and heteroarylalkenyl, carboaralkoxy.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a carboxylic acid ester in which the non-carbonyl moiety of the ester group is selected from the group consisting of straight, branched, or cyclic alkyl or lower alkyl, alkoxyalkyl, including, but not limited to methoxymethyl, aralkyl, including, but not limited to, benzyl, aryloxyalkyl, such as phenoxymethyl, aryl, including, but not limited to, phenyl, optionally substituted with halogen (F, Cl, Br, or I), alkyl (including but not limited to C₁, C₂, C₃, and C₄) or alkoxy (including but not limited to C₁, C₂, C₃, and C₄), sulfonate esters such as alkyl or aralkyl sulphonyl including but not limited to methanesulfonyl, the mono, di or triphosphate ester, trityl or monomethoxytrityl, substituted benzyl, trialkylsilyl (e.g., dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals can be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “heteroatom,” as used herein, refers to oxygen, sulfur, nitrogen and phosphorus.

The terms “heteroaryl” or “heteroaromatic,” as used herein, refer to an aromatic that includes at least one sulfur, oxygen, nitrogen or phosphorus in the aromatic ring.

The term “heterocyclic,” “heterocyclyl,” and cycloheteroalkyl refer to a nonaromatic cyclic group wherein there is at least one heteroatom, such as oxygen, sulfur, nitrogen, or phosphorus in the ring.

Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic group can be optionally substituted as described above for aryl. The heterocyclic or heteroaromatic group can be optionally substituted with one or more substituents selected from the group consisting of halogen, haloalkyl, alkyl, alkoxy, hydroxy, carboxyl derivatives, amido, amino, alkylamino, and dialkylamino. The heteroaromatic can be partially or totally hydrogenated as desired. As a nonlimiting example, dihydropyridine can be used in place of pyridine. Functional oxygen and nitrogen groups on the heterocyclic or heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl. The heterocyclic or heteroaromatic group can be substituted with any moiety that does not adversely affect the reaction, including but not limited to but not limited to those described above for aryl.

The term “host,” as used herein, refers to a unicellular or multicellular organism in which the virus can replicate, including but not limited to cell lines and animals, and, preferably, humans. Alternatively, the host can be carrying a part of the viral genome, whose replication or function can be altered by the compounds described herein. The term host specifically refers to infected cells, cells transfected with all or part of the viral genome and animals, in particular, primates (including but not limited to chimpanzees) and humans. In most animal applications described herein, the host is a human being. Veterinary applications, in certain indications, however, are clearly contemplated by the present disclosure (such as for use in treating chimpanzees).

The term nucleoside also includes ribonucleosides, and representative ribonucleosides are disclosed, for example, in the Journal of Medicinal Chemistry, 43(23), 4516-4525 (2000), Antimicrobial Agents and Chemotherapy, 45(5), 1539-1546 (2001), and PCT WO 2000069876.

The term “peptide” refers to a natural or synthetic compound containing two to one hundred amino acids linked by the carboxyl group of one amino acid to the amino group of another.

The term “pharmaceutically acceptable salt or prodrug” is used throughout the specification to describe any pharmaceutically acceptable form (such as an ester) compound which, upon administration to a patient, provides the compound. Pharmaceutically-acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art.

Pharmaceutically acceptable prodrugs refer to a compound that is metabolized, for example hydrolyzed or oxidized, in the host to form an active compound. Typical examples of prodrugs include compounds that have biologically labile protecting groups on functional moieties of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. The prodrug forms of the compounds described herein can possess antiviral activity, can be metabolized to form a compound that exhibits such activity, or both.

### II. Active Compounds

In one embodiment, the compounds are compounds of Formula (A):

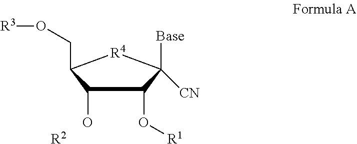

or a pharmaceutically acceptable salt or prodrug thereof, wherein:
R⁴ is O, CH₂, S, Se, or

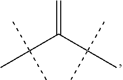

R¹ and R² are independently selected from the group consisting of H, an L-amino acid ester, a D-amino acid ester, an N-substituted L-amino acid ester, an N-substituted D-amino acid ester, an N,N-disubstituted L-amino acid ester, an N,N-disubstituted D-amino acid ester, (acyloxybenzyl)ester, (acyloxybenzyl)ether, optionally substituted bis-acyloxybenzyl)esters, optionally substituted (acyloxybenzyl)esters, an optionally substituted —C(O)—C₁₋₁₂R′, an optionally substituted —C(O)O—R′, an optionally substituted —C(O)S—R′, an optionally substituted —C(S)S—R′, an optionally substituted —C(NR′)OR′, an optionally substituted —C(NR′)SR′, an optionally substituted —C(NR′)N(R′)₂, and an optionally substituted —O—C(O)N(R′)₂, PEG ester, PEG carbonate, an optionally substituted —CH₂—O—C(O)—R′, an optionally substituted —CH₂—O—C(O)O—R′, an optionally substituted —CH₂—CH₂—S—C(O)—R′, a lipid ester, a lipid carbonate, (in which the lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy),
R′ is C₁₋₁₆ alkyl, C₂₋₁₆ alkenyl, C₂₋₁₆ alkynyl, or C₃₋₇ cycloalkyl,
wherein optional substituents are selected from the group consisting of halo, C₁₋₁₂ haloalkyl, C₁₋₁₆ alkyl, C₂₋₁₆ alkenyl, C₂₋₁₆ alkynyl, C₃₋₇ cycloalkyl, hydroxyl, carboxyl, C₁₋₁₂ acyl, aryl, heteroaryl, C₁₋₆ acyloxy, amino, amido, carboxyl derivatives, alkylamino, di-C₁₋₁₂-alkylamino, arylamino, C₁₋₁₂ alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, boronic acid and boronic ester;
R³ is H, an L-amino acid ester, a D-amino acid ester, an N-substituted L-amino acid ester, an N-substituted D-amino acid ester, an N,N-disubstituted L-amino acid ester, an N,N-disubstituted D-amino acid ester, (acyloxybenzyl)ester, (acyloxybenzyl)ether, optionally substituted bis-acyloxybenzyl)ester, optionally substituted (acyloxybenzyl)ester, an optionally substituted —C(O)—R′, an optionally substituted —C(O)O—R′, an optionally substituted —C(O)SR′, an optionally substituted —C(S)SR′, PEG ester, PEG carbonate, an optionally substituted —CH₂—O—C(O)—R′, an optionally substituted —CH₂—O—C(O)O—R′, an optionally substituted —CH₂—CH₂—S—C(O)—R′, an optionally substituted —C(NR′)OR′, an optionally substituted —C(NR′)SR′, an optionally substituted —C(NR′)N(R′)₂, an optionally substituted —O—C(O)N(R′)₂, a lipid ester, a lipid carbonate (in which a lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy), O—P(O)R⁶R⁷, or a mono-, di-, or triphosphate, wherein, when chirality exists at the phosphorous center, it may be wholly or partially Rₚ or Sₚ or any mixture thereof,
R⁶ and R⁷ are independently selected from the group consisting of:
(a) OR¹⁵ where R¹⁵ selected from the group consisting of H,

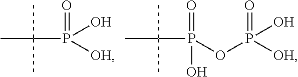

Li, Na, K, substituted or unsubstituted C₁₋₂₀alkyl, substituted or unsubstituted C₃₋₆cycloalkyl, optionally substituted —C(NR′)OR′, optionally substituted —C(NR′)SR′, optionally substituted —C(NR′)N(R′)₂, optionally substituted —O—C(O)N(R′)₂, C₁₋₄(alkyl)aryl, benzyl, C₁₋₆ haloalkyl, C₂₋₃(alkyl)OC₁₋₂₀alkyl, C₂₋₃(alkyl)OC₁₋₂₀alkene, C₂₋₃(alkyl)OC₁₋₂₀alkyne, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, substituted or unsubstituted C₁₋₂₀ alkene, substituted or unsubstituted C₁₋₂₀ alkyne, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, C₁₋₅ alkene, C₁₋₅ alkyne, C₃₋₇ cycloalkyl or C₁₋₅ alkyl substituted with a C₁₋₆ alkyl, alkoxy, di(C₁₋₆ alkyl)-amino, fluoro, C₃₋₁₀ cycloalkyl, or cycloalkyl; and
(b) the ester of a D- or L-amino acid

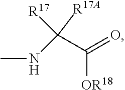

wherein R¹⁷ and R¹⁸ are, independently, H, C₁₋₂₀ alkyl, C₁₋₂₀ alkene, C₁₋₂₀ alkyne, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and R^{17A} is H or C₁₋₂alkyl;
Base is

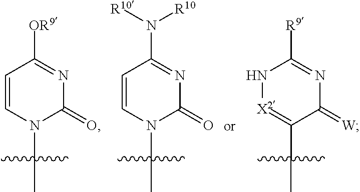

R^{9′} is H, an L-amino acid ester, a D-amino acid ester, an N-substituted L-amino acid ester, an N-substituted D-amino acid ester, an N,N-disubstituted L-amino acid ester, an N,N-disubstituted D-amino acid ester, an (acyloxybenzyl)ester, an (acyloxybenzyl)ether, an optionally substituted bis-acyloxybenzyl)ester, an optionally substituted (acyloxybenzyl)ester, an optionally substituted —C(O)—R′, an optionally substituted —C(O)O—R′, an optionally substituted —C(O)S—R′, an optionally substituted —C(S)S—R′, an optionally substituted C₁₋₁₂-alkyl, an optionally substituted C₂₋₁₂ alkenyl, an optionally substituted C₂₋₁₂ alkynyl, an optionally substituted C₃₋₆ cycloalkyl, an optionally substituted —C(NR′)OR′, an optionally substituted —C(NR′)SR′, an optionally substituted —C(NR′)N(R′)₂, an optionally substituted —O—C(O)N(R′)₂, a PEG ester, a PEG carbonate, an optionally substituted —CH₂—O—C(O)—R′, an optionally substituted —CH₂—O—C(O)O—R′, an optionally substituted —CH₂—CH₂—S—C(O)—R′, a lipid ester, or a lipid carbonate,
wherein a lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy),
R¹⁰ and R^{10′} are independently H, OH, an L-amino acid amide, a D-amino acid amide, (acyloxybenzyl)amide, (acyloxybenzyl)amine, optionally substituted (acyloxybenzyl)esters, an optionally substituted —C(O)—R′, an optionally substituted —C(O)O—R′, an optionally substituted —C(O)S—R′, an optionally substituted —C(S)S—R′, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, PEG amide, PEG carbamate, an optionally substituted —CH₂—O—C(O)—R′, an optionally substituted —CH₂—O—C(O)O—R′, an optionally substituted —CH₂—CH₂—S—C(O)—R′, a lipid amide, an optionally substituted —C(NR′)OR′, an optionally substituted —C(NR′)SR′, an optionally substituted —C(NR′)N(R′)₂, an optionally substituted —O—C(O)N(R′)₂, or a lipid carbamate, wherein a lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy), with the proviso that R¹⁰ and R^{10′} cannot both be OH.

In one embodiment, R⁴ is O.

In one embodiment, neither R¹⁰ or R^{10′} are OH.

In one embodiment, both R¹⁰ and R^{10′} are H.

In one embodiment, R⁴ is CH₂.

In one embodiment, R⁴ is S.

In one embodiment, R⁴ is O and R¹, R², R³, R⁹, R¹⁰ and R^{10′} are not all H.

In one embodiment, R¹ and R² are H, an L-amino acid ester, a D-amino acid ester or an optionally substituted —C(O)—C₁₋₁₂ alkyl.

In one embodiment, R³ is H, an L-amino acid ester, a D-amino acid ester or an optionally substituted —C(O)—C₁₋₁₂ alkyl.

In one embodiment, R¹⁰ and R^{10′} are independently H, OH, an L-amino acid amide, a D-amino acid amide, an optionally substituted —C(O)—C₁₋₁₂ alkyl, with the proviso that R¹⁰ and R^{10′} cannot both be OH.

In one embodiment, R⁹ is H, an L-amino acid ester with the oxygen to which it is attached, a D-amino acid ester with the oxygen to which it is attached, or an optionally substituted —C(O)—C₁₋₁₂ alkyl.

Representative compounds include the following:

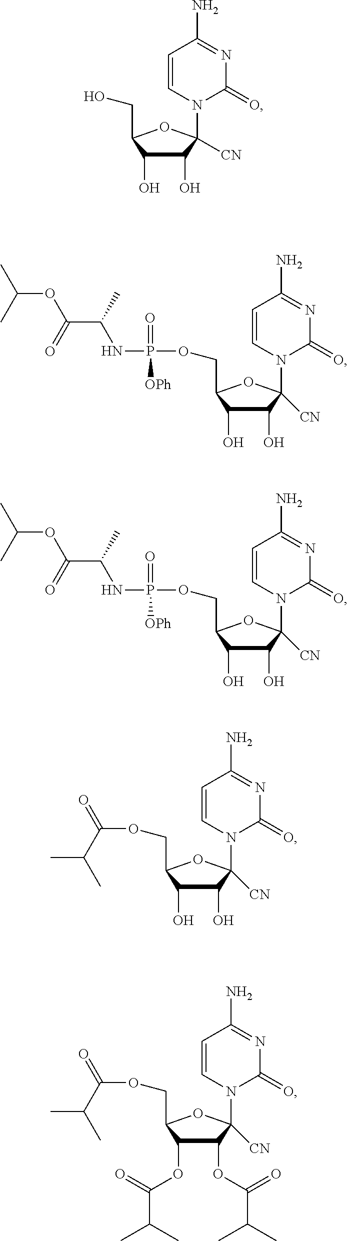

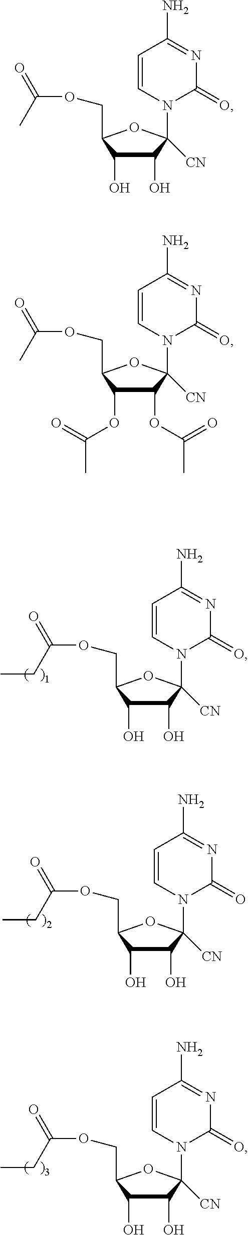

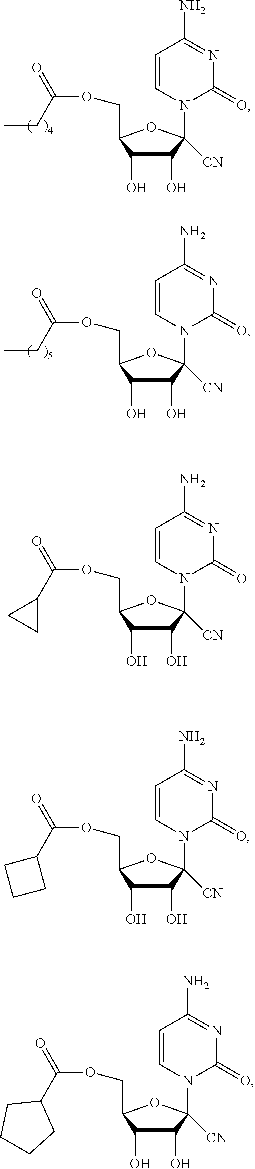

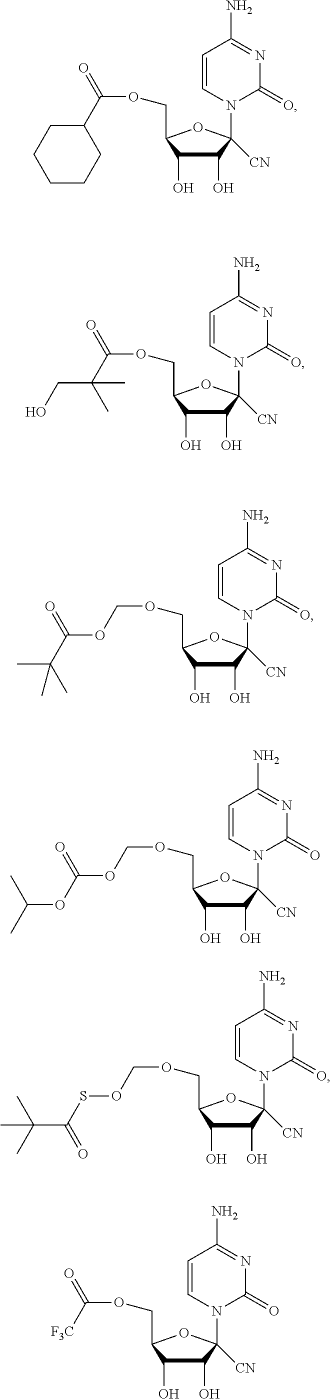

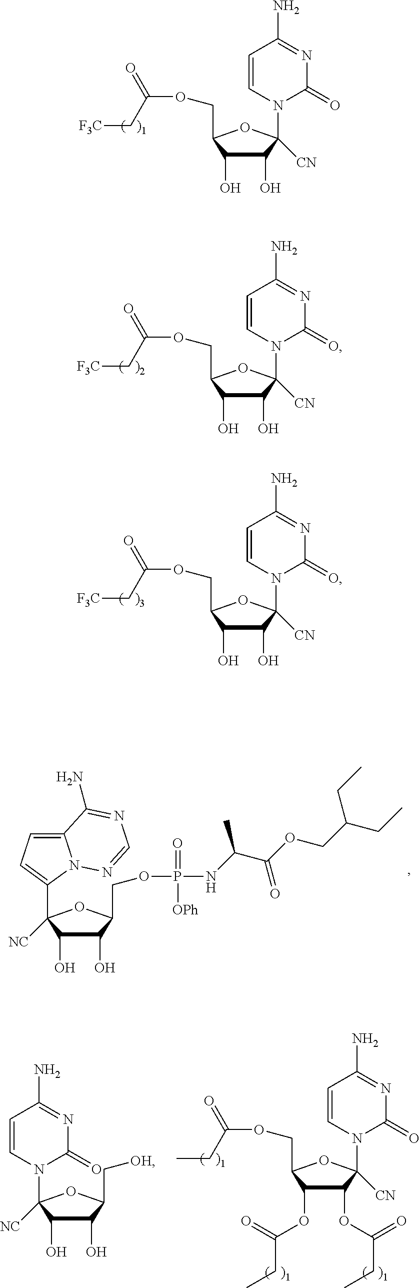

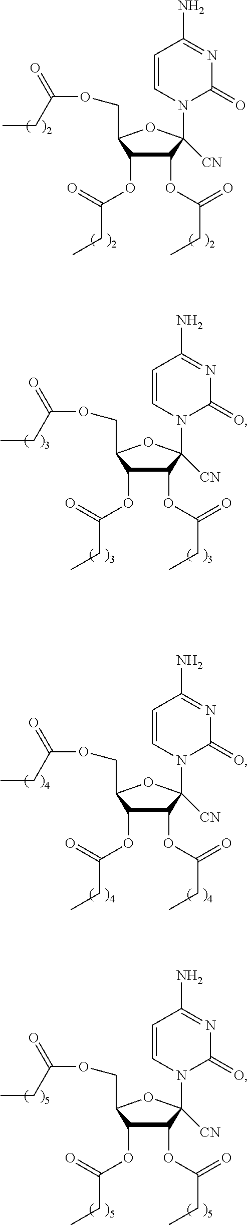

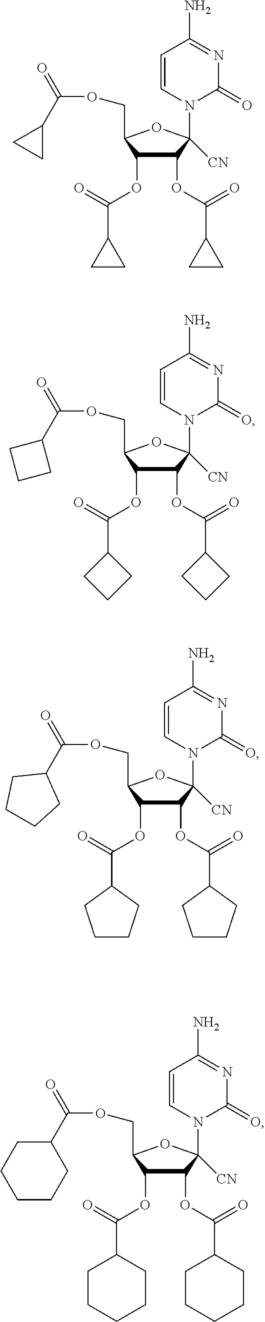

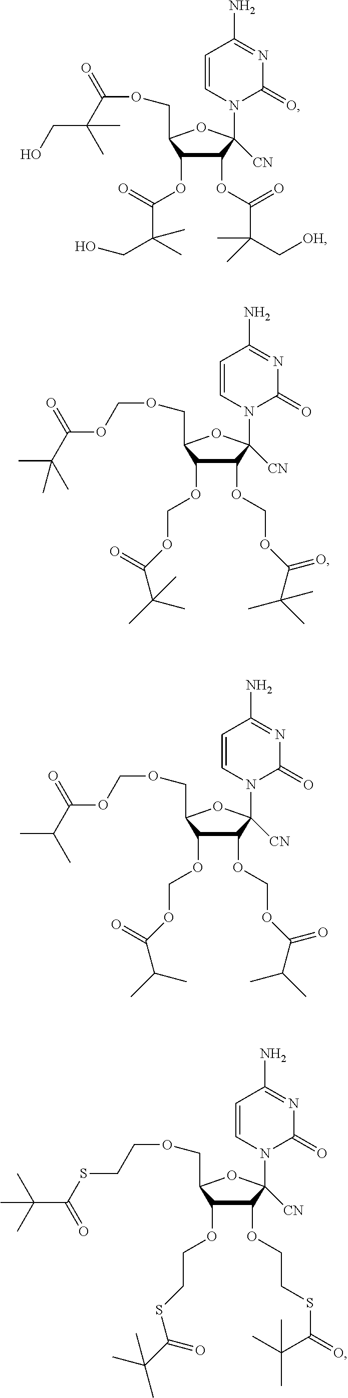

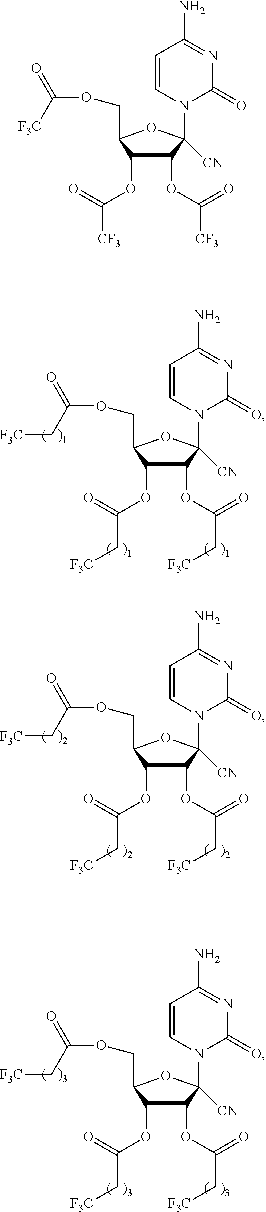

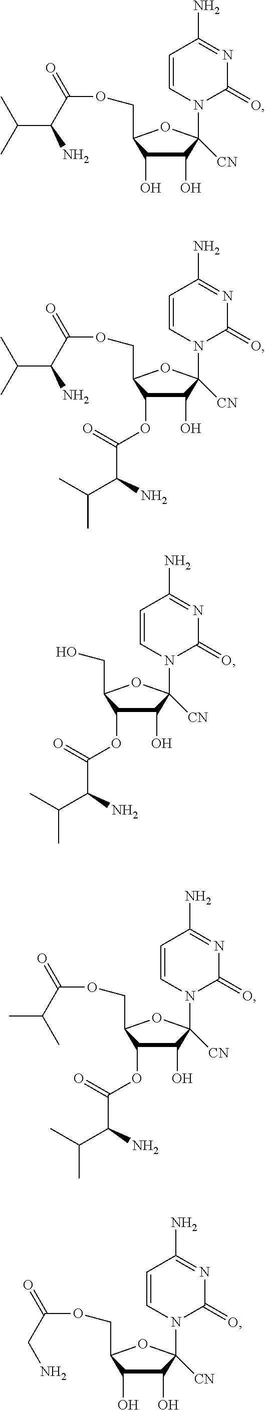

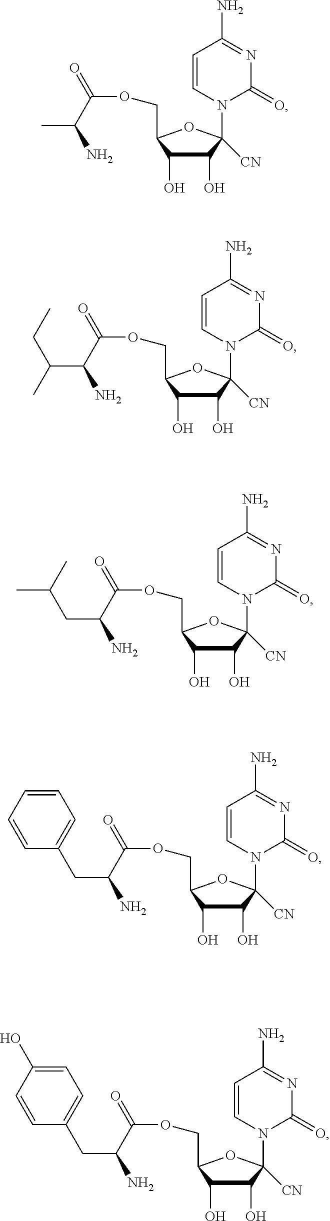

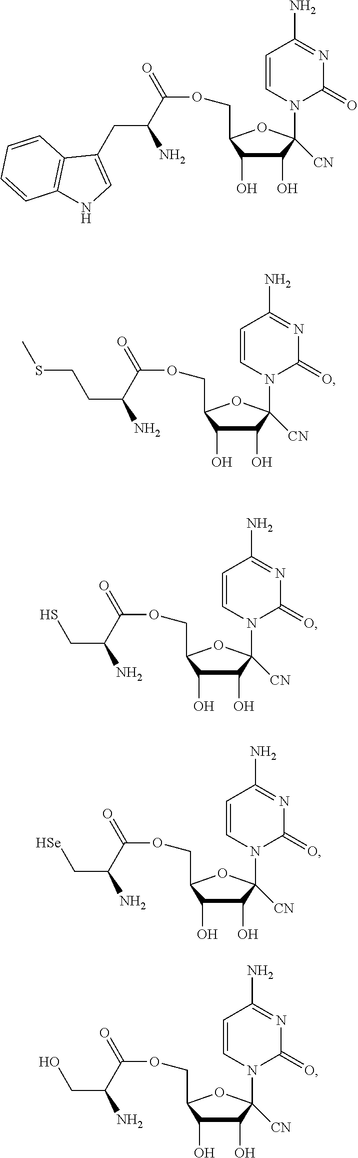

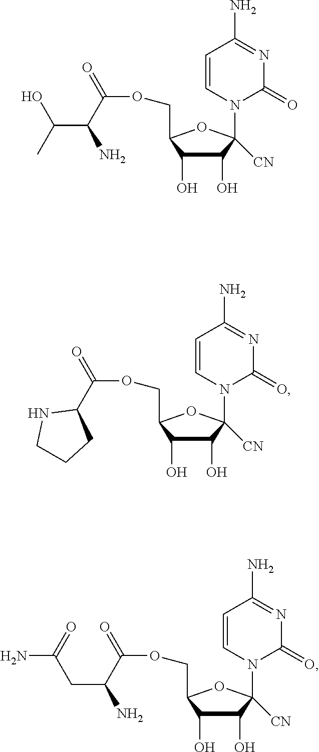

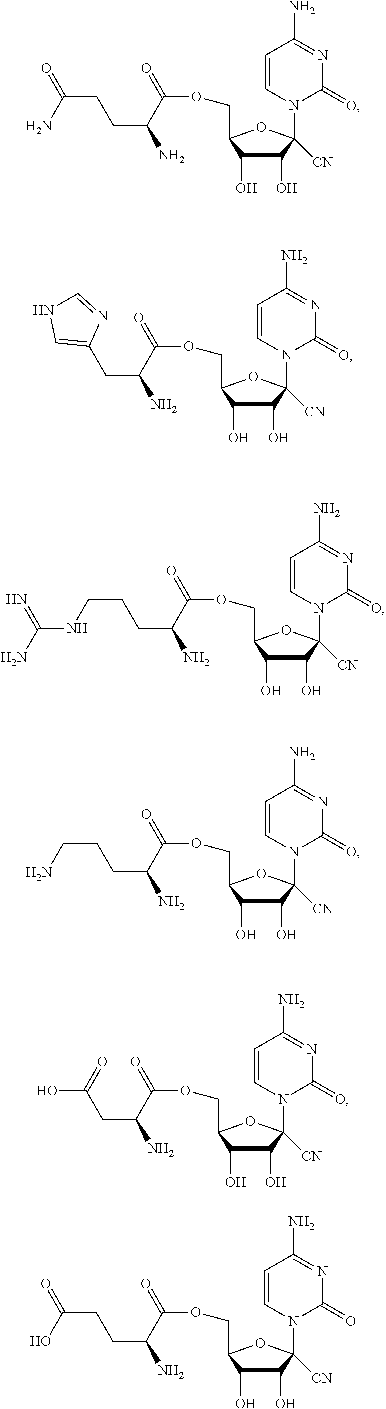

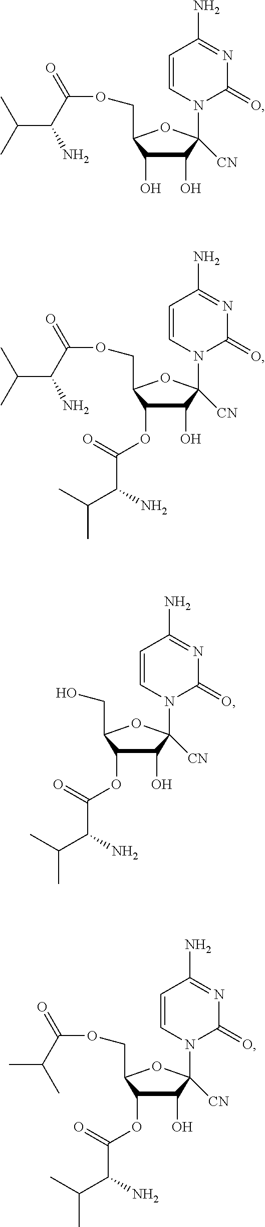

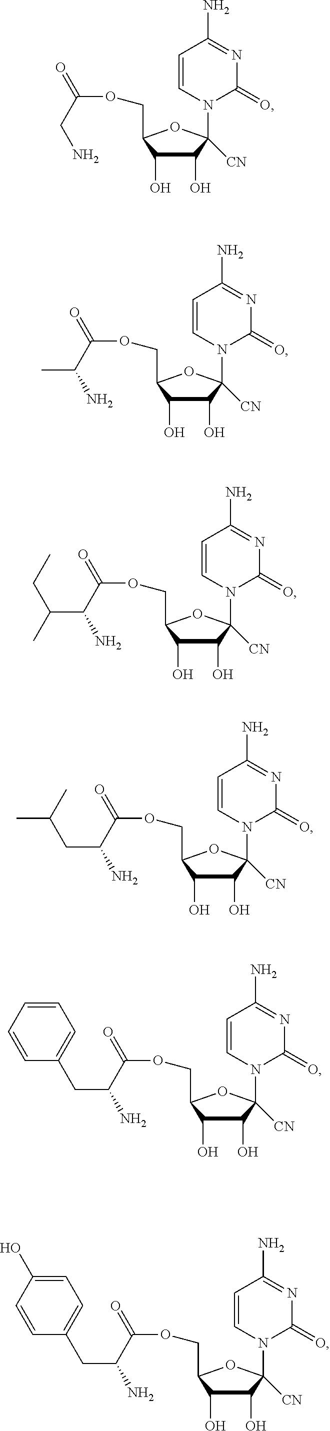

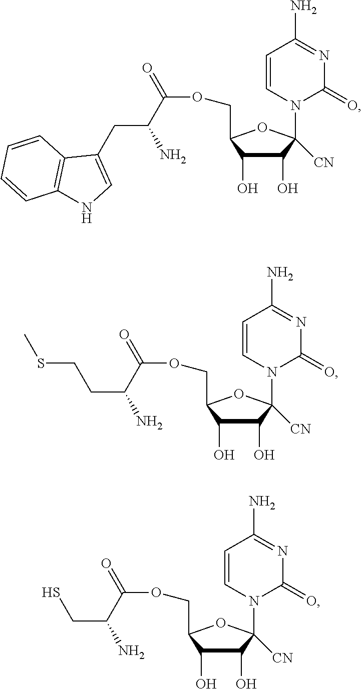

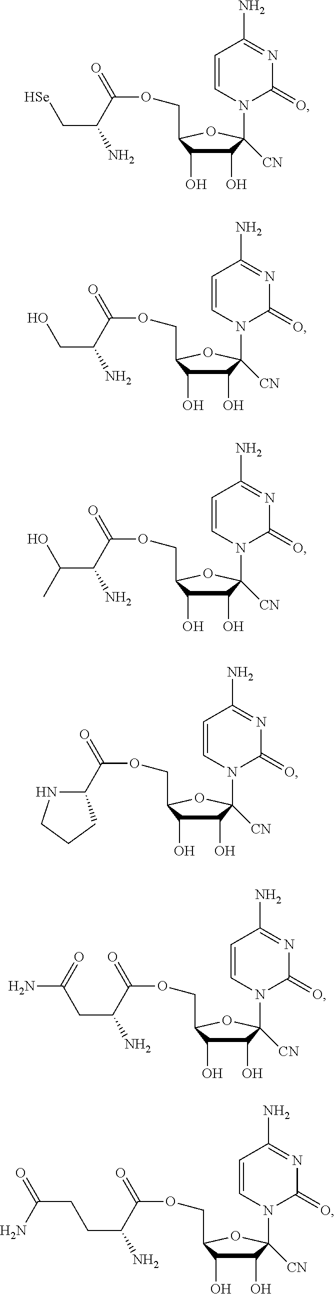

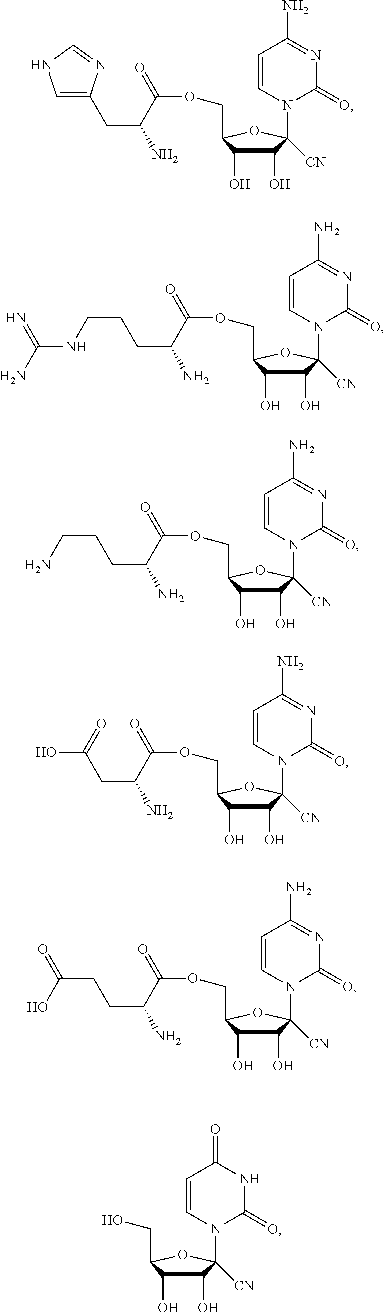

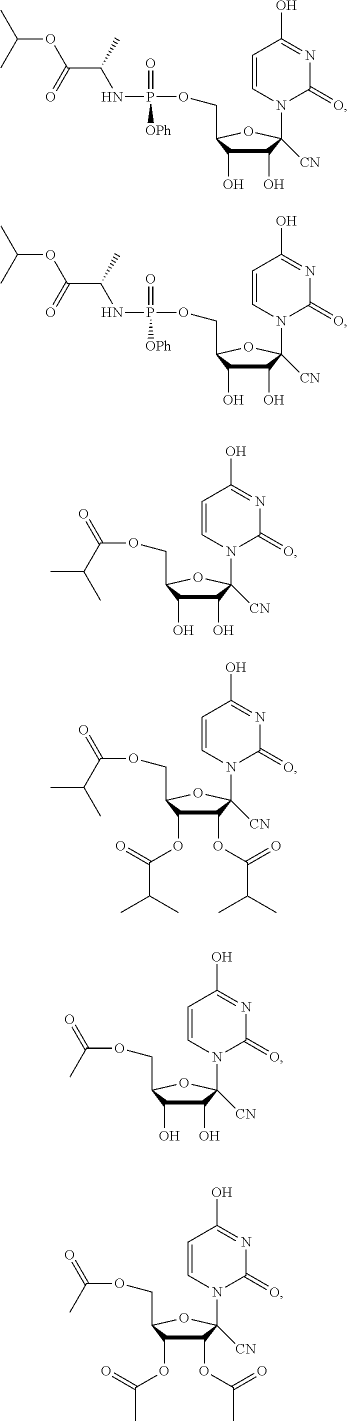

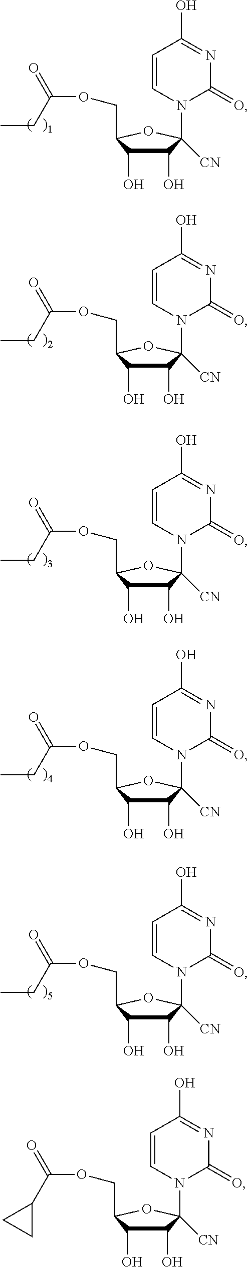

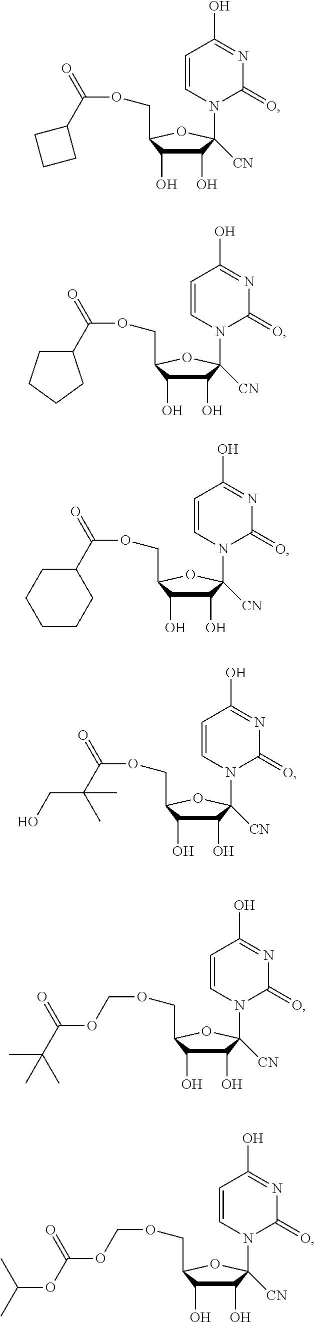

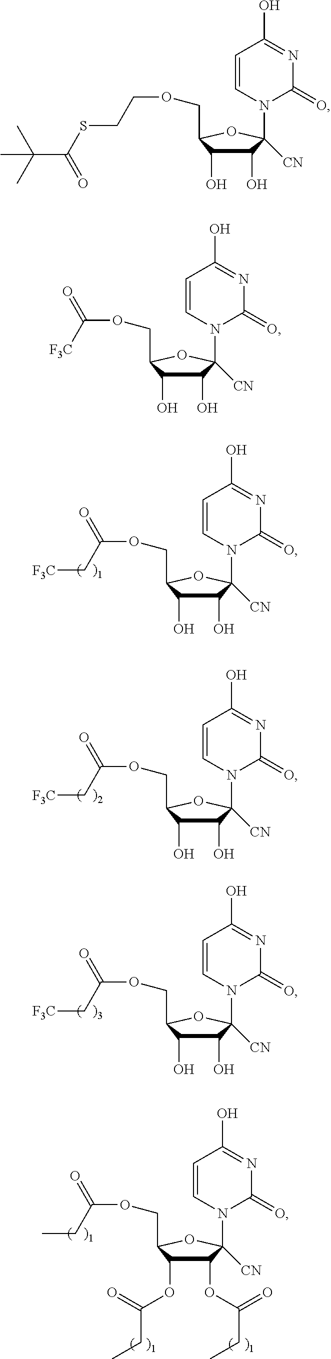

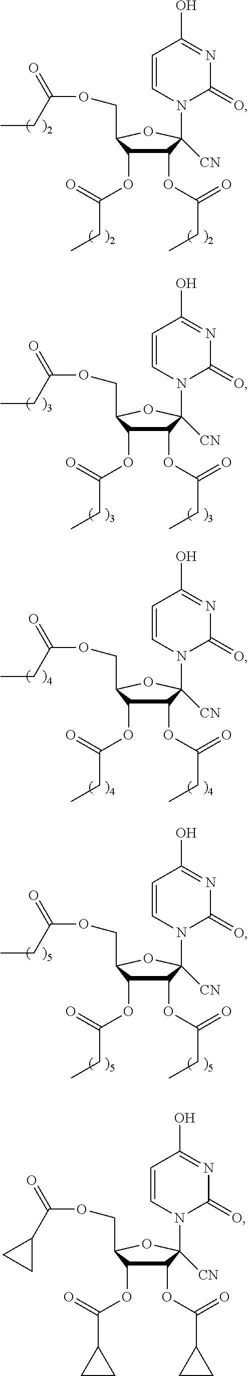

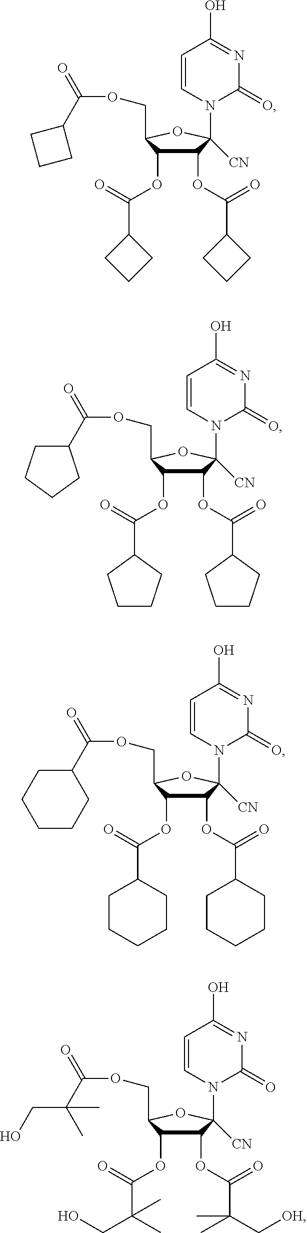

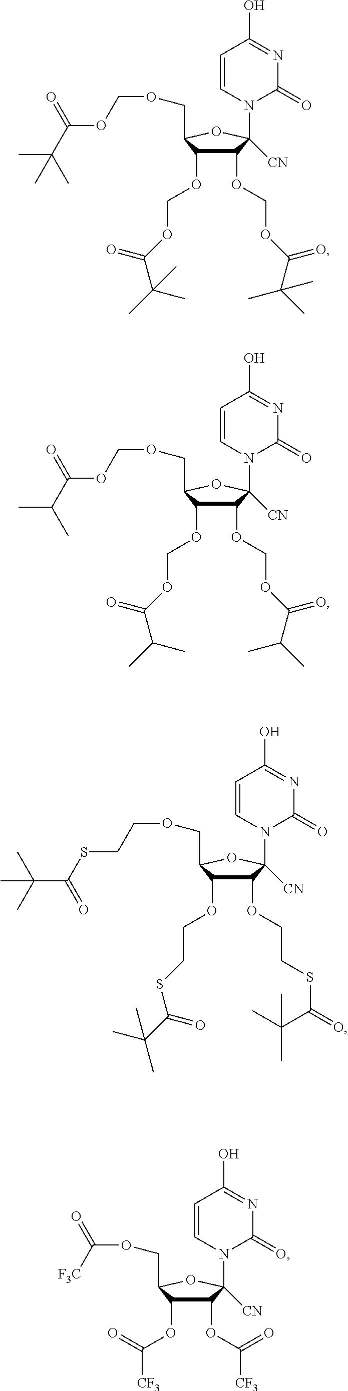

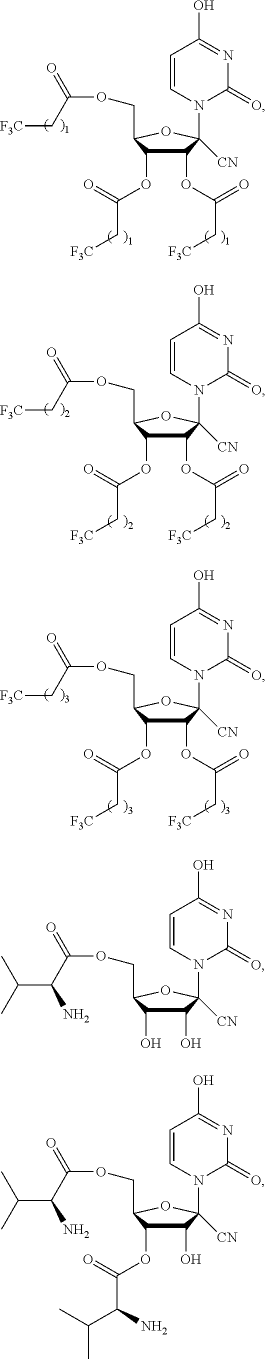

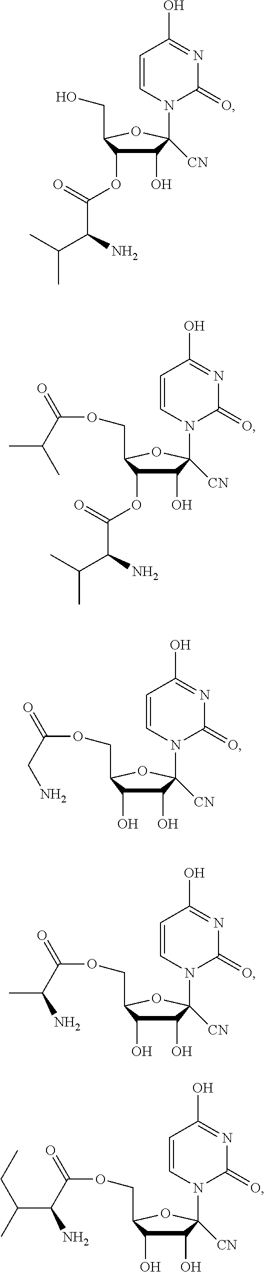

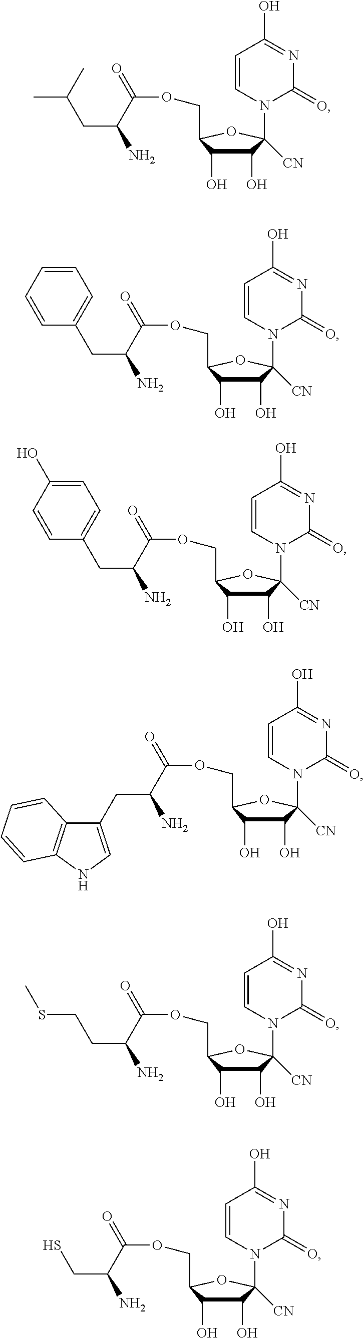

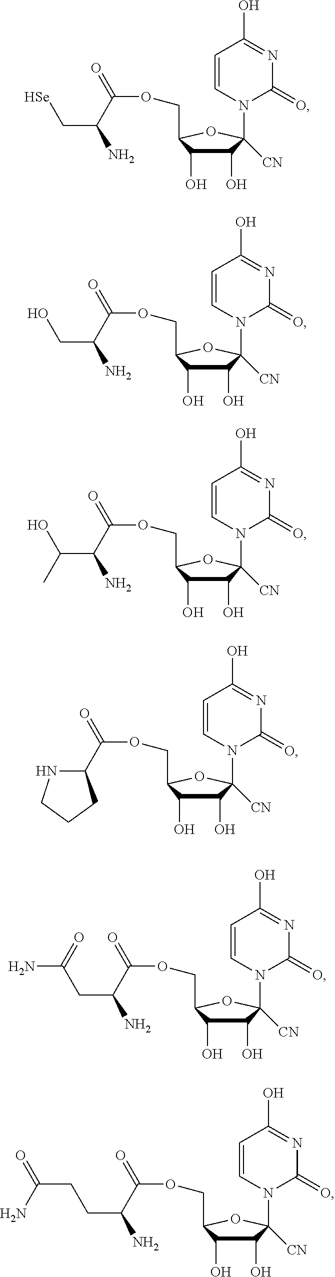

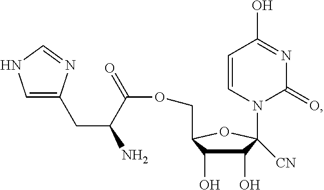

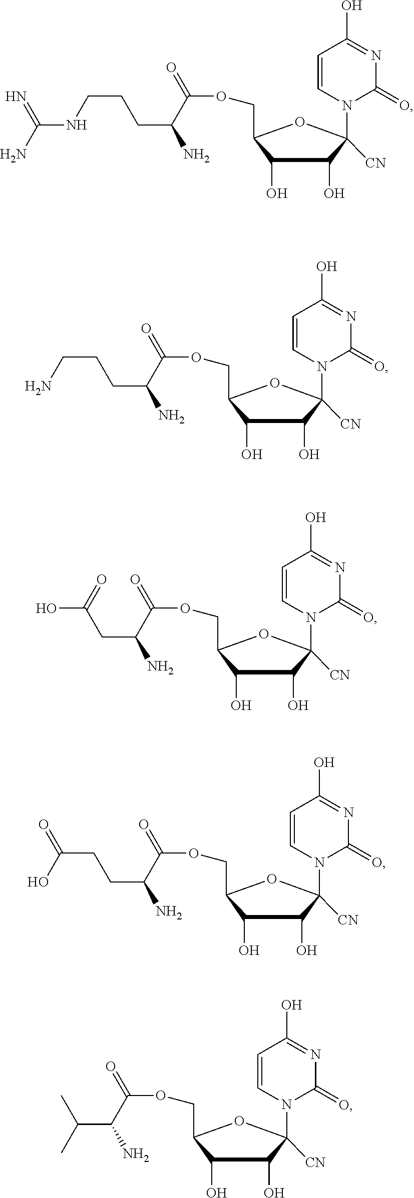

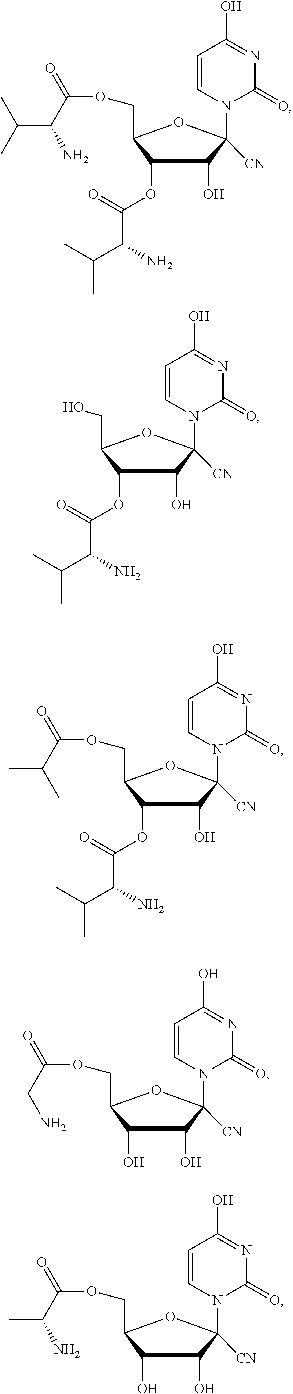

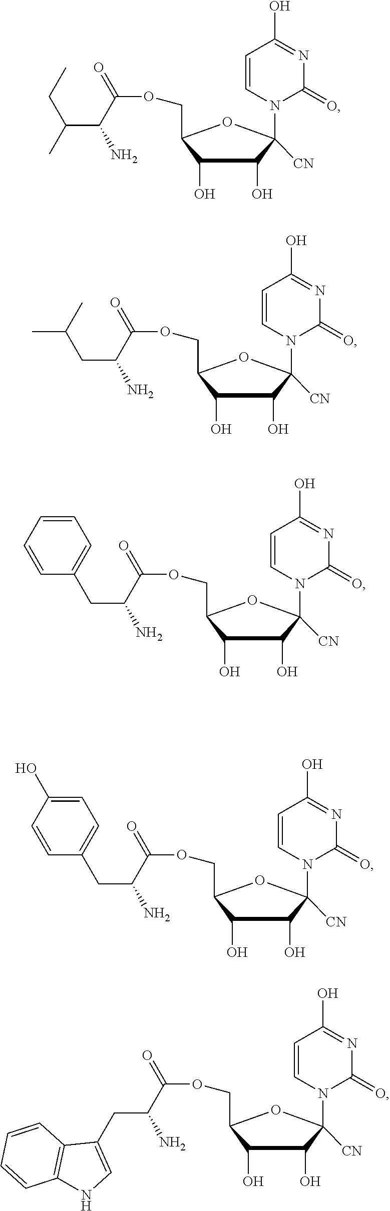

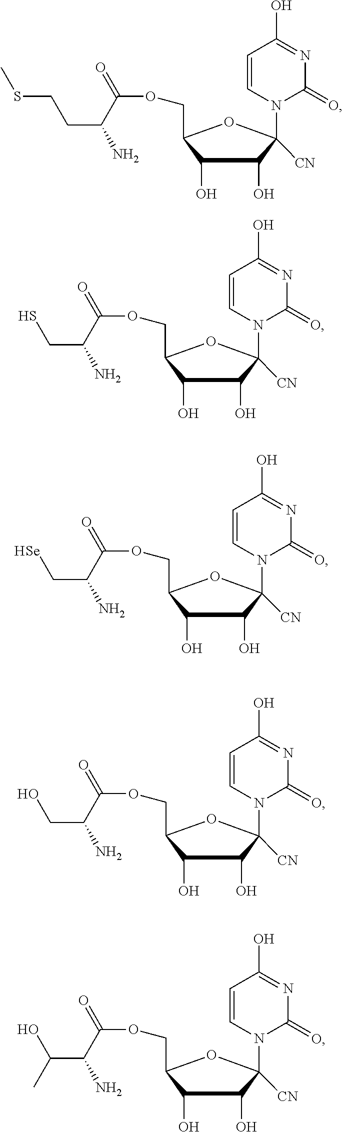

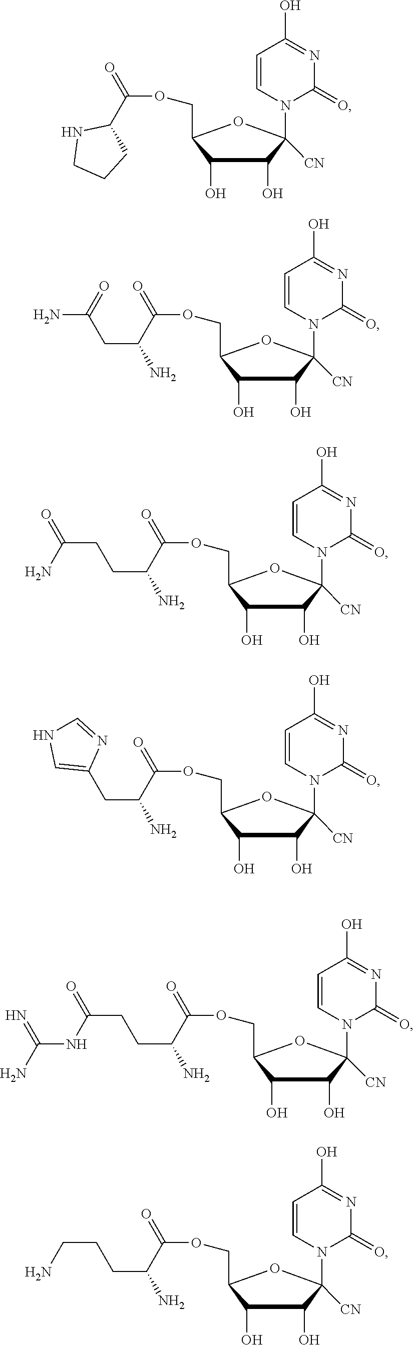

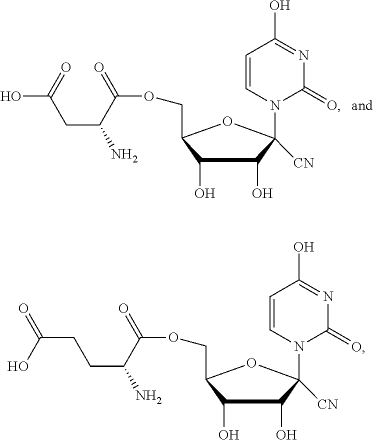

or a pharmaceutically acceptable salt or prodrug thereof.

Representative compounds also include following:

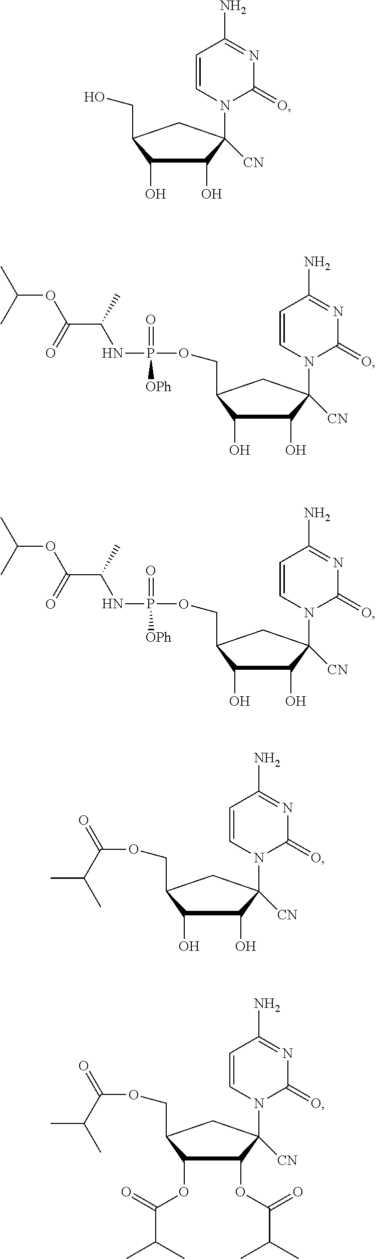

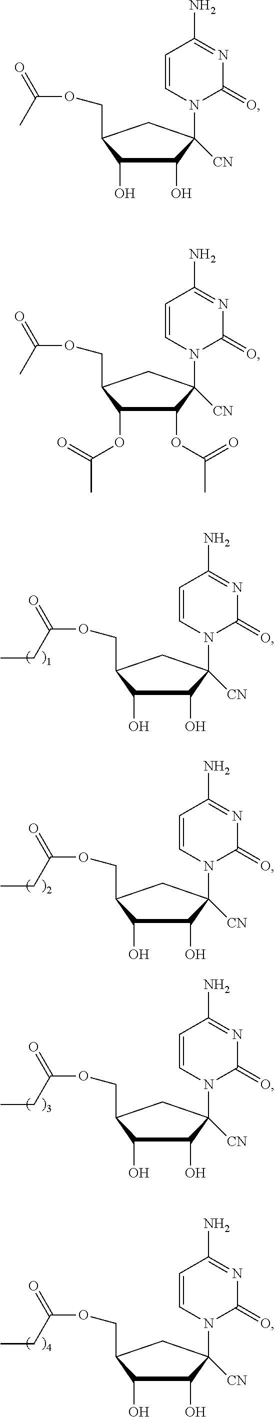

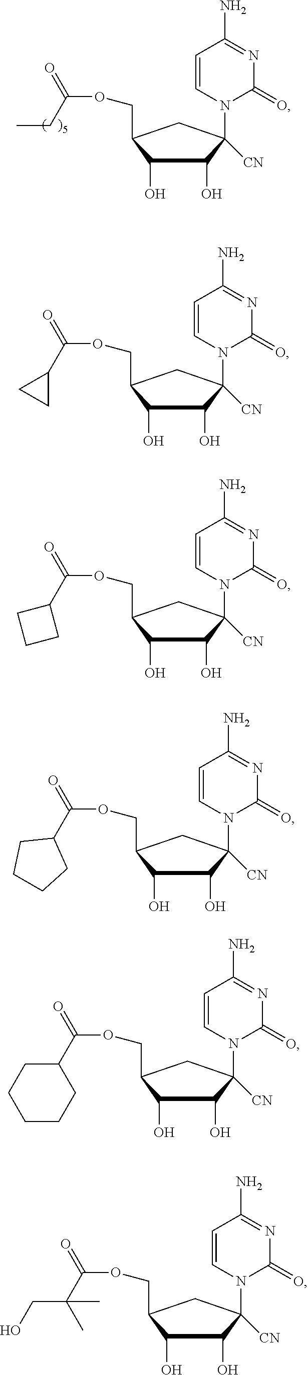

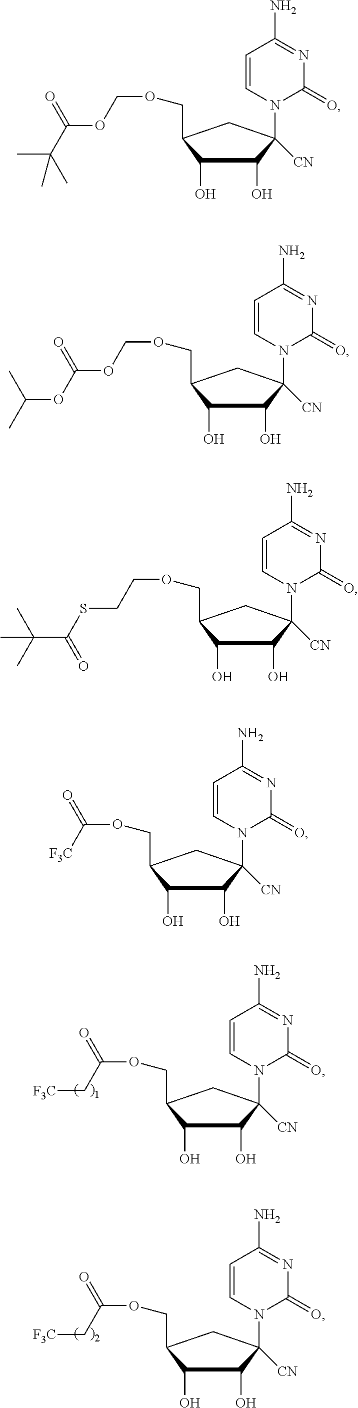

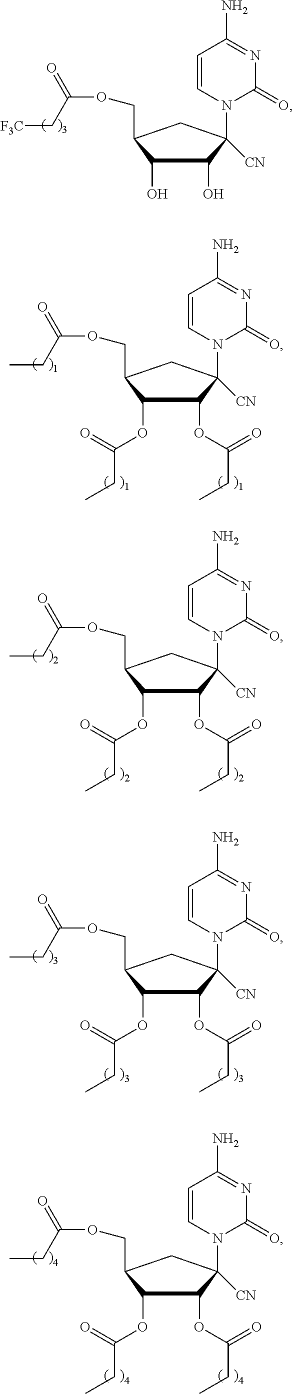

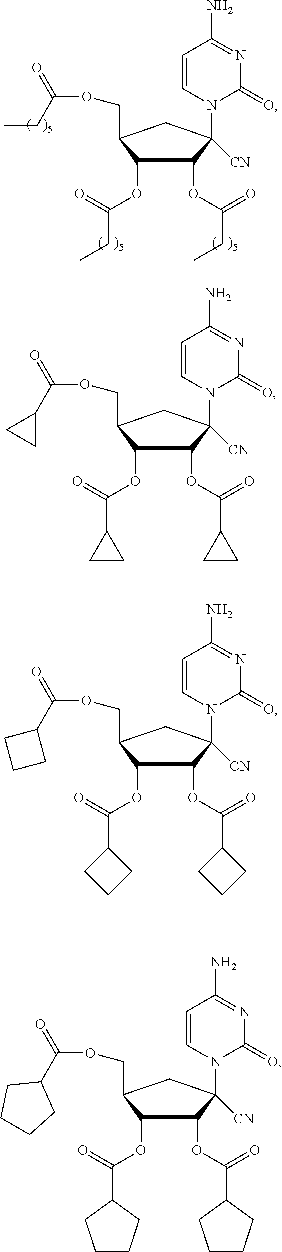

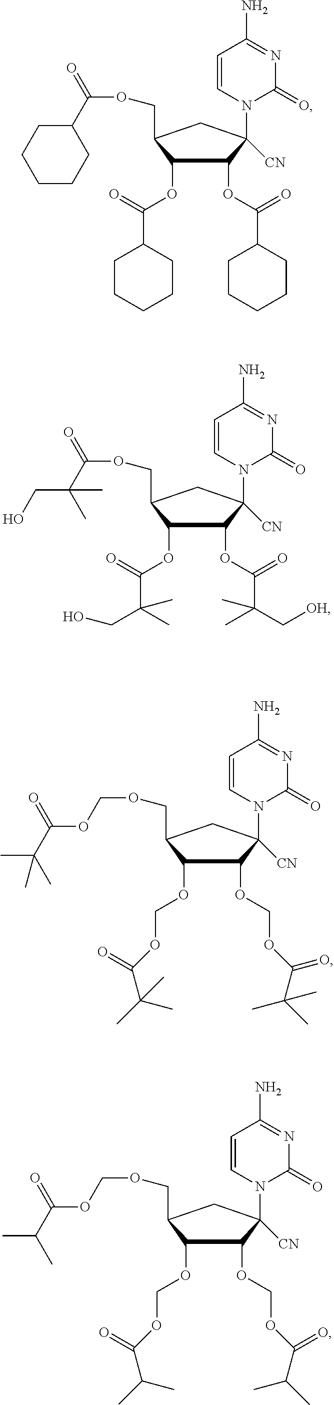

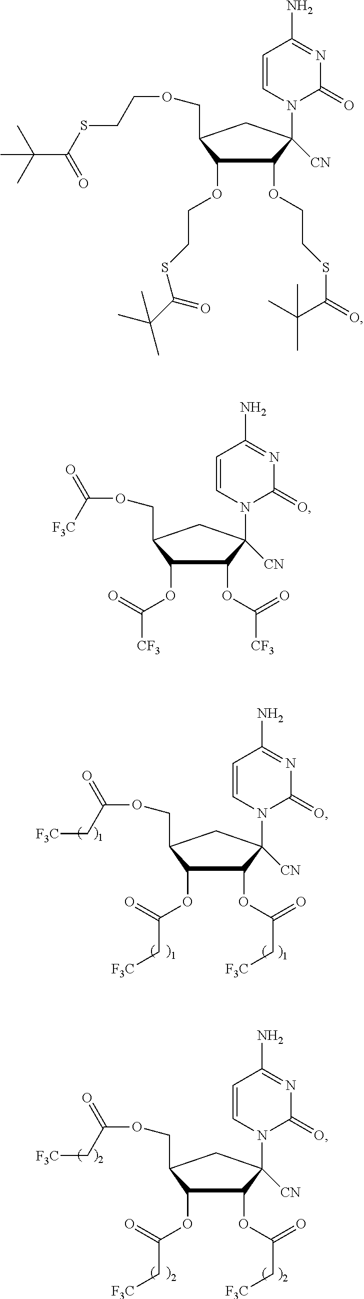

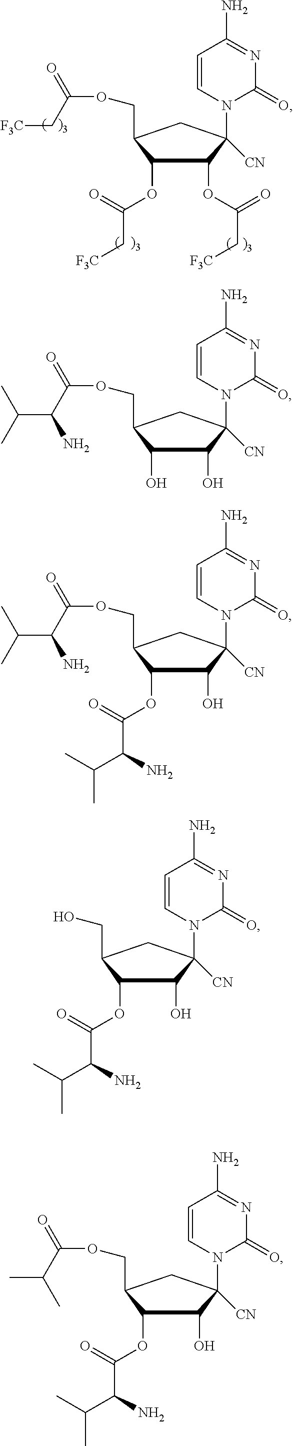

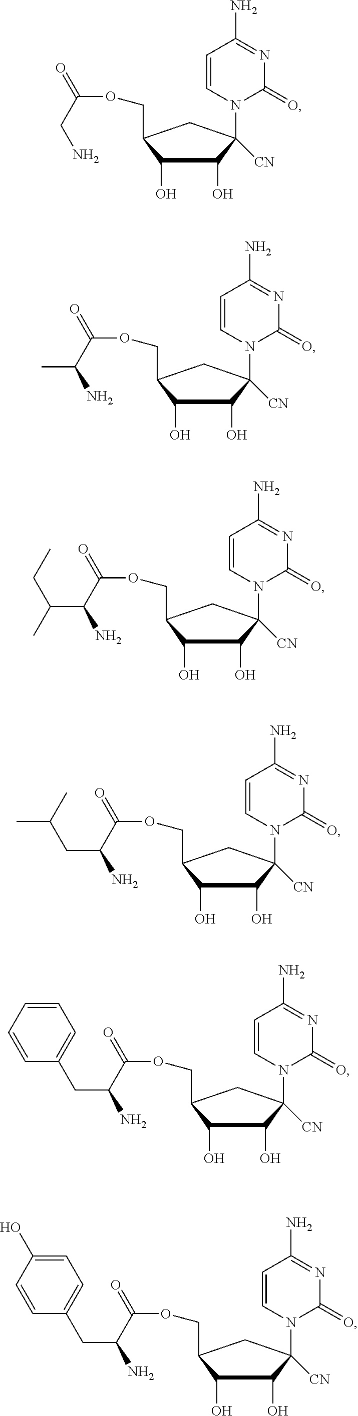

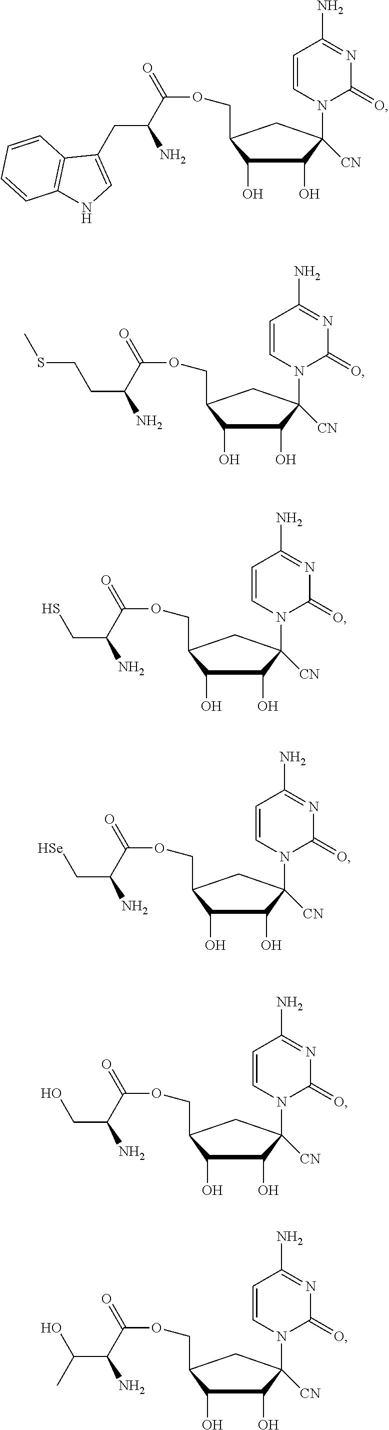

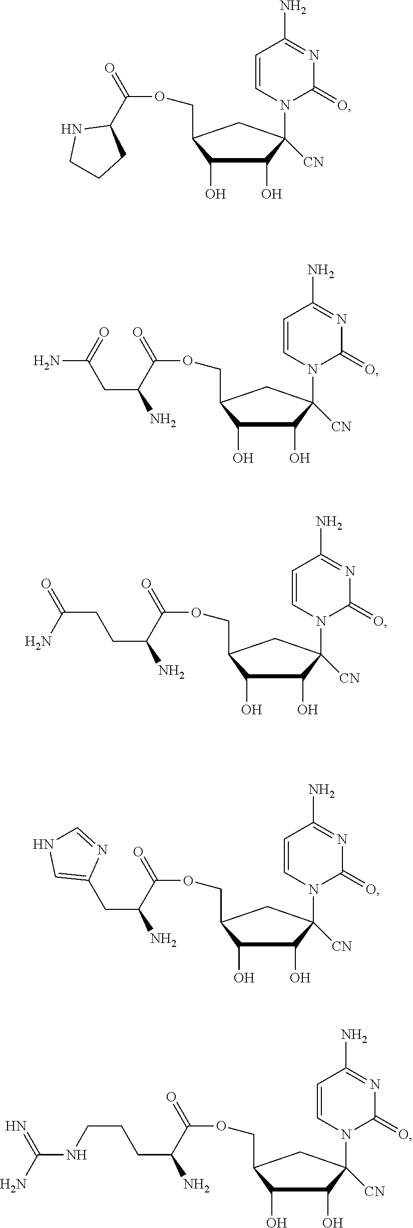

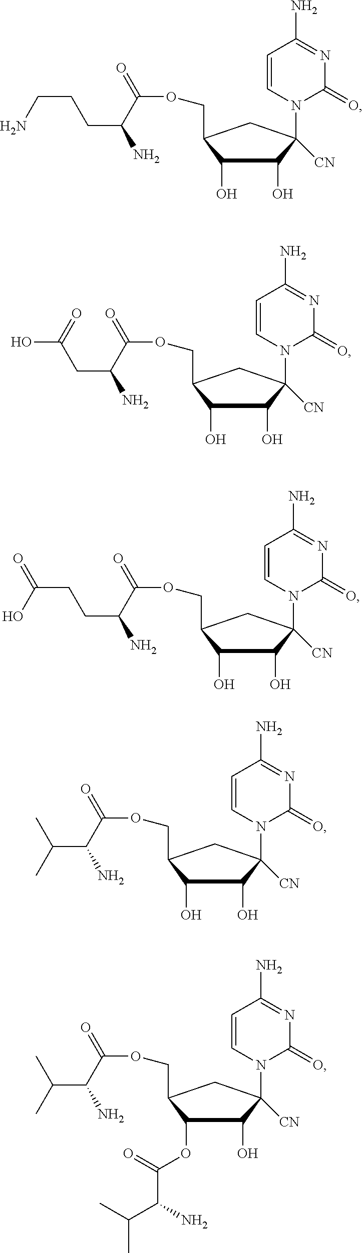

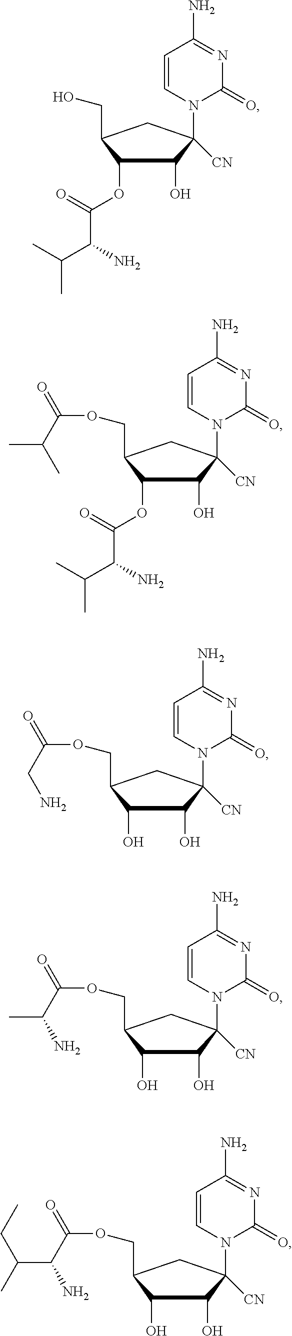

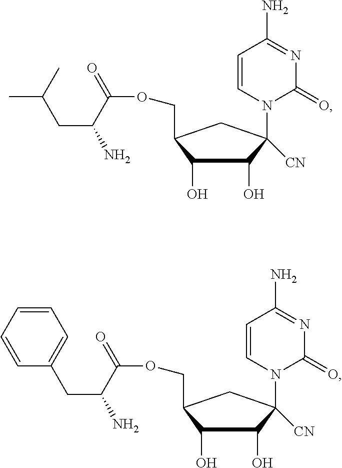

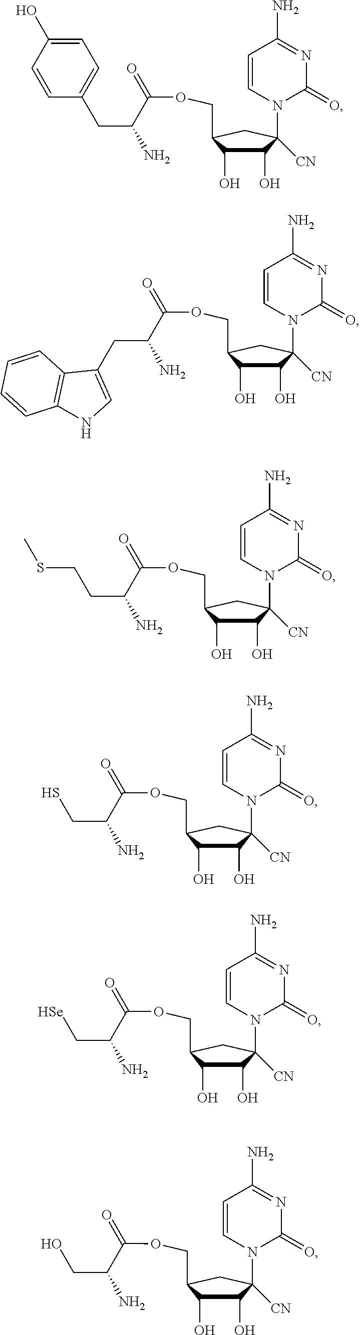

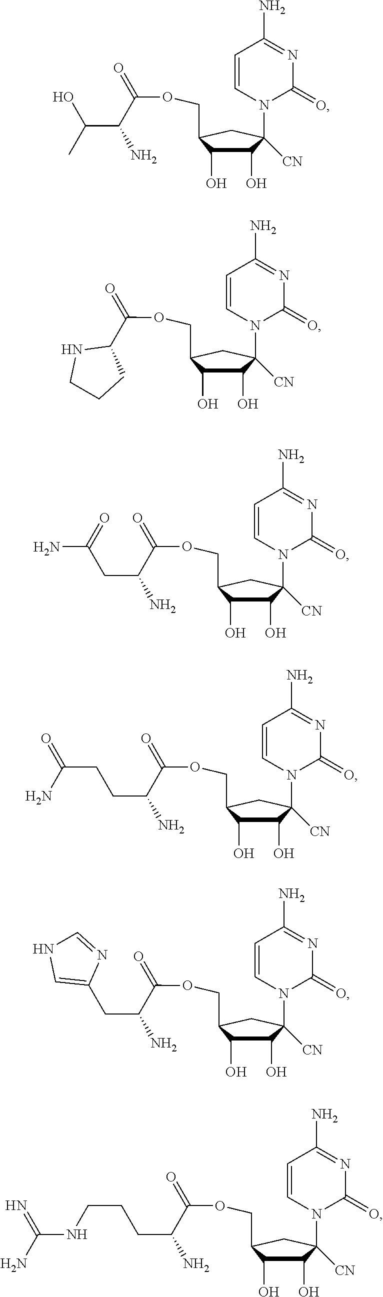

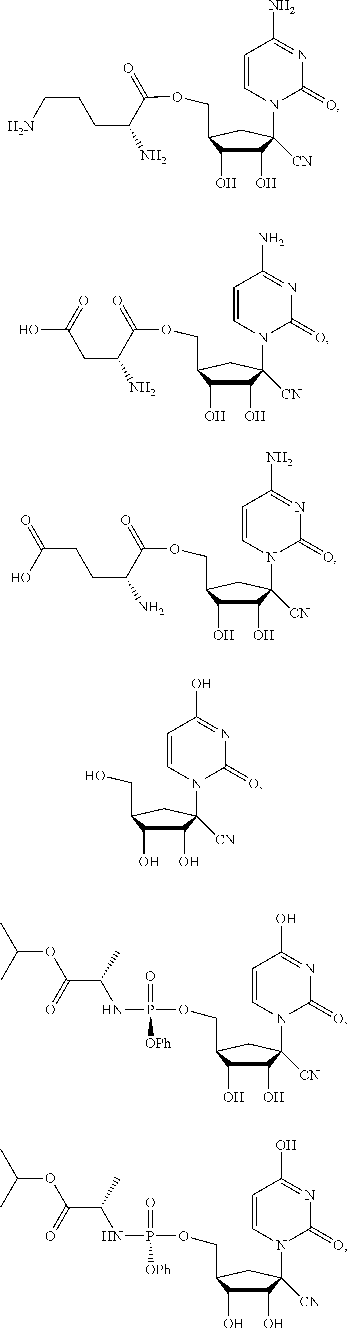

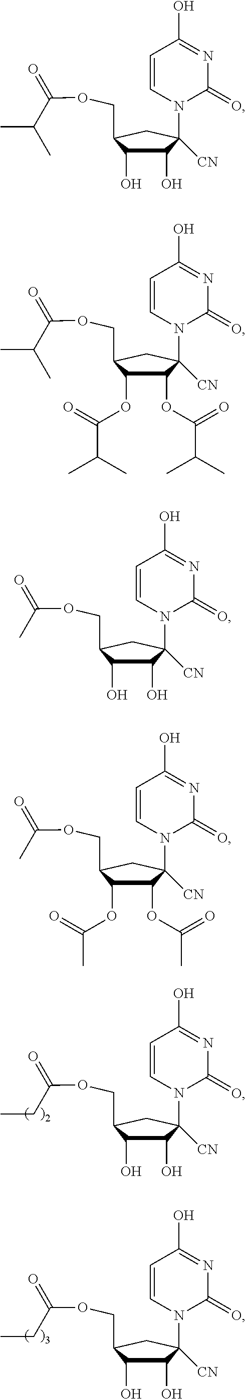

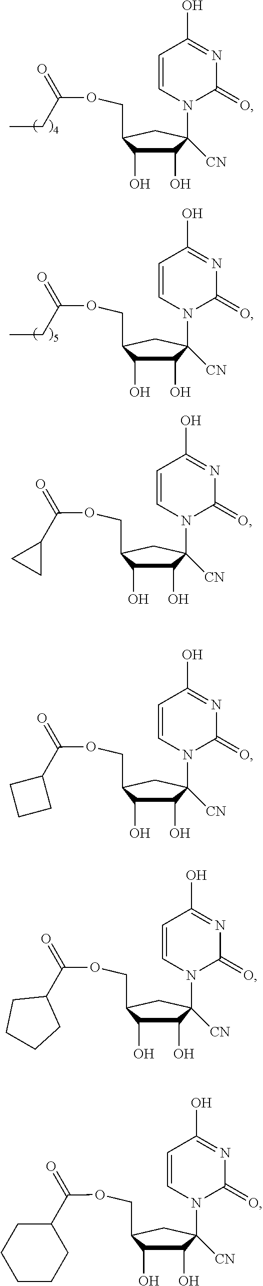

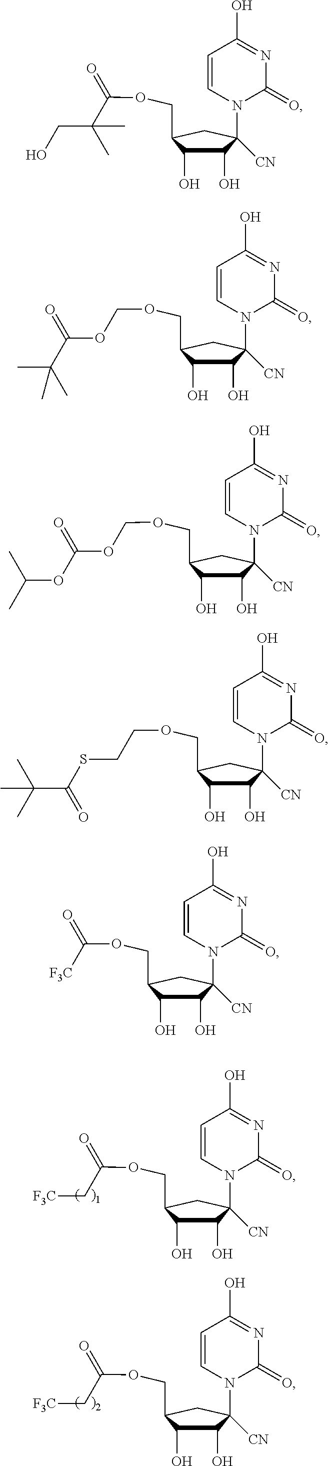

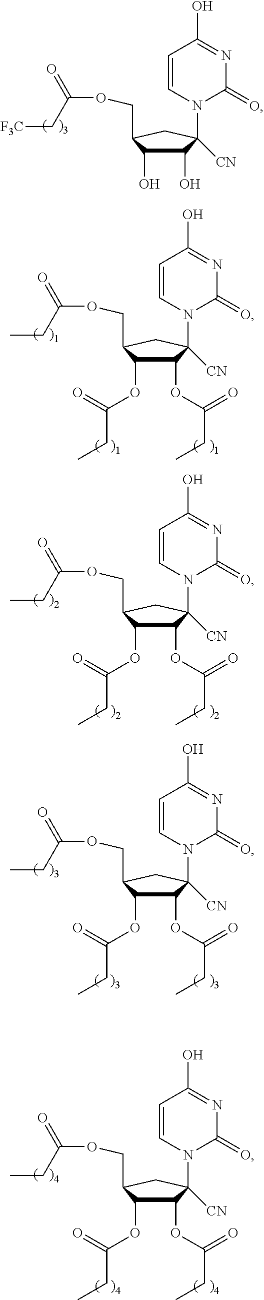

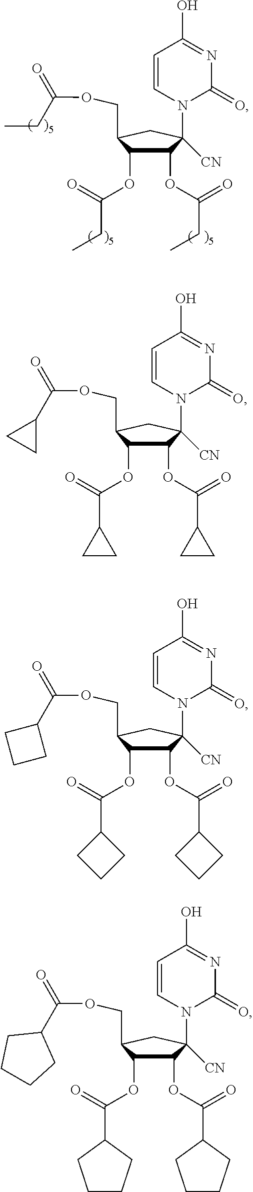

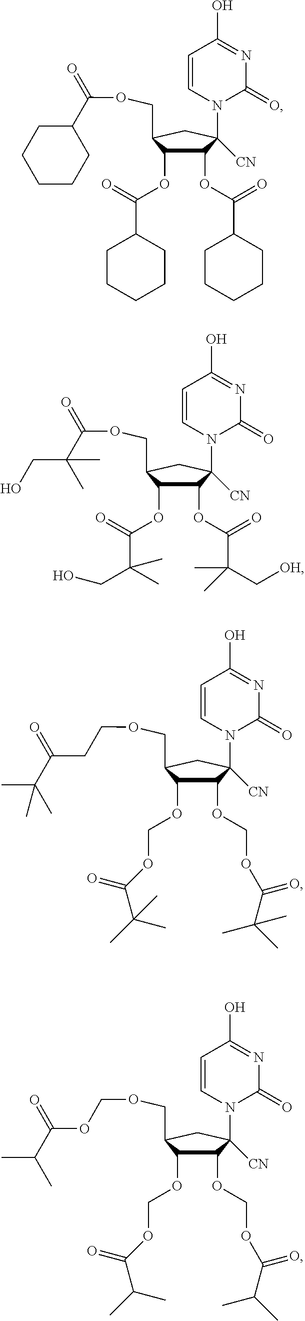

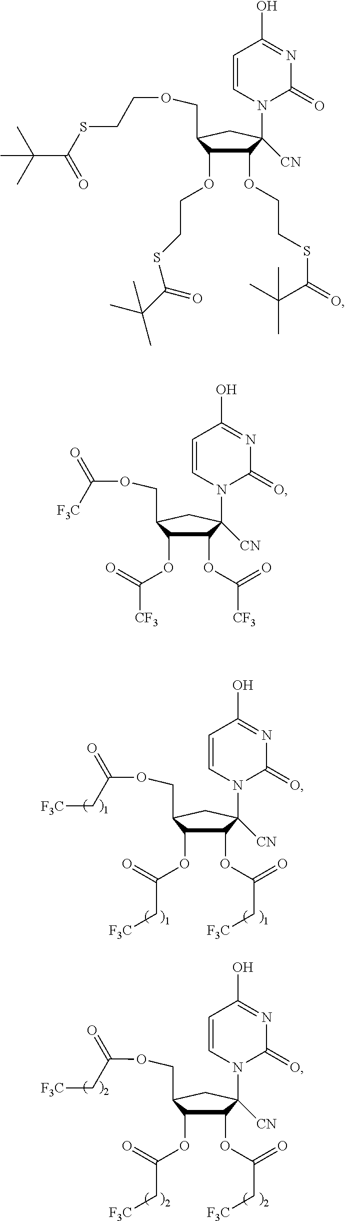

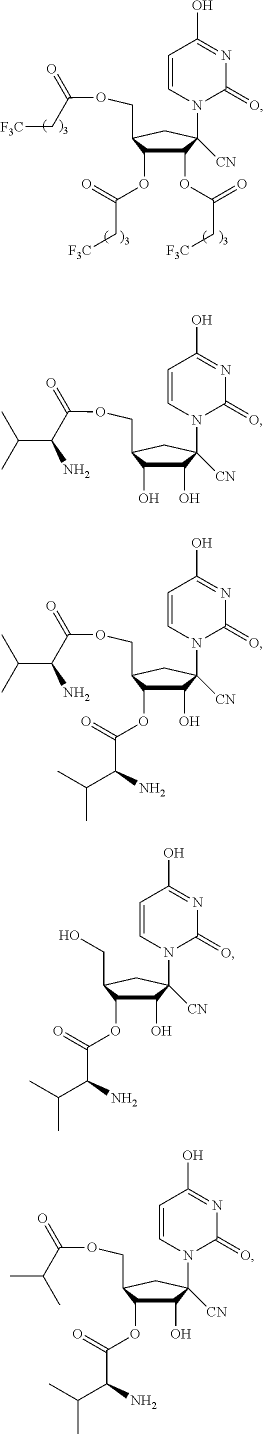

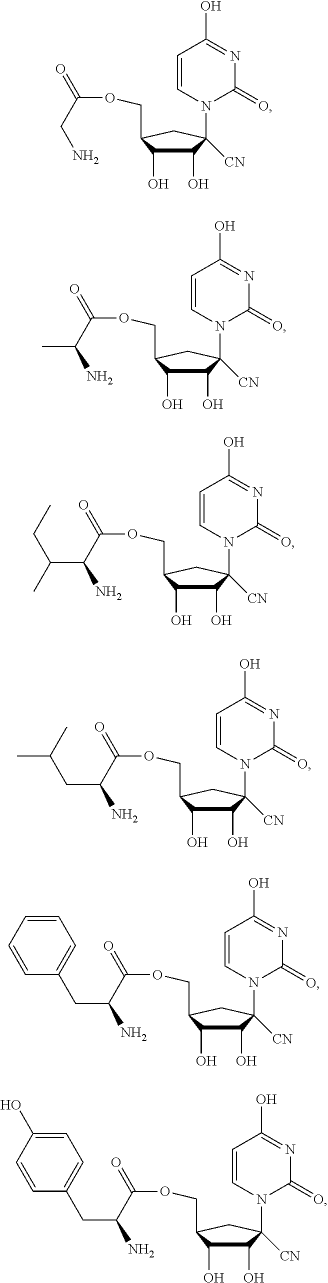

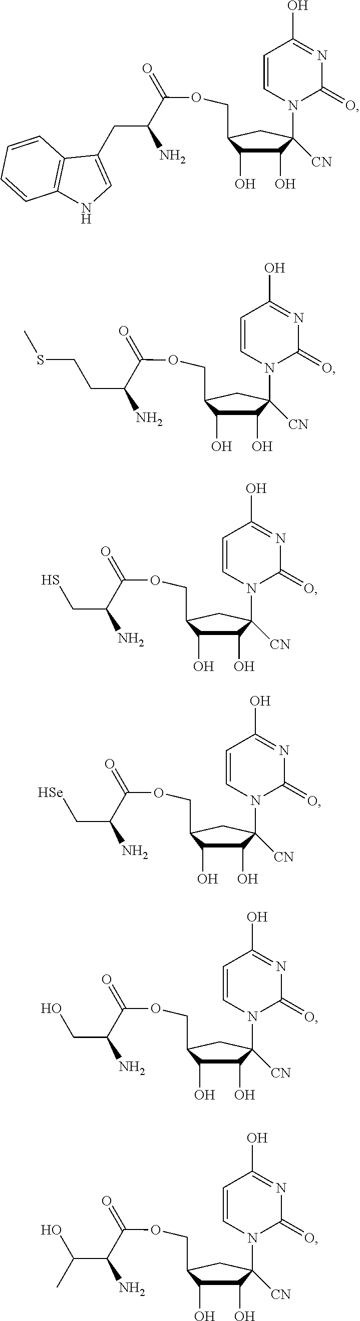

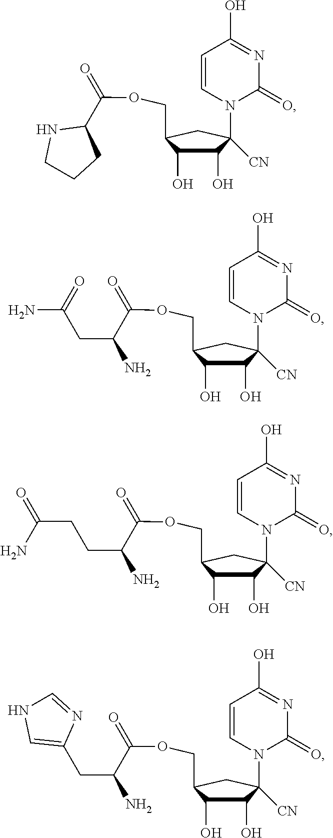

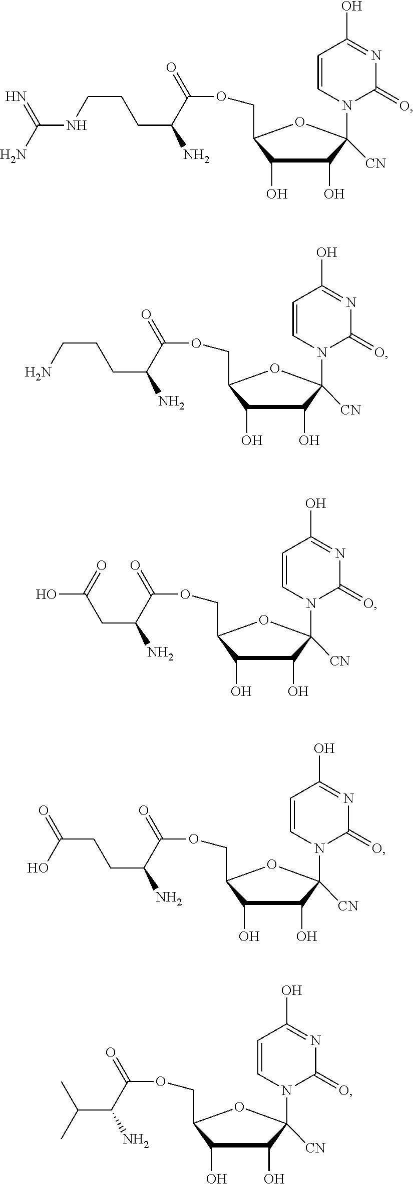

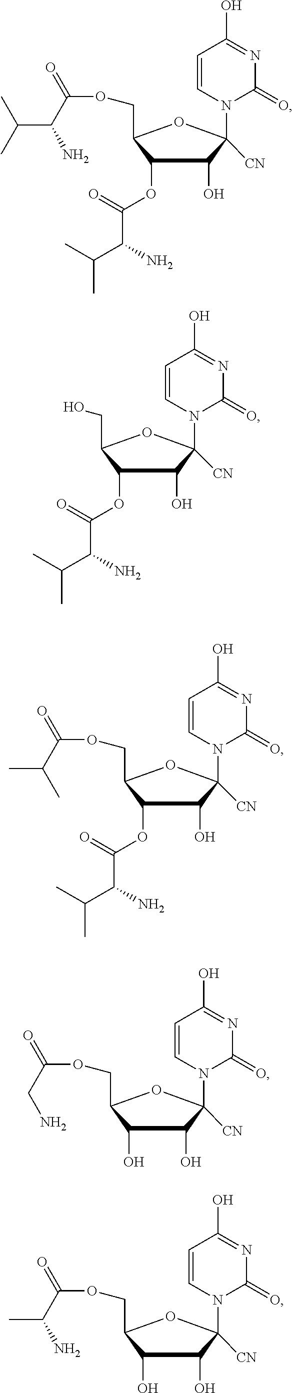

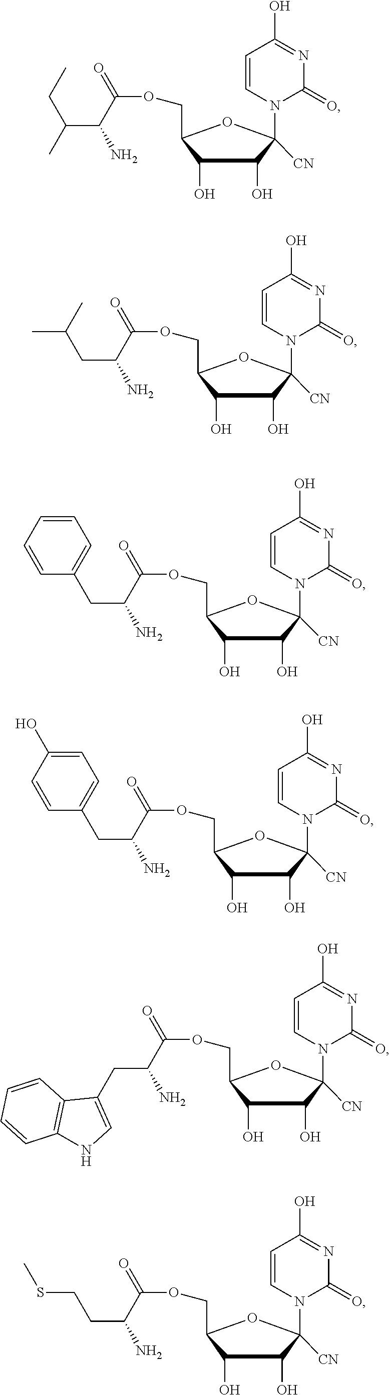

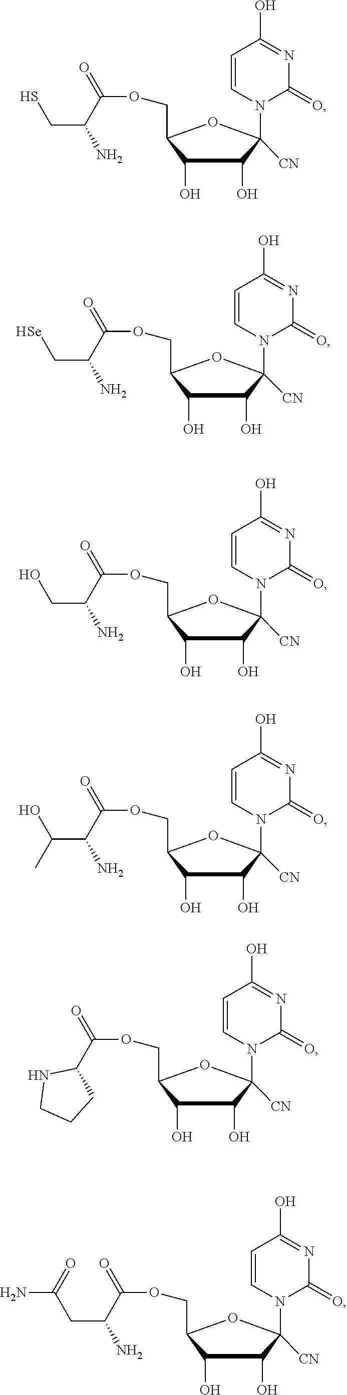

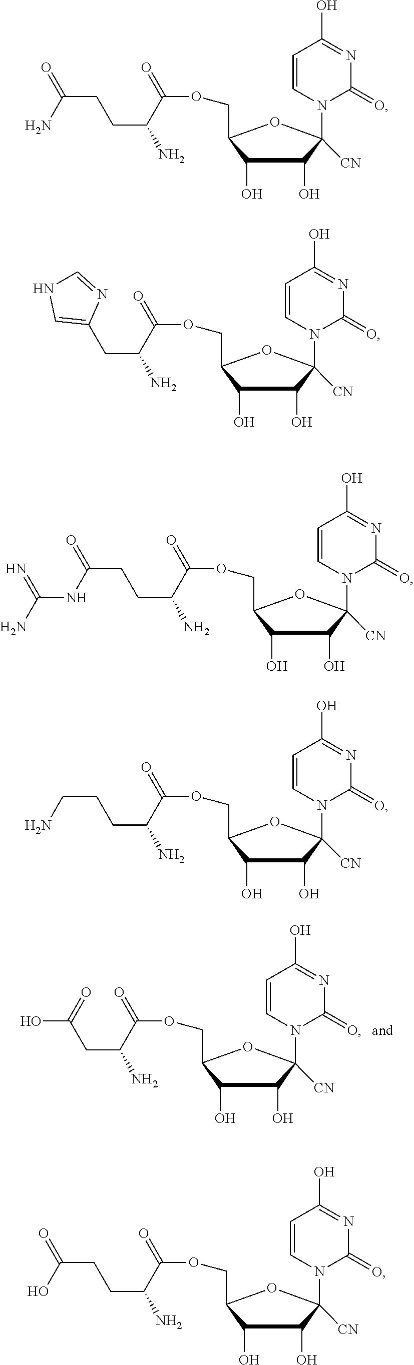

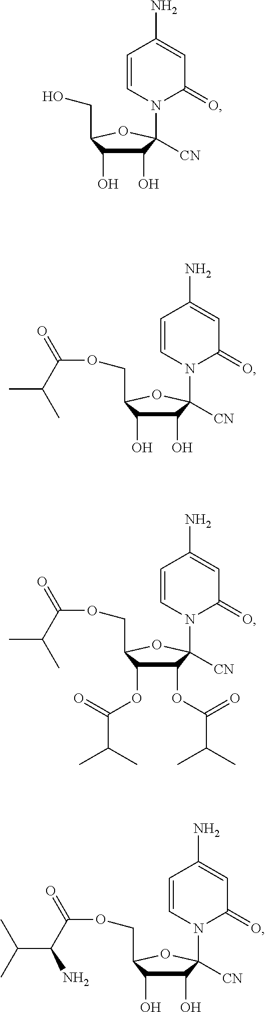

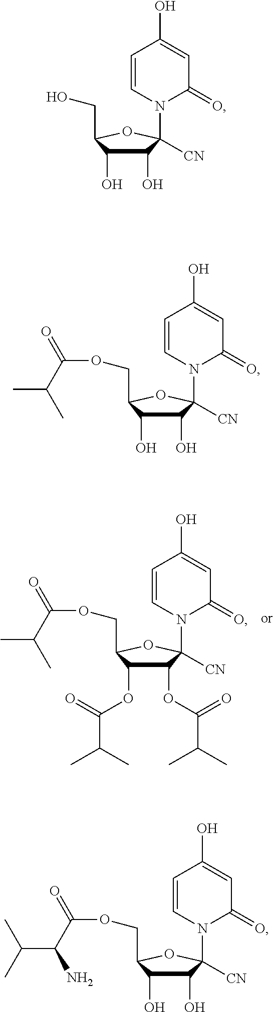

or a pharmaceutically acceptable salt or prodrug thereof.

Representative compounds also include the following:

and pharmaceutically-acceptable salts and prodrugs thereof.

Additional representative compounds include the following:

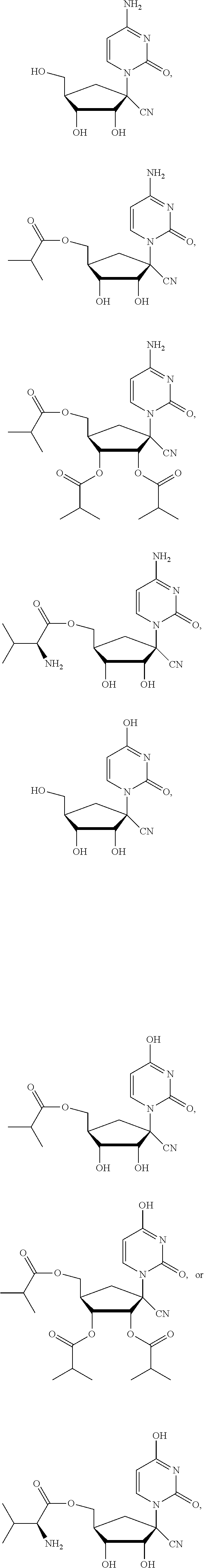

and pharmaceutically-acceptable salts and prodrugs thereof.

Additional representative compounds include the following:

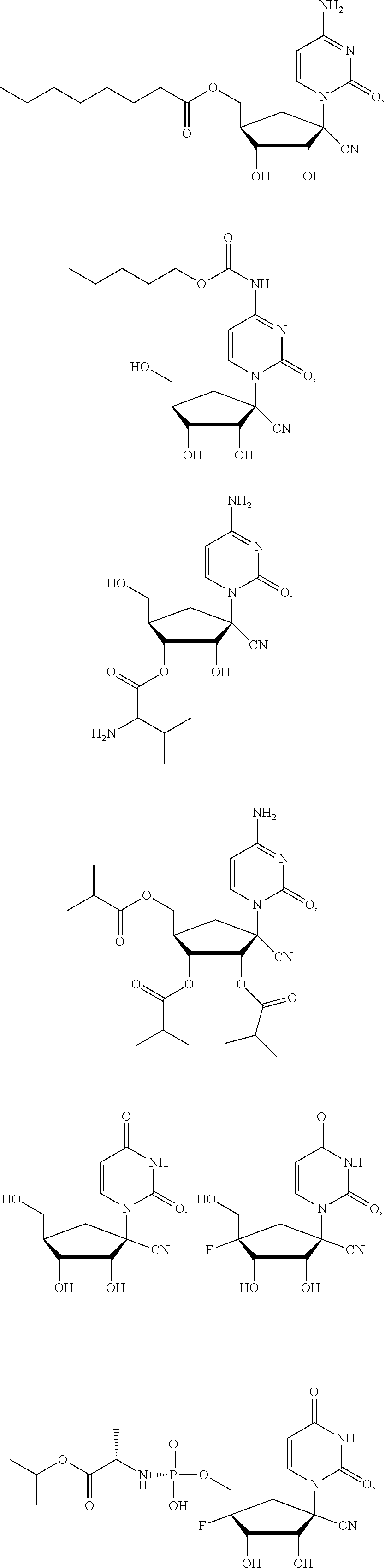

and pharmaceutically-acceptable salts and prodrugs thereof.

In any of these embodiments, the compounds can be present in the 3-D or 3-L configuration.

### III Stereoisomerism and Polymorphism

The compounds described herein can have asymmetric centers and occur as racemates, racemic mixtures, individual diastereomers or enantiomers, with all isomeric forms being included in the present disclosure. Compounds described herein having a chiral center can exist in and be isolated in optically active and racemic forms. Some compounds can exhibit polymorphism. The present disclosure encompasses racemic, optically-active, polymorphic, or stereoisomeric forms, or mixtures thereof, of a compound described herein, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective compound, then derivatize the compound to form the compounds described herein, or purify the compound themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.
i) physical separation of crystals: a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization: a technique whereby the individual enantiomers are separately crystallized from a solution of the racemate, possible only if the latter is a conglomerate in the solid state;
iii) enzymatic resolutions: a technique whereby partial or complete separation of a racemate by virtue of differing rates of reaction for the enantiomers with an enzyme; iv) enzymatic asymmetric synthesis: a synthetic technique whereby at least one step of the synthesis uses an enzymatic reaction to obtain an enantiomerically pure or enriched synthetic precursor of the desired enantiomer;
v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;
vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors: a synthetic technique whereby the desired enantiomer is obtained from non-chiral starting materials and where the stereochemical integrity is not or is only minimally compromised over the course of the synthesis;
x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;
xi) chiral gas chromatography: a technique whereby the racemate is volatilized and enantiomers are separated by virtue of their differing interactions in the gaseous mobile phase with a column containing a fixed non-racemic chiral adsorbent phase;
xii) extraction with chiral solvents: a technique whereby the enantiomers are separated by virtue of preferential dissolution of one enantiomer into a particular chiral solvent;
xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

### IV. Salt or Prodrug Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts can also be formed, including but not limited to, sulfate, nitrate, bicarbonate and carbonate salts. For certain transdermal applications, it can be preferred to use fatty acid salts of the compounds described herein. The fatty acid salts can help penetrate the stratum corneum. Examples of suitable salts include salts of the compounds with stearic acid, oleic acid, lineoleic acid, palmitic acid, caprylic acid, and capric acid.

Pharmaceutically acceptable salts can be obtained using standard procedures well known in the art, for example by reacting a sufficiently basic compound such as an amine with a suitable acid, affording a physiologically acceptable anion. In those cases where a compound includes multiple amine groups, the salts can be formed with any number of the amine groups. Alkali metal (e.g., sodium, potassium or lithium) or alkaline earth metal (e.g., calcium) salts of carboxylic acids can also be made.

A prodrug is a pharmacological substance that is administered in an inactive (or significantly less active) form and subsequently metabolized in vivo to an active metabolite. Getting more drug to the desired target at a lower dose is often the rationale behind the use of a prodrug and is generally attributed to better absorption, distribution, metabolism, and/or excretion (ADME) properties. Prodrugs are usually designed to improve oral bioavailability, with poor absorption from the gastrointestinal tract usually being the limiting factor. Additionally, the use of a prodrug strategy can increase the selectivity of the drug for its intended target thus reducing the potential for off target effects.

### V. Methods of Treatment

In one embodiment, the compounds described herein can be used to prevent, treat or cure coronavirus infections, specifically including SARS-CoV2 infections, such as SARS-CoV2, MERS, SARS, and OC-43. In other embodiments, the compounds described herein can be used to prevent, treat or cure infections by Flaviviruses, Picornaviridae, Togavirodae and Bunyaviridae.

The methods involve administering a therapeutically or prophylactically-effective amount of at least one compound as described herein to treat, cure or prevent an infection by, or an amount sufficient to reduce the biological activity of, a coronavirus infection, or a Flavivirus, Picornavus, Togavirus, or Bunyavirus infection.

In another embodiment, the compounds described herein can be used to inhibit a coronoviral, flaviviral, picornaviral, togaviral, or bunyaviral protease in a cell. The method includes contacting the cell with an effective amount of a compound described herein, Hosts, including but not limited to humans infected with a coronavirus, flavivirus, picornavirus, togavirus, or bunyavirus, or a gene fragment thereof, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, transdermally, subcutaneously, or topically, in liquid or solid form.

There are several species within the Coronavirus genus including, but not limited to, Middle East respiratory syndrome coronavirus (MERS-CoV), SARS coronavirus (SARS-CoV) and SARS-Cov2. In some embodiments, a compound described herein can ameliorate and/or treat a MERS-CoV infection, SARS-CoV infection, or SARS-Cov2 infection. An effective amount of a compound described herein can be administered to a subject infected with these viruses, and/or by contacting a cell infected with these viruses with an effective amount of a compound described herein. In some embodiments, a compound described herein can inhibit replication of these viruses. In some embodiments, a compound described herein can ameliorate one or more symptoms of these infections. Symptoms include, but are not limited to, extreme fatigue, malaise, headache, high fever (e.g., >100.4° F.), lethargy, confusion, rash, loss of appetite, myalgia, chills, diarrhea, dry cough, runny nose, sore throat, shortness of breath, breathing problems, gradual fall in blood-oxygen levels (such as, hypoxia) and pneumonia.

Some embodiments disclosed herein relate to a method of treating and/or ameliorating an infection caused by a Togaviridae virus that can include administering to a subject an effective amount of one or more compounds described herein, or a pharmaceutical composition that includes a compound described herein. Some embodiments described herein relate to using one or more compounds described herein in the manufacture of a medicament for ameliorating and/or treating an infection caused by a Togaviridae virus that can include administering to a subject an effective amount of one or more compounds described herein.

Some embodiments disclosed herein relate to methods of ameliorating and/or treating an infection caused by a Togaviridae virus that can include contacting a cell infected with the virus with an effective amount of one or more compounds described herein, or a pharmaceutical composition that includes one or more compounds described herein. Other embodiments described herein relate to using one or more compounds described herein in the manufacture of a medicament for ameliorating and/or treating an infection caused by a Togaviridae virus that can include contacting a cell infected with the virus with an effective amount of said compound(s).

In some embodiments, the Togaviridae virus can be an Alphavirus. One species of an Alphavirus is a Venezuelan equine encephalitis virus (VEEV). In some embodiments, a compound described herein can ameliorate and/or treat a VEEV infection. In other embodiments, one or more compounds described herein, can be manufactured into a medicament for ameliorating and/or treating an infection caused by a VEEV that can include contacting a cell infected with the virus with an effective amount of said compound(s). In still other embodiments, one or more compounds described herein, can be used for ameliorating and/or treating an infection caused by a VEEV that can include contacting a cell infected with the virus with an effective amount of said compound(s). In some embodiment, the VEEV can be an epizootic subtype. In some embodiment, the VEEV can be an enzootic subtype. As described herein, the Venezuelan equine encephalitis complex of viruses includes multiple subtypes that are further divided by antigenic variants. In some embodiments, a compound described herein can be effective against more than one subtype of a VEEV, such as 2, 3, 4, 5 or 6 subtypes. In some embodiments, a compound can be used to treat, ameliorate and/or prevent VEEV subtype I. In some embodiments, a compound described herein can be effective against more than one antigenic variants of a VEEV. In some embodiments, a compound can ameliorate one or more symptoms of a VEEV infection. Examples of symptoms manifested by a subject infected with VEEV include flu-like symptoms, such as high fever, headache, myalgia, fatigue, vomiting, nausea, diarrhea, and pharyngitis. Subjects with encephalitis show one or more of the following symptoms: somnolence, convulsions, confusion, photophobia, coma and bleeding of the brain, lung(s) and/or gastrointestinal tract. In some embodiments, the subject can be human. In other embodiments, the subject can be a horse.

Chikungunya (CHIKV) is another Alphavirus species. In some embodiments, a compound described herein can ameliorate and/or treat a CHIKV infection. In other embodiments, one or more compounds described herein can be manufactured into a medicament for ameliorating and/or treating an infection caused by a CHIKV that can include contacting a cell infected with the virus with an effective amount of said compound(s). In still other embodiments, one or more compounds described herein, can be used for ameliorating and/or treating an infection caused by a CHIKV that can include contacting a cell infected with the virus with an effective amount of said compound(s). In some embodiments, one or more symptoms of a CHIKV infection can be ameliorated by administering an effective amount of a compound to a subject infected with CHIKV and/or by contacting an CHIKV infected cell with an effective amount of a compound described herein. Clinical symptoms of a CHIKV infection include fever, rash (such as petechial and/or maculopapular rash), muscle pain, joint pain, fatigue, headache, nausea, vomiting, conjunctivitis, loss of taste, photophobia, insomnia, incapacitating joint pain and arthritis.

Other species of Alphaviruses include Barmah Forest virus, Mayaro virus (MAYV), O'nyong'nyong virus, Ross River virus (RRV), Semliki Forest virus, Sindbis virus (SINV), Una virus, Eastern equine encephalitis virus (EEE) and Western equine encephalomyelitis (WEE). In some embodiments, one or more compounds described herein, can be used for ameliorating and/or treating an infection caused by an Alphavirus that can include contacting a cell infected with the virus with an effective amount of one or more of said compound(s) and/or administering to a subject (such as, a subject infected with the virus) an effective amount of one or more of said compound(s), wherein the Alphavirus can be selected from Barmah Forest virus, Mayaro virus (MAYV), O'nyong'nyong virus, Ross River virus (RRV), Semliki Forest virus, Sindbis virus (SINV), Una virus, Eastern equine encephalitis virus (EEE) and Western equine encephalomyelitis (WEE).

Another genus of a Coronaviridae virus is a Rubivirus. Some embodiments disclosed herein relate to methods of ameliorating and/or treating an infection caused by a Rubivirus that can include contacting a cell infected with the virus with an effective amount of one or more compounds described herein, or a pharmaceutical composition that includes one or more compounds described herein. Other embodiments described herein relate to using one or more compounds described herein, in the manufacture of a medicament for ameliorating and/or treating an infection caused by a Rubivirus that can include contacting a cell infected with the virus with an effective amount of said compound(s). Still other embodiments described herein relate to one or more compounds described herein, that can be used for ameliorating and/or treating an infection caused by a Rubivirus by contacting a cell infected with the virus with an effective amount of said compound(s).

Some embodiments disclosed herein relate to a method of treating and/or ameliorating an infection caused by a Bunyaviridae virus that can include administering to a subject an effective amount of one or more compounds described herein, or a pharmaceutical composition that includes a compound described herein. Other embodiments disclosed herein relate to a method of treating and/or ameliorating an infection caused by a Bunyaviridae virus that can include administering to a subject identified as suffering from the viral infection an effective amount of one or more compounds described herein, or a pharmaceutical composition that includes a compound described herein.

Some embodiments disclosed herein relate to methods of ameliorating and/or treating an infection caused by a Bunyaviridae virus that can include contacting a cell infected with the virus with an effective amount of one or more compounds described herein, or a pharmaceutical composition that includes one or more compounds described herein. Other embodiments described herein relate to using one or more compounds described herein, in the manufacture of a medicament for ameliorating and/or treating an infection caused by a Bunyaviridae virus that can include contacting a cell infected with the virus with an effective amount of said compound(s). Still other embodiments described herein relate to one or more compounds described herein, that can be used for ameliorating and/or treating an infection caused by a Bunyaviridae virus by contacting a cell infected with the virus with an effective amount of said compound(s).

Some embodiments disclosed herein relate to methods of inhibiting replication of a Bunyaviridae virus that can include contacting a cell infected with the virus with an effective amount of one or more compounds described herein, or a pharmaceutical composition that includes one or more compounds described herein. Other embodiments described herein relate to using one or more compounds described herein, in the manufacture of a medicament for inhibiting replication of a Bunyaviridae virus that can include contacting a cell infected with the virus with an effective amount of said compound(s). Still other embodiments described herein relate to a compound described herein, that can be used for inhibiting replication of a Bunyaviridae virus by contacting a cell infected with the virus with an effective amount of said compound(s). In some embodiments, a compound described herein can inhibit a RNA dependent RNA polymerase of a Bunyaviridae virus, and thereby, inhibit the replication of RNA. In some embodiments, a polymerase of a Bunyaviridae virus can be inhibited by contacting a cell infected with the Bunyaviridae virus with a compound described herein.

In some embodiments, the Bunyaviridae virus can be a Bunyavirus. In other embodiments, the Bunyaviridae virus can be a Hantavirus. In still other embodiments, the Bunyaviridae virus can be a Nairovirus. In yet still other embodiments, the Bunyaviridae virus can be a Phlebovirus. In some embodiments, the Bunyaviridae virus can be an Orthobunyavirus. In other embodiments, the Bunyaviridae virus can be a Tospovirus.

A species of the Phlebovirus genus is Rift Valley Fever virus. In some embodiments, a compound described herein can ameliorate and/or treat a Rift Valley Fever virus infection. In other embodiments, one or more compounds described herein, can be manufactured into a medicament for ameliorating and/or treating an infection caused by a Rift Valley Fever virus that can include contacting a cell infected with the virus with an effective amount of said compound(s). In still other embodiments, one or more compounds described herein can be used for ameliorating and/or treating an infection caused by a Rift Valley Fever virus that can include contacting a cell infected with the virus with an effective amount of said compound(s). In some embodiments, a compound described herein can inhibit replication of Rift Valley Fever virus, wherein said compound is administering to a subject infected with Rift Valley Fever virus and/or wherein said compound contacts a cell infected with Rift Valley Fever.

In some embodiments, a compound described herein can ameliorate, treat, and/or inhibit replication of one or more of the ocular form, the meningoencephalitis form, or the hemorrhagic fever form of Rift Valley Fever virus. In some embodiments, one or more symptoms of a Rift Valley Fever virus infection can be ameliorated. Examples of symptoms of a Rift Valley Fever viral infection include headache, muscle pain, joint pain, neck stiffness, sensitivity to light, loss of appetite, vomiting, myalgia, fever, fatigue, back pain, dizziness, weight loss, ocular form symptoms (for example, retinal lesions, blurred vision, decreased vision and/or permanent loss of vision), meningoencephalitis form symptoms (such as, intense headache, loss of memory, hallucinations, confusion, disorientation, vertigo, convulsions, lethargy and coma) and hemorrhagic fever form symptoms (for example, jaundice, vomiting blood, passing blood in the feces, a purpuric rash, ecchymoses, bleeding from the nose and/or gums, menorrhagia and bleeding from a venepuncture site).

Another species of the Phlebovirus genus is thrombocytopenia syndrome virus. In some embodiments, a compound described herein can ameliorate, treat, and/or inhibit replication thrombocytopenia syndrome virus. In some embodiments, a compound can ameliorate and/or treat severe fever with thrombocytopenia syndrome (SFTS). In some embodiments, a compound described herein can ameliorate one or more symptoms of SFTS. Clinical symptoms of include the following: fever, vomiting, diarrhea, multiple organ failure, thrombocytopenia, leucopenia, and elevated liver enzyme levels.

Crimean-Congo hemorrhagic fever virus (CCHF) is a species within the Nairovirus genus. In some embodiments, a compound described herein can ameliorate, treat, and/or inhibit replication of Crimean-Congo hemorrhagic fever virus. Subjects infected with CCHF have one or more of the following symptoms: flu-like symptoms (such as high fever, headache, myalgia, fatigue, vomiting, nausea, diarrhea, and/or pharyngitis), hemorrhage, mood instability, agitation, mental confusion, throat petechiae, nosebleeds, bloody urine, vomiting, black stools, swollen and/or painful liver, disseminated intravascular coagulation, acute kidney failure, shock and acute respiratory distress syndrome. In some embodiments, a compound described herein can ameliorate one or more symptoms of CCHF.

California encephalitis virus is another virus of the Bunyaviridae family, and is a member of the Orthobunavirus genus. Symptoms of a California encephalitis virus infection include, but are not limited to fever, chills, nausea, vomiting, headache, abdominal pain, lethargy, focal neurologic findings, focal motor abnormalities, paralysis, drowsiness, lack of mental alertness and orientation and seizures. In some embodiments, a compound described herein can ameliorate, treat, and/or inhibit replication of California encephalitis virus. In some embodiments, a compound described herein can ameliorate one or more symptoms of a California encephalitis viral infection.

Viruses within the Hantavirus genus can cause hantavirus hemorrhagic fever with renal syndrome (HFRS) (caused by viruses such as Hantaan River virus, Dobrava-Belgrade virus, Saaremaa virus, Seoul virus, and Puumala virus) and hantavirus pulmonary syndrome (HPS). Viruses that can cause HPS include, but are not limited to, Black Creek Canal virus (BCCV), New York virus (NYV), Sin Nombre virus (SNV). In some embodiments, a compound described herein can ameliorate and/or treat HFRS or HPS. Clinical symptoms of HFRS include redness of cheeks and/or nose, fever, chills, sweaty palms, diarrhea, malaise, headaches, nausea, abdominal and back pain, respiratory problems, gastro-intestinal problems, tachycardia, hypoxemia, renal failure, proteinuria and diuresis. Clinical symptoms of HPS include flu-like symptoms (for example, cough, myalgia, headache, lethargy and shortness-of-breath that can deteriorate into acute respiratory failure). In some embodiments, a compound described herein can ameliorate one or more symptoms of IFRS or HPS.

Various indicators for determining the effectiveness of a method for treating and/or ameliorating a Coronaviridae, a Togaviridae, a Hepeviridae and/or a Bunyaviridae viral infection are known to those skilled in the art. Example of suitable indicators include, but are not limited to, a reduction in viral load, a reduction in viral replication, a reduction in time to seroconversion (virus undetectable in patient serum), a reduction of morbidity or mortality in clinical outcomes, and/or other indicator(s) of disease response. Further indicators include one or more overall quality of life health indicators, such as reduced illness duration, reduced illness severity, reduced time to return to normal health and normal activity, and reduced time to alleviation of one or more symptoms. In some embodiments, a compound described herein can result in the reduction, alleviation or positive indication of one or more of the aforementioned indicators compared to a subject who is untreated subject.

### VI. Combination or Alternation Therapy

In one embodiment, the compounds described herein can be employed together with at least one other active agent, which can be an antiviral agent. In one aspect of this embodiment, the at least one other active agent is selected from the group consisting of fusion inhibitors, entry inhibitors, protease inhibitors such as PF-07304814 (Pfizer) or PF-07321332 (Pfizer), optionally co-administered with a relatively low dose of ritonavir, polymerase inhibitors, antiviral nucleosides, such as remdesivir, GS-441524, AT-527 (ATEA), N4-hydroxycytidine, Molnupiravir (an N⁴-hydroxycytidine prodrug), and other compounds disclosed in U.S. Pat. No. 9,809,616, and their prodrugs, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

Umifenovir (also known as Arbidol) is a representative fusion inhibitor.

Representative entry inhibitors include Camostat, luteolin, MDL28170, SSAA09E2, SSAA09E1 (which acts as a cathepsin L inhibitor), SSAA09E3, and tetra-O-galloyl-β-D-glucose (TGG). The chemical formulae of certain of these compounds are provided below:

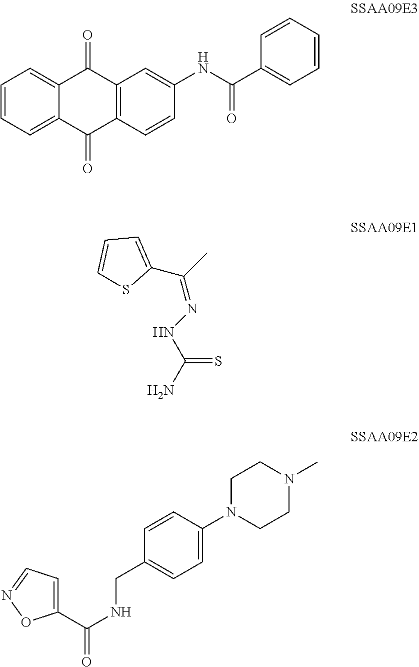 [SMILES: O=C(Nc1ccc2c(c1)C(=O)c1ccccc1C2=O)c1ccccc1 ; C/C(=N/NC(N)=S)c1cccs1 ; CN1CCN(c2ccc(CNC(=O)c3ccno3)cc2)CC1]

Other entry inhibitors include the following:

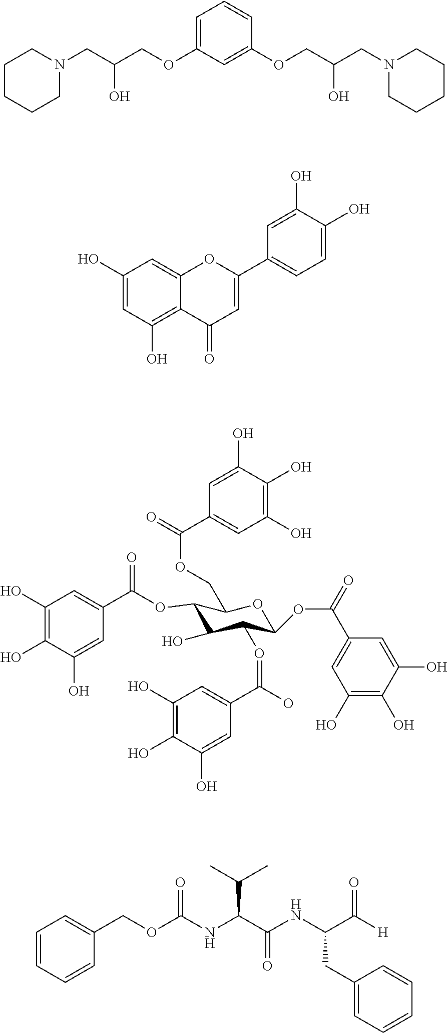

Remdesivir, Sofosbuvir, ribavirin, IDX-184 and GS-441524 have the following formulas:

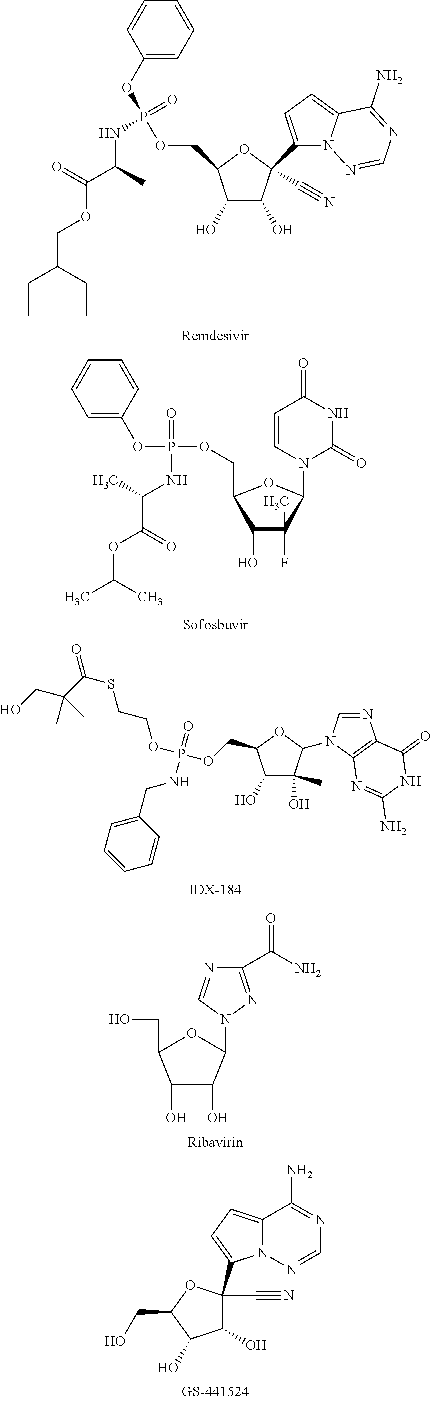

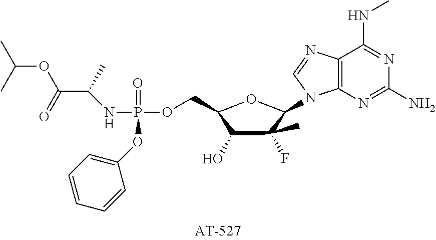

Additionally, one can administer compounds which inhibit the cytokine storm, anti-coagulants and/or platelet aggregation inhibitors that address blood clots, compounds which chelate iron ions released from hemoglobin by viruses such as COVID-19, cytochrome P-450 (CYP450) inhibitors and/or NOX inhibitors.

Representative NOX inhibitors are disclosed in PCT/US2018/067674, and include AEBSF, Apocyanin, DPI, GK-136901, ML171, Plumbagin, S17834, VAS2870, VAS3947, GKT-831, GKT771, GTL003 or amido thiadiazole derivatives thereof, as described in AU2015365465, EP20140198597; and WO2015/59659, Schisandrin B, as described in CN104147001 and CN20131179455), bi-aromatic and tri-aromatic compounds described in U.S. Publication No. 2015045387, GB 20110016017, and WO201200725, methoxyflavone derivatives described in JP 2015227329, JP 20140097875, and JP 20150093939, peptides, such as NOX2ds-tat and PR-39, as described in U.S. Publication No. 2015368301, TN 2015000295, U.S. Publication No. 201514689803, U.S. Publication No. 201462013916, PCT WO 201450063, and EP 20130150187, piperazine derivatives described in U.S. Publication No. 2014194422, U.S. Pat. No. 9,428,478, U.S. Publication No. 201214123877, U.S. Publication No. 201161496161, and PCT WO 2012US41988, pyrazole derivatives disclosed in KR101280198, KR20110025151, and KR20090082518, pyrazoline dione derivatives disclosed in HK 1171748, PCT WO201054329, and EP 20090171466, pyrazolo piperidine derivatives disclosed in KR20130010109, KR20130002317, EP20100153927, PCT WO201150667, EP20100153929, and PCT WO2011IB50668, pyrazolo pyridine derivatives described in KR20170026643, HK1158948, HK1141734, HK1159096, HK1159092, EP20080164857, PCT WO200954156, PCT WO200954150, EP20080164853, PCT WO200853390, U.S. Publication No. 20070896284, EP20070109555, PCT WO 200954148, EP20080164847, PCT WO200954155, and EP20080164849, quinazoline and quinoline derivatives disclosed in EP2886120, U.S. Publication No. 2014018384, U.S. Publication No. 20100407925, EP20110836947, GB20110004600, and PCT WO 201250586, tetrahydroindole derivatives disclosed in U.S. Publication No. 2010120749, U.S. Pat. No. 8,288,432, U.S. Publication No. 20080532567, EP20070109561, U.S. Publication No. 20070908414, and PCT WO 200853704, tetrahydroisoquinoline derivatives disclosed in U.S. Publication No. 2016083351, U.S. Publication No. 201414888390, U.S. Publication No. 201361818726, and PCT WO 201436402, Scopoletin, described in TW201325588 and TW20110147671, and 2,5-disubstituted benzoxazole and benzothiazole derivatives disclosed in TW201713650 and PCT WO 201554662. Representative NOX inhibitors also include those disclosed in PCT WO2011062864.

Exemplary Nox inhibitors also include 2-phenylbenzo[d]isothiazol-3(2H)-one, 2-(4-methoxyphenyl)benzo[d]isothiazol-3(2H)-one, 2-(benzo[d][1,3]dioxol-5-yl)benzo[d]isothiazol-3(2H)-one, 2-(2,4-dimethylphenyl)benzo[d]isothiazol-3(2H)-one, 2-(4-fluorophenyl)benzo[d]isothiazol-3(2H)-one, 2-(2,4-dimethylphenyl)-5-fluorobenzo[d]isothiazol-3(2H)-one, 5-fluoro-2-(4-fluorophenyl)benzo[d]isothiazol-3(2H)-one, 2-(2-chloro-6-methylphenyl)-5-fluorobenzo[d]isothiazol-3(2H)-one, 5-fluoro-2-phenylbenzo[d]isothiazol-3(2H)-one, 2-(benzo[d][1,3]dioxol-5-yl)-5-fluorobenzo[d]isothiazol-3(2H)-one, methyl 4-(3-oxobenzo[d]isothiazol-2(3H)-yl)benzoate, methyl 4-(5-fluoro-3-oxobenzo[d]isothiazol-2(3H)-yl)benzoate, ethyl 4-(3-oxobenzo[d]isothiazol-2(3H)-yl)benzoate, tert-butyl 4-(3-oxobenzo[d]isothiazol-2(3H)-yl)benzoate, methyl 2-methoxy-4-(3-oxobenzo[d]isothiazol-2(3H)-yl)benzoate, methyl 3-chloro-4-(3-oxobenzo[d]isothiazol-2(3H)-yl)benzoate, 4-(3-oxobenzo[d]isothiazol-2(3H)-yl)benzonitrile, methyl 2-(3-oxobenzo[d]isothiazol-2(3H)-yl)benzoate, 2-(4-acetylphenyl)benzo[d]isothiazol-3(2H)-one, 2-(4-nitrophenyl)benzo[d]isothiazol-3(2H)-one, 2-(4-hydroxyphenyl)benzo[d]isothiazol-3(2H)-one, methyl 6-(3-oxobenzo[d]isothiazol-2(3H)-yl)nicotinate, 6-(3-oxobenzo[d]isothiazol-2(3H)-yl)nicotinonitrile, 2-(4-(hydroxymethyl)phenyl)benzo[d]isothiazol-3(2H)-one, 2-benzylbenzo[d]isothiazol-3(2H)-one, N-methyl-4-(3-oxobenzo[d]isothiazol-2(3H)-yl)benzamide, 2-(4-hydroxyphenyl)benzo[d]isothiazol-3(2H)-one, 2-(2,4-dimethylphenyl)-1-methyl-1H-indazol-3(2H)-one, 2-(4-fluorophenyl)-1-methyl-1H-indazol-3 (2H)-one, 2-(2,4-dimethylphenyl)-1H-indazol-3(2H)-one, 1-methyl-2-phenyl-1H-indazol-3 (2H)-one, 2-(1,3,4-thiadiazol-2-yl)benzo[d]isothiazol-3(2H)-one, 2-(5-phenyl-1,3,4-thiadiazol-2-yl)benzo[d]isothiazol-3(2H)-one, 2-(5-(ethylthio)-1,3,4-thiadiazol-2-yl)benzo[d]isothiazol-3(2H)-one, 2-(5-(methylthio)-1,3,4-thiadiazol-2-yl)benzo[d]isothiazol-3(2H)-one, 5-fluoro-2-(1,3,4-thiadiazol-2-yl)benzo[d]isothiazol-3(2H)-one, 2-(5-(tert-butyl)-1,3,4-thiadiazol-2-yl)benzo[d]isothiazol-3(2H)-one, 2-(5-(4-bromophenyl)-1,3,4-thiadiazol-2-yl)benzo[d]isothiazol-3(2H)-one 2-(4-methylthiazol-2-yl)benzo[d]isothiazol-3(2H)-one, 2-(4,5-dimethylthiazol-2-yl)benzo[d]isothiazol-3(2H)-one, 2-(benzo[d][1,3]dioxol-5-yl)-4,5-difluorobenzo[d][1,2]selenazol-3(2H)-one, 2-(benzo[d][1,3]dioxol-5-yl)-5-fluorobenzo[d][1,2]selenazol-3(2H)-one, 2-(2,3-dihydrobenzo[b][1,4]dioxin-6-yl)-5-fluorobenzo[d][1,2]selenazol-3(2H)-2-(4-(1,3-dioxolan-2-yl)phenyl)benzo[d][1,2]selenazol-3(2H)-one, 2-(benzo[d][1,3]dioxol-5-yl)-6, 7-dimethoxybenzo[d][1,2]selenazol-3(2H)-one, methyl 4-(3-oxobenzo[d][1,2]selenazol-2(3H)-yl)benzoate, methyl 4-(3-oxoisothiazolo[5,4-b]pyridin-2(3H)-yl)benzoate, and ethyl 4-(3-oxoisothiazol-2(3H)-yl)benzoate, and pharmaceutically acceptable salts and prodrugs thereof.

Additional representative NOX inhibitors include:

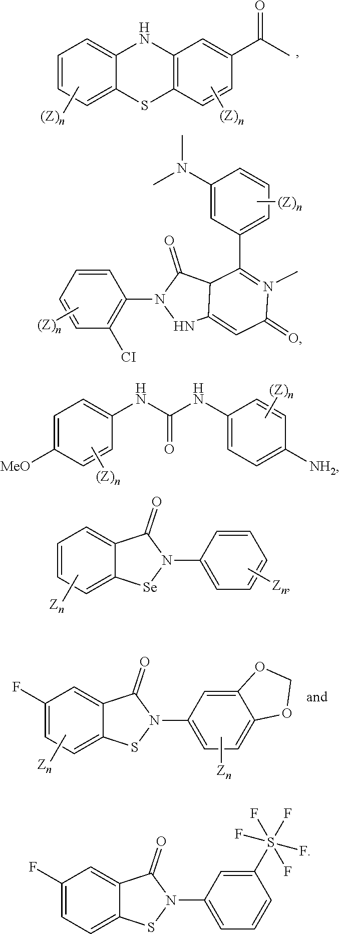

Specific examples of these compounds include

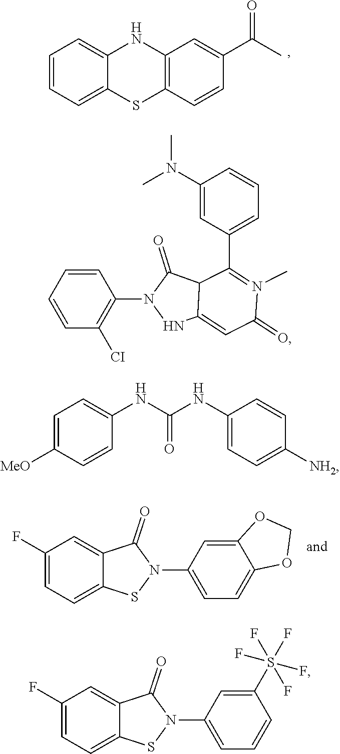

deuterated analogs thereof, or a pharmaceutically acceptable salt or prodrug thereof.

In one embodiment, the NOX inhibitor is Ebselen, Neopterin, APBA, Diapocynin, or a deuterated analog thereof, or a pharmaceutically-acceptable salt or prodrug thereof.

In another embodiment, the NOX compounds are those disclosed in PCT WO 2010/035221.

In still another embodiment, the compounds are NOX inhibitors disclosed in PCT WO 2013/068972, which are selected from the group consisting of:
4-(2-fluoro-4-methoxyphenyl)-2-(2-methoxyphenyl)-5-(pyridin-3-ylmethyl)-1H-pyrazolo[4,3-c]pyridine-3,6(2H,5H)-dione;
2-(2-chlorophenyl)-4-(4-methoxyphenyl)-5-(pyrazin-2-ylmethyl)-1H-pyrazolo[4,3-c]pyridine-3,6(2H,5H)-dione;
4-(4-chlorophenyl)-2-(2-methoxyphenyl)-5-(pyrazin-2-ylmethyl)-1H-pyrazolo[4,3-c]pyridine-3,6(2H,5H)-dione;
2-(2-chlorophenyl)-4-(2-fluoro-4-methoxyphenyl)-5-[(1-methyl-1H-pyrazol-3-yl) methyl]-1H-pyrazolo[4,3-c]pyridine-3,6(2H,5H)-dione;
4-(2-fluoro-5-methoxyphenyl)-2-(2-methoxyphenyl)-5-(pyridin-3-ylmethyl)-1H-pyrazolo[4,3-c]pyridine-3,6(2H,5H)-dione;
2-(2-chlorophenyl)-5-[(2-methoxypyridin-4-yl)methyl]-4-methyl-1H-pyrazolo[4,3-c]pyridine-3,6(2H,5H)-dione;
2-(2-methoxyphenyl)-4-methyl-5-(pyridin-3-ylmethyl)-1H-pyrazolo[4,3-c]pyridine-3,6(2H,5H)-dione;
4-(4-chloro-2-fluorophenyl)-2-(2-methoxyphenyl)-5-(pyridin-3-ylmethyl)-1H-pyrazolo
[4,3-c]pyridine-3,6(2H,5H)-dione;
4-(5-chloro-2-fluorophenyl)-2-(2-chlorophenyl)-5-(pyridin-3-ylmethyl)-1H-pyrazolo [4,3-c]pyridine-3,6(2H,5H)-dione;
2-(2-chlorophenyl)-5-[(6-methoxypyridin-3-yl)methyl]-4-methyl-1H-pyrazolo[4,3-c]pyridine-3,6 (2H,5H)-dione;
4-(4-chloro-2-fluorophenyl)-2-(2-chlorophenyl)-5-(pyridin-3-ylmethyl)-1H-pyrazolo [4,3-c]pyridine-3,6(2H,5H)-dione;
4-(5-chloro-2-fluorophenyl)-2-(2-chlorophenyl)-5-(pyridin-4-ylmethyl)-1H-pyrazolo [4,3-c]pyridine-3,6(2H,5H)-dione;
4-(2-fluoro-5-methoxyphenyl)-2-(2-methoxyphenyl)-5-[(1-methyl-1H-pyrazo-1-3-yl) methyl]-1H-pyrazolo[4,3-c]pyridine-3,6(2H,5H)-dione;
4-(5-chloro-2-fluorophenyl)-2-(2-methoxyphenyl)-5-(pyridin-3-ylmethyl)-1H-pyrazolo [4,3-c]pyridine-3,6(2H,5H)-dione;
2-(2-chlorophenyl)-4-methyl-5-(pyridin-3-ylmethyl)-1H-pyrazolo[4,3-c]pyridine-3,6 (2H,5H)-dione;
2-(2-chlorophenyl)-4-(4-chlorophenyl)-5-(pyrazin-2-ylmethyl)-1H-pyrazolo[4,3-c]pyridine-3,6 (2H,5H)-dione;
2-(2-chlorophenyl)-4-(2-fluorophenyl)-5-(pyridin-3-ylmethyl)-1H-pyrazolo[4,3-c]pyridine-3,6(2H,5H)-dione;
2-(2-chlorophenyl)-4-(4-chlorophenyl)-5-(pyridin-4-ylmethyl)-1H-pyrazolo[4,3-c]pyridine-3,6(2H,5H)-dione;
4-(4-chloro-2-fluorophenyl)-2-(2-chlorophenyl)-5-(pyridin-4-ylmethyl)-1H-pyrazolo [4,3-c]pyridine-3,6(2H,5H)-dione;
2-(2-methoxyphenyl)-4-(3-methoxyphenyl)-5-[(1-methyl-1H-pyrazo-1-3-yl)methyl]-1H-pyrazolo[4,3-c]pyridine-3,6(2H,5H)-dione;
2-(2-chlorophenyl)-4-(2-fluoro-4-methoxyphenyl)-5-(pyridin-3-ylmethyl)-1H-pyrazolo [4,3-c]pyridine-3,6(2H,5H)-dione;
4-(2-fluoro-4-methoxyphenyl)-2-(2-methoxyphenyl)-5-[(1-methyl-1H-pyrazo-1-3-yl) methyl]-1H-pyrazolo[4,3-c]pyridine-3,6(2H,5H)-dione;
2-(2-methoxyphenyl)-4-(4-methoxyphenyl)-5-[(1-methyl-1H-pyrazo-1-3-yl)methyl]-1H-pyrazolo[4,3-c]pyridine-3,6(2H,5H)-dione;
2-(2-methoxyphenyl)-4-(3-methoxyphenyl)-5-(pyridin-3-ylmethyl)-1H-pyrazolo[4,3-c]pyridine-3,6(2H,5H)-dione;
2-(2-chlorophenyl)-4-(4-chlorophenyl)-5-(pyridin-3-ylmethyl)-1H-pyrazolo[4,3-c]pyridine-3,6(2H,5H)-dione;
4-(4-chloro-2-fluorophenyl)-2-(2-chlorophenyl)-5-[(2-methoxypyridin-4-yl)methyl]-1H-pyrazolo[4,3-c]pyridine-3,6(2H,5H)-dione;
2-(2-chlorophenyl)-4-(2-fluoro-4-methoxyphenyl)-5-(pyridin-4-ylmethyl)-1H-pyrazolo [4,3-c]pyridine-3,6(2H,5H)-dione;
2-(2-chlorophenyl)-4-(2,6-difluorophenyl)-5-(pyridin-4-ylmethyl)-1H-pyrazolo[4,3-c]pyridine-3,6(2H,5H)-dione;
2-(2-chlorophenyl)-4-(2-fluorophenyl)-5-(pyridin-4-ylmethyl)-1H-pyrazolo[4,3-c]pyridine-3,6(2H,5H)-dione;
2-(2-chlorophenyl)-4-methyl-5-[(1-methyl-1H-pyrazol-3-yl)methyl]-1H-pyrazolo[4,3-c]pyridine-3,6(2H,5H)-dione;
4-(3-chloro-2-fluorophenyl)-2-(2-chlorophenyl)-5-(pyridin-4-ylmethyl)-1H-pyrazolo [4,3-c]pyridine-3,6(2H,5H)-dione;
2-(2-chlorophenyl)-5-methyl-4-[3-(methylamino)phenyl]-1H-pyrazolo [4,3-c]pyridine-3,6(2H,5H)-dione;
2-(2-methoxyphenyl)-4-(4-methoxyphenyl)-5-(pyridin-3-ylmethyl)-1H-pyrazolo[4,3-c]pyridine-3,6(2H,5H)-dione;
2-(2-chlorophenyl)-4-(2-fluorophenyl)-5-(pyridin-2-ylmethyl)-1H-pyrazolo[4,3-c]pyridine-3,6(2H,5H)-dione;
2-(2-chlorophenyl)-4-(2,5-difluorophenyl)-5-(pyridin-4-ylmethyl)-1H-pyrazolo[4,3-c]pyridine-3,6(2H,5H)-dione;
2-(2-chlorophenyl)-4-(4-chlorophenyl)-5-(1,3-thiazol-2-ylmethyl)-1H-pyrazolo[4,3-c]pyridine-3,6(2H,5H)-dione;
2-(2-chlorophenyl)-4-[3-(dimethylamino)phenyl]-5-[(1-methyl-1H-pyrazol-3-yl) methyl]-1H-pyrazolo[4,3-c]pyridine-3,6(2H,5H)-dione;
2-(2-chlorophenyl)-4-(3,5-dichlorophenyl)-5-(pyridin-4-ylmethyl)-1H-pyrazolo[4,3-c]pyridine-3,6(2H,5H)-dione;
4-(3-chloro-2-fluorophenyl)-2-(2-chlorophenyl)-5-(pyridin-3-ylmethyl)-1H-pyrazolo [4,3-c]pyridine-3,6(2H,5H)-dione;
2-(2-chlorophenyl)-4-[3-(dimethylamino)phenyl]-5-(pyridin-3-ylmethyl)-1H-pyrazolo [4,3-c]pyridine-3,6(2H,5H)-dione;
2-(2-chlorophenyl)-4-(2,6-difluorophenyl)-5-(pyridin-3-ylmethyl)-1H-pyrazolo[4,3-c]pyridine-3,6(2H,5H)-dione;
4-(2-fluoro-5-methoxyphenyl)-2-(2-methoxyphenyl)-5-(pyrazin-2-ylmethyl)-1H-pyrazolo[4,3-c]pyridine-3,6(2H,5H)-dione;
2-(2-chlorophenyl)-4-(2,5-difluorophenyl)-5-(pyridin-3-ylmethyl)-1H-pyrazolo[4,3-c]pyridine-3,6(2H,5H)-dione; and
2-(2-chlorophenyl)-4-[3-(dimethylamino)phenyl]-5-[(1-methyl-1H-pyrazol-3-yl) methyl]-1H-pyrazolo[4,3-c]pyridine-3,6(2H,5H)-dione.

Representative CYP450 inhibitors include, but are not limited to, amiodarone, amlodipine, apigenin, aprepitant, bergamottin (grapefruit), buprenorphine, bupropion, caffeine, cafestol, cannabidiol, celecoxib, chloramphenicol, chlorphenamine, chlorpromazine, cimetidine, cinacalcet, ciprofloxacin, citalopram, clarithromycin, clemastine, clofibrate, clomipramine, clotrimazole, cobicistat, cocaine, curcumin (turmeric), cyclizine, delavirdine, desipramine, disulfiram, diltiazem, diphenhydramine, dithiocarbamate, domperidone, doxepin, doxorubicin, duloxetine, echinacea, entacapone, erythromycin, escitalopram, felbamate, fenofibrate, flavonoids (grapefruit), fluoroquinolones (e.g., ciprofloxacin), fluoxetine, fluvoxamine, fluconazole, fluvastatin, gabapentin, gemfibrozil, gestodene, halofantrine, haloperidol, hydroxyzine, imatinib, indomethacin, indinavir, interferon, isoniazid, itraconazole, JWH-018, ketoconazole, letrozole, lovastatin, levomepromazine, memantine, methylphenidate, metoclopramide, methadone, methimazole, methoxsalen, metyrapone, mibefradil, miconazole, midodrine, mifepristone, milk thistle, moclobemide, modafinil, montelukast, moclobemide, naringenin (grapefruit), nefazodone, nelfinavir, niacin, niacinamide, nicotine, nicotinamide, nilutamide, norfloxacin, orphenadrine, paroxetine, perphenazine, pilocarpine, piperine, phenylbutazone, probenecid, promethazine, proton pump inhibitors (e.g., lansoprazole, omeprazole, pantoprazole, rabeprazole), quercetin, quinidine, ranitidine, risperidone, ritonavir, saquinavir, selegiline, sertraline, star fruit, St. John's wort, sulconazole, sulfamethoxazole, sulfaphenazole, telithromycin, teniposide, terbinafine, thiazolidinediones, thioridazine, ticlopidine, tioconazole, thiotepa, trimethoprim, topiramate, tranylcypromine, tripelennamine, valerian, valproic acid, verapamil, voriconazole, zafirlukast, and zuclopenthixol.

Representative ACE-2 inhibitors include sulfhydryl-containing agents, such as alacepril, captopril (capoten), and zefnopril, dicarboxylate-containing agents, such as enalapril (vasotec), ramipril (altace), quinapril (accupril), perindopril (coversyl), lisinopril (listril), benazepril (lotensin), imidapril (tanatril), trandolapril (mavik), and cilazapril (inhibace), and phosphonate-containing agents, such as fosinopril (fositen/monopril).

For example, when used to treat or prevent infection, the active compound or its prodrug or pharmaceutically acceptable salt can be administered in combination or alternation with another antiviral agent including, but not limited to, those of the formulae above. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

A number of agents for combination with the compounds described herein are disclosed in Ghosh et al., “Drug Development and Medicinal Chemistry Efforts Toward SARS-Coronavirus and Covid-19 Therapeutics,” ChemMedChem 10.1002/cmdc.202000223.

Nonlimiting examples of antiviral agents that can be used in combination with the compounds disclosed herein include those listed below.

### Compounds for Inhibiting the Cytokine Storm

Throughout its activation, the inflammatory response must be regulated to prevent a damaging systemic inflammation, also known as a “cytokine storm.” A number of cytokines with anti-inflammatory properties are responsible for this, such as IL-10 and transforming growth factor β (TGF-β). Each cytokine acts on a different part of the inflammatory response. For example, products of the Th2 immune response suppress the Th1 immune response and vice versa.

By resolving inflammation, one can minimize collateral damage to surrounding cells, with little or no long-term damage to the patient. Accordingly, in addition to using the compounds described herein to inhibit the viral infection, one or more compounds which inhibit the cytokine storm can be co-administered.

Compounds which inhibit the cytokine storm include compounds that target fundamental immune pathways, such as the chemokine network and the cholinergic anti-inflammatory pathway.

JAK inhibitors, such as JAK 1 and JAK 2 inhibitors, can inhibit the cytokine storm, and in some cases, are also antiviral. Representative JAK inhibitors include those disclosed in U.S. Pat. No. 10,022,378, such as Jakafi, Tofacitinib, and Baricitinib, as well as LY3009104/INCB28050, Pacritinib/SB1518, VX-509, GLPG0634, INC424, R-348, CYT387, TG 10138, AEG 3482, and pharmaceutically acceptable salts and prodrugs thereof.

Still further examples include CEP-701 (Lestaurtinib), AZD1480, INC424, R-348, CYT387, TG 10138, AEG 3482, 7-iodo-N-(4-morpholinophenyl)thieno[3,2-d]pyrimidin-2-amine, 7-(4-aminophenyl)-N-(4-morpholinophenyl)thieno[3,2-d]pyrimidin-2-amine, N-(4-(2-(4-morpholinophenylamino)thieno[3,2-d]pyrimidin-7-yl)phenyl) acrylamide, 7-(3-aminophenyl)-N-(4-morpholinophenyl)thieno[3,2-d]pyrimidin-2-amine, N-(3-(2-(4-morpholinophenylamino)thieno[3,2-d]pyrimidin-7-yl)phenyl) acrylamide, N-(4-morpholinophenyl)thieno[3,2-d]pyrimidin-2-amine, methyl 2-(4-morpholinophenylamino)thieno[3,2-d]pyrimidine-7-carboxylate, N-(4-morpholinophenyl)-5H-pyrrolo[3,2-d]pyrimidin-2-amine, 7-(4-amino-3-methoxyphenyl)-N-(4-morpholinophenyl)thieno[3,2-d]pyrimidin-2-amine, 4-(2-(4-morpholinophenylamino)thieno[3,2-d]pyrimidin-7-yl)benzene-sulfonamide, N,N-dimethyl-3-(2-(4-morpholinophenylamino)thieno[3,2-d]pyrimidin-7-yl)benzenesulfonamide, 1-ethyl-3-(2-methoxy-4-(2-(4-morpholinophenylamino)thieno[3,2-d]pyrimidin-7-yl)phenyl)urea, N-(4-(2-(4-morpholinophenylamino)thieno[3,2-d]pyrimidin-7-yl)phenyl)metha-nesulfonamide, 2-methoxy-4-(2-(4-morpholinophenylamino)thieno[3,2-d]pyrimidin-7-yl)pheno-1, 2-cyano-N-(3-(2-(4-morpholinophenylamino)thieno[3,2-d]pyrimidin-7-yl)phenyl)acetamide, N-(cyanomethyl)-2-(4-morpholinophenylamino)thieno[3,2-d]pyrimidine-7-carboxamide, N-(3-(2-(4-morpholinophenylamino)thieno[3,2-d]pyrimidin-7-yl)phenyl)methanesulfonamide, 1-ethyl-3-(4-(2-(4-morpholinophenylamino)thieno[3,2-d]pyrimidin-7-yl)-2-(trifluoromethoxy)phenyl)urea, N-(3-nitrophenyl)-7-phenylthieno[3,2-d]pyrimidin-2-amine, 7-iodo-N-(3-nitrophenyl)thieno[3,2-d]pyrimidin-2-amine, N1-(7-(2-ethylphenyl)thieno[3,2-d]pyrimidin-2-yl)benzene-1,3-diamine, N-tert-butyl-3-(2-(4-morpholinophenylamino)thieno[3,2-d]pyrimidin-7-yl)benzenesulfonamide, N1-(7-iodothieno[3,2-d]pyrimidin-2-yl)benzene-1,3-diamine, 7-(4-amino-3-(trifluoromethoxy)phenyl)-N-(4-morpholinophenyl)thieno[3,2-d]pyrimidin-2-amine, 7-(2-ethylphenyl)-N-(4-morpholinophenyl)thieno[3,2-d]pyrimidin-2-amine, N-(3-(2-(4-morpholinophenylamino)thieno[3,2-d]pyrimidin-7-yl)phenyl)aceta-mide, N-(cyanomethyl)-N-(3-(2-(4-morpholinophenylamino)thieno[3,2-d]pyrimidin-7-yl)phenyl)methanesulfonamide, N-(cyanomethyl)-N-(4-(2-(4-morpholinophenylamino)thieno[3,2-d]pyrimidin-7-yl)phenyl)methanesulfonamide, N-(3-(5-methyl-2-(4-morpholinophenylamino)-5H-pyrrolo[3,2-d]pyrimidin-7-yl)phenyl)methanesulfonamide, 4-(5-methyl-2-(4-morpholinophenylamino)-5H-pyrrolo[3,2-d]pyrimidin-7-yl)b-enzenesulfonamide, N-(4-(5-methyl-2-(4-morpholinophenylamino)-5H-pyrrolo[3,2-d]pyrimidin-7-y-1)phenyl)methanesulfonamide, 7-iodo-N-(4-morpholinophenyl)-5H-pyrrolo[3,2-d]pyrimidin-2-amine, 7-(2-isopropylphenyl)-N-(4-morpholinophenyl)thieno[3,2-d]pyrimidin-2-amine, 7-bromo-N-(4-morpholinophenyl)thieno[3,2-d]pyrimidin-2-amine, N7-(2-isopropylphenyl)-N2-(4-morpholinophenyl)thieno[3,2-d]pyrimidine-2,7-diamine, N7-(4-isopropylphenyl)-N2-(4-morpholinophenyl)thieno[3,2-d]pyrimidine-2,7-diamine, 7-(5-amino-2-methylphenyl)-N-(4-morpholinophenyl)thieno[3,2-d]pyrimidin-2-amine, N-(cyanomethyl)-4-(2-(4-morpholinophenylamino)thieno[3,2-d]pyrimidin-7-yl)benzamide, 7-iodo-N-(3-morpholinophenyl)thieno[3,2-d]pyrimidin-2-amine, 7-(4-amino-3-nitrophenyl)-N-(4-morpholinophenyl)thieno[3,2-d]pyrimidin-2-amine, 7-(2-methoxypyridin-3-yl)-N-(4-morpholinophenyl)thieno[3,2-d]pyrimidin-2-amine, (3-(7-iodothieno[3,2-d]pyrimidin-2-ylamino)phenyl)methanol, N-tert-butyl-3-(2-(3-morpholinophenylamino)thieno[3,2-d]pyrimidin-7-yl)benzenesulfonamide, N-tert-butyl-3-(2-(3-(hydroxymethyl)phenylamino)thieno[3,2-d]pyrimidin-7-yl)benzenesulfonamide, N-(4-morpholinophenyl)-7-(4-nitrophenylthio)-5H-pyrrolo[3,2-d]pyrimidin-2-amine, N-tert-butyl-3-(2-(3,4,5-trimethoxyphenylamino)thieno[3,2-d]pyrimi-din-7-yl)benzenesulfonamide, 7-(4-amino-3-nitrophenyl)-N-(3,4-dimethoxyphenyl)thieno[3,2-d]pyrimidin-2-amine, N-(3,4-dimethoxyphenyl)-7-(2-methoxypyridin-3-yl)thieno[3,2-d]pyrimidin-2-amine, N-tert-butyl-3-(2-(3,4-dimethoxyphenylamino)thieno[3,2-d]pyrimidin-7-yl)benzenesulfonamide, 7-(2-aminopyrimidin-5-yl)-N-(3,4-dimethoxyphenyl)thieno[3,2-d]pyrimidin-2-amine, N-(3,4-dimethoxyphenyl)-7-(2,6-dimethoxypyridin-3-yl)thieno[3,2-d]-pyrimidin-2-amine, N-(3,4-dimethoxyphenyl)-7-(2,4-dimethoxypyrimidin-5-yl)thieno[3,2-d]pyrim-idin-2-amine, 7-iodo-N-(4-(morpholinomethyl)phenyl)thieno[3,2-d]pyrimidin-2-amine, N-tert-butyl-3-(2-(4-(morpholinomethyl)phenylamino)thieno[3,2-d]pyrimidin-7-yl)benzenesulfonamide, 2-cyano-N-(4-methyl-3-(2-(4-morpholinophenylamino)thieno[3,2-d]pyrimidin-7-yl)phenyl)acetamide, ethyl 3-(2-(4-morpholinophenylamino)thieno[3,2-d]pyrimidin-7-yl)benzoate, 7-bromo-N-(4-(2-(pyrrolidin-1-yl)ethoxy)phenyl)thieno[3,2-d]pyrimidin-2-amine, N-(3-(2-(4-(2-(pyrrolidin-1-yl)ethoxy)phenylamino)thieno[3,2-d]pyrimidin-7-yl)phenyl)acetamide, N-(cyanomethyl)-3-(2-(4-morpholinophenylamino)thieno[3,2-d]pyrimidin-7-yl)benzamide, N-tert-butyl-3-(2-(4-morpholinophenylamino)thieno[3,2-d]pyrimidin-7-yl)benzamide, N-tert-butyl-3-(2-(4-(1-ethylpiperidin-4-yloxy)phenylamino)thieno-[3,2-d]pyrimidin-7-yl)benzenesulfonamide, tert-butyl-4-(2-(4-(morpholinomethyl)phenylamino)thieno[3,2-d]pyrimidin-7-yl)-1H-pyrazole-1-carboxylate, 7-bromo-N-(4-((4-ethylpiperazin-1-yl)methyl)phenyl)thieno[3,2-d]pyrimidin-2-amine, N-tert-butyl-3-(2-(4-((4-ethylpiperazin-1-yl)methyl)phenylamino)-thieno[3,2-d]pyrimidin-7-yl)benzenesulfonamide, N-(4-((4-ethylpiperazin-1-yl)methyl)phenyl)-7-(1H-pyrazol-4-yl)thieno[3,2-d]pyrimidin-2-amine, N-(cyanomethyl)-3-(2-(4-(morpholinomethyl)phenylamino)thieno[3,2-d]pyrimi-din-7-yl)benzamide, N-tert-butyl-3-(2-(4-(2-(pyrrolidin-1-yl)ethoxy)phenylamino)thieno[3,2-d]-pyrimidin-7-yl)benzenesulfonamide, tert-butyl pyrrolidin-1-yl)ethoxy)phenylamino)thieno[3,2-d]pyrimidin-7-yl)benzylcarb-amate, 3-(2-(4-(2-(pyrrolidin-1-yl)ethoxy)phenylamino)thieno[3,2-d]pyrimidin-7-yl)benzenesulfonamide, 7-(3-chloro-4-fluorophenyl)-N-(4-(2-(pyrrolidin-1-yl)ethoxy)phenyl)thieno-[3,2-d]pyrimidin-2-amine, tert-butyl 4-(2-(4-(1-ethylpiperidin-4-yloxy)phenylamino)thieno[3,2-d]pyrimidin-7-yl-)-1H-pyrazole-1-carboxylate, 7(benzo[d][1,3]dioxol-5-yl)-N-(4-(morpholinomethyl)phenyl)thieno[3,2-d]pyrimidin-2-amine, tert-butyl 5-(2-(4-(morpholinomethyl)phenylamino)thieno[3,2-d]pyrimidin-7-yl)-1H-indole-1-carboxylate, 7-(2-aminopyrimidin-5-yl)-N-(4-(morpholinomethyl)phenyl)thieno[3,2-d]pyrimidin-2-amine, tert-butyl 4-(2-(-4-(morpholinomethyl)phenylamino)thieno[3,2-d]pyrimidin-7-yl)-5, 6-di-hydropyridine-1(2H)-carboxylate, tert-butyl morpholinomethyl)phenylamino)thieno[3,2-d]pyrimidin-7-yl)benzylcarbamate, N-(3-(2-(4-(morpholinomethyl)phenylamino)thieno[3,2-d]pyrimidin-7-yl)phen-yl)acetamide, N-(4-(2-(4-(morpholinomethyl)phenylamino)thieno[3,2-d]pyrimidin-7-yl)phen-yl)acetamide, N-(3-(2-(4-(morpholinomethyl)phenylamino)thieno[3,2-d]pyrimidin-7-yl)phen-yl)methanesulfonamide, 7-(4-(4-methylpiperazin-1-yl)phenyl)-N-(4-(morpholinomethyl)phenyl)thieno-[3,2-d]pyrimidin-2-amine, N-(2-methoxy-4-(2-(4-(morpholinomethyl)phenylamino)thieno[3,2-d]pyrimidin-7-yl)phenyl)acetamide, 7-bromo-N-(3,4,5-trimethoxyphenyl)thieno[3,2-d]pyrimidin-2-amine, (3-(2-(3,4,5-trimethoxyphenylamino)thieno[3,2-d]pyrimidin-7-yl)phenyl)met-hanol, (4-(2-(3,4,5-trimethoxyphenylamino)thieno[3,2-d]pyrimidin-7-yl)phen-yl)methanol, (3-(2-(4-morpholinophenylamino)thieno[3,2-d]pyrimidin-7-yl)phenyl)methano-1, (4-(2-(4-morpholinophenylamino)thieno[3,2-d]pyrimidin-7-yl)phenyl)methanol, N-(pyrrolidin-1-yl)ethoxy)phenylamino)thieno[3,2-d]pyrimidin-7-yl)benzyl)methanesulfonamide, tert-butyl morpholinomethyl)phenylamino)thieno[3,2-d]pyrimidin-7-yl)benzylcarbamate, N-(4-(morpholinomethyl)phenyl)-7-(3-(piperazin-1-yl)phenyl)thieno[3,2-d]pyrimidin-2-amine, 7-(6-(2-morpholinoethylamino)pyridin-3-yl)-N-(3,4,5-trimethoxyphenyl)thieno[3,2-d]pyrimidin-2-amine, 7-(2-ethylphenyl)-N-(4-(2-(pyrrolidin-1-yl)ethoxy)phenyl)thieno[3,2-d]pyrimidin-2-amine, 7-(4-(aminomethyl)phenyl)-N-(4-(morpholinomethyl)phenyl)thieno[3,2-d]pyrimidin-2-amine, N-(4-(1-ethylpiperidin-4-yloxy)phenyl)-7-(1H-pyrazol-4-yl)thieno[3,2-d]pyrimidin-2-amine, N-(2,4-dimethoxyphenyl)-7-phenylthieno[3,2-d]pyrimidin-2-amine, 7-bromo-N-(3,4-dimethoxyphenyl)thieno[3,2-d]pyrimidin-2-amine, N-(3,4-dimethoxyphenyl)-7-phenylthieno[3,2-d]pyrimidin-2-amine, and pharmaceutically acceptable salts and prodrugs thereof.

HMGB1 antibodies and COX-2 inhibitors can be used, which downregulate the cytokine storm. Examples of such compounds include Actemra (Roche). Celebrex (celecoxib), a COX-2 inhibitor, can be used. IL-8 (CXCL8) inhibitors can also be used.

Chemokine receptor CCR2 antagonists, such as PF-04178903 can reduce pulmonary immune pathology.

Selective α7Ach receptor agonists, such as GTS-21 (DMXB-A) and CNI-1495, can be used. These compounds reduce TNF-α. The late mediator of sepsis, HMGB1, downregulates IFN-7 pathways, and prevents the LPS-induced suppression of IL-10 and STAT 3 mechanisms.

### Compounds for Treating or Preventing Blood Clots

Viruses that cause respiratory infections, including Coronaviruses such as Covid-19, can be associated with pulmonary blood clots, and blood clots that can also do damage to the heart.

The compounds described herein can be co-administered with compounds that inhibit blood clot formation, such as blood thinners, or compounds that break up existing blood clots, such as tissue plasminogen activator (TPA), Integrilin (eptifibatide), abciximab (ReoPro) or tirofiban (Aggrastat).

Blood thinners prevent blood clots from forming, and keep existing blood clots from getting larger. There are two main types of blood thinners. Anticoagulants, such as heparin or warfarin (also called Coumadin), slow down biological processes for producing clots, and antiplatelet aggregation drugs, such as Plavix, aspirin, prevent blood cells called platelets from clumping together to form a clot.

By way of example, Integrilin® is typically administered at a dosage of 180 mcg/kg intravenous bolus administered as soon as possible following diagnosis, with 2 mcg/kg/min continuous infusion (following the initial bolus) for up to 96 hours of therapy.

Representative platelet aggregation inhibitors include glycoprotein IIB/IIIA inhibitors, phosphodiesterase inhibitors, adenosine reuptake inhibitors, and adenosine diphosphate (ADP) receptor inhibitors. These can optionally be administered in combination with an anticoagulant.

Representative anti-coagulants include coumarins (vitamin K antagonists), heparin and derivatives thereof, including unfractionated heparin (UFH), low molecular weight heparin (LMWH), and ultra-low-molecular weight heparin (ULMWH), synthetic pentasaccharide inhibitors of factor Xa, including Fondaparinux, Idraparinux, and Idrabiotaparinux, directly acting oral anticoagulants (DAOCs), such as dabigatran, rivaroxaban, apixaban, edoxaban and betrixaban, and antithrombin protein therapeutics/thrombin inhibitors, such as bivalent drugs hirudin, lepirudin, and bivalirudin and monovalent argatroban.

Representative platelet aggregation inhibitors include pravastatin, Plavix (clopidogrel bisulfate), Pletal (cilostazol), Effient (prasugrel), Aggrenox (aspirin and dipyridamole), Brilinta (ticagrelor), caplacizumab, Kengreal (cangrelor), Persantine (dipyridamole), Ticlid (ticlopidine), Yosprala (aspirin and omeprazole).

### Small Molecule Covalent CoV 3CLpro Inhibitors

Representative small molecule covalent CoV 3CLpro inhibitors include the following compounds:

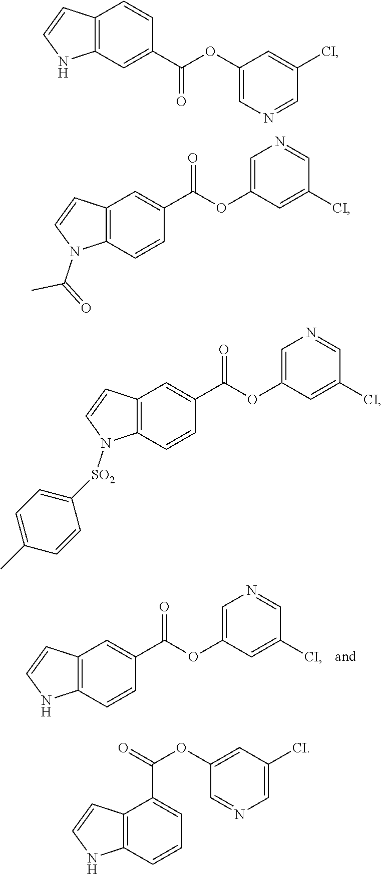

### Non-Covalent CoV 3CLpro Inhibitors

Representative non-covalent CoV 3CLpro inhibitors include the following:

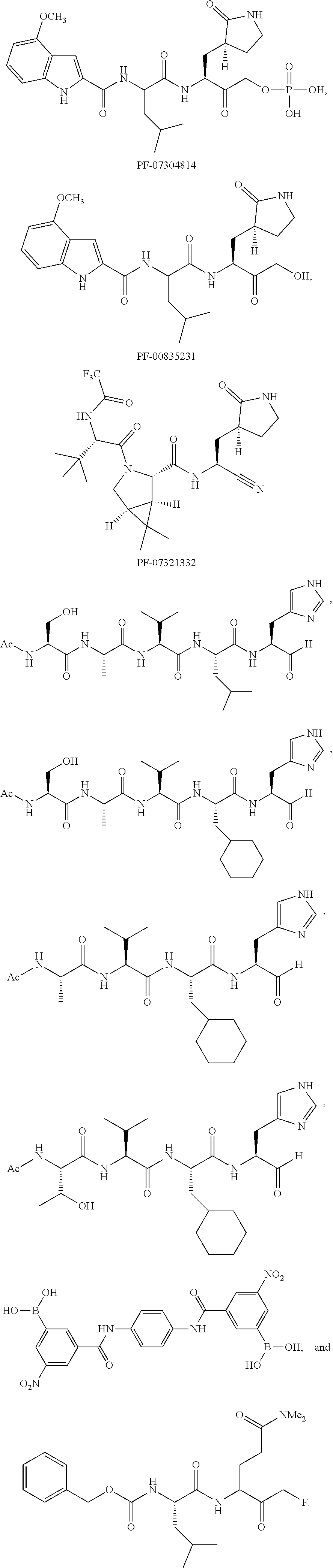

### SARS-CoV PLpro Inhibitors

Representative SARS-Cov PLpro inhibitors include the following:

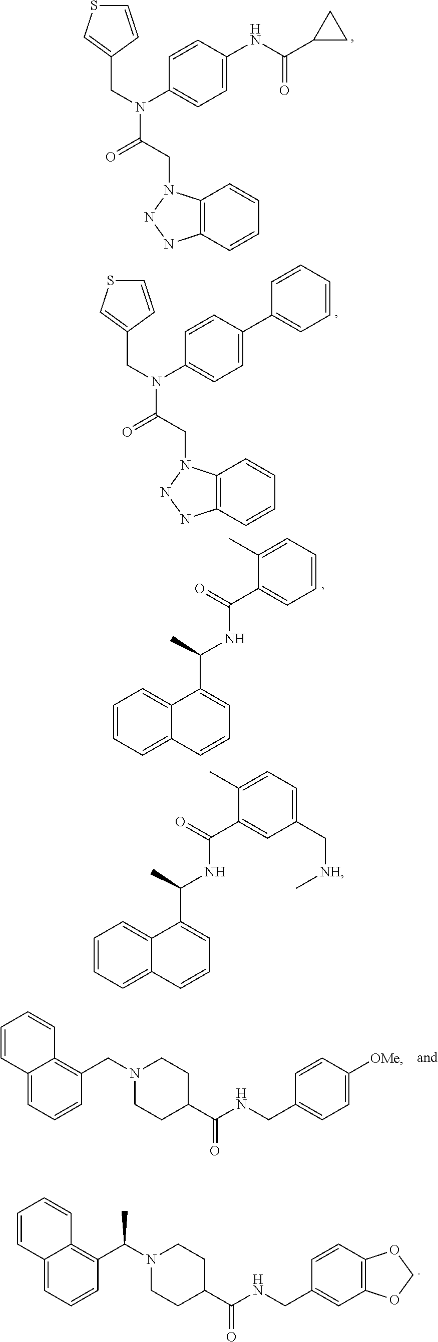

Additional compounds include the following:

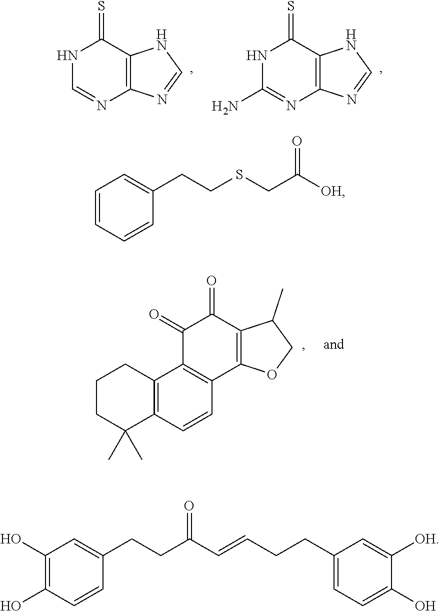

Additional Compounds that can be Used

Additional compounds and compound classes that can be used in combination therapy include the following: Antibodies, including monoclonal antibodies (mAb), Arbidol (umifenovir), Actemra (tocilizumab), APN01 (Aperion Biologics), ARMS-1 (which includes Cetylpyridinium chloride (CPC)), ASC09 (Ascletis Pharma), AT-001 (Applied Therapeutics Inc.) and other aldose reductase inhibitors (ARI), ATYR1923 (aTyr Pharma, Inc.), Aviptadil (Relief Therapeutics), Azvudine, Bemcentinib, BLD-2660 (Blade Therapeutics), Bevacizumab, Brensocatib, Calquence (acalabrutinib), Camostat mesylate (a TMPRSS2 inhibitor), Camrelizumab, CAP-1002 (Capricor Therapeutics), CD24Fcm, Clevudine, (Oncolmmune), CM4620-IE (CalciMedica Inc., CRAC channel inhibitor), Colchicine, convalescent plasma, CYNK-001 (Sorrento Therapeutics), DAS181 (Ansun Pharma), Desferal, Dipyridamole (Persantine), Dociparstat sodium (DSTAT), Duvelisib, Eculizumab, EIDD-2801 (Ridgeback Biotherapeutics), Emapalumab, Fadraciclib (CYC065) and seliciclib (roscovitine) (Cyclin-dependent kinase (CDK) inhibitors), Farxiga (dapagliflozin), Favilavir/Favipiravir/T-705/Avigan, Galidesivir, Ganovo (danoprevir), Gilenya (fingolimod) (sphingosine 1-phosphate receptor modulator), Gimsilumab, IFX-1, Ilaris (canakinumab), intravenous immunoglobulin, Ivermectin (importin α/β inhibitor), Kaletra/Aluvia (lopinavir/ritonavir), Kevzara (sarilumab), Kineret (anakinra), LAU-7b (fenretinide), Lenzilumab, Leronlimab (PRO 140), LY3127804 (an anti-Ang2 antibody), Leukine (sargramostim, a granulocyte macrophage colony stimulating factor), Losartan, Valsartan, and Telmisartan (Angiotensin II receptor antagonists), Meplazumab, Metablok (LSALT peptide, a DPEP1 inhibitor), Methylprednisolone and other corticosteroids, MN-166 (ibudilast, Macrophage migration inhibitory factor (MIF) inhibitor), MRx-4DP0004 (a strain of Bifidobacterium breve, 4D Pharma), Nafamostat (a serine protease inhibitor), Neuraminidase inhibitors like Tamiflu (oseltamivir), Nitazoxanide (nucleocapsid (N) protein inhibitor), Nivolumab, OT-101 (Mateon), Novaferon (man-made Interferon), HCV NS5A drugs such as Daclastavir, Opaganib (yeliva) (Sphingosine kinase-2 inhibitor), Otilimab, PD-1 blocking antibody, peginterferons, such as peginterferon lambda, Pepcid (famotidine), Piclidenoson (A3 adenosine receptor agonist), Prezcobix (darunavir), PUL-042 (Pulmotect, Inc., toll-like receptor (TLR) binder), Rebif (interferon beta-la), RHB-107 (upamostat) (serine protease inhibitor, RedHill Biopharma Ltd.), Selinexor (selective inhibitor of nuclear export (SINE)), SNG001 (Synairgen, inhaled interferon beta-la), Solnatide, stem cells, including mesenchymal stem cells, MultiStem (Athersys), and PLX (Pluristem Therapeutics), Sylvant (siltuximab), Thymosin, TJM2 (TJ003234), Tradipitant (neurokinin-1 receptor antagonist), Truvada (emtricitabine and tenofovir), Ultomiris (ravulizumab-cwvz), Vazegepant (CGRP receptor antagonist or blocker), and Xofluza (baloxavir marboxil).

### Repurposed Antiviral Agents

A number of pharmaceutical agents, including agents active against other viruses, have been evaluated against Covid-19, and found to have activity. Any of these compounds can be combined with the compounds described herein. Representative compounds include lopinavir, ritonavir, niclosamide, promazine, PNU, UC2, cinanserin (SQ 10,643), Calmidazolium (C3930), tannic acid, 3-isotheaflavin-3-gallate, theaflavin-3,3′-digallate, glycyrrhizin, S-nitroso-N-acetylpenicillamine, nelfinavir, niclosamide, chloroquine, hydroxychloroquine, 5-benzyloxygramine, ribavirin, Interferons, such as Interferon (IFN)-α, IFN-β, and pegylated versions thereof, as well as combinations of these compounds with ribavirin, chlorpromazine hydrochloride, triflupromazine hydrochloride, gemcitabine, imatinib mesylate, dasatinib, and imatinib.

### VIII. Pharmaceutical Compositions

Hosts, including but not limited to humans, infected with a Coronviridae virus, or the other viruses described, herein can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

A preferred dose of the compound for will be in the range of between about 0.01 and about 50 mg/kg, more generally, between about 0.1 and 1 5 mg/kg, and, preferably, between about 0.5 and about 2 mg/kg, of body weight of the recipient per day, until the patient has recovered. In some cases, a compound may be administered at a dosage of up to 10 μM, which might be considered a relatively high dose if administered for an extended period of time, but which can be acceptable when administered for the duration of an infection with one or more of the viruses described herein, which is typically on the order of several days to several weeks. In general, the compounds can be administered for up to 14 days, but preferably are administered for between 3 and 5 days.

The effective dosage range of the pharmaceutically acceptable salts and prodrugs can be calculated based on the weight of the parent compound to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to but not limited to one containing 7 to 600 mg, preferably 70 to 600 mg of active ingredient per unit dosage form. An oral dosage of 5-400 mg is usually convenient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

A preferred mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antiviral compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

### Transdermal Formulations

In some embodiments, the compositions are present in the form of transdermal formulations, such as that used in the FDA-approved agonist rotigitine transdermal (Neupro patch). Another suitable formulation is that described in U.S. Publication No. 20080050424, entitled “Transdermal Therapeutic System for Treating Parkinsonism.” This formulation includes a silicone or acrylate-based adhesive, and can include an additive having increased solubility for the active substance, in an amount effective to increase dissolving capacity of the matrix for the active substance.

The transdermal formulations can be single-phase matrices that include a backing layer, an active substance-containing self-adhesive matrix, and a protective film to be removed prior to use. More complicated embodiments contain multiple-layer matrices that may also contain non-adhesive layers and control membranes. If a polyacrylate adhesive is used, it can be crosslinked with multivalent metal ions such as zinc, calcium, aluminum, or titanium ions, such as aluminum acetylacetonate and titanium acetylacetonate.

When silicone adhesives are used, they are typically polydimethylsiloxanes. However, other organic residues such as, for example, ethyl groups or phenyl groups may in principle be present instead of the methyl groups. Because the active compounds are amines, it may be advantageous to use amine-resistant adhesives. Representative amine-resistant adhesives are described, for example, in EP 0 180 377.

Representative acrylate-based polymer adhesives include acrylic acid, acrylamide, hexylacrylate, 2-ethylhexylacrylate, hydroxyethylacrylate, octylacrylate, butylacrylate, methylacrylate, glycidylacrylate, methacrylic acid, methacrylamide, hexylmethacrylate, 2-ethylhexylmethacrylate, octylmethacrylate, methylmethacrylate, glycidylmethacrylate, vinylacetate, vinylpyrrolidone, and combinations thereof.

The adhesive must have a suitable dissolving capacity for the active substance, and the active substance most be able to move within the matrix, and be able to cross through the contact surface to the skin. Those of skill in the art can readily formulate a transdermal formulation with appropriate transdermal transport of the active substance.

Certain pharmaceutically acceptable salts tend to be more preferred for use in transdermal formulations, because they can help the active substance pass the barrier of the stratum corneum. Examples include fatty acid salts, such as stearic acid and oleic acid salts. Oleate and stearate salts are relatively lipophilic, and can even act as a permeation enhancer in the skin.

Permeation enhancers can also be used. Representative permeation enhancers include fatty alcohols, fatty acids, fatty acid esters, fatty acid amides, glycerol or its fatty acid esters, N-methylpyrrolidone, terpenes such as limonene, alpha-pinene, alpha-terpineol, carvone, carveol, limonene oxide, pinene oxide, and 1,8-eucalyptol.

The patches can generally be prepared by dissolving or suspending the active agent in ethanol or in another suitable organic solvent, then adding the adhesive solution with stirring. Additional auxiliary substances can be added either to the adhesive solution, the active substance solution or to the active substance-containing adhesive solution. The solution can then be coated onto a suitable sheet, the solvents removed, a backing layer laminated onto the matrix layer, and patches punched out of the total laminate.

### Nanoparticulate Compositions

The compounds described herein can also be administered in the form of nanoparticulate compositions. In one embodiment, controlled release nanoparticulate formulations comprise a nanoparticulate active agent to be administered and a rate-controlling polymer which prolongs the release of the agent following administration. In this embodiment, the compositions can release the active agent, following administration, for a time period ranging from about 2 to about 24 hours or up to 30 days or longer. Representative controlled release formulations including a nanoparticulate form of the active agent are described, for example, in U.S. Pat. No. 8,293,277.

Nanoparticulate compositions can comprise particles of the active agents described herein, having a non-crosslinked surface stabilizer adsorbed onto, or associated with, their surface.

The average particle size of the nanoparticulates is typically less than about 800 nm, more typically less than about 600 nm, still more typically less than about 400 nm, less than about 300 nm, less than about 250 nm, less than about 100 nm, or less than about 50 nm. In one aspect of this embodiment, at least 50% of the particles of active agent have an average particle size of less than about 800, 600, 400, 300, 250, 100, or 50 nm, respectively, when measured by light scattering techniques.

A variety of surface stabilizers are typically used with nanoparticulate compositions to prevent the particles from clumping or aggregating. Representative surface stabilizers are selected from the group consisting of gelatin, lecithin, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol, polyvinylpyrrolidone, tyloxapol, poloxamers, poloxamines, poloxamine 908, dialkylesters of sodium sulfosuccinic acid, sodium lauryl sulfate, an alkyl aryl polyether sulfonate, a mixture of sucrose stearate and sucrose distearate, p-isononylphenoxypoly-(glycidol), SA9OHCO, decanoyl-N-methylglucamide, n-decyl-D-glucopyranoside, n-decyl-D-maltopyranoside, n-dodecyl-D-glucopyranoside, n-dodecyl-D-maltoside, heptanoyl-N-methylglucamide, n-heptyl-D-glucopyranoside, n-heptyl-D-thioglucoside, n-hexyl-D-glucopyranoside, nonanoyl-N-methylglucamide, n-nonyl-D-glucopyranoside, octanoyl-N-methylglucamide, n-octyl-D-glucopyranoside, and octyl-D-thioglucopyranoside. Lysozymes can also be used as surface stabilizers for nanoparticulate compositions. Certain nanoparticles such as poly(lactic-co-glycolic acid) (PLGA)-nanoparticles are known to target the liver when given by intravenous (IV) or subcutaneously (SQ).

Representative rate controlling polymers into which the nanoparticles can be formulated include chitosan, polyethylene oxide (PEO), polyvinyl acetate phthalate, gum arabic, agar, guar gum, cereal gums, dextran, casein, gelatin, pectin, carrageenan, waxes, shellac, hydrogenated vegetable oils, polyvinylpyrrolidone, hydroxypropyl cellulose (HPC), hydroxyethyl cellulose (HEC), hydroxypropyl methylcelluose (HPMC), sodium carboxymethylcellulose (CMC), poly(ethylene) oxide, alkyl cellulose, ethyl cellulose, methyl cellulose, carboxymethyl cellulose, hydrophilic cellulose derivatives, polyethylene glycol, polyvinylpyrrolidone, cellulose acetate, cellulose acetate butyrate, cellulose acetate phthalate, cellulose acetate trimellitate, polyvinyl acetate phthalate, hydroxypropylmethyl cellulose phthalate, hydroxypropylmethyl cellulose acetate succinate, polyvinyl acetaldiethylamino acetate, poly(alkylmethacrylate), poly(vinyl acetate), polymers derived from acrylic or methacrylic acid and their respective esters, and copolymers derived from acrylic or methacrylic acid and their respective esters.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive nanoparticulate compositions having cationic surface stabilizers;” U.S. Pat. No. 6,431,478 for “Small Scale Mill;” and U.S. Pat. No. 6,432,381 for “Methods for targeting drug delivery to the upper and/or lower gastrointestinal tract,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

Certain nanoformulations can enhance the absorption of drugs by releasing drug into the lumen in a controlled manner, thus reducing solubility issues. The intestinal wall is designed to absorb nutrients and to act as a barrier to pathogens and macromolecules. Small amphipathic and lipophilic molecules can be absorbed by partitioning into the lipid bilayers and crossing the intestinal epithelial cells by passive diffusion, while nanoformulation absorption may be more complicated because of the intrinsic nature of the intestinal wall. The first physical obstacle to nanoparticle oral absorption is the mucus barrier which covers the luminal surface of the intestine and colon. The mucus barrier contains distinct layers and is composed mainly of heavily glycosylated proteins called mucins, which have the potential to block the absorption of certain nanoformulations. Modifications can be made to produce nanoformulations with increased mucus-penetrating properties (Ensign et al., “Mucus penetrating nanoparticles: biophysical tool and method of drug and gene delivery,” Adv Mater 24: 3887-3894 (2012)).

Once the mucus coating has been traversed, the transport of nanoformulations across intestinal epithelial cells can be regulated by several steps, including cell surface binding, endocytosis, intracellular trafficking and exocytosis, resulting in transcytosis (transport across the interior of a cell) with the potential involvement of multiple subcellular structures. Moreover, nanoformulations can also travel between cells through opened tight junctions, defined as paracytosis. Non-phagocytic pathways, which involve clathrin-mediated and caveolae-mediated endocytosis and macropinocytosis, are the most common mechanisms of nanoformulation absorption by the oral route.

Non-oral administration can provide various benefits, such as direct targeting to the desired site of action and an extended period of drug action. Transdermal administration has been optimized for nanoformulations, such as solid lipid nanoparticles (SLNs) and NEs, which are characterized by good biocompatibility, lower cytotoxicity and desirable drug release modulation (Cappel and Kreuter, “Effect of nanoparticles on transdermal drug delivery. J Microencapsul 8: 369-374 (1991)). Nasal administration of nanoformulations allows them to penetrate the nasal mucosal membrane, via a transmucosal route by endocytosis or via a carrier- or receptor-mediated transport process (Illum, “Nanoparticulate systems for nasal delivery of drugs: a real improvement over simple systems?” J. Pharm. Sci 96: 473-483 (2007)), an example of which is the nasal administration of chitosan nanoparticles of tizanidine to increase brain penetration and drug efficacy in mice (Patel et al., “Improved transnasal transport and brain uptake of tizanidine HCl-loaded thiolated chitosan nanoparticles for alleviation of pain,” J. Pharm. Sci 101: 690-706 (2012)). Pulmonary administration provides a large surface area and relative ease of access. The mucus barrier, metabolic enzymes in the tracheobronchial region and macrophages in the alveoli are typically the main barriers for drug penetration.

Particle size is a major factor determining the diffusion of nanoformulation in the bronchial tree, with particles in the nano-sized region more likely to reach the alveolar region and particles with diameters between 1 and 5 m expected to deposit in the bronchioles (Musante et al., “Factors affecting the deposition of inhaled porous drug particles,” J Pharm Sci 91: 1590-1600 (2002)). A limit to absorption has been shown for larger particles, presumably because of an inability to cross the air-blood barrier. Particles can gradually release the drug, which can consequently penetrate into the blood stream or, alternatively, particles can be phagocytosed by alveolar macrophages (Bailey and Berkland, “Nanoparticle formulations in pulmonary drug delivery,” Med. Res. Rev., 29: 196-212 (2009)).

Certain nanoformulations have a minimal penetration through biological membranes in sites of absorption and for these, i.v. administration can be the preferred route to obtain an efficient distribution in the body (Wacker, “Nanocarriers for intravenous injection—The long hard road to the market,” Int. J. Pharm., 457: 50-62., 2013).

The distribution of nanoformulations can vary widely depending on the delivery system used, the characteristics of the nanoformulation, the variability between individuals, and the rate of drug loss from the nanoformulations. Certain nanoparticles, such as solid drug nanoparticles (SDNs), improve drug absorption, which does not require them to arrive intact in the systemic circulation. Other nanoparticles survive the absorption process, thus altering the distribution and clearance of the contained drug.

Nanoformulations of a certain size and composition can diffuse in tissues through well-characterized processes, such as the enhanced permeability and retention effect, whereas others accumulate in specific cell populations, which allows one to target specific organs. Complex biological barriers can protect organs from exogenous compounds, and the blood-brain barrier (BBB) represents an obstacle for many therapeutic agents. Many different types of cells including endothelial cells, microglia, pericytes and astrocytes are present in the BBB, which exhibits extremely restrictive tight junctions, along with highly active efflux mechanisms, limiting the permeation of most drugs. Transport through the BBB is typically restricted to small lipophilic molecules and nutrients that are carried by specific transporters. One of the most important mechanisms regulating diffusion of nanoformulations into the brain is endocytosis by brain capillary endothelial cells.

Recent studies have correlated particle properties with nanoformulation entry pathways and processing in the human BBB endothelial barrier, indicating that uncoated nanoparticles have limited penetration through the BBB and that surface modification can influence the efficiency and mechanisms of endocytosis (Lee et al., “Targeting rat anti-mouse transferrin receptor monoclonal antibodies through blood-brain barrier in mouse,” J. Pharmacol. Exp. Ther. 292: 1048-1052 (2000)). Accordingly, surface-modified nanoparticles which cross the BBB, and deliver one or more of the compounds described herein, are within the scope of the disclosure.

Macrophages in the liver are a major pool of the total number of macrophages in the body. Kupffer cells in the liver possess numerous receptors for selective phagocytosis of opsonized particles (receptors for complement proteins and for the fragment crystallizable part of IgG). Phagocytosis can provide a mechanism for targeting the macrophages, and providing local delivery (i.e., delivery inside the macrophages) of the compounds described herein (TRUE?).

Nanoparticles linked to polyethylene glycol (PEG) have minimal interactions with receptors, which inhibits phagocytosis by the mononuclear phagocytic system (Bazile et al., “Stealth Me.PEG-PLA nanoparticles avoid uptake by the mononuclear phagocytes system,” J. Pharm. Sci. 84: 493-498 (1995)).

Representative nanoformulations include inorganic nanoparticles, SDNs, SLNs, NEs, liposomes, polymeric nanoparticles and dendrimers. The compounds described herein can be contained inside a nanoformulation, or, as is sometimes the case with inorganic nanoparticles and dendrimers, attached to the surface. Hybrid nanoformulations, which contain elements of more than one nanoformulation class, can also be used.

SDNs are lipid-free nanoparticles, which can improve the oral bioavailability and exposure of poorly water-soluble drugs (Chan, “Nanodrug particles and nanoformulations for drug delivery,” Adv. Drug. Deliv. Rev. 63: 405 (2011)). SDNs include a drug and a stabilizer, and are produced using ‘top-down’ (high pressure homogenization and wet milling) or bottom-up (solvent evaporation and precipitation) approaches.

SLNs consist of a lipid (or lipids) which is solid at room temperature, an emulsifier and water. Lipids utilized include, but are not limited to, triglycerides, partial glycerides, fatty acids, steroids and waxes. SLNs are most suited for delivering highly lipophilic drugs.

Liquid droplets of less than a 1000 nm dispersed in an immiscible liquid are classified as NEs. NEs are used as carriers for both hydrophobic and hydrophilic agents, and can be administered orally, transdermally, intravenously, intranasally, and ocularly. Oral administration can be preferred for chronic therapy, and NEs can effectively enhance oral bioavailability of small molecules, peptides and proteins.

Polymeric nanoparticles are solid particles typically around 200-800 nm in size, which can include synthetic and/or natural polymers, and can optionally be pegylated to minimize phagocytosis. Polymeric nanoparticles can increase the bioavailability of drugs and other substances, compared with traditional formulations. Their clearance depends on several factors, including the choice of polymers (including polymer size, polymer charge and targeting ligands), with positively charged nanoparticles larger than 100 nm being eliminated predominantly via the liver (Alexis et al., Factors affecting the clearance and biodistribution of polymeric nanoparticles. Mol Pharm 5: 505-515 (2008)).

Dendrimers are tree-like, nanostructured polymers which are commonly 10-20 nm in diameter.

Liposomes are spherical vesicles which include a phospholipid bilayer. A variety of lipids can be utilized, allowing for a degree of control in degradation level. In addition to oral dosing, liposomes can be administered in many ways, including intravenously (McCaskill et al., 2013), transdermally (Pierre and Dos Santos Miranda Costa, 2011), intravitreally (Honda et al., 2013) and through the lung (Chattopadhyay, 2013). Liposomes can be combined with synthetic polymers to form lipid-polymer hybrid nanoparticles, extending their ability to target specific sites in the body. The clearance rate of liposome-encased drugs is determined by both drug release and destruction of liposomes (uptake of liposomes by phagocyte immune cells, aggregation, pH-sensitive breakdown, etc.) (Ishida et al., “Liposome clearance,” Biosci Rep 22: 197-224 (2002)).

One of more of these nanoparticulate formulations can be used to deliver the active agents described herein to the macrophages, across the blood brain barrier, and other locations as appropriate.

### Controlled Release Formulations

In a preferred embodiment, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

The terms used in describing the invention are commonly used and known to those skilled in the art. As used herein, the following abbreviations have the indicated meanings:
DMSO dimethylsulfoxide
EtOAc ethyl acetate
h hour
Liq. liquid
M molar
MeOH Methanol
min minute
rt or RT room temperature
TBAF Tetrabutylammonium fluoride
THE tetrahydrofuran

### IX. General Methods for Preparing Active Compounds

Methods for the facile preparation of active compounds are known in the art and result from the selective combination known methods. The compounds disclosed herein can be prepared as described in detail below, or by other methods known to those skilled in the art. It will be understood by one of ordinary skill in the art that variations of detail can be made without departing from the spirit and in no way limiting the scope of the present invention.

For some compounds, the syntheses described herein are exemplary and can be used as a starting point to prepare additional compounds of the formulas described herein. These compounds can be prepared in various ways, including those synthetic schemes shown and described herein. Those skilled in the art will be able to recognize modifications of the disclosed syntheses and to devise routes based on the disclosures herein; all such modifications and alternate routes are within the scope of the claims.

The various reaction schemes are summarized below.
Scheme 1 is a synthetic approach to nucleosides 8. (Base and other variables listed in the Scheme are as defined in active compound section)
Scheme 2 is an alternate synthetic approach to nucleosides 11. (Base and other variables listed in the Scheme are as defined in active compound section)

In the schemes described herein, if a nucleoside base includes functional groups that might interfere with, or be decomposed or otherwise converted during the reaction steps, such functional groups can be protected using suitable protecting groups that can be removed. Protected functional groups, if any, can be deprotected later on.

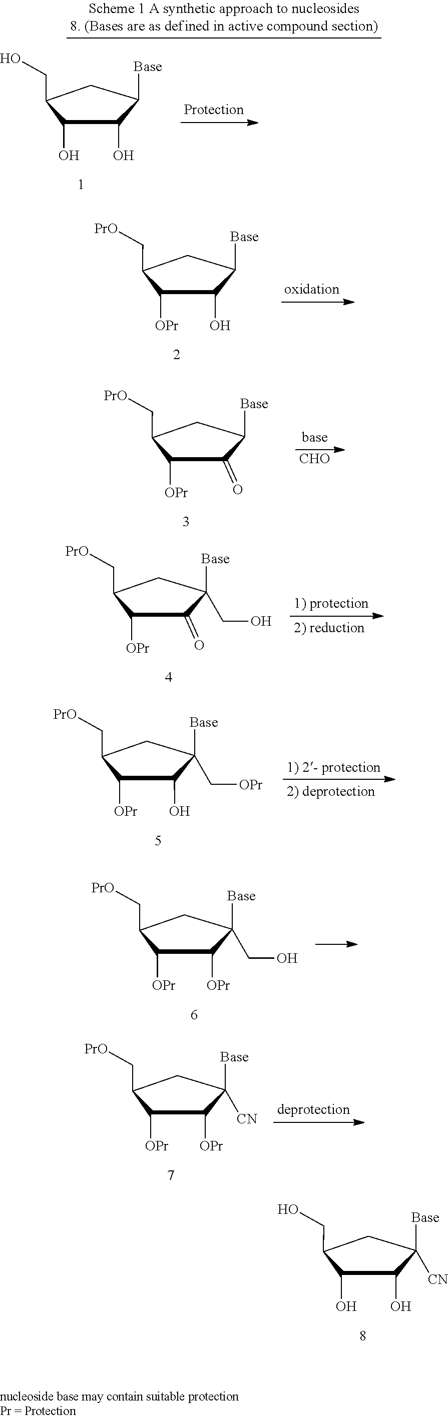

Compounds of Formula 8 can be prepared by first preparing nucleosides 1, which in turn can be accomplished by one of ordinary skill in the art, using methods outlined in: Bioorganic & Medicinal Chemistry Letters (2010), 20(8), 2601-2604; Nucleosides & Nucleotides (1992), 11(2-4), 351-63; Chemical Communications (Cambridge) (1996), (14), 1623-1624, Tetrahedron (1994), 50(33), 9961-74; Journal of Medicinal Chemistry (1988), 31(2), 484-6; Journal of the Chemical Society, Perkin Transactions 1: Organic and Bio-Organic Chemistry (1972-1999) (1986), (3), 399-404. Protection of the 3′- and 5′-positions of compound 1 can be achieved by treatment with, for instance, 1,3-dichloro-1,1,3,3-tetraisopropyldisiloxane in presence of a base such as pyridine or trielthylamine. Oxidation of the 2′-hydroxyl group with, for instance, CrO₃, followed by reaction of the newly formed ketone 3 with a base such as LDA in presence of paraformaldehyde can lead to compound 4. Protection of the hydroxyl group and reduction of the 2′-ketone with, for instance, NaBH₄ can give access to compound 5 which can be reprotected. After further deprotection of the 1′-CH₂—OH, primary alcohol in compound 6 can be oxidized to the corresponding aldehyde by using, for instance, Dess-Martin periodinane and then reacted with hydroxylamine to give, after complete deprotection, the desired compounds 8.

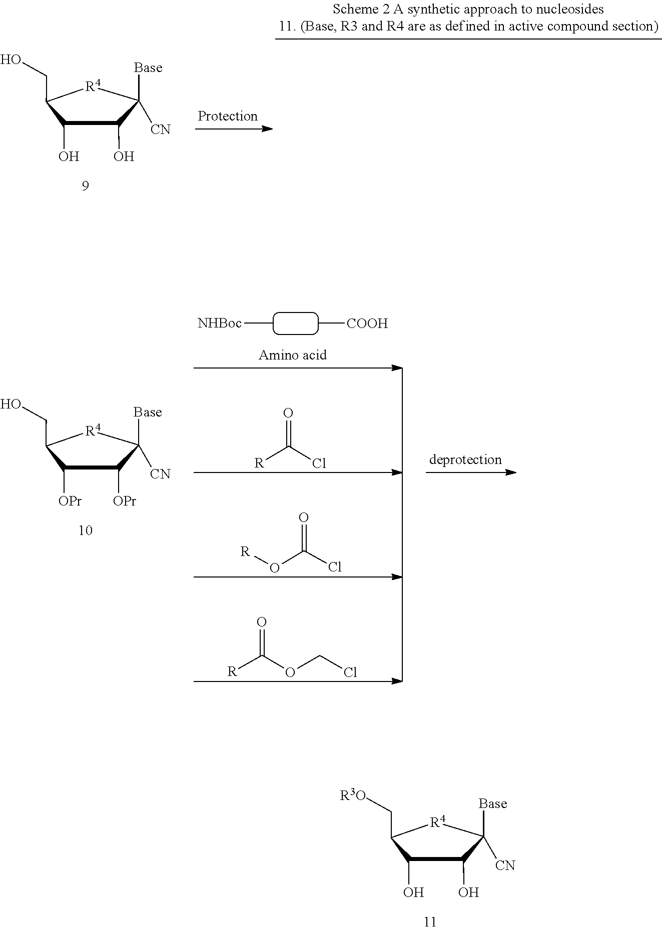

Compounds of general formula 11 can be prepared from nucleosides 9 by selective protection of the 2′,3′ hydroxyl groups, with for instance, acetone in presence of H₂SO₄, followed by coupling with a protected aninoacid in present of a coupling agent, such as EDC, or with an acid chloride, a carbonate chloride or a chloromethylester derivative in presence of a base such as Et₃N or NaH followed by appropriate deprotection.

Compounds of general Formula A can also be prepared by adapting the chemistry described in: WO2021/159044 A1; Journal of the Chemical Society, Perkin Transactions 1: Organic and Bio-Organic Chemistry (1972-1999) (1991), (1), 43-8; Bioorganic Chemistry (2015), 58, 18-25; Tetrahedron Letters (1985), 26(37), 4467-70; Journal of Medicinal Chemistry (2006), 49(22), 6614-6620; Collection of Czechoslovak Chemical Communications (1969), 34(12), 3755-68; Journal of the Chemical Society, Perkin Transactions 1: Organic and Bio-Organic Chemistry (1972-1999) (1973), (7), 665-9; Organic & Biomolecular Chemistry (2011), 9(3), 676-678;

Compounds can be prepared by first preparing nucleosides 1, which in turn can be accomplished by one of ordinary skill in the art, using methods outlined in: (a) Rajagopalan, P.; Boudinot, F. D; Chu, C. K.; Tennant, B. C.; Baldwin, B. H.; Antiviral Nucleosides: Chiral Synthesis and Chemotheraphy: Chu, C. K.; Eds. Elsevier: 2003. b) Recent Advances in Nucleosides: Chemistry and Chemotherapy: Chu, C. K.; Eds. Elsevier: 2002. c) Frontiers in Nucleosides & Nucleic Acids, 2004, Eds. R. F. Schinazi & D. C. Liotta, IHL Press, Tucker, GA, USA, pp: 319-37 d) Handbook of Nucleoside Synthesis: Vorbruggen H. & Ruh-Pohlenz C. John Wiley & sons 2001), and by general Schemes 3-4. Specifically, nucleosides 3 can be prepared by coupling sugar 1 with a protected, silylated or free nucleoside base in the presence of Lewis acid such as TMSOTf Deprotection of the 3′- and 5′-hydroxyls gives nucleoside 3.

Analogous compounds can be prepared using compounds like Compound 1, but with a fluorine rather than OPr at the 2′-position. Representative synthetic methods are described, for example, in U.S. Pat. No. 8,716,262.

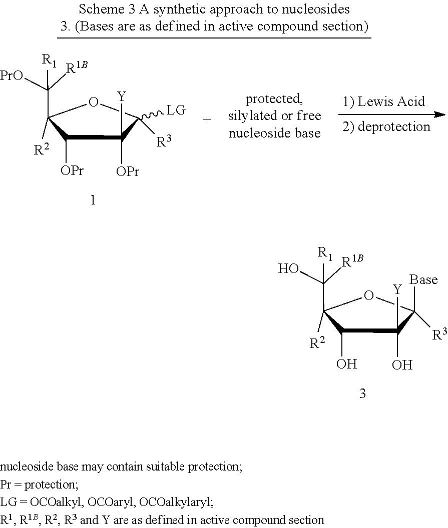

Similarly, compounds like Compound 1, but with a Y substituent at the 2′-position and/or an R substituent at the 3′-position, can be used to prepare nucleosides similar to Compound 3, but with Y or R substitution at the 2′- and/or 3′-positions, respectively.

Also, analogous compounds where the oxygen in the sugar ring is replaced with one of the other variables defined by R⁵ can also be prepared.

In the schemes described herein, if a nucleoside base includes functional groups that might interfere with, or be decomposed or otherwise converted during the coupling steps, such functional groups can be protected using suitable protecting groups. After the coupling step, protected functional groups, if any, can be deprotected.

Alternatively, nucleosides 3 can be prepared from 1′-halo, 1′-sulfonate or 1′-hydroxy compounds 2. For the case of 1′-halo or 1′-sulfonate a protected or free nucleoside base in the presence of a base such as triethyl amine or sodium hydride followed by deprotection would give nucleosides 3. For the case of 1′-hydroxy a protected or free nucleoside base in the presence of a Mitsunobu coupling agent such as diisopropyl azodicarboxylate followed by deprotection would give nucleosides 3.

Analogous compounds of Formula B can be prepared using compounds like Compound 1, but with a fluorine rather than OPr at the 2′-position. Representative synthetic methods are described for example, in U.S. Pat. No. 8,716,262.

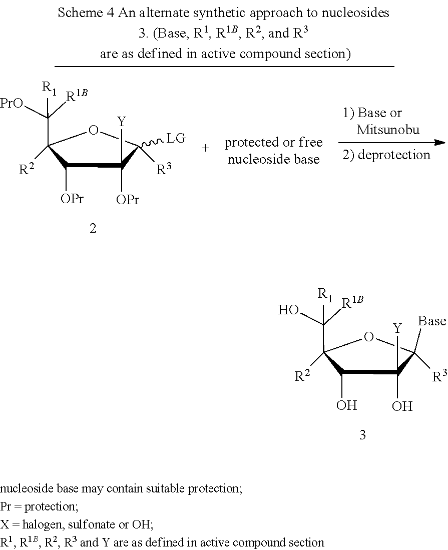

Similarly, compounds like Compound 2, but with a Y substituent at the 2′-position and/or an R substituent at the 3′-position, can be used to prepare nucleosides similar to Compound 3, but with Y or R substitution at the 2′- and/or 3′-positions, respectively.

Also, analogous compounds where the oxygen in the sugar ring is replaced with one of the other variables defined by R⁵ can also be prepared.

In the case of C-nucleosides prepared from bases:

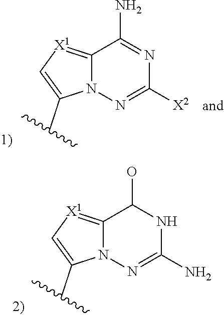

methods outlined in WO09132123, WO09132135, WO2011150288 and WO2011035250 can be used.

In the case of C-nucleosides prepared from other bases, methods outlined in Temburnikar K, Seley-Radtke K L. Recent advances in synthetic approaches for medicinal chemistry of C-nucleosides. Beilstein J Org Chem. 2018; 14:772-785 can be used.

In the case of carbocyclic nucleosides, methods outlined in the following references can be used:
Advances in the enantioselective synthesis of carbocyclic nucleosides, Chem. Soc. Rev., 2013, 42, 5056
The latest progress in the synthesis of carbocyclic nucleosides”. Nucleosides, Nucleotides & Nucleic Acids. 2000, 19 (3): 651-690
New progresses in the enantioselective synthesis and biological properties of carbocyclic nucleosides”. Mini Reviews in Medicinal Chemistry 2003, 3(2): 95-114.
Chemical synthesis of carbocyclic analogues of nucleosides”. Chemical Synthesis of Nucleoside Analogues. Hoboken: John Wiley & Sons. 2003 pp. 535-604

1′-CN nucleosides can be prepared by adapting the chemistry outlined in Synthesis of 1′-C-cyano pyrimidine nucleosides and characterization as HCV polymerase inhibitors. Nucleosides Nucleotides Nucleic Acids. 2015; 34(11):763-85 or by using the chemistry outlined in the schemes 5 and 6.

1′-CN nucleosides can be synthesized by cyanation of a protected sugar intermediate with reagents such AgCN or TMSCN in the presence of a Lewis acid, such as but not limited to SnCl₄, to give CN intermediate 5. Subsequent bromination, for instance though a radical reaction using bromine in presence of light, can give access to intermediate 6 which can be reacted in a glycosylation reaction using conditions described above

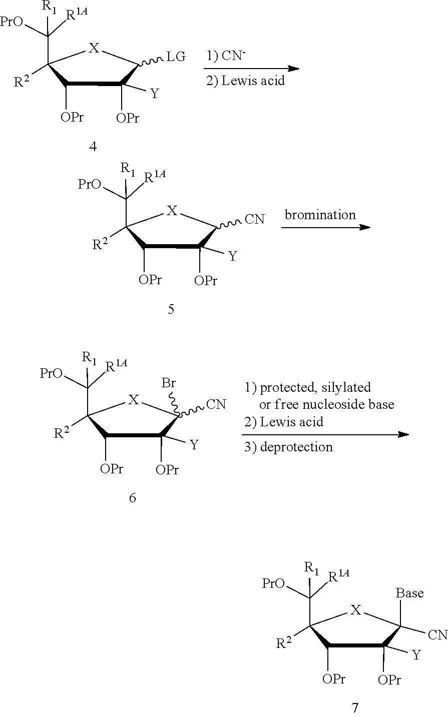

nucleoside base may contain suitable protection
Pr=protection
LG=leaving group—OCOalkyl, OCOAryl, halogen, OSO₂Aryl, OSO₂alkyl
X═O, S
R¹, R^{1A}, R² and Y are as defined in active compound section

### Scheme 5 A Synthetic Approach to Nucleosides 7. (Base are as Defined in Active Compound Section)

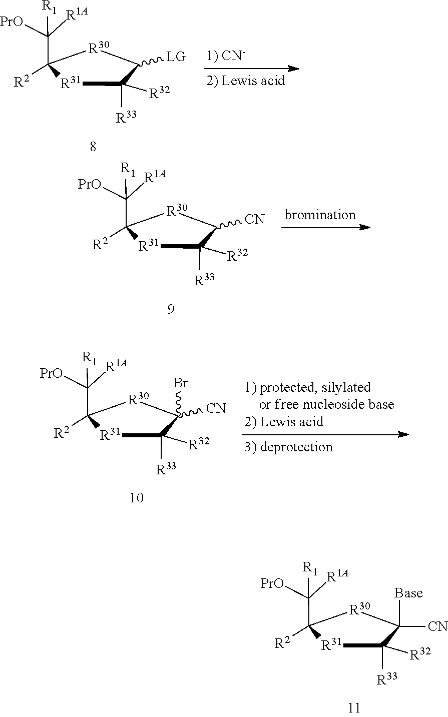

nucleoside base may contain suitable protection
Pr=protection
LG=leaving group—OCOalkyl, OCOAryl, halogen, OSO₂Aryl, OSO₂alkyl
X═O, S
R¹, R^{1A}, R² R30, R31, R32 and R33 are as defined in active compound section

### Scheme 6 A Synthetic Approach to Nucleosides 11. (Bases are as Defined in Active Compound Section)

Other carbocyclic nucleoside can be prepared by the chemistry outlined in Scheme 7 and 8. Correctly protected cyclopentyl amine scaffold 12 can be prepared by adapting the chemistry described in Nair V, Zhang F. Synthesis of a novel carbocyclic analog of bredinin. Molecules. 2013; 18(9):11576-11585. Oxidation of amine 12 can give access to nitro derivative 13 which can then undergo electrophilic cyanation (Org. Chem. Front., 2016, 3, 1535-1540). Reduction of the nitro group followed by construction of the purine or pyrimidine nucleobase and final deprotection will give the desired compounds 16.

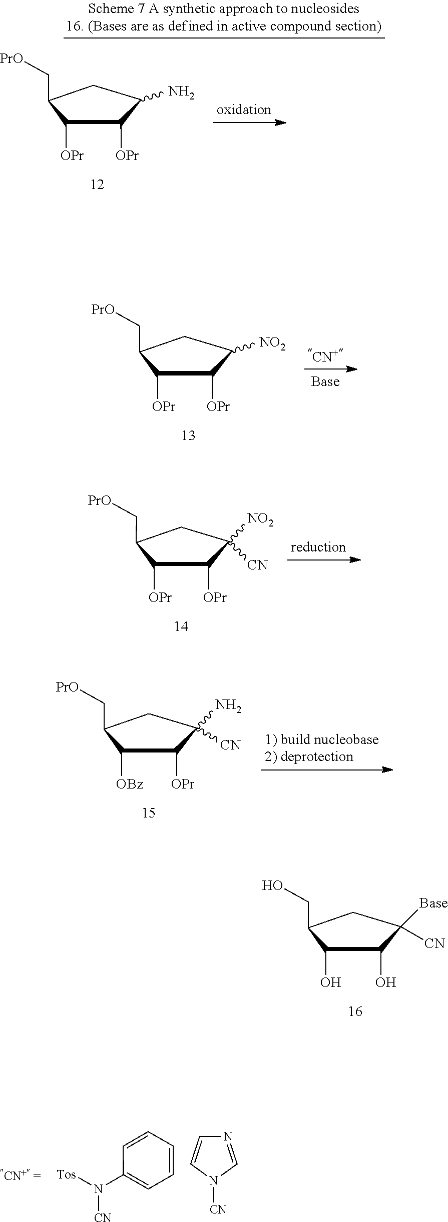

Alternatively, nitrocyclopentane derivative 13 can be reacted with formaldehyde in presence of a base such as LDA and then oxidized using an oxidant such as Dess Martin periodinane to give aldehyde 18. Subsequent reaction with hydroxylamine can give access to 20. Reduction of 20 followed by construction of the purine or pyrimidine nucleobase and final deprotection will give the desired compounds 16.

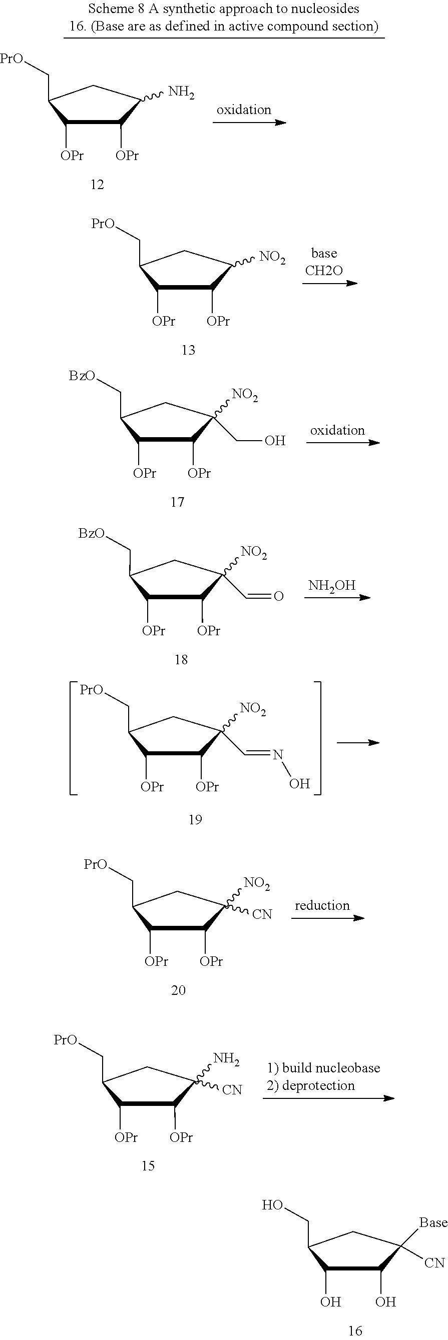

### Incorporation of Deuterium:

It is expected that single or multiple replacement of hydrogen with deuterium (carbon-hydrogen bonds to carbon-deuterium bond) at site(s) of metabolism in the sugar portion of a nucleoside antiviral agent will slow down the rate of metabolism. This can provide a relatively longer half-life, and slower clearance from the body. The slow metabolism of a therapeutic nucleoside is expected to add extra advantage to a therapeutic candidate, while other physical or biochemical properties are not affected. Intracellular hydrolysis or deuterium exchanges my result in liberation of deuterium oxide (D₂O).

Methods for incorporating deuterium into amino acids, phenol, sugars, and bases, are well known to those of skill in the art. Representative methods are disclosed in U.S. Pat. No. 9,045,521.

A large variety of enzymatic and chemical methods have been developed for deuterium incorporation at both the sugar and nucleoside stages to provide high levels of deuterium incorporation (D/H ratio). The enzymatic method of deuterium exchange generally has low levels of incorporation. Enzymatic incorporation has further complications due to cumbersome isolation techniques which are required for isolation of deuterated mononucleotide blocks. Schmidt et al., Ann. Chem. 1974, 1856; Schmidt et al., Chem. Ber., 1968, 101, 590, describes synthesis of 5′,5′-²H₂-adenosine which was prepared from 2′,3′-O-isopropylideneadenosine-5′-carboxylic acid or from methyl-2,3-isopropylidene-beta-D-ribofuranosiduronic acid, Dupre, M. and Gaudemer, A., Tetrahedron Lett. 1978, 2783. Kintanar, et al., Am. Chem. Soc. 1998, 110, 6367 reported that diastereoisomeric mixtures of 5′-deuterioadenosine and 5′(R/S)-deuteratedthymidine can be obtained with reduction of the appropriate 5′-aldehydes using sodium borodeuteride or lithium aluminum deuteride (98 atom % ²H incorporation). Berger et al., Nucleoside & Nucleotides 1987, 6, 395 described the conversion of the 5′-aldehyde derivative of 2′deoxyguanosine to 5′ or 4′-deuterio-2′-deoxyguanosine by heating the aldehyde in ²H₂O/pyridine mixture (1:1) followed by reduction of the aldehyde with NaBD₄.

Ajmera et al., Labelled Compd. 1986, 23, 963 described procedures to obtain 4′-deuterium labeled uridine and thymidine (98 atom % ²H). Sinhababu, et al., J. Am. Chem. Soc. 1985, 107, 7628) demonstrated deuterium incorporation at the C₃′ (97 atom % ²H) of adenosine during sugar synthesis upon stereoselective reduction of 1,2:5,6-di-O-isopropylidene-β-D-hexofuranos-3-ulose to 1,2:5,6-di-O-isopropylidene-3-deuterio-β-D-ribohexofuranose using sodium borodeuteride and subsequently proceeding further to the nucleoside synthesis. Robins, et al., Org. Chem. 1990, 55, 410 reported synthesis of more than 95% atom ²H incorporation at C3′ of adenosine with virtually complete stereoselectivity upon reduction of the 2′-O-tert-butyldimethylsilyl(TBDMS) 3-ketonucleoside by sodium borodeuteride in acetic acid. David, S. and Eustache, J., Carbohyd. Res. 1971, 16, 46 and David, S. and Eustache, J., Carbohyd. Res. 1971, 20, 319 described syntheses of 2′-deoxy-2′(S)-deuterio-uridine and cytidine. The synthesis was carried out by the use of 1-methyl-2-deoxy-2′-(S)-deuterio ribofuranoside.

Radatus, et al., J. Am. Chem. Soc. 1971, 93, 3086 described chemical procedures for synthesizing 2′-monodeuterated (R or S)-2′-deoxycytidines. These structures were synthesized from selective 2-monodeuterated-2-deoxy-D-riboses, which were obtained upon stereospecific reduction of a 2,3-dehydro-hexopyranose with lithium aluminum deuteride and oxidation of the resulting glycal. Wong et al. J. Am. Chem. Soc. 1978, 100, 3548 reported obtaining deoxy-1-deuterio-D-erythro-pentose, 2-deoxy-2(S)-deuterio-D-erythro-pentose and 2-deoxy-1,2(S)-dideuterio-D-erythro-pentose from D-arabinose by a reaction sequence involving the formation and LiAlD₄ reduction of ketene dithioacetal derivatives.

Pathak et al. J., Tetrahedron 1986, 42, 5427) reported stereospecific synthesis of all eight 2′ or 2′-deuterio-2′-deoxynucleosides by reductive opening of appropriate methyl 2,3-anhydro-beta-D-ribo or beta-D-lyxofuranosides with LiAlD₄. Wu et al. J. Tetrahedron 1987, 43, 2355 described the synthesis of all 2′,2″-dideuterio-2′-deoxynucleosides, for both deoxy and ribonucleosides, starting with oxidation of C2′ of sugar and subsequent reduction with NaBD₄ or LiAlD₄ followed by deoxygenation by tributyltin deuteride. Roy et al. J. Am. Chem. Soc. 1986, 108, 1675, reported 2′,2′-dideuterio-2′-deoxyguanosine and thymidine can be prepared from 2-deoxyribose 5-phosphate using 2-deoxyribose 5-phosphate aldolase enzyme in ²H₂O achieving some 90 atom % deuteration. Similarly, the synthesis of 4′,5′,5′-²H₃-guanosine can be carried out.

Therefore, it is clear that each position of the sugar residue can be selectively labeled.

A useful alternative method of stereospecific deuteration was developed to synthesize polydeuterated sugars. This method employed exchange of hydrogen with deuterium at the hydroxyl bearing carbon (i.e. methylene and methine protons of hydroxyl bearing carbon) using deuterated Raney nickel catalyst in ²H₂O.

Various techniques are available to synthesize fully deuterated deoxy and ribonucleosides. Thus, in one method, exchange reaction of deuterated Raney nickel-²H₂O with sugars, a number of deuterated nucleosides specifically labeled at 2′, 3′ and 4′ positions were prepared. The procedure consisted of deuteration at 2′, 3′ and 4′ positions of methyl beta-D-arabinopyranoside by Raney nickel-²H₂O exchange reaction followed by reductive elimination of ′2-hydroxyl group by tributyltin deuteride to give methyl beta-D-2′,2′,3′,4′-²H₄-2-deoxyribopyranoside, which was converted to methyl beta-D-2′,2′,3′,4′-²H₄-2′-deoxyribofuranoside and glycosylated to give various 2′,2′,3′,4′-²H₄-nucleosides (>97 atom % ²H incorporation for H3′ & H4′.

The synthesis of deuterated phenols is described, for example, in Hoyer, H. (1950), Synthese des pan-deutero-o-nitro-phenols. Chem. Ber., 83: 131-136. This chemistry can be adapted to prepare substituted phenols with deuterium labels. Deuterated phenols, and substituted analogs thereof, can be used, for example, to prepare phenoxy groups in phosphoramidate prodrugs.

The synthesis of deuterated amino acids is described, for example, in Matthews et al., Biochimica et Biophysica Acta (BBA)—General Subjects, Volume 497, Issue 1, 29 Mar. 1977, Pages 1-13. These and similar techniques can be used to prepare deuterated amino acids, which can be used to prepare phosphoramidate prodrugs of the nucleosides described herein.

One method for synthesizing a deuterated analog of the compounds described herein involves synthesizing a deuterated ribofuranoside with a 1′-CN substitution; and attaching a nucleobase to the deuterated ribofuranoside to form a deuterated nucleoside. A prodrug, such as a phosphoramidate prodrug, can be formed by modifying the 5′-OH group on the nucleoside. Where a deuterated phenol and/or deuterated amino acid is used, one can prepare a deuterated phosphoramidate prodrug.

Another method involves synthesizing a ribofuranoside with 1′-CN substitution, and attaching a deuterated nucleobase to form a deuterated nucleoside. This method can optionally be performed using a deuterated furanoside to provide additional deuteration. As with the method described above, the nucleoside can be converted into a prodrug form, which prodrug form can optionally include additional deuteration.

A third method involves synthesizing a ribofuranoside with 1′-CN substitution, attaching a nucleobase to form a nucleoside, and converting the nucleoside to a phosphoramidate prodrug using one or both of a deuterated amino acid or phenol analog in the phosphoramidate synthesis.

Accordingly, using the techniques described above, one can provide one or more deuterium atoms in the sugar, base, and/or prodrug portion of the nucleoside compounds described herein.

### SPECIFIC EXAMPLES

Specific compounds which are representative of this disclosure were prepared as per the following examples and reaction sequences; the examples and the diagrams depicting the reaction sequences are offered by way of illustration, to aid in the understanding of the disclosure and should not be construed to limit in any way the invention set forth in the claims which follow thereafter. The present compounds can also be used as intermediates in subsequent examples to produce additional compounds of the present invention. No attempt has necessarily been made to optimize the yields obtained in any of the reactions. One skilled in the art would know how to increase such yields through routine variations in reaction times, temperatures, solvents and/or reagents.

Anhydrous solvents were purchased from Aldrich Chemical Company, Inc. (Milwaukee, WI) and EMD Chemicals Inc. (Gibbstown, NJ). Reagents were purchased from commercial sources. Unless noted otherwise, the materials used in the examples were obtained from readily available commercial suppliers or synthesized by standard methods known to one skilled in the art of chemical synthesis. Melting points (mp) were determined on an Electrothermal digit melting point apparatus and are uncorrected. ¹H and ¹³C NMR spectra were taken on a Varian Unity Plus 400 spectrometer at room temperature and reported in ppm downfield from internal tetramethylsilane. Deuterium exchange, decoupling experiments or 2D-COSY were performed to confirm proton assignments. Signal multiplicities are represented by s (singlet), d (doublet), dd (doublet of doublets), t (triplet), q (quadruplet), br (broad), bs (broad singlet), m (multiplet). All J-values are in Hz. Mass spectra were determined on a Micromass Platform LC spectrometer using electrospray techniques. Elemental analyses were performed by Atlantic Microlab Inc. (Norcross, GA). Analytic TLC was performed on Whatman LK6F silica gel plates, and preparative TLC on Whatman PK5F silica gel plates. Column chromatography was carried out on Silica Gel or via reverse-phase high performance liquid chromatography.

### Experimental

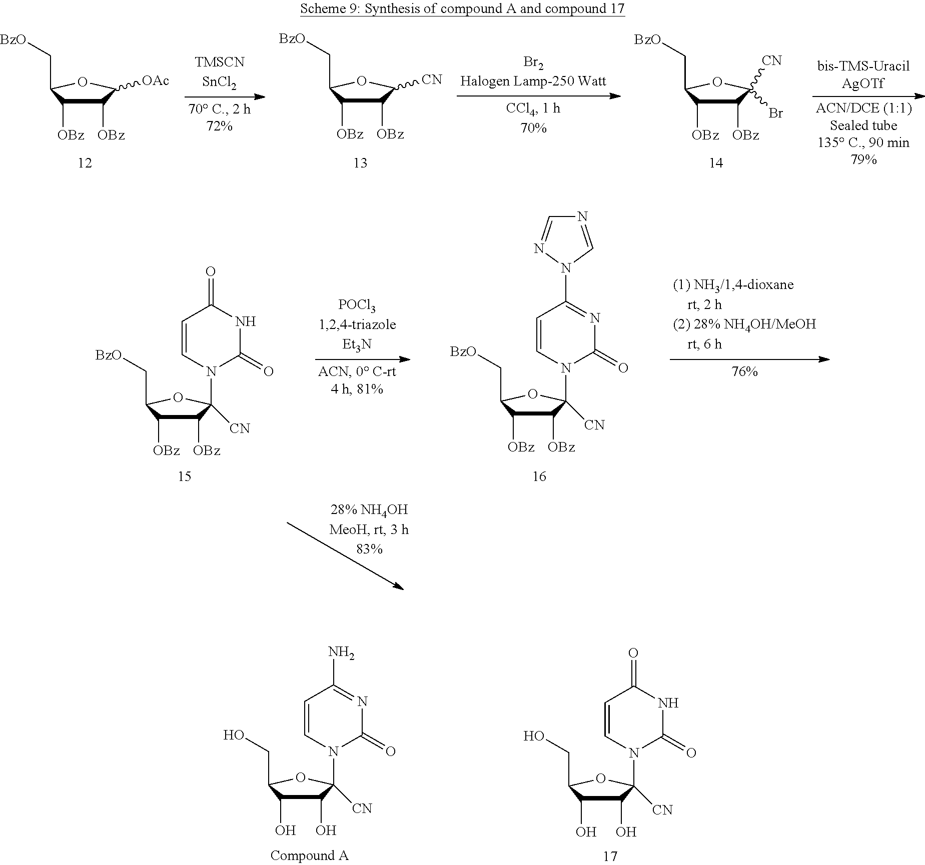

(2R, 3R, 4S)-2-((Benzoyloxy)methyl)-5-cyanotetrahydrofuran-3,4-diyl dibenzoate (13): To a suspension of dry (3R,4R,5R)-2-acetoxy-5-((benzoyloxy)methyl)tetrahydrofuran-3,4-diyl dibenzoate, 12 (1.0 g, 1.98 mmol) in TMSCN (1.06 mL, 8.5 mmol) under nitrogen, SnCl₂ (79 mg, 0.4 mmol) was added then heated to 70° C. for 2 h. The reaction mixture was cooled to room temperature and diluted with ethyl acetate (100 mL), quenched with saturated aq NaHCO₃ (100 mL) and filtered through a celite bed. The organic layer was separated, the aqueous layer was extracted with ethyl acetate (50 mL), the combined organic layers were washed with brine (50 mL), dried over sodium sulphate, filtered and concentrated under reduced pressure. The crude product was purified by flash column chromatography (0-40% ethyl acetate in hexane). Compound 13 was obtained (665 mg, 72%) as a yellowish thick liquid. ¹H NMR (CDCl₃, 400 MHz): δ 8.15-8.11 (m, 2H), 7.98-7.90 (m, 4H), 7.61-7.55 (m, 3H), 7.48-7.36 (m, 6H), 6.02-6.00 (m, 1H), 5.87-5.85 (m, 1H), 4.98 (d, 1H), 4.75-4.71 (m, 1H), 4.63-4.58 (m, 1H).

(3R,4R,5R)-5-((Benzoyloxy)methyl)-2-bromo-2-cyanotetrahydrofuran-3,4-diyl dibenzoate (14): To a solution of compound 13 (8.88 g, 18.83 mmol) in CCl4 (60 mL), bromine (3.46 mL, 69.7 mmol) was added at room temperature. The resultant reaction mixture was exposed to a 250-Watt halogen light bulb (tempered glass lens removed) for 1 h (reaction flask was equipped with condenser). After 1 h the light was removed and the solution was allowed to cool to room temperature. The solvents were evaporated under reduced pressure (water bath temperature at 35° C.). The resulting crude product was diluted with DCM (50 mL), washed with saturated aq NaHCO₃ (50 mL), water (50 mL), the organic layer was dried over sodium sulphate, filtered and concentrated under reduced pressure. The crude material was purified by flash column chromatography (0-50% of ethyl acetate in hexane) to give 7.29 g (70%) of compound 14 as a white solid. ¹H NMR (CDCl₃, 400 MHz): δ 8.11-8.02 (m, 5H), 7.91-7.88 (m, 1H), 7.52-7.28 (m, 9H), 6.36-6.35, 6.23-6.21, 5.89-5.80 (m, 2H), 5.02-4.61 (m, 3H).

Preparation of bis-TMS-Uracil: To a suspension of uracil (20.0 g) in excess hexamethyldisilazane (120 mL) was added a catalytic amount of NH₄SO₄ (100 mg). The resulting reaction mixture was refluxed for 3-4 h until clear a solution was observed. Then, the excess hexamethyldisilazane was removed by fractional distillation at 127° C. and the bis-TMS-uracil was distilled under vacuum (boiling point 116° C./12 mmHg). The desired bis-TMS-uracil 32 g (72%) was obtained as a colorless oil. (Studies on Synthetic Nucleosides. I. Trimethylsilyl derivatives of pyrimidines and purines. Chem. Pharm. Bull, 1961, 12 (3), 352-356.)

(2R,3R,4R,5R)-5-((benzoyloxy)methyl)-2-cyano-2-(2,4-dioxo-3,4-dihydropyrimidin-1(2H)-yl)tetrahydrofuran-3,4-diyl dibenzoate (15): Compound 14 (0.57 g, 1.034 mmol), bis-TMS-uracil (0.99 g, 3.86 mmol) and silver triflate (0.4 g, 1.5 mmol) were suspended in a 1:1 mixture of acetonitrile (3 mL) and dichloroethane (3 mL) in a sealed tube under nitrogen. The resulting reaction mixture was heated at 135° C. for 90 min. After the solution was cooled to room temperature, the reaction mixture was slowly added to saturated aqueous NaHCO₃ (50 mL) stirred for 10 min, then diluted with ethyl acetate (50 mL) and filtered through a celite bed. The organic layer was separated, the aqueous layer was extracted with ethyl acetate (2×20 mL), the combined organic layers were washed with brine (50 mL), dried over sodium sulphate, concentrated and purified by flash column chromatography (0-5% methanol in dichloromethane) to give 472 mg (79%) of compound 15 as a pale yellow solid. ¹H NMR (CDCl₃, 400 MHz) δ 8.05 (m, 4H), 7.96 (m, 3H), 7.63 (m, 3H), 7.3-7.6 (m, 6H), 6.46 (d, J=5.2 Hz, 1H), 6.03 (t, J=4.8 Hz, 1H), 5.61 (d, J=8.4 Hz, 1H), 5.28 (m, 1H), 4.94 (dd, J=13.2, 2.8 Hz, 1H), 4.69 (dd, J=12.8, 2.8 Hz, 1H). LC-MS: m/z: 582 [M+H], 602 [M+Na].

(2R,3R,4R,5R)-5-((Benzoyloxy)methyl)-2-cyano-2-(2-oxo-4-(1H-1,2,4-triazol-1-yl)pyrimidin-1(2H)-yl)tetrahydrofuran-3,4-diyl dibenzoate (16): To a solution of 1,2,4-triazole (731.9 mg, 10.6 mmol) in acetonitrile (20 mL) at 0° C., phosphoryl chloride (163.5 mL, 1.75 mmol) was added dropwise, followed by addition of triethylamine (1.6 mL, 11.5 mmol). The reaction mixture was stirred for 1 h at 0° C. A solution of compound 15 (200 mg, 0.34 mmol) in acetonitrile (4.2 mL) was added to the reaction mixture, which was allowed to room temperature and stirred at this temperature for 3 h. The reaction was quenched by addition of saturated aqueous NaHCO₃. The aqueous phase was extracted with dichloromethane (2×30 mL). The combined organic layers were dried over sodium sulphate, concentrated and purified by flash column chromatography (0-5% methanol in dichloromethane) to give compound 16 (176 mg, 81%) as a pale yellow solid. ¹H NMR (CDCl₃, 400 MHz) δ 9.10 (s, 1H), 8.31 (d, J=8.05 (m, 4H), 8.13 (m 3H), 7.97 (m, 4H), 7.60 (m, 2H), 7.3-7.6 (m, 7H), 6.94 (d, J=8.0 Hz, 1H), 6.38 (d, J=5.6 Hz, 1H), 5.91 (t, J=4.8 Hz, 1H), 5.17 (m, 1H), 5.03 (dd, J=12.8, 2.4 Hz, 1H), 4.60 (dd, J=13.2, 3.2 Hz, 1H). LC-MS: m/z: 633 [M+H], 655 [M+Na].

(2R,3R,4S,5R)-2-(4-amino-2-oxopyrimidin-1(2H)-yl)-3,4-dihydroxy-5-(hydroxymethyl)-tetrahydrofuran-2-carbonitrile (1′-Cyanocytidine), Compound A: Compound 16 (155 mg, 0.245 mmol) was dissolved in a saturated solution of ammonia in 1,4-dioxane (8 mL) and stirred at room temperature for 2 h. The solvents were evaporated under reduced pressure and the residue re-dissolved in a 1:1 mixture of aqueous 28% NH₄OH (3 mL) and methanol (3 mL) and the resulting mixture was stirred at room temperature for 6 h. (5.0 g batch was monitored by TLC until completion, nearly 24 h) The volatiles were evaporated under reduced pressure and the crude product was purified by flash column chromatography (0-15% methanol in dichloromethane) to give 50 mg (76%) of compound A as a white solid. ¹H NMR (400 MHz, MeOH-d₄): δ 8.16 (d, J=8 Hz, 1H), 5.9 (d, J=8 Hz, 1H), 4.45 (d, J=4.4 Hz, 1H), 4.29 (dt, J=8.4 Hz, J=2 Hz, 1H), 4.03 (m, 1H), 3.97 (dd, J=12.8, 2.4 Hz, 1H), 3.76 (dd, J=12.8, 2.8 Hz, 1H). LC-MS: m/z: 269.0 [M+H].

(2R,3R,4S,5R)-2-(2,4-Dioxo-3,4-dihydropyrimidin-1(2H)-yl)-3,4-dihydroxy-5-(hydroxymethyl)tetrahydrofuran-2-carbonitrile (1′-Cyanouridine) (17): To a solution of 2′,3′,5′-tri-O-benzoyl-1′-cyano-uridine (195 mg, 0.34 mmol) in MeOH (5 mL) was added 28% NH₄OH (4.5 mL), and the solution was stirred at RT for 3 h. The solvent was evaporated under reduced pressure, and the residue was purified by flash chromatography (0-15% methanol in dichloromethane) to give 75 mg (83%) of 17 as a white solid. ¹H-NMR (MeOH-d₄): δ 8.21 (d, J=8.4 Hz, 1H), 5.72 (d, J=8.4 Hz, 1H), 4.55-4.54 (m, 1H), 4.33-4.29 (m, 1H), 4.12-4.09 (m, 1H), 4.03-4.00 (m, 1H), 3.79-3.75 (m, 1H). LC-MS m/z: 292.1 (M+Na).

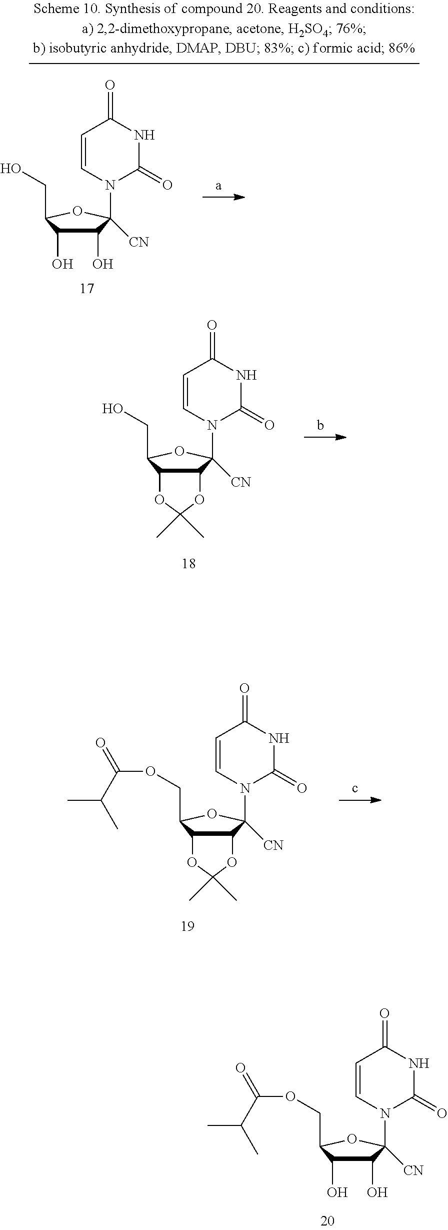

(3aR,4R,6R,6aR)-4-(2,4-Dioxo-3,4-dihydropyrimidin-1(2H)-yl)-6-(hydroxymethyl)-2,2-dimethyltetrahydrofuro[3,4-d][1,3]dioxole-4-carbonitrile (18): To a solution of 17 (75 mg, 0.28 mmol) in 5 mL of acetone was added 2,2-dimethoxypropane (1 mL, 8.16 mmol) and 20 μL of concentrated sulfuric acid. The mixture was stirred for 4 h at room temperature and then triethylamine (0.2 mL) was added. After stirring the solution at room temperature for 10 minutes, the volatils were removed under vacuum. The residue was purified flash chromatography (DCM to DCM/methanol=10:1) to give product 18 (65 mg, 76%) as a white solid. ¹H NMR (CD₃OD, 400 MHz): 7.91 (d, J=8.3 Hz, 1H), 5.73 (d, J=8.3 Hz, 1H), 4.8-5.1 (m, 3H), 3.81 (dd, J=12.1, 2.4 Hz, 1H), 3.70 (dd, J=12.1, 2.3 Hz, 1H), 1.68 (s, 3H), 1.41 (s, 3H). ¹³C NMR (CD₃OD, 400 MHz): d 164.8, 150.3, 139.0, 114.0, 100.9, 94.6, 89.8, 88.9, 81.9, 25.1, 23.6.

((3aR,4R,6R,6aR)-6-Cyano-6-(2,4-dioxo-3,4-dihydropyrimidin-1(2H)-yl)-2,2-dimethyltetrahydrofuro[3,4-d][1,3]dioxol-4-yl)methyl isobutyrate (19): To a suspension of 18 (65 mg, 0.21 mmol) in 2 mL of acetonitrile was added DMAP (5.1 mg, 0.04 mmol), DBU (66 mL, 0.44 mmol) and isobutyric anhydride (42 mL, 0.25 mmol). The mixture was stirred overnight and volatils removed under vacuum. The residue was purified by flash chromatography (DCM to DCM/MeOH=10:1) to give compound 19 (66 mg, 83%) as a white solid. ¹H NMR (CDCl₃, 400 MHz): δ 9.73 (bs, 1H), 7.62 (d, J=8. Hz, 1H), 5.81 (d, J=8.4 Hz, 1H), 5.08 (d, J=5.7 Hz, 1H), 4.95 (bs, 1H), 4.80 (d, J=5.2 Hz, 1H), 4.30 (m, 2H), 2.43 (m, 1H), 1.75 (s, 3H), 1.42 (s, 3H), 1.12 (d, J=7.7 Hz, 3H), 1.10 (d, J=7.6 Hz, 3H). ¹³C NMR (CDCl₃, 400 MHz): d 176.0, 163.0, 149.5, 137.7, 115.8, 113.2, 102.7, 94.2, 88.6, 86.3, 81.2, 63.4, 33.9, 26.2, 24.9, 18.9, 18.6.

((2R,3S,4R,5R)-5-Cyano-5-(2,4-dioxo-3,4-dihydropyrimidin-1(2H)-yl)-3,4-dihydroxy-tetrahydrofuran-2-yl)methyl isobutyrate (20): A solution of compound 19 (62 mg, 0.16 mmol) in 5 mL of formic acid and 0.5 mL of water was stirred at room temperature for 6 hours. Volatiles were removed under vacuum and the residue purified by flash chromatography (DCM to DCM/MeOH=10:1) to give compound 20 (48 mg, 86%) as a white solid. ¹H NMR (CD₃OD, 400 MHz): δ 7.93 (d, J=8.3 Hz, 1H), 5.78 (d, J=8.3 Hz, 1H), 4.62 (d, J=4.6 Hz, 1H), 4.46 (m, 3H), 4.04 (dd, J=8.8, 4.6 Hz, 1H), 2.62 (m, 1H), 1.20 (d, J=7.0 Hz, 3H), 1.19 (d, J=7.0 Hz, 3H). 13C NMR (CD₃OD, 400 MHz): d 176.6, 165.4, 150.8, 138.0, 113.9, 101.8, 91.6, 82.7, 76.2, 68.3, 61.5, 33.7, 18.0, 17.8.

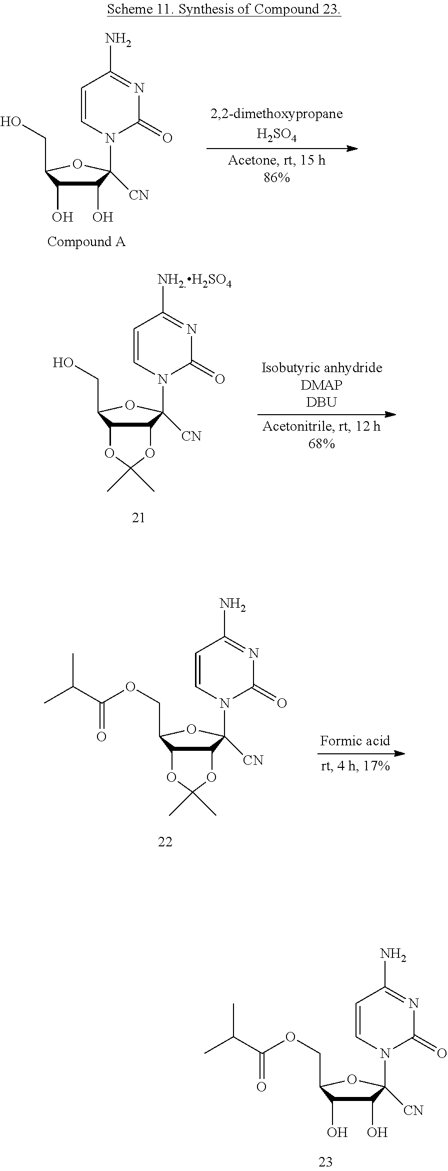

(3aR,4R,6R,6aR)-4-(4-Amino-2-oxopyrimidin-1(2H)-yl)-6-(hydroxymethyl)-2,2-dimethyltetrahydrofuro[3,4-d][1,3]dioxole-4-carbonitrile. H₂SO₄ salt (21): To a suspension of Compound A (66 mg, 0.24 mmol) in acetone was added 2,2-dimethoxypropane (168 μL, 1.37 mmol) and sulfuric acid (35 μL) at room temperature. The reaction mixture was stirred at room temperature for 15 h. The solid was filtered, washed with acetone and diethyl ether. The half white solid (79.8 mg, 86%) was dried under high vacuum overnight and used in the next step without any further purification. LC-MS: m/z: 309.0 [M+H].

((3aR,4R,6R,6aR)-6-(4-Amino-2-oxopyrimidin-1(2H)-yl)-6-cyano-2,2-dimethyltetra-hydrofuro[3,4-d][1,3]dioxol-4-yl)methyl isobutyrate (22): To a suspension of 21 (53.6 mg, 0.14 mmol) in acetonitrile was added isobutyric anhydride (25.5 μL, 0.15 mmol), DBU (43.8 μL, 0.29 mmol) and DMAP (3.5 mg, 0.028 mmol) at room temperature. The resulting reaction mixture was stirred at room temperature for 12 h. Solvents were evaporated and the crude solid was purified by flash column chromatography (methanol:dichloromethane: 0 to 5%) to afford compound 22 (36.2 mg, 68%). ¹H NMR (400 MHz, CDCl₃) δ 7.63 (d, J=8 Hz, 1H), 5.77 (d, J=7.6 Hz, 1H), 5.07 (d, J=6 Hz, 1H), 4.89 (q, J=4 Hz, 1H), 4.74 (dd, J=5.6 Hz, J=1.6 Hz, 1H), 4.29 (dd, J=54.8 Hz, J=2.8 Hz, 2H), 2.37 (q, J=7.2 Hz, 1H), 1.74 (s, 3H), 1.40 (s, 3H), 1.10 (t, J=6.8 Hz, 6H). LC-MS: m/z: 309.0 [M+H]. 401.0 [M+Na].

((2R,3S,4R,5R)-5-(4-Amino-2-oxopyrimidin-1(2H)-yl)-5-cyano-3,4-dihydroxytetrahydro-furan-2-yl)methyl isobutyrate (23): Compound 22 (34 mg, 0.089 mmol) was treated with neat formic acid (1.5 mL) at room temperature. The resulting reaction mixture was stirred for 4 h. Volatils were removed and the crude product was purified by flash column chromatography (methanol:dichloromethane: 0 to 10%) to afford compound 23 as white solid (5.1 mg, 17%). ¹H NMR (400 MHz, MeOH-d₄) δ 7.83 (d, J=8 Hz, 1H), 5.86 (d, J=7.6 Hz, 1H), 4.44 (d, J=1.2 Hz, 1H), 4.42 (m, 1H), 4.31 (t, J=3.6 Hz, 1H), 3.89 (m, 1H), 2.49 (q, J=7.2 Hz, 1H). LC-MS: m/z: 339.1 [M+H]. 361.0 [M+Na].

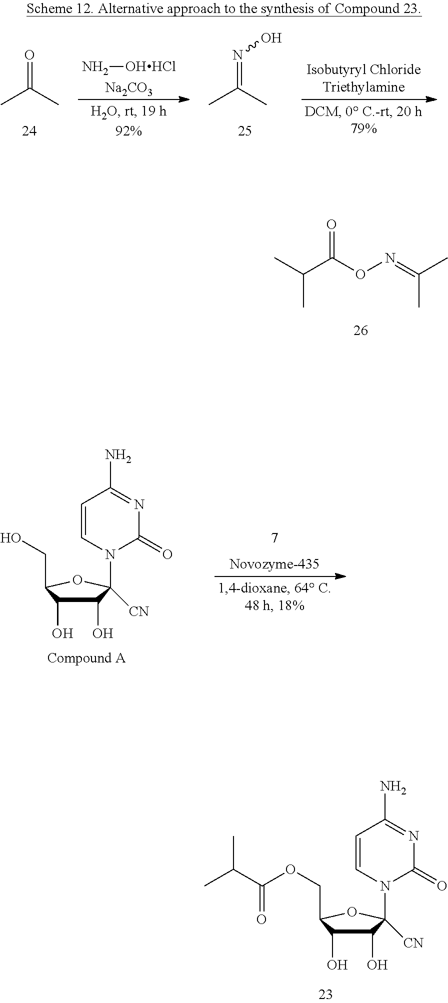

Acetone oxime (25): To a solution of acetone (11.0 mL, 150 mmol) and hydroxylamine hydrochloride (15.6 g. 225 mmol) in HPLC grade water (300 mL) was added Na₂CO₃ (28.6 g, 270 mmol) at room temperature. The resulting reaction mixture was stirred at room temperature for 20 h, after which it was extracted with diethyl ether (5×100 mL). The combined organic layers were washed with brine (100 mL) and dried over sodium sulphate, filtered and concentrated under reduced pressure to obtained acetone oxime 25 as a white solid (6.8 g, 61.7%). ¹H NMR (400 MHz, MeOH-d₄) δ 9.22 (bro.s, 1H), 1.90 (s, 3H), 1.89 (s, 3H).

Acetone oxime O-isobutyryl ester (26): To an ice cold solution of acetone oxime 25 (1.0 g, 13.6 mmol) in dichloromethane (30 mL) was added isobutyryl chloride (1.56 mL, 149 mmol) and triethylamine (0.89 mL, 163 mmol). The resulting reaction mixture was warmed to room temperature slowly and stirred for 20 h. The reaction mixture was washed with water (2×10 mL), 5% NaHCO₃ (2×10 mL), water (1×10 mL), 1 N aq HCl (2×10 mL), water (1×15 mL) and saturated brine (1×10 mL), dried over Na₂SO₄, concentrated to give acetone oxime 0-isobutyryl ester 26 as a colourless liquid (1.56 g, 79%). ¹H NMR (400 MHz, MeOH-d₄) δ 2.67 (q, J=7 Hz, 1H), 2.05 (s, 3H), 2.0 (s, 3H), 1.25 (s, 3H), 1.23 (s, 3H).

((2R,3S,4R,5R)-5-(4-Amino-2-oxopyrimidin-1(2H)-yl)-5-cyano-3,4-dihydroxytetrahydrofuran-2-yl)methyl isobutyrate (23): Novozyme-435 (45 mg) was rinsed with 1,4-dioxane (2×1 mL) and dried under high vacuum for 20 min. To a suspension of compound A (30 mg, 0.119 mmol) in 1,4-dioxane (1.5 mL) was added above rinsed Novozyme-435 and acetone oxime 0-isobutyryl ester 7 (70.65 mg, 0.476 mmol) under nitrogen atmosphere. The resulting reaction mixture was stirred at 64° C. for 48 h. The reaction mixture was filtered, washed with 1,4-dioxane (5 mL) and concentrated under reduced pressure. The crude product was purified by flash column chromatography (methanol:dichloromethane: 0 to 5%) to give 23 as white solid (8.6 mg, 18%). ¹H NMR (400 MHz, MeOH-d₄) δ 7.83 (d, J=8 Hz, 1H), 5.86 (d, J=7.6 Hz, 1H), 4.44 (d, J=1.2 Hz, 1H), 4.42 (m, 1H), 4.31 (t, J=3.6 Hz, 1H), 3.89 (m, 1H), 2.49 (q, J=7.2 Hz, 1H). LC-MS: m/z: 339.1 [M+H]. 361.0 [M+Na].

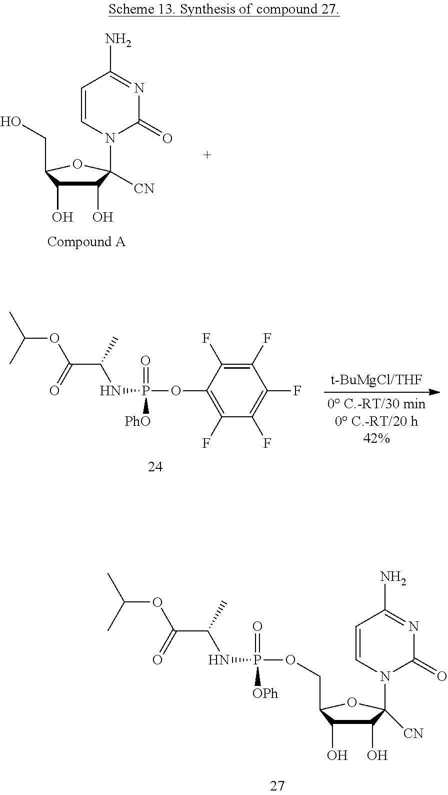

Isopropyl ((S)-(((2R,3S,4R,5R)-5-(4-amino-2-oxopyrimidin-1(2H)-yl)-5-cyano-3,4-dihydroxytetrahydrofuran-2-yl)methoxy)(phenoxy)phosphoryl)-L-alaninate (27): To a stirring suspension of compound A (45 mg, 0.17 mmol) in THF (1.5 mL) at 0° C. was added t-BuMgCl (1 M solution in THF, 0.2 mL, 0.2 mmol) dropwise. The mixture was stirred at rt for 30 min, and then cooled to 0° C. A solution of (S)-2-[1s]-(2,3,4,5,6-pentafluorophenoxy)-phenoxyphosphorylamino]propionic acid isopropyl ester (2, 91 mg, 0.2 mmol) in THF (1.5 mL) was added dropwise. After the addition, the mixture was stirred at rt overnight. The reaction was quenched by addition of isopropanol (0.2 mL). The solvent was evaporated, and the residue was purified by flash chromatography on silica gel eluting with CH₂Cl₂-MeOH (from 95:5 to 9:1) to give 38 mg (42%) of product 25 as a white powder. LC-MS m/e: 538.2 (M+1)⁺. ¹H-NMR (CD₃OD) d: 7.95 (d, J=7.7 Hz, 1H), 7.38-7.22 (m, 5H), 5.87 (d, J=7.7 Hz, 1H), 5.01-4.93 (m, 1H), 4.55-4.45 (m, 3H), 4.37-4.32 (m, 1H), 4.06-4.02 (m, 1H), 3.94-3.87 (m, 1H), 1.37-1.35 (d, J=7.0 Hz, 3H), 1.29-1.20 (m, 6H). ¹³C-NMR (CD₃OD) d 172.9, 166.4, 155.9, 150.6, 139.0, 129.5, 124.9, 120.1, 114.2, 95.4, 92.0, 83.4, 76.5, 68.8, 68.2, 64.2, 50.3, 20.6, 20.0. ³¹P-NMR (CD₃OD) d: 3.7.

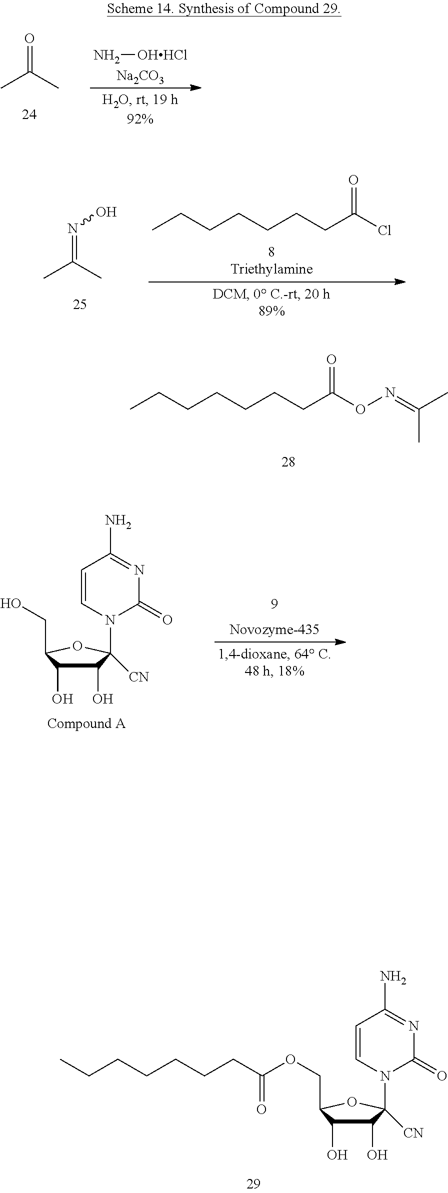

Propan-2-one O-octanoyl oxime (28): To an ice-cold solution of acetone oxime-25 (449 mg, 6.14 mmol) in dichloromethane (30 mL) was added octanoyl chloride (1.04 mL, 6.14 mmol) and triethylamine (1.02 mL, 7.37 mmol). The resulting reaction mixture was warmed to room temperature slowly and stirred for 20 h. The reaction mixture was washed with water (2×5 mL), 5% NaHCO₃ (2×5 mL), water (1×5 mL), 1 N aq HCl (2×5 mL), water (1×10 mL), brine (1×10 mL), dried over Na₂SO₄, concentrated to give propan-2-one O-octanoyl oxime 28 as a pale yellow liquid (878 mg, 72%). ¹H NMR (400 MHz, CDCl₃) δ 2.4 (t, J=8 Hz, 2H), 2.05 (s, 3H), 1.99 (s, 3H), 1.69 (m, 2H), 1.33 (m, 8H), 0.88 (t, J=6.8 Hz, 3H). ¹³C NMR (101 MHz, CDCl₃) δ 171.1, 163.5, 32.7, 31.5, 28.9, 24.7, 22.4, 21.7, 21.3, 16.7, 14.8, 13.8.

((2R,3S,4R,5R)-5-(4-amino-2-oxopyrimidin-1(2H)-yl)-5-cyano-3,4-dihydroxytetrahydrofuran-2-yl)methyl octanoate (29): Novozym-435 (150 mg) was rinsed with 1,4-dioxane (2×5 mL) and dried under high vacuum for 20 min. To a suspension of compound A (100 mg, 0.37 mmol) in 1,4-dioxane (5 mL) was added Novozym-435 and propan-2-one O-octanoyl oxime 28 (297.4 mg, 1.49 mmol) under a nitrogen atmosphere. The resulting reaction mixture was heated to 64° C. for 48 h. The reaction mixture was filtered, washed with 1,4-dioxane (5 mL) and concentrated under reduced pressure. The crude product was purified by flash column chromatography (methanol:dichloromethane: 0 to 5%) to give 29 as a white solid (67.8 mg, 46%). ¹H NMR (400 MHz, MeOH-d₄) δ 7.96 (d, J=8 Hz, 1H), 5.99 (d, J=8 Hz, 1H), 4.56 (d, J=4.8 Hz, 1H), 4.52 (m, 1H), 4.48 (m, 2H), 3.98 (m, 1H), 2.37 (m, 2H), 1.62 (m, 2H), 1.32 (m, 8H), 0.93 (t, J=4 Hz, 1H 2H). ¹³C NMR (101 MHz, MeOH-d₄) δ 173.2, 166.5, 155.9, 138.9, 114.2, 95.2, 92.0, 82.9, 76.5, 68.7, 61.5, 33.5, 31.4, 28.7, 28.6, 24.5, 22.2, 13.0. LC-MS: m/z: 395.3 [M+H].

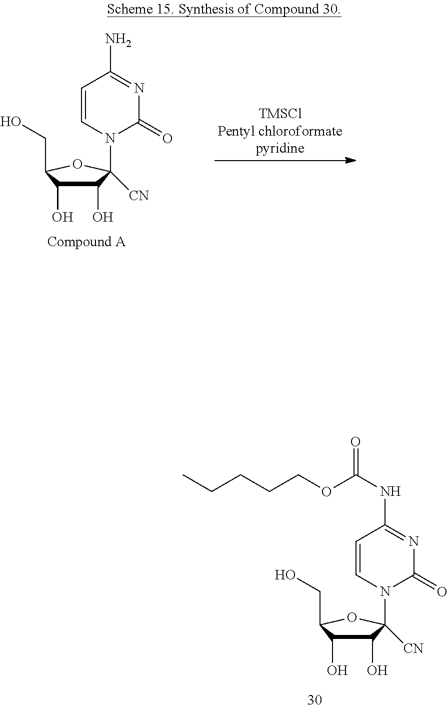

Pentyl (1-((2R,3R,4S,5R)-2-Cyano-3,4-dihydroxy-5-(hydroxymethyl)tetrahydrofuran-2-yl)-2-oxo-1,2-dihydropyrimidin-4-yl)carbamate (30). 1′-Cyano-cytidine (134 mg, 0.5 mmol) was co-evaporated with pyridine and then dissolved in dry pyridine (4 mL). TMSCl (0.33 mL, 2.5 mmol) was added, and the solution was stirred at RT under argon for 2 h. Pentyl chloroformate (0.37 mL, 2.5 mmol) was added, and the mixture was stirred at RT overnight. The mixture was cooled to 0° C., and water (0.5 mL) was added, and the mixture was stirred for 20 min. The solvent was evaporated under reduced pressure, and the residue was purified by flash chromatography on silica gel eluting with DCM-MeOH (95:5 to 9:1) to give 144 mg (75%) of product 30 as a white powder. LCMS (ES+): 383.1 (M+1)*; H¹—NMR (MeOH-d₄): δ 8.42 (d, J=19.5 Hz, 1H), 7.21 (d, J=19.5 Hz, 1H), 4.36 (d, J=13.5 Hz, 1H), 4.21-4.18 (m, 1H), 4.05 (t, J=16.6 Hz, 2H), 3.93-3.86 (m, 2H), 3.65-3.61 (m, 1H), 1.58-1.53 (m, 2H), 1.27-1.22 (m, 4H), 0.81-0.78 (m, 3H). ¹³C-NMR δ 164.5, 155.5, 153.2, 143.0, 113.9, 95.6, 92.1, 85.8, 76.4, 67.3, 65.9, 58.5, 28.1, 27.6, 22.0, 12.9.

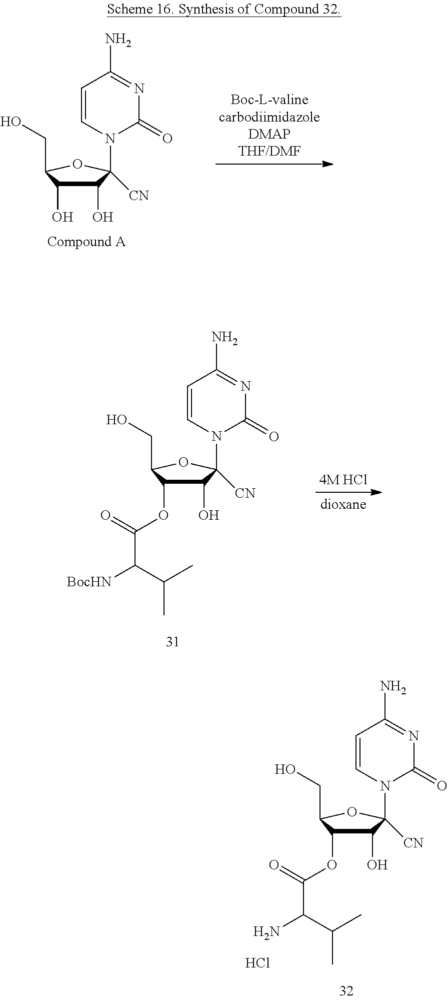

1′-Cyano-3′-Boc-L-valine ester-cytidine (31): A solution of Boc-L-valine (90.8 mg, 0.42 mmol) and carbodiimidazole (67.9 mg, 0.42 mmol) in THE (2 mL) was stirred at room temperature for 1.5 h then heated at 40-50° C. for 30 minutes. The above mixture was added via a cannula to a mixture of 1′-cyano-cytidine (102 mg, 0.38 mmol), DMAP (5 mg, 0.05 mmol) and triethylamine (1.5 ml, 10.8 mmol) in DMF (6 mL) at 70-80° C. and the resulting mixture was stirred at this temperature for 2 hours. After being cooled down to room temperature, volatiles were removed under vacuum. The residue was purified by flash chromatography (100% DCM to DCM/MeOH=5:1) to give the desired product 31 (140 mg, 79%). ¹H NMR (CD₃OD, 400 MHz) δ 8.15 (d, J=7.76 Hz, 1H), 5.96 (d, J=7.76 Hz, 1H), 4.83 (d, J=4.76 Hz, 1H), 4.59 (d, J=7.64 Hz, 1H), 4.12 (d, 5.76 Hz, 1H), 4.0 (dd, J=2.2, 13.0 Hz, 1H), 3.72 (dd, J=2.68, 13.0 Hz, 1H), 2.19 (m, 1H), 1.47 (s, 9H), 1.01 (d, J=6.88 Hz, 3H), 0.96 (d, J=6.8 Hz, 3H). LC-MS calcdt for C₂₀H₃₀N₅O₈ (M+H): 468.2, found: 468.3.

1′-Cyano-3′-L-valine ester-cytidine HCl salt 32: To a solution of compound 31 (49 mg, 0.10 mmol) in dioxane (3 mL) was added 4M HCl in dioxane (3 mL). The mixture was stirred for 2 hours at 0° C., the precipitate was collected and washed with dioxane twice to give the desired product 32 (40 mg, 95%) as a white solid. ¹H NMR (CD₃OD, 400 MHz) δ: 8.40 (d, J=8.08 Hz, 1H), 7.61 (s, 2H), 6.22 (d, J=8.08 Hz, 1H), 5.28 (m, 1H), 5.02 (d, J=4.84 Hz, 1H), 4.73 (d, J=6.8 Hz. 1H), 4.08 (m, 2H), 3.8 (d, J=11.4 Hz, 1H), 2.40 (m, 1H), 1.13 (d, J=6.92 Hz, 6H), ¹³C NMR (CD₃OD, 400 MHz) d: 167.5, 160.4, 147.6, 112.9, 94.2, 92.3, 85.0, 75.6, 71.9, 66.7, 58.5, 58.0, 29.6, 17.0, 16.9. LC-MS calcdt for C₁₅H₂₂N₅O₆ (M+H): 368.15, found: 368.1.

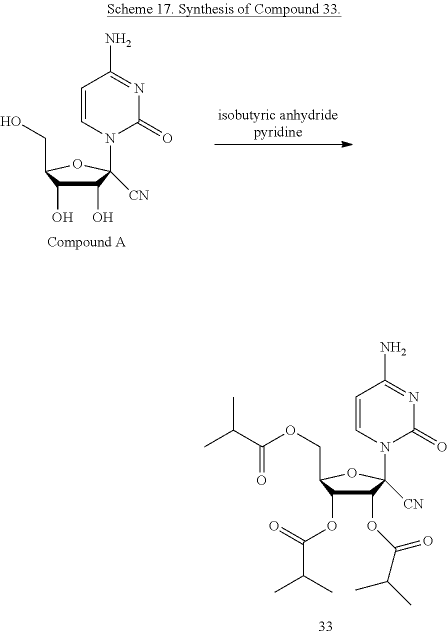

(2R,3R,4R,5R)-2-(4-Amino-2-oxopyrimidin-1(2H)-yl)-2-cyano-5-((isobutyryloxy)methyl)-tetrahydrofuran-3,4-diyl bis(2-methylpropanoate) (33): A solution 1′-cyano-cytidine (60 mg, 0.22 mmol) and isobutyric anhydride (0.3 mL, 1.8 mmol), in pyridine (2 mL) was heated at 80° C. for 1 hour. Volatiles were removed under vacuum and the residue was dissolved in methanol (8 mL) and heated at 105° C. overnight. Volatiles were removed under vacuum and the residue was purified by flash chromatography (100% DCM to DCM/MeOH=10:1) to give product 33 (74 mg, 69%). ¹H NMR (CD₃OD, 400 MHz) δ: 7.92 (d, J=7.76 Hz, 1H), 6.01 (d, J=7.6 Hz, 1H), 5.94 (d, J=5.04 Hz, 1H), 5.30 (dd, J=5.08, 6.64 Hz, 1H), 4.83 (m, 1H), 4.45 (m, 2H), 2.75 (m, 1H), 2.57 (m, 2H), 1.31 (d, J=6.9 Hz, 3H), 1.27 (d, J=6.96 Hz, 3H), 1.17 (m, 12H). ¹³C NMR (CD₃OD, 400 MHz) δ: 176.1, 175.2, 174.2, 166.6, 155.5, 138.5, 118.2, 95.7, 89.3, 82.1, 75.1, 68.8, 61.2, 33.9, 33.7, 33.6, 18.0, 17.82, 17.75, 17.73, 17.69, 17.65. LC-MS calcd for C₂₂H₃₁N₄O₈ (M+H): 479.21, found: 479.2.

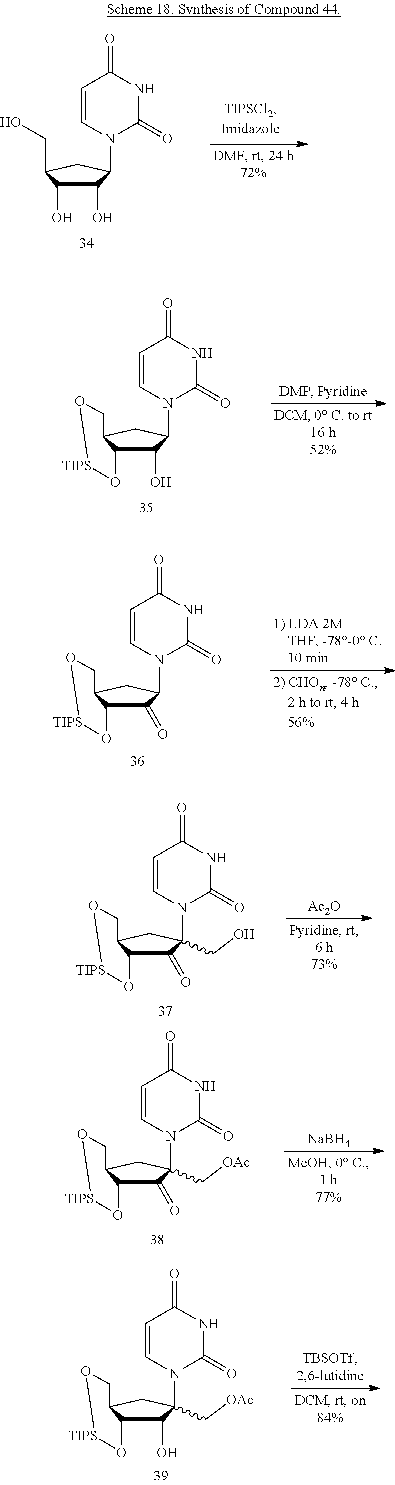

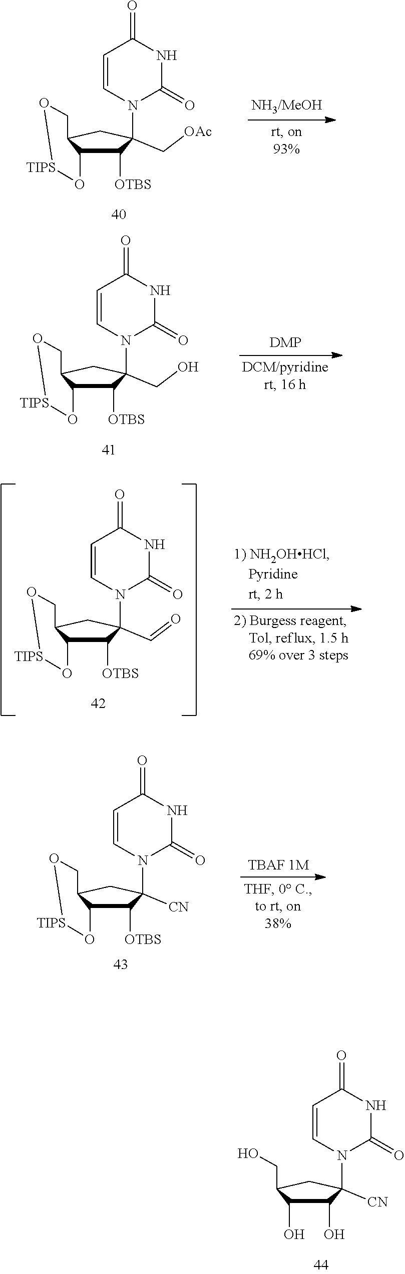

Carbocyclic uridine 10 was prepared according to the chemistry described in Nucleosides, Nucleotides and Nucleic Acids, 2012, 31:4, 277-285.

1-((6aR,8R,9S,9aR)-9-Hydroxy-2,2,4,4-tetraisopropylhexahydro-cyclopenta[f][1,3,5]-trioxocin-8-yl)pyrimidine-2,4(1H,3H)-dione 35. To a solution of carbocyclic uridine 34 (1 g, 4.13 mmol) in pyridine (40 ml, 0.1M) at 0° C. was added 1,3-dichloro-1,1,3,3-tetraisopropyldisiloxane (1.48 ml, 4.51 mmol, 1.1 eq.) dropwise. The reaction mixture was stirred at room temperature for 12 h and evaporated to dryness. The residue was diluted in a saturated solution of NaHCO₃ (100 ml), extracted with ethyl acetate (3×75 ml). The organic phases were combined, washed with brine (60 ml), dried over MgSO₄, filtered, and concentrated in vacuo to dryness. The crude product was purified by silica gel column chromatography (Hexane/Ethyl acetate 100/0 to 0/100) to give the title compound (1.44 g, 72%) as a white foam. ¹H NMR (400 MHz, Acetone-d₆) 9.93 (s, 1H), 7.54 (dd, 1H, J=8.4, 3.5 Hz), 5.54 (d, 1H, J=7.8 Hz,), 5.46 (dd, 1H, J=6.4, 3.7 Hz), 4.63 (dd, 1H, J=9.4, 6.2 Hz), 4.48 (ddd, 1H, J=10.4, 8.5, 3.8 Hz), 4.02 (dd, 1H, J=11.8, 3.4 Hz), 3.84 (dd, 1H, J=11.7, 3.9 Hz), 3.09 (q, 2H, J=5.4 Hz), 2.18-2.10 (m, 1H), 2.05-1.89 (m, 1H), 1.14-0.92 (m, 28H). ¹³C NMR (101 MHz, Acetone-d₆) δ 170.2 163.7, 151.5, 145.4, 102.2, 76.4, 71.7, 64.8, 61.4, 46.5, 20.8, 17.9, 17.9, 17.8, 17.8, 17.5, 17.5, 14.1, 13.7, 13.6. HRMS-ESI (m/z) [M+Na]⁺ calcd. 507.2425. for C₂₂H₄₀N₂O₆Si₂Na, found 507.4026.

(1S,2S,3R,4R)-2-((tert-Butyldimethylsilyl)oxy)-1-(2,4-dioxo-3,4-dihydropyrimidin-1(2H)-yl)-3-hydroxy-4-(hydroxymethyl)cyclopentane-1-carbonitrile 36. Under argon, at 0° C., Dess-Martin periodinane (3.5 g, 8.2 mmol, 15 eq.) was added to a solution of 35 (2.66 g, 5.49 mmol, 1 eq.) in CH₂Cl₂ (42 ml, 0.13M) and pyridine (4.5 ml, 54.9 mmol, 10 eq.). The resulting mixture was stirred at room temperature for 16 h before addition of dichloromethane (100 ml) and a saturated solution of NaHCO₃ (70 ml). The precipitate was filtered off, and the aqueous layer was extracted with dichloromethane (5×60 ml). The organic phases were combined, dried over MgSO₄, filtered, and concentrated in vacuo. The crude product was purified by silica gel column chromatography (hexane/ethyl acetate 100/0 to 0/100) to give the title compound (1.31 g).

1-((6aR,8R,9aR)-2,2,4,4-Tetraisopropyl-9-oxohexahydro-cyclopenta[f][1,3,5]trioxocin-8-yl)pyrimidine-2,4(1H,3H)-dione 37. Under argon, to a solution of compound 36 (1.31 g, 2.71 mmol, 1 eq.) in THE (30 ml, 0.09 M) was LDA (2.5 M, 2.28 ml, 5.7 mmol, 2.1 eq.) dropwise at −78° C. The temperature was raised to 0° C. for 10 min, before being cooled down to −78° C. Paraformaldehyde (CHOₙ 0.65 g, 21.68 mmol, 8 eq.) was added. Reaction was allowed to rise at room temperature over a period of 6 hours. After completion, the mixture was neutralized with HCl (1M), diluted with a saturated solution of NaHCO₃ (100 ml), extracted with ethyl acetate (3×75 ml). The organic phases were combined, washed with brine (60 ml), dried over MgSO₄, filtered, and concentrated in vacuo to dryness. The crude product was purified by flash chromatography (Hexane/Ethyl acetate 100/0 to 30/70) to give the title compound (0.77 g, 56%) as a white foam. ¹H NMR (400 MHz, Acetone-d₆) 10.09 (s, 1H), 8.03 (d, 1H, J=8.4 Hz), 5.64 (d, 1H, J=8.3 Hz), 5.04 (t, 1H, J=5.4 Hz,), 4.98 (d, 1H, J=10.4 Hz), 4.10-3.84 (m, 4H), 2.45-2.41 (m, 1H), 2.19-1.96 (m, 2H), 1.14-0.92 (m, 28H). ¹³C NMR (101 MHz, Acetone-d₆) δ 171.8 164.2, 152.7, 144.7, 103.1, 76.4, 69.5, 63.4, 61.8, 61.4, 44.0, 28.4, 18.8, 18.7, 18.7, 18.6, 18.5, 18.3, 18.3, 15.1, 14.8, 14.3, 14.0. HRMS-ESI (m/z) [M+H]⁺ calcd. 513.2374. for C₂₃H₄₁N₂O₇ Si₂, found 513.2441.

1-((6aR,8S,9aR)-8-(hydroxymethyl)-2,2,4,4-Tetraisopropyl-9-oxohexahydrocyclo-penta[f][1,3,5]trioxocin-8-yl)pyrimidine-2,4(1H,3H)-dione 38. At 0° C., to a solution of 37 (0.54 g, 1.05 mmol, 1 eq.) in pyridine (15 ml) was added Ac₂O (0.16 ml, 1.68 mmol, 1.6 eq.). The reaction was stirred at room temperature for 4 hours before being diluted with ethyl acetate (100 ml). The organic layer was washed with HCl 1 M (25 ml), sat. NaHCO₃ (40 ml) and brine (30 ml). The organic phase was dried over MgSO₄, filtered, and concentrated in vacuo to dryness. The crude product was purified by flash chromatography (hexane/ethyl acetate 100/0 to 50/50) to give the title compound (0.42 g, 73%) as a white foam. ¹H NMR (400 MHz, Acetone-d₆) δ) 10.15 (s, 1H), 7.81 (d, 1H, J=8.2 Hz), 5.63 (d, 1H, J=8.2 Hz), 5.01-4.95 (m, 1H), 4.60 (d, 1H, J=13.4 Hz), 4.31 (d, 1H, J=13.4 Hz), 4.08 (d, 1H, J=14.1 Hz), 3.91 (d, 1H, J=14.1 Hz), 2.29-2.21 (m, 3H), 2.12 (s, 3H), 1.29-0.86 (m, 28H). ¹³C NMR (101 MHz, Acetone-d₆) δ 206.1, 169.4 164.1, 150.9, 142.0, 101.8, 74.6, 66.0, 63.4, 61.7, 60.9, 42.2, 27.9, 19.7, 17.0, 16.9, 16.9, 16.8, 16.5, 16.4, 13.4, 13.3, 13.0, 12.6, 12.3. HRMS-ESI (m/z) [M+H]⁺ calcd. 555.2480. for C₂₅H₄₃N₂O₈ Si₂, found 555.2574.

((6aR,8S,9aR)-8-(2,4-Dioxo-3,4-dihydropyrimidin-1(2H)-yl)-2,2,4,4-tetraisopropyl-9-oxohexahydrocyclopenta[f][1,3,5]trioxocin-8-yl)methyl acetate 39. To a solution of 38 (1.01 g, 1.82 mmol, 1 eq.) in methanol (52 ml, 0.035M) at 0° C. was added NaBH₄ (0.19 g, 5.18 mmol, 2.85 eq.). After 1.5 hours, the mixture was diluted with sat. NH₄Cl (100 ml) and extracted with ethyl acetate (3×50 ml). The organic phases were combined, washed with brine (40 ml), dried over MgSO₄, filtered, and concentrated in vacuo to dryness. The crude product was purified by flash chromatography (Hexane/Ethyl acetate 100/0 to 40/60) to give the title compound (0.77 g, 77%) as a white foam. ¹H NMR (400 MHz, Acetone-d₆) δ 9.89 (s, 1H), 7.98 (d, 1H, J=8.3 Hz), 5.56 (d, 1H, J=8.4 Hz), 5.73 (d, 1H, J=12.1 Hz), 4.53-4.49 (m, 2H), 4.40-4.37 (m, 1H), 4.14 (d, 1H, J=7.5 Hz), 4.00 (dd, 1H, J=11.6, 3.8 Hz), 3.84 (dd, 1H, J=11.8, 1.9 Hz), 2.81-2.74 (m, 1H), 2.38-2.33 (m, 1H), 1.94 (s, 3H), 1.61-1.64 (m, 1H), 1.13-0.86 (m, 28H). ¹³C NMR (101 MHz, Acetone-d₆) δ 170.0, 162.6, 151.5, 143.6, 100.7, 76.9, 72.9, 70.6, 64.7, 63.0, 46.4, 32.4, 19.8, 17.8, 17.7, 17.7, 17.7, 17.6, 17.3, 14.3, 14.1, 13.6, 13.5. HRMS-ESI (m/z) [M+H]⁺ calcd. 557.2636. for C₂₅H₄₅N²O₈Si₂, found 557.2723.

((6aR,8S,9S,9aR)-8-(2,4-Dioxo-3,4-dihydropyrimidin-1(2H)-yl)-9-hydroxy-2,2,4,4-tetraisopropylhexahydrocyclopenta[f][1,3,5]trioxocin-8-yl)methyl acetate 40. 2,6-lutidine (0.12 ml, 1 mmol, 4 eq.) and TBSOTf (0.12 ml, 0.5 mmol, 2 eq.) were added to a solution of 39 (140 mg, 0.25 mmol, 1 eq.) in THE (2.5 ml, 0.1M) at 0° C. After 12 hours at room temperature, the reaction was diluted with water (20 ml), extracted with ethyl acetate (3×20 ml). The organic layers were combined, washed with brine (15 ml), dried over MgSO₄, filtered, and concentrated in vacuo to dryness. The crude product was purified by flash chromatography (Hexane/Ethyl acetate 100/0 to 50/50) to give the title compound (140 mg, 84%) as a white foam. ¹H NMR (400 MHz, Acetone-d₆) ¹H NMR (400 MHz, Acetone-d₆) δ 9.90 (s, 1H), 7.75 (d, 1H, J=8.3 Hz), 5.53 (dt, 1H, J=8.4, 1.2 Hz), 5.12 (d, 1H, J=4.0 Hz), 4.68 (dd, 1H, J=11.4, 1.3 Hz), 4.40 (d, 1H, J=11.4 Hz), 4.02 (dd, 1H, J=11.9, 2.7 Hz), 3.96 (dd, 1H, J=10.2, 4.0 Hz), 3.84 (dd, 1H, J=11.8, 1.9 Hz), 2.41-2.23 (m, 2H), 2.17-2.06 (m, 1H), 1.96 (s, 3H), 1.13-0.86 (m, 37H), 0.23 (s, 3H), 0.21 (s, 3H). ¹³C NMR (101 MHz, Acetone-d₆) δ 206.1, 170.7, 163.3, 152.2, 143.7, 101.7, 77.6, 73.3, 71.4, 65.3, 60.0, 42.4, 32.1, 26.5, 20.7, 19.0, 17.8, 17.7, 17.7, 17.7, 17.6, 17.3, 14.3, 14.1, 13.6, 13.5, −3.9, −4.1. HRMS-ESI (m/z) [M+H]⁺ calcd. 671.3501. for C₃₁H₅₉N²O₈Si₃, found 671.3593.

((6aR,8S,9S,9aR)-9-((tert-Butyldimethylsilyl)oxy)-8-(2,4-dioxo-3,4-dihydropyrimidin-1(2H)-yl)-2,2,4,4-tetraisopropylhexahydrocyclopenta[f][1,3,5]trioxocin-8-yl)methyl acetate 41. To a solution of 40 (0.43 g, 0.43 mmol, 1 eq.) in methanol (13 ml) was bubbled NH_{3(g)}. The reaction was stirred at room temperature for 24 hours, then evaporated to dryness and the residue purified by flash chromatography (Hexane/Ethyl acetate 100/0 to 50/50) to give the title compound (0.37 g, 93%) as a white foam. ¹H NMR (400 MHz, Acetone-d₆) δ 9.76 (s, 1H), 7.65 (d, 1H, J=8.4 Hz), 5.53-5.45 (m, 1H), 5.06 (d, 1H, J=4.2 Hz), 4.20 (dd, 1H, J=11.4, 5.6 Hz), 4.13-3.98 (m, 2H), 3.93 (dd, 1H, J=10.3, 4.3 Hz), 3.84 (dd, 1H, J=11.8, 2.2 Hz), 3.73 (dd, 1H, J=11.4, 6.1 Hz), 2.33-2.24 (m, 1H), 2.15 (d, 2H, J=9.0 Hz), 1.16-0.88 (m, 37H), 0.21 (s, 3H), 0.18 (s, 3H). ¹³C NMR (101 MHz, Acetone-d₆) δ 163.6, 152.3, 144.8, 101.0, 77.5, 73.6, 63.1, 60.3, 42.8, 31.7, 26.6, 19.1, 17.8, 17.8, 17.7, 17.7, 17.6, 17.3, 14.3, 14.1, 13.6, 13.5, −3.1, −3.2. HRMS-ESI (m/z) [M+H]⁺ calcd. 629.3395. for C₂₉H₅₇N₂O₇Si₃, found 629.3485.

1-((6aR,8S,9S,9aR)-9-((tert-Butyldimethylsilyl)oxy)-8-(hydroxymethyl)-2,2,4,4-tetraisopropylhexahydrocyclopenta[f][1,3,5]trioxocin-8-yl)pyrimidine-2,4(1H,3H)-dione. 43. To a solution of 41 (70 mg, 0.11 mmol, 1 eq.) in dichloromethane (2 ml, 0.13M) and pyridine (0.075 ml, 0.88 mmol, 8 eq.) at 0° C., under argon was added Dess-Martin periodinane (56 mg, 0.132 mmol, 1.2 eq.). The reaction mixture was stirred at room temperature for 16 h. The reaction mixture was then diluted with dichloromethane (20 ml) and sat. NaHCO₃ (15 ml). The precipitate was filtered off and the aqueous layer was extracted with dichloromethane (5×20 ml). The organic layers were combined, dried over MgSO₄, filtered, and concentrated in vacuo. The residue was purified by flash chromatography (Hexane/Ethyl acetate 100/0 to 50/50) to give compound 42. To a solution of compound 42 in pyridine (2 ml) was added NH₂OH. HCl (32 mg, 0.46 mmol, 5 eq.). After 2 hours at room temperature, volatiles were removed under vacuum and the residue was dissolved in ethyl acetate (50 ml), washed with water (15 ml) and brine (15 ml). The organic layer was dried over MgSO₄, filtered, and concentrated in vacuo to dryness. The crude product was then dissolved in toluene (2.5 ml) and the Burgess reagent (0.1 g, 0.42 mmol, 5 eq.) was added to the solution. The reaction mixture was heated at 110° C. and after 2 hours at this temperature, the volatiles were removed under vacuum. The residue was dissolved in ethyl acetate (50 ml) and the organic layer washed with sat. NaHCO₃ (15 ml) and brine (15 ml), dried over MgSO₄, and concentrated in vacuo. The crude product was purified by flash chromatography (Hexane/Ethyl acetate 100/0 to 50/50) to give the title compound (47.9 mg, 69% over 3 steps) as a white foam. ¹H NMR (400 MHz, Acetone-d₆) δ 10.33 (s, 1H), 7.89 (d, 1H, J=8.4 Hz), 5.66 (d, 1H, J=8.4 Hz), 4.77 (d, 1H, J=3.6 Hz), 4.07 (ddd, 2H, J=20.5, 11.3, 3.4 Hz), 3.97 (dd, 1H, J=12.1, 1.9 Hz), 2.79-2.65 (m, 2H), 2.56 (tdd, 1H, J=9.0, 3.4, 1.7 Hz), 1.19-0.88 (m, 37H), 0.29 (s, 3H), 0.21 (s, 3H). ¹³C NMR (101 MHz, Acetone-d₆) δ 162.8, 151.1, 140.8, 118.7, 103.0, 78.7, 72.4, 66.0, 60.1, 42.7, 34.9, 26.3, 18.8, 18.0, 17.8, 17.8, 17.7, 17.5, 17.5, 17.4, 17.3, 14.1, 14.1, 13.9, 13.5, −3.9, −3.9. HRMS-ESI (m/z) [M+H]⁺ calcd. 624.3242. for C₂₉H₅₄N₂O₆Si₃, found 624.3331.

(6aR,8S,9S,9aR)-9-((tert-Butyldimethylsilyl)oxy)-8-(2,4-dioxo-3,4-dihydropyrimidin-1(2H)-yl)-2,2,4,4-tetraisopropylhexahydrocyclopenta[f][1,3,5]trioxocine-8-carbonitrile 44. TBAF 1M (0.33 ml, 0.33 mmol, 3 eq.) was added at 0° C. to a solution of 43 (70 mg, 0.11 mmol, 1 eq.) in THF (1.5 ml, 0.1M). After 12 hours at room temperature, the volatiles were removed under vacuum and the crude product was purified by flash chromatography (dichloromethane/methanol 100/0 to 90/10) to give the title compound (11.4 mg, 38%) as a white foam. ¹H NMR (400 MHz, methanol-d₄) δ 8.28 (d, 1H, J=7.8 Hz), 7.33 (d, 1H, J=7.8 Hz), 4.48 (d, 1H, J=6.3 Hz), 4.04 (dd, 1H, J=6.3, 4.0 Hz), 3.60 (h, 2H, J=5.4 Hz), 3.29-3.21 (m, 1H), 2.35 (tt, 1H, J=9.3, 4.6 Hz), 1.84 (dd, 1H, J=14.1, 9.6 Hz). ¹³C NMR (101 MHz, Methanol-d₄) δ 163.7, 156.2, 153.6, 145.3, 116.5, 95.7, 75.8, 71.1, 66.8, 61.7, 45.3, 34.8.

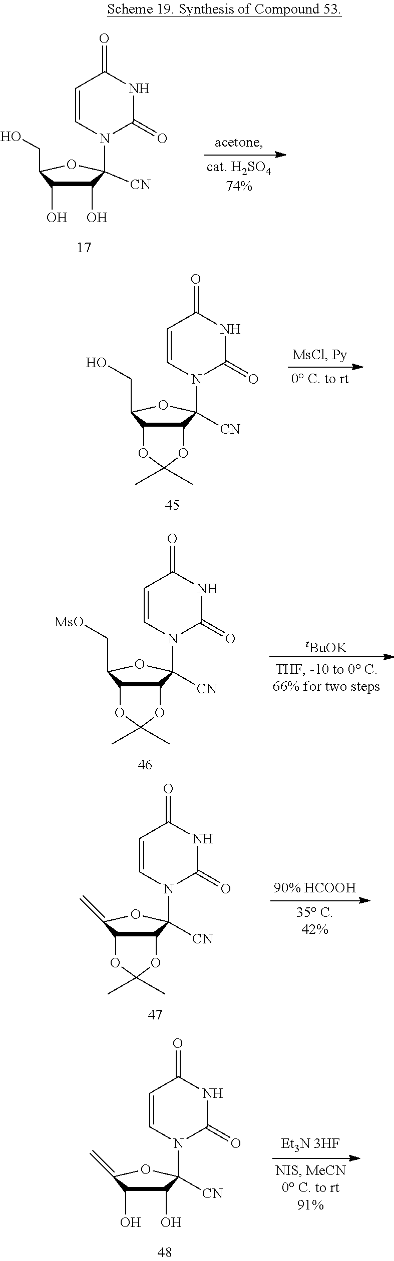

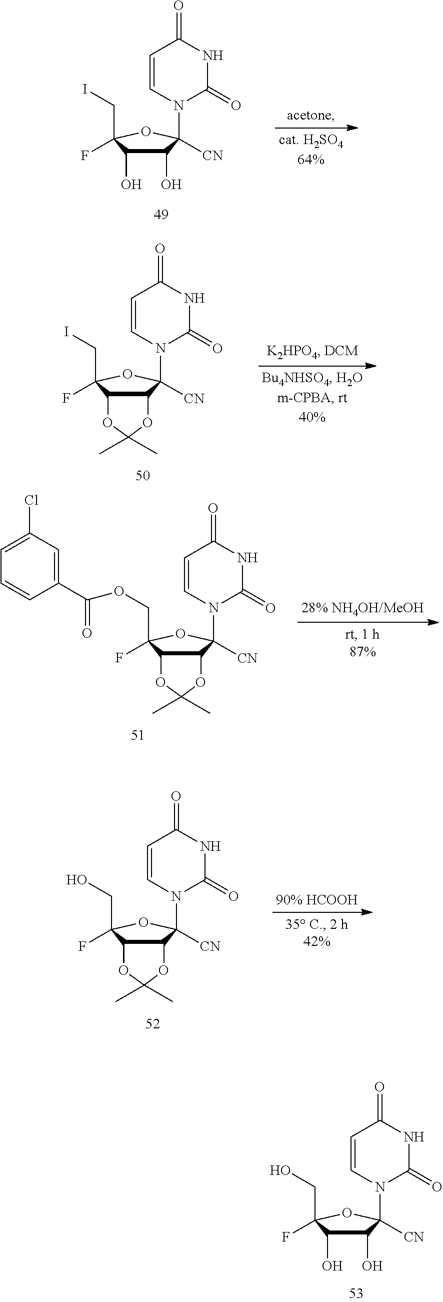

(3aR,4R,6R,6aR)-4-(2,4-Dioxo-3,4-dihydropyrimidin-1(2H)-yl)-6-(hydroxymethyl)-2,2-dimethyltetrahydrofuro[3,4-d][1,3]dioxole-4-carbonitrile 45: To a suspension of 17 (1.0 g, 3.7 mmol) in dry acetone (50 mL) and 2,2-dimethoxypropane, was added 10 drops of sulfuric acid. The reaction mixture was stirred at room temperature overnight until completion. Triethylamine (0.5 mL) was added and the mixture was stirred at rt for 10 min. After removal of the volatiles under vacuum, the solvents, the residue was purified by column chromatography (0-10% MeOH/DCM) to give compound 45 (850 mg, 74%). ¹H NMR (400.3 MHz): 1.39 (s, 3H), 1.66 (s, 3H), 3.69 (dd, J=12.2, 3.6 Hz, 1H), 3.79 (dd, J=12.2, 2.6 Hz, 1H), 4.81 (m, 1H), 4.90 (d, J=5.4 Hz, 1H), 5.02 (d, J=5.4 Hz, 1H), 5.72 (d, J=8.3 Hz, 1H), 7.89 (d, J=8.3 Hz, 1H).

(3aR,4R,6aS)-4-(2,4-Dioxo-3,4-dihydropyrimidin-1(2H)-yl)-2,2-dimethyl-6-methylenetetrahydrofuro[3,4-d][1,3]dioxole-4-carbonitrile 47: To a solution of 45 (960 mg, 3.1 mmol) in dry pyridine (15 mL) was added MsC1 (750 uL, 9.4 mmol) dropwise at 0° C. After the addition was completed, the ice-bath was removed and the mixture was stirred at rt until completion (˜1.5 h). After removal of the volatiles under vacuum, EtOAc (200 mL) was added and the organic layer was washed with water (2×50 mL), brine (50 mL) and dried over sodium sulfate. After removal of the solvent, the crude compound 46 was dried under high vacuum (˜1.03 g). Crude compound 3 was dissolved in anhydrous THE (30 mL), and the resulting solution was cooled down to −10° C. KO^{t}Bu (1.0 g) was then added portionwise (5 portions) and the mixture was stirred from −10° C. to 0° C. for 1.5 h until completion. The reaction mixture was filtered through a celite pad and the filtrate was dried and purified by column chromatography (0-5% MeOH/DCM) to give compound 47 (660 mg, 73%). ¹H NMR (400.3 MHz): 1.41 (s, 3H), 1.59 (s, 3H), 4.79 (d, J=3.1 Hz, 1H), 5.04 (d, J=3.0 Hz, 1H), 5.13 (d, J=5.8 Hz, 1H), 5.20 (d, J=5.8 Hz, 1H), 5.76 (d, J=8.2 Hz, 1H), 7.64 (d, J=8.3 Hz, 1H).

(2R,3R,4S)-2-(2,4-Dioxo-3,4-dihydropyrimidin-1(2H)-yl)-3,4-dihydroxy-5-methylenetetrahydrofuran-2-carbonitrile 48: A solution of 47 (660 mg) in 90% formic acid (10 mL) was stirred at rt for 10 h. After removal the volatiles under vacuum, the residue was purified by column chromatography (0-10% MeOH/DCM) to give compound 48 (239 mg, 42%). ¹H NRM (400.3 MHz): 4.49-4.51 (m, 1H), 4.55-4.56 (m, 1H), 4.62 (d, J=4.8 Hz, 1H), 4.85 (m, 1H), 5.78 (d, J=8.3 Hz, 1H), 7.51 (d, J=8.3 Hz, 1H).

(3aR,4R,6R,6aS)-4-(2,4-Dioxo-3,4-dihydropyrimidin-1(2H)-yl)-6-fluoro-6-(iodomethyl)-2,2-dimethyltetrahydrofuro[3,4-d][1,3]dioxole-4-carbonitrile 50: To a solution of 48 (225 mg, 0.9 mmol) in anhydrous acetonitrile (10 mL) was added NIS and Et₃N³HF at 0° C. The mixture was stirred at 0° C. for 1 h, and at rt for 5 h until completion. After removal of the volatiles under vacuum, EtOAc (200 mL) was added and the organic layer was washed with sat Na₂S₂O₃ (30 mL), water (30 mL) and brine (50 mL) and dried over sodium sulfate. After evaporation of the volatiles, the residue was purified by column chromatography (0-8% MeOH/DCM) to give compound 49 (325 mg, 91%).

A mixture of 49 (300 mg) in dry acetone (6 mL) and 2,2-dimethoxypropane was added 3 drops of sulfuric acid. The reaction mixture was stirred at room temperature overnight until completion. Triethylamine (0.2 mL) was then added and the mixture was stirred at rt for 1 h. After removal of the volatiles under vacuum, the residue was purified by column chromatography (0-6% MeOH/DCM) to give compound 50 (211 mg, 64%). ¹H NMR (400.3 MHz): 1.42 (s, 3H), 1.64 (s, 3H), 3.64 (d, J=15.2 Hz, 2H), 5.08 (dd, J=12.2, 6.7 Hz, 1H), 5.24 (d, J=6.6 Hz, 1H), 5.80 (d, J=8.4 Hz, 1H), 7.83 (d, J=8.3 Hz, 1H).

((3aS,4S,6R,6aR)-6-Cyano-6-(2,4-dioxo-3,4-dihydropyrimidin-1(2H)-yl)-4-fluoro-2,2-dimethyltetrahydrofuro[3,4-d][1,3]dioxol-4-yl)methyl 3-chlorobenzoate 51. To a mixture of 50 (210 mg, 0.48 mmol), Bu₄NHSO₄ (165 mg, 0.48 mmol), K₂HPO₄ (170 mg, 0.72 mmol) in DCM (20 mL) and water (4 mL) was added m-CPBA (810 mg, 3.6 mmol). The mixture was stirred vigorously at rt overnight before addition of sat Na₂S₂O₃ (6 mL) dropwise, and then sat NaHCO₃ (6 mL). After extraction, the organic layer was dried over sodium sulfate and the volatiles were removed under vacuum. The residue was purified by column chromatography (0-60% EtOAc/Hexane) to give compound 51 (89 mg, 40%). ¹H NMR (400.3 MHz): 1.46 (s, 3H), 1.72 (s, 3H), 4.61 (d, J=9.2 Hz, 1H), 5.29 (m, 2H), 5.85 (dd, J=8.3, 2.3 Hz, 1H), 7.43 (t, J=7.9 Hz, 1H), 7.60 (m, 1H), 7.87 (d, J=12.4 Hz, 1H), 7.94-8.06 (m, 2H), 8.38 (s, 1H).

(3aR,4R,6S,6aS)-4-(2,4-Dioxo-3,4-dihydropyrimidin-1(2H)-yl)-6-fluoro-6-(hydroxymethyl)-2,2-dimethyltetrahydrofuro[3,4-d][1,3]dioxole-4-carbonitrile 52: A mixture of 51 (70 mg) in 28% ammonium hydroxide (3 mL) and methanol (3 mL) was stirred for 30 min at rt until completion. After removal of the volatiles under vacuum, the residue was purified by column chromatography (0-10% MeOH/DCM) to give compound 52 (42.3 mg, 87%). ¹H NMR (400.3 MHz): 1.42 (s, 3H), 1.64 (s, 3H), 3.74-3.86 (m, 2H), 5.04 (dd, J=12.5, 6.4 Hz, 1H), 5.12 (d, J=6.4 Hz, 1H), 5.75 (d, J=8.4 Hz, 1H), 7.93 (d, J=8.4 Hz, 1H).

(2R,3R,4S,5S)-2-(2,4-Dioxo-3,4-dihydropyrimidin-1(2H)-yl)-5-fluoro-3,4-dihydroxy-5-(hydroxymethyl)tetrahydrofuran-2-carbonitrile 53: A solution of 52 (42 mg) in 90% formic acid (2 mL) was stirred at 35° C. for 2 h. After removal of the volatiles under vacuum, the residue was purified by column chromatography (0-12% MeOH/DCM) to give compound 53 (15.6 mg, 42%) and starting material 52 (25 mg). ¹HNRM (400.3 MHz): 3.82 (m, 2H), 4.22 (dd, J=22.0, 5.8 Hz, 1H), 4.58 (d, 5.8 Hz, 1H), 4.62 (brs, 1H), 5.70 (d, J=8.4 Hz, 1H), 8.08 (d, J=8.4 Hz, 1H).

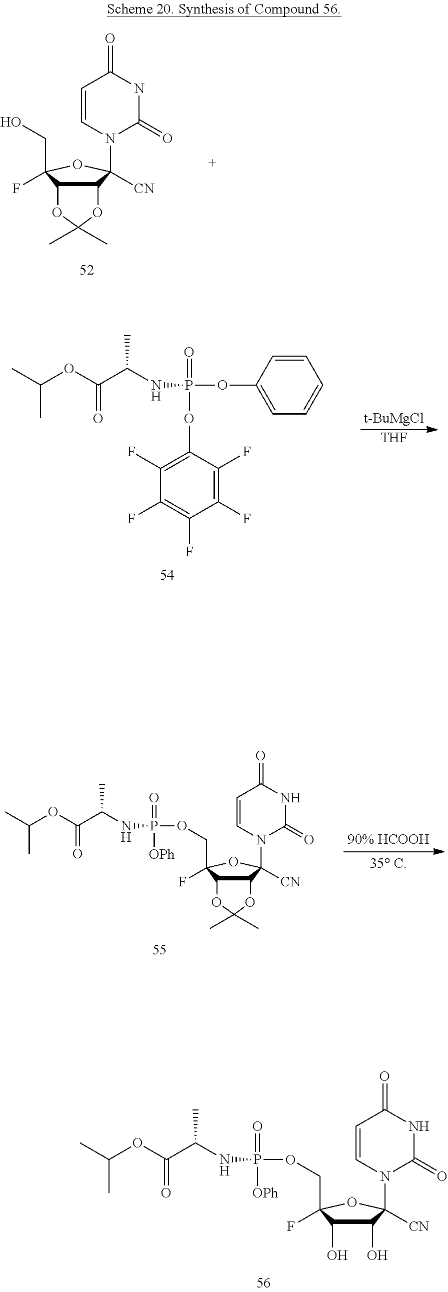

Isopropyl ((S)-(((3aS,4S,6R,6aR)-6-cyano-6-(2,4-dioxo-3,4-dihydropyrimidin-1(2H)-yl)-4-fluoro-2,2-dimethyltetrahydrofuro[3,4-d][1,3]dioxol-4-yl)methoxy)(phenoxy)-phosphoryl)-L-alaninate 55: To a solution of 52 (25 mg) in THE (1 mL) was added dropwise a solution of ^{t}BuMgCl at 0° C. The mixture was stirred at rt for 30 min, before addition of a solution of 54 (99 mg) in THE (1.5 mL). The mixture was stirred at rt overnight. Water was added to quench the reaction and the mixture was extracted with EtOAc (3×30 mL). The combined organic layers were washed with brine and dried over sodium sulfate. After removal of the volatiles under vacuum, the residue was purified by column chromatography (0-7% MeOH/DCM) to give compound 55 (25.6 mg, 60%). ¹H NMR (400.3 MHz): 1.23 (d, J=5.3 Hz, 3H), 1.24 (d, J=5.3 Hz, 3H), 1.35 (dd, J=7.2, 0.6 Hz, 3H), 1.65 (s, 3H), 3.90-3.95 (m, 1H), 4.37 (m, 2H), 4.98-5.02 (m, 1H), 5.06-5.11 (m, 1H), 5.16 (d, J=6.6 Hz, 1H), 5.69 (d, J=8.3 Hz, 1H), 7.19-7.39 (m, 5H), 7.83 (d, J=8.3 Hz, 1H).

Isopropyl ((S)-(((2S,3S,4R,5R)-5-cyano-5-(2,4-dioxo-3,4-dihydropyrimidin-1(2H)-yl)-2-fluoro-3,4-dihydroxytetrahydrofuran-2-yl)methoxy)(phenoxy)phosphoryl)-L-alaninate 56: A solution of 55 (25 mg) in 90% formic acid (1 mL) was stirred at 35° C. for 4 h. After removal of the volatiles under vacuum, the residue was purified by column chromatography (0-12% MeOH/DCM) to give compound 56 (8 mg) and starting material 55 (14 mg). ¹H NMR (400.3 MHz): 1.21-1.35 (m, 9H), 3.90-3.94 (m, 1H), 4.23 (dd, J=21.8, 6.0 Hz, 1H), 3.84-4.42 (m, 2H), 4.60-4.64 (m, 2H), 4.90-4.99 (m, 1H), 5.61 (d, J=8.3 Hz, 1H), 7.20-7.40 (m, 5H), 7.82 (d, J=8.3 Hz, 1H).

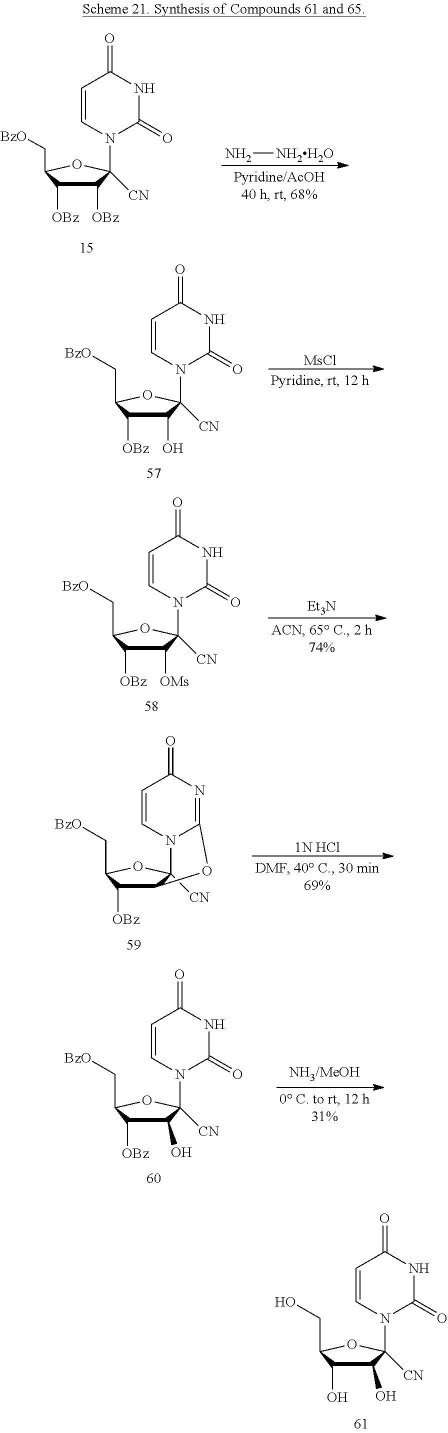

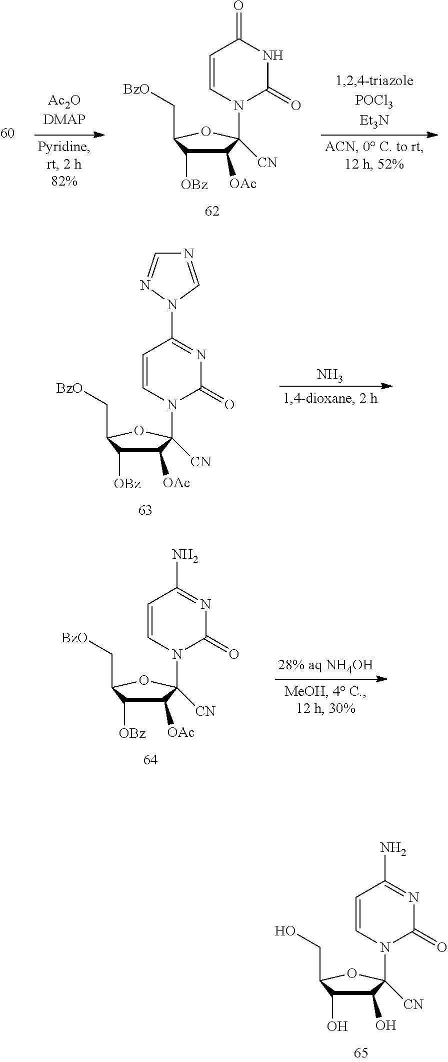

((2R,3S,4R,5R)-3-(Benzoyloxy)-5-cyano-5-(2,4-dioxo-3,4-dihydropyrimidin-1(2H)-yl)-4-hydroxytetrahydrofuran-2-yl)methyl benzoate-57: To a solution of 15 (2.8 g, 4.8 mmol) in pyridine/acetic acid (33.6 mL/8.4 mL), was added hydrazine monohydrate (0.38 mL, 11.8 mmol). The reaction mixture was stirred at room temperature for 40 h. Then, 2 mL of acetone was added and the reaction was stirred for 1 h. Solvents were evaporated, the crude residue was diluted with ethyl acetate (250 mL), washed with 1N aq HCl, saturated sodium bicarbonate/brine (1/1). The organic layer was dried over sodium sulphate, filtered, concentrated under reduced pressure. The crude material was purified by flash column chromatography (0-5% Methanol in Dichloromethane) to afford compound 57 as white solid (1.49 g, 68%). ¹H NMR (400 MHz, CDCl₃) δ 8.12 (d, J=7.12 Hz, 2H), 8.0 (d, J=7.4 Hz, 2H), 7.95 (d, J=8.2 Hz, 1H), 7.65 (m, 2H), 7.52 (m, 4H), 5.59 (d, J=8.3 Hz, 1H), 5.50 (s, 1H), 5.42 (m, 1H), 5.12 (d, J=4.7 Hz, 1H), 5.08 (m, 1H), 4.78 (dd, J=13.2 Hz, J=49.6 Hz, 1H).

((2R,3R,4R,5R)-3-(Benzoyloxy)-5-cyano-5-(2,4-dioxo-3,4-dihydropyrimidin-1(²H)-yl)-4-((methylsulfonyl)oxy)tetrahydrofuran-2-yl)methyl benzoate-58: To a solution of 57 (2.0 g, 4.2 mmol) in pyridine (25 mL), was added methanesulfonyl chloride (0.96 mL, 12.5 mmol) at room temperature and the resulting reaction mixture was stirred for 12 h. Then, the solvents were evaporated under reduced pressure. The crude product was diluted with ethyl acetate (200 mL), washed with aqueous 1N HCl (25 mL), saturated aqueous sodium bicarbonate/brine solution (1/1). The organic layer was dried over sodium sulphate, filtered, concentrated under reduced pressure. The crude material was directly used in the next step without further purification.

((2R,3R,3aR,9aR)-3-(Benzoyloxy)-9a-cyano-6-oxo-3,3a,6,9a-tetrahydro-2H-furo[2′,3′:4,5]oxazolo[3,2-a]pyrimidin-2-yl)methyl benzoate-59: To a solution of 58 (1.73 g, 3.1 mmol) in acetonitrile (30 mL), was added triethylamine (2.32 mL, 16.6 mmol) at room temperature. The resulting reaction mixture was heated to 65° C. for 2 h. Then, solvents were evaporated, the residue was diluted with ethyl acetate (250 mL), washed with 1N aq HCl and saturated sodium bicarbonate/brine (1/1). The organic layer was dried over sodium sulphate, filtered and concentrated under reduced pressure. The crude material was purified by flash column chromatography (0-5% methanol in dichloromethane) to afford compound 59 as white solid (1.49 g, 93%). ¹H NMR (400 MHz, MeOH-d₄) δ 8.11 (d, J=8.48 Hz, 2H), 7.95 (d, J=8.48 Hz, 2H), 7.73 (d, J=7.5 Hz, 1H), 7.64 (m, 2H), 7.50 (m, 4H), 6.06 (d, J=7.48 Hz, 1H), 5.92 (m, 1H), 5.86 (s, 1H), 5.19 (m, 1H), 4.58 (m, 2H).

((2R,3S,5R)-3-(Benzoyloxy)-5-cyano-5-(2,4-dioxo-3,4-dihydropyrimidin-1(2H)-yl)-4-hydroxytetrahydrofuran-2-yl)methyl benzoate-60: To a solution of 59 (1.32 g, 2.8 mmol) in DMF (30 mL) was added 1N aq HCl solution (7.6 mL) at room temperature. Then, the resulting reaction mixture was stirred at 40° C. for 30 min, then cooled down to rt before being diluted with ethyl acetate. The organic layer was washed with saturated sodium bicarbonate/brine (1/1), dried over sodium sulphate, filtered and concentrated under reduced pressure. The crude material was purified by flash column chromatography (0-5% Methanol in Dichloromethane) to afford compound 60 as white solid (0.95 g, 69.3%). ¹H NMR (400 MHz, DMSO-d₆) δ 11.75 (s, 1H), 8.07 (m, 4H), 7.69 (m, 2H), 7.59 (m, 4H), 7.20 (d, J=5.2 Hz, 1H), 5.70 (d, J=8.28 Hz, 1H), 5.46 (s, 1H), 5.03 (t, J=4.76 Hz, 1H), 4.89 (d, J=5.28 Hz, 1H), 4.67 (m, 2H).

(2R,4S,5R)-2-(2,4-Dioxo-3,4-dihydropyrimidin-1(2H)-yl)-3,4-dihydroxy-5-(hydroxymethyl)tetrahydrofuran-2-carbonitrile-61: To an ice cold solution of 60 (100 mg, 0.2 mmol) in methanol (5 mL), ammonia gas was bubbled through for 5 min and the resulting reaction mixture was stirred at room temperature for 6 h. Solvents were evaporated under reduced pressure, and the crude product was purified by flash column chromatography (0-10% methanol in dichloromethane) to afford compound 61 as white solid (17.4 mg, 31%). ¹H NMR (400 MHz, MeOH-d₄) δ 7.93 (d, J=8.28 Hz, 1H), 5.72 (d, J=8.28 Hz, 1H), 4.73 (d, J=1.16 Hz, 1H), 4.42 (m, 1H), 4.16 (t, J=1.4 Hz, 1H), 3.38 (m, 2H). ¹³C NMR (101 MHz, MeOH-d₄) δ 164.7, 149.8, 139.5, 115.4, 100.6, 89.7, 81.1, 76.8, 60.8. LC-MS: m/z: 292.0 [M+Na].

(2R,3R,5R)-4-Acetoxy-2-((benzoyloxy)methyl)-5-cyano-5-(2,4-dioxo-3,4-dihydropyrimidin-1(2H)-yl)tetrahydrofuran-3-yl benzoate-62: To a solution of 60 (200 mg, 0.42 mmol) in pyridine (3.5 mL) was added DMAP (1.7 mg, 0.014 mmol) and acetic anhydride (60 μL, 0.63 mmol) at room temperature. After 2 h, the volatiles were removed under reduced pressure. The crude product was diluted with ethyl acetate, washed with 1N aq HCl and saturated sodium bicarbonate/brine (1/1). The organic layer was dried over sodium sulphate, filtered and concentrated under reduced pressure. The crude material was purified by flash column chromatography (0-5% Methanol in Dichloromethane) to afford compound 62 as white form (178.4 mg, 82%). ¹H NMR (400 MHz, CDCl₃) δ 8.36 (bro,s, 1H), 8.14 (d, J=8.44 Hz, 2H), 8.05 (d, J=8.08 Hz, 2H), 7.72 (d, J=8.36 Hz, 1H), 7.62 (m, 2H), 7.49 (m, 4H), 6.15 (s, 1H), 5.82 (d, J=8.4 Hz, 1H), 5.48 (d, J=2.72 Hz, 1H), 5.0 (dd, J=2.9 Hz, J=12.4 Hz, 1H), 4.86 (m, 1H), 4.77 (m, 1H). 1.69 (s, 3H).

(2R,3R,5R)-4-Acetoxy-2-((benzoyloxy)methyl)-5-cyano-5-(2-oxo-4-(1H-1,2,4-triazol-1-yl)pyrimidin-1(2H)-yl)tetrahydrofuran-3-yl benzoate-63: To an ice cold suspension of 1,2,4-triazole (0.74 g, 10.6 mmol) in acetonitrile (18 mL) was added triethylamine (1.6 mL, 11.5 mmol) and POCl₃ (165 μL, 1.76 mmol). The resulting reaction mixture was stirred for 1 h at 0° C. A solution of 62 (180 mg, 0.34 mmol) in acetonitrile (3.6 mL) was added and the resulting reaction mixture was stirred for 12 h at room temperature. The reaction was quenched with saturated sodium bicarbonate and extracted with ethyl acetate. The combined organic layers were washed with brine, dried over sodium sulphate, filtered and concentrated under reduced pressure to obtain compound 63 as a pale-yellow foam (102.8 mg, 52%). The crude material was directly used in the next step without further purification. LC-MS: m/z: 571.5 [M+H].

(2R,4S,5R)-2-(4-Amino-2-oxopyrimidin-1(2H)-yl)-3,4-dihydroxy-5-(hydroxymethyl)-tetrahydrofuran-2-carbonitrile-65: To an ice cold solution of 63 (90 mg, 0.15 mmol) in 1,4-dioxane (5 mL) ammonia gas was bubbled through for 5 min and the resulting reaction mixture was stirred at room temperature for 2 h. Solvents were then evaporated under reduced pressure to obtained compound 64. The crude product 64 was dissolved in a 1:1 mixture of methanol and 28% aq ammonium hydroxide (4 mL) at 4° C. and stirred for 12 h. Solvents were evaporated and the crude product was purified by flash column chromatography (0-10% methanol in dichloromethane) to afford compound 65 as white solid (12.6 mg, over two steps 30%). ¹H NMR (400 MHz, MeOH-d₄) δ 7.81 (d, J=7.72 Hz, 1H), 5.80 (d, J=7.72 Hz, 1H), 4.67 (s, 1H), 4.28 (m, 1H), 4.01 (m, 1H), 3.68 (m, 2H). ¹³C NMR (101 MHz, MeOH-d₄) δ 166.5, 163.6, 155.4, 140.3, 115.9, 94.2, 89.9, 89.2, 80.9, 77.1, 60.9. LC-MS: m/z: 291.0 [M+Na].

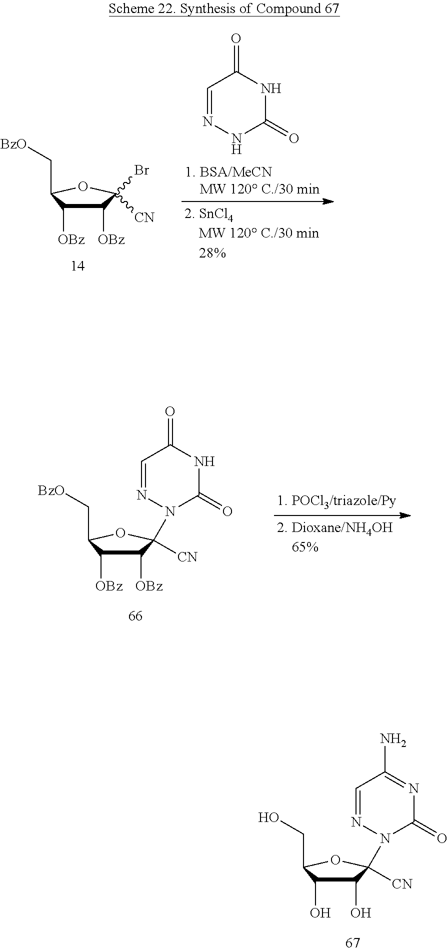

### (2R,3R,4R,5R)-5-((benzoyloxy)methyl)-2-cyano-2-(3,5-dioxo-4,5-dihydro-1,2,4-triazin-2(3H)-yl)tetrahydrofuran-3,4-diyl dibenzoate (66)

6-Azauracil (678 mg, 6 mmol) was suspended in acetonitrile (5 mL) in a microwave vial, and BSA (4.5 mL) was added. The suspension was heated under microwave irradiation at 120° C. for 30 min. The solution was then cooled down to room temperature and compound 14 (1.10 g, 2 mmol) was added, followed by SnCl₄ (2 mL). The vial was heated under microwave irradiation at 120° C. for 30 min. The reaction mixture was slowly added to a saturated aq NaHCO₃ solution (50 mL) and stirred for 15 min. After dilution with ethyl acetate (60 mL), and filtration through a pad of celite, the organic layer was separated, and the aqueous layer was extracted with ethyl acetate (2×40 mL). The combined organic layers were washed with brine (50 mL), and concentrated under reduced pressure. The residue was purified by flash column chromatography on silica gel eluting with hexane-EtOAc (4:1 to 1:1) to give 331 mg of product (28%) as a light yellow foam. ¹H-NMR (CDCl₃): δ 8.04-8.01 (m, 6H), 7.66-7.35 (m, 9H), 7.12 (s, 1H), 6.84 (d, 1H), 6.06 (m, 1H), 4.99 (m, 1H), 4.90-4.86 (m, 1H), 4.62-4.54 (m, 1H).

### (2R,3R,4S,5R)-2-(5-amino-3-oxo-1,2,4-triazin-2(3H)-yl)-3,4-dihydroxy-5-(hydroxymethyl)tetrahydrofuran-2-carbonitrile (67)

To a stirred mixture of 66 (121 mg, 0.21 mmol) and triazole (116 mg, 1.68 mmol) in pyridine (3 mL) was added POCl₃ (39 μL, 0.42 mmol) dropwise, and the mixture was stirred at RT overnight. The volatiles were evaporated and the residue was diluted in 1,4-dioxane (2 mL) and conc. NH₄OH (3 mL). The mixture was stirred at RT overnight. The solvent was evaporated and co-evaporated with EtOH under reduced pressure. The residue was purified by flash chromatography on silica gel eluting with CH₂Cl₂-MeOH (9:1 to 4:1) to give 36.5 mg (65%) of product. ¹H-NMR (CD₃OD): δ 7.55 (s, 1H), 4.98-4.97 (d, 1H), 4.26-4.23 (m, 2H), 3.76-3.61 (m, 2H). HRMS calc for C₉H₁₂N₅O₅ (M+H+): 270.0838, found 270.0827.

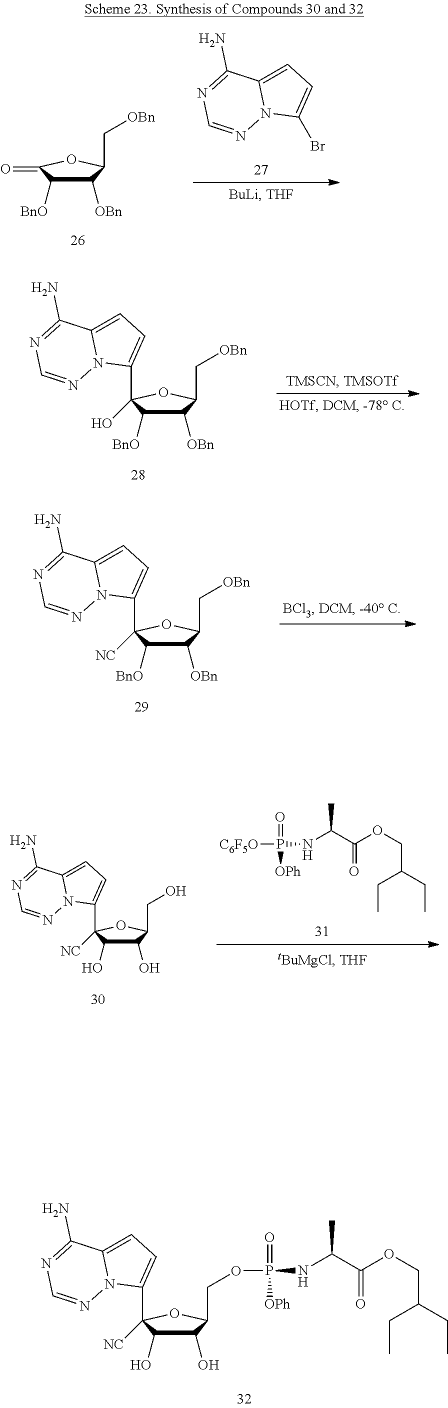

### (2R,3R,4R,5S)-2-(4-aminopyrrolo[2,1-f][1,2,4]triazin-7-yl)-3,4-bis(benzyloxy)-5-((benzyloxy)methyl) tetrahydrofuran-2-ol (28)

To a solution of compound 27 (0.72 g, 3.37 mmol) in THF (35 mL) was added 1,2-bis(chlorodimethylsilyl) ethane (800 mg, 3.70 mmol) and DIPEA (0.53 mL, 3.7 mmol) at rt. After being stirred for 10 min, the reaction mixture was cooled to −78° C. nBuLi (2.0 M, 7.2 mL, 1.4 mmol) was dropwise added into the reaction over 45 min. After the dropping funnel was rinsed by THE (1 mL), a solution of lactone 26 (2.2 g, 5.25 mmol) in THE (5 mL) was added dropwise over 20 min. The reaction was allowed to stir at −78° C. for 2 h, and then warmed slowly to 0° C. The reaction was quenched by adding 1 M citric acid solution (50 mL) and stirred vigorously for 10 min. The organic layer was separated, and the aqueous layer was extracted with ethyl acetate (50 mL×3). The combined organic layers were washed with water (50 mL), saturated aqueous NaHCO₃ (50 mL), and brine (50 mL). The organic phase was dried (Na₂SO₄), filtered, and concentrated. The residue was subjected to silica gel chromatography (40% EtOAc in DCM) to get 28 as a white foam. ESI-MS: m/z 553 [M+H].

### (2S,3R,4R,5S)-2-(4-aminopyrrolo[2,1-f][1,2,4]triazin-7-yl)-3,4-bis(benzyloxy)-5-((benzyloxy)methyl) tetrahydrofuran-2-carbonitrile (29)

To a solution of compound 28 (820 mg, 1.49 mmol) in DCM (60 mL) was added trifluoromethanesulfonic acid (0.27 mL, 3 mmol) at −78° C. After the reaction was stirred for 10 min, TMSOTf (1.1 mL, 6.07 mmol) was slowly added and the resulting mixture was stirred for 30 min at −78° C. TMSCN (0.78 mL, 6.2 mmol) was then added slowly and the mixture was stirred for 2 h. Triethylamine (0.8 mL) was added and the reaction mixture was allowed to warm to room temperature. Solid sodium bicarbonate (1 g) was then added followed by the slow addition of water (300 mL) and the resulting mixture was stirred for 10 min. The layers were then separated and the aqueous layer was extracted with DCM. The combined organic extracts were washed with brine, dried over Na₂SO₄ and concentrated under reduced pressure. The crude residue was chromatographed on silica gel (40-100% EtOAc in hexanes) to afford 29 (680 mg, 82%) as an off-white solid. ¹H NMR (400 MHz, CDCl₃) δ 7.85 (s, 1H), 7.21-7.34 (m, 15H), 6.96 (d, J=4.8 Hz, 1H), 6.54 (d, J=4.8 Hz, 1H), 5.54 (s, br, 2H), 4.79-4.88 (m, 3H), 4.49-4.59 (m, 4H), 4.36-4.40 (m, 1H), 4.04-4.07 (m, 1H), 3.77-3.81 (m, 1H), 3.61-3.65 (m, 1H); ESI-MS: m/z 562 [M+H].

### (2S,3R,4S,5S)-2-(4-aminopyrrolo[2,1-f][1,2,4]triazin-7-yl)-3,4-dihydroxy-5-(hydroxymethyl) tetrahydrofuran-2-carbonitrile (30)

To a solution of 29 (400 mg, 0.71 mmol) in DCM (10 mL) was added boron trichloride (1 M, 2.8 mL, 2.8 mmol) at −78° C. under an argon atmosphere. The reaction mixture was allowed to warm to −40° C. and was stirred for 2 h at that temperature. The reaction mixture was cooled down to −78° C. and quenched by slow addition of methanol (1 mL). A solution of triethylamine (2.5 mL) in methanol (4 mL) was added dropwise and the reaction mixture was allowed to warm to room temperature. The resulting mixture was concentrated under reduced pressure. The solid residue was slurried with hexanes (10 mL) and the supernatant was then decanted. The remaining solid residue was suspended into methanol (10 mL) and was heated to 45° C. After the volatiles were removed in vacuo, the residue was purified by preparative HPLC to afford 30 (137 mg, 66%). ¹H NMR (400 MHz, MeOH-d₄) δ 7.92 (s, 1H), 7.02-7.06 (m, 2H), 4.80-4.82 (m, 1H), 4.25-4.28 (m, 1H), 4.17-4.20 (m, 1H), 3.84-3.88 (m, 1H), 3.70-3.74 (m, 1H); ESI-MS: m/z 292 [M+H].

### 2-ethylbutyl ((S)-(((2S,3S,4R,5S)-5-(4-aminopyrrolo[2,1-f][1,2,4]triazin-7-yl)-5-cyano-3,4-dihydroxytetrahydrofuran-2-yl)methoxy)(phenoxy)phosphoryl)-L-alaninate (32)

To a solution of 30 (50 mg, 0.17 mmol) in THF (8 mL) was added tert-butylmagnesium chloride (1 M, 0.5 mL, 0.5 mmol) at 0° C. After being stirred for 30 min, a solution of 2-ethylbutyl ((perfluorophenoxy)(phenoxy) phosphoryl)-L-alaninate 31 (120 mg, 0.27 mmol) in THE (2 mL) was added at 0° C. The reaction mixture was then stirred at room temperature for 24 h before being quenched by adding iPrOH (0.5 mL). After removal of the volatile under reduced pressure, the residue was purified on preparative TLC (DCM:MeOH=10:1) to afford 32 (15 mg, 14%) as a white solid. ¹H NMR (400 MHz, MeOH-d₄) δ 7.76 (s, 1H), 7.18-7.26 (m, 2H), 7.05-7.10 (m, 2H), 6.77-6.82 (m, 1H), 4.69 (d, J=5.2 Hz, 1H), 3.90-3.94 (m, 3H), 3.80-3.84 (m, 1H), 3.91-3.94 (m, 1H), 3.80-3.84 (m, 2H), 1.17-1.25 (m, 8H), 0.73-0.80 (m, 6H); ESI-MS: m/z 603 [M+H].

### Example 2

### Cellular Toxicity Assays

The toxicity of the compounds was assessed in Vero, human PBM, CEM (human lymphoblastoid), MT-2, and HepG2 cells, as described previously (see Schinazi R. F., Sommadossi J.-P., Saalmann V., Cannon D. L., Xie M.-Y., Hart G. C., Smith G. A. & Hahn E. F. Antimicrob. Agents Chemother. 1990, 34, 1061-67). Cycloheximide was included as positive cytotoxic control, and untreated cells exposed to solvent were included as negative controls. The cytotoxicity IC₅₀ was obtained from the concentration-response curve using the median effective method described previously (see Chou T.-C. & Talalay P. Adv. Enzyme Regul. 1984, 22, 27-55; Belen'kii M. S. & Schinazi R. F. Antiviral Res. 1994, 25, 1-11). The results are shown in Table 8 below:

### Example 3

### Mitochondrial Toxicity Assays in HepG2 Cells:

i) Effect of Compounds on Cell Growth and Lactic Acid Production: The effect on the growth of HepG2 cells can be determined by incubating cells in the presence of 0 μM, 0.1 μM, 1 μM, 10 μM and 100 μM drug. Cells (5×10⁴ per well) can be plated into 12-well cell culture clusters in minimum essential medium with nonessential amino acids supplemented with 10% fetal bovine serum, 1% sodium pyruvate, and 1% penicillin/streptomycin and incubated for 4 days at 37° C. At the end of the incubation period the cell number can be determined using a hemocytometer. Also taught by Pan-Zhou X—R, Cui L, Zhou X-J, Sommadossi J-P, Darley-Usmer V M. “Differential effects of antiretroviral nucleoside analogs on mitochondrial function in HepG2 cells,” Antimicrob. Agents Chemother. 2000; 44: 496-503.

To measure the effects of the compounds on lactic acid production, HepG2 cells from a stock culture can be diluted and plated in 12-well culture plates at 2.5×10⁴ cells per well. Various concentrations (0 μM, 0.1 μM, 1 μM, 10 μM and 100 μM) of compound can be added, and the cultures can be incubated at 37° C. in a humidified 5% CO₂ atmosphere for 4 days. At day 4, the number of cells in each well can be determined and the culture medium collected. The culture medium can then be filtered, and the lactic acid content in the medium determined using a colorimetric lactic acid assay (Sigma-Aldrich). Since lactic acid product can be considered a marker for impaired mitochondrial function, elevated levels of lactic acid production detected in cells grown in the presence of test compounds indicates a drug-induced cytotoxic effect.
ii) Effect on Compounds on Mitochondrial DNA Synthesis: a real-time PCR assay to accurately quantify mitochondrial DNA content has been developed (see Stuyver L J, Lostia S, Adams M, Mathew J S, Pai B S, Grier J, Tharnish P M, Choi Y, Chong Y, Choo H, Chu C K, Otto M J, Schinazi R F. Antiviral activities and cellular toxicities of modified 2′,3′-dideoxy-2′,3′-didehydrocytidine analogs. Antimicrob. Agents Chemother. 2002; 46: 3854-60). This assay can be used in all studies described in this application that determine the effect of compounds on mitochondrial DNA content. In this assay, low-passage-number HepG2 cells are seeded at 5,000 cells/well in collagen-coated 96-well plates. Test compounds are added to the medium to obtain final concentrations of 0 μM, 0.1 μM, 10 μM and 100 μM. On culture day 7, cellular nucleic acids can be prepared by using commercially available columns (RNeasy 96 kit; Qiagen). These kits co-purify RNA and DNA, and hence, total nucleic acids are eluted from the columns. The mitochondrial cytochrome c oxidase subunit II (COXII) gene and the ß-actin or rRNA gene can be amplified from 5 μl of the eluted nucleic acids using a multiplex Q-PCR protocol with suitable primers and probes for both target and reference amplifications. For COXII the following sense, probe and antisense primers can be used, respectively: 5′-TGCCCGCCATCATCCTA-3′, 5′-tetrachloro-6-carboxyfluorescein-TCCTCATCGCCCTCCCATCCC-TAMRA-3′ and 5′-CGTCTGTTATGTAAAGGATGCGT-3′. For exon 3 of the ß-actin gene (GenBank accession number E01094) the sense, probe, and antisense primers are 5′-GCGCGGCTACAGCTTCA-3′, 5′-6-FAMCACCACGGCCGAGCGGGATAMRA-3′ and 5′-TCTCCTTAATGTCACGCACGAT-3′, respectively. The primers and probes for the rRNA gene are commercially available from Applied Biosystems. Since equal amplification efficiencies are obtained for all genes, the comparative CT method can be used to investigate potential inhibition of mitochondrial DNA synthesis. The comparative CT method uses arithmetic formulas in which the amount of target (COXII gene) is normalized to the amount of an endogenous reference (the ß-actin or rRNA gene) and is relative to a calibrator (a control with no drug at day 7). The arithmetic formula for this approach is given by 2-ΔΔCT, where ΔΔCT is (CT for average target test sample−CT for target control)−(CT for average reference test −CT for reference control) (see Johnson M R, K Wang, JB Smith, MJ Heslin, RB Diasio. Quantitation of dihydropyrimidine dehydrogenase expression by real-time reverse transcription polymerase chain reaction. Anal. Biochem. 2000; 278:175-184). A decrease in mitochondrial DNA content in cells grown in the presence of drug indicates mitochondrial toxicity.

### Example 4

### Mitochondrial Toxicity-Glu/Gal

### Protocol Summary

HepG2 cells are plated on 96 or 384 well tissue culture polystyrene plates. After 24 hr the cells are dosed with test compound at a range of concentrations and incubated for 72 hr in medium supplemented with either galactose or glucose. Test compounds are said to cause mitochondrial toxicity if the cells grown in galactose-containing medium are more sensitive to the test compound than the cells grown in glucose-containing medium.

Objective: To measure the sensitivity of HepG2 cells grown in medium containing either galactose or glucose to the test compound.

### Experimental Procedure

HepG2 human hepatocellular carcinoma cells are plated on 96 or 384-well tissue culture polystyrene plates containing either galactose or glucose containing medium supplemented with 10% fetal bovine serum and antibiotics and incubated overnight. The cells are dosed with increasing concentrations of the test compound (final DMSO concentration 0.5%; typical final test compound concentrations of 100, 30, 10, 3, 1, 0.3, 0.1, 0.03 μM for an eight point dose response curve; n=3 replicates per concentration) and the cells are incubated for 72 hr. Appropriate controls are simultaneously used as quality controls. Cell viability is measured using Hoechst staining and cell counting by a HCS reader.

### Example 5

### Mitochondrial Toxicity Assays in Neuro2A Cells

To estimate the potential of the compounds described herein to cause neuronal toxicity, mouse Neuro2A cells (American Type Culture Collection 131) can be used as a model system (see Ray A S, Hernandez-Santiago B I, Mathew J S, Murakami E, Bozeman C, Xie M Y, Dutschman G E, Gullen E, Yang Z, Hurwitz S, Cheng Y C, Chu C K, McClure H, Schinazi R F, Anderson K S. Mechanism of anti-human immunodeficiency virus activity of beta-D-6-cyclopropylamino-2′,3′-didehydro-2′,3′-dideoxyguanosine. Antimicrob. Agents Chemother. 2005, 49, 1994-2001). The concentrations necessary to inhibit cell growth by 50% (CC50) can be measured using the 3-(4,5-dimethyl-thiazol-2-yl)-2,5-diphenyltetrazolium bromide dye-based assay, as described. Perturbations in cellular lactic acid and mitochondrial DNA levels at defined concentrations of drug can be carried out as described above. ddC and AZT can be used as control nucleoside analogs.

### Example 6

### Assay for Bone Marrow Cytotoxicity

Primary human bone marrow mononuclear cells can be obtained commercially from Cambrex Bioscience (Walkersville, MD). CFU-GM assays is carried out using a bilayer soft agar in the presence of 50 units/mL human recombinant granulocyte/macrophage colony-stimulating factor, while BFU-E assays used a ethylcellulose matrix containing 1 unit/mL erythropoietin (see Sommadossi J P, Carlisle R. Toxicity of 3′-azido-3′-deoxythymidine and 9-(1,3-dihydroxy-2-propoxymethyl) guanine for normal human hepatopoietic progenitor cells in vitro. Antimicrob. Agents Chemother. 1987; 31: 452-454; Sommadossi, JP, Schinazi, RF, Chu, C K, and Xie, MY. Comparison of cytotoxicity of the (−) and (+) enantiomer of 2′,3′-dideoxy-3′-thiacytidine in normal human bone marrow progenitor cells. Biochem. Pharmacol. 1992; 44:1921-1925). Each experiment can be performed in duplicate in cells from three different donors. AZT is used as a positive control. Cells can be incubated in the presence of the compound for 14-18 days at 37° C. with 5% CO₂, and colonies of greater than 50 cells can be counted using an inverted microscope to determine the IC₅₀. The 50% inhibitory concentration (IC₅₀) can be obtained by least-squares linear regression analysis of the logarithm of drug concentration versus BFU-E survival fractions. Statistical analysis can be performed with Student's t test for independent non-paired samples.

### Example 7

### In Vitro Human Mitochondrial RNA Polymerase (POLRMT) Assay

In vitro RNA nucleotide incorporation assays with POLRMT (INDIGO Biosciences) can be performed as previously described (Arnold et al. 2012). Briefly, ³²P-radiolabeled RNA primer (5′-UUUUGCCGCGCC) can be hybridized to 3 molar excess of the appropriate DNA template (5′-GGGAATGCANGGCGCGGC where position N can be replaced by A, T, or C). 125 nM of POLRMT can be incubated with 500 nM of 5′-radiolabled RNA/DNA hybrid, 10 mM MgCl₂ and 100 μM of the corresponding nucleoside triphosphate. For non-nucleoside analogs, 100 μM of inhibitor can be added at the same time as 100 μM UTP. Incorporation can be allowed to proceed for 2 h at 30° C. and reactions are stopped by the addition of 10 mM EDTA and formamide. Samples are visualized on 20% denaturing polyacrylamide gel. Data can be analyzed by normalizing the product fraction for each nucleoside triphosphate analog to that of the corresponding natural nucleoside triphosphate.

### Example 8

### Effect of Nucleotide Analogs on the DNA Polymerase and Exonuclease Activities of Mitochondrial DNA Polymerase γ

i) Purification of Human Polymerase γ: The recombinant large and small subunits of polymerase γ can be purified as described previously (see Graves S W, Johnson A A, Johnson K A. Expression, purification, and initial kinetic characterization of the large subunit of the human mitochondrial DNA polymerase. Biochemistry. 1998, 37, 6050-8; Johnson A A, Tsai Y, Graves S W, Johnson K A. Human mitochondrial DNA polymerase holoenzyme: reconstitution and characterization. Biochemistry 2000; 39: 1702-8). The protein concentration can be determined spectrophotometrically at 280 nm, with extinction coefficients of 234,420, and 71,894 M-1 cm-1 for the large and the small subunits of polymerase γ, respectively.
ii) Kinetic Analyses of Nucleotide Incorporation: Pre-steady-state kinetic analyses can be performed to determine the catalytic efficiency of incorporation (k/K) for DNA polymerase γ for nucleoside-TP and natural dNTP substrates. This allowed determination of the relative ability of this enzyme to incorporate modified analogs and predict toxicity. Pre-steady-state kinetic analyses of incorporation of nucleotide analogs by DNA polymerase γ would be carried out essentially as described previously (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004, 62, 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004, 48, 1300-6). Briefly, a pre-incubated mixture of large (250 nM) and small (1.25 mM) subunits of polymerase γ and 60 nM DNA template/primer in 50 mM Tris-HCl, 100 mM NaCl, pH 7.8, can be added to a solution containing MgCl₂ (2.5 mM) and various concentrations of nucleotide analogs. Reactions can be quenched and analyzed as described previously. Data can be fit to the same equations as described above.
iii) Assay for Human Polymerase γ 3′ 5′ Exonuclease Activity: The human polymerase γ exonuclease activity can be studied by measuring the rate of formation of the cleavage products in the absence of dNTP. The reaction can be initiated by adding MgCl₂ (2.5 mM) to a pre-incubated mixture of polymerase γ large subunit (40 nM), small subunit (270 nM), and 1,500 nM chain-terminated template/primer in 50 mM Tris-HCl, 100 mM NaCl, pH 7.8, and quenched with 0.3M EDTA at the designated time points. All reaction mixtures would be analyzed on 20% denaturing polyacrylamide sequencing gels (8M urea), imaged on a Bio-Rad GS-525 molecular image system, and quantified with Molecular Analyst (Bio-Rad). Products formed from the early time points would be plotted as a function of time. Data would be fitted by linear regression with Sigma Plot (Jandel Scientific). The slope of the line can be divided by the active enzyme concentration in the reaction to calculate the kexo for exonuclease activity (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004; 62: 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004; 48: 1300-6).

### Example 9

### Inhibition of Human DNA Polymerases by NTP's

### Study Objectives

To determine whether a nucleoside-triphosphate analog inhibits human DNA polymerases Alpha, Beta and Gamma and to calculate IC₅₀ values.

### Materials and Methods

Human DNA Polymerase Alpha—Enzyme can be purchased from Chimerx (cat #1075) and assayed based on their recommendations with some modifications. The 2′-Me-UTP was treated with Inorganic Pyrophosphatase (Sigma) to remove any pyrophosphate contamination. A final concentration of 500 μM 2′-Me-UTP can be incubated with 1 mM DTT, 50 mM Tris, 50 mM NaCl, 6 mM MgCl₂, and 1 unit of pyrophosphatase for 1 hour at 37° C. followed by inactivation at 95° C. for 10 minutes. A mixture of 0.05 units of Human DNA Polymerase Alpha and a 5′end radiolabeled 24 nt DNA primer (5′-TCAGGTCCCTGTTCGGGCGCCACT) anneal to a 48 nt DNA template (5′-CAGTGTGGAAAATCTCTAGCAGTGGCGCCCGAACAGGGACCTGAAAGC) can be mixed with increasing concentrations of compound from 0 to 100 μM in 60 mM Tris-HCl (pH 8.0), 5 mM magnesium acetate, 0.3 mg/ml bovine serum albumin, 1 mM dithiothreitol, 0.1 mM spermine, 0.05 mM of each dCTP, dGTP, dTTP, dATP in a final reaction volume of 20 μl for 5 min at 37° C. (all concentrations represent final concentrations after mixing). The reactions can be stopped by mixing with 0.3 M (final) EDTA. Products are separated on a 20% polyacrylamide gel and quantitated on a Bio-Rad Molecular Imager FX. Results from the experiments can be fit to a dose response equation, (y min+((y max)−(y min)))/(1+(compound concentration)/IC₅₀)\hat{ }slope) to determine IC₅₀ values using Graphpad Prism or SynergySoftware Kaleidagraph. Data can be normalized to controls.

Human DNA Polymerase Beta—Enzyme can be purchased from Chimerx (cat #1077) and assayed based on their recommendations with some modifications. A mixture of 0.1 units of Human DNA Polymerase Beta and a 5′end radiolabeled 24 nt DNA primer (5′-TCAGGTCCCTGTTCGGGCGCCACT) anneal to a 48 nt DNA template (5′-CAGTGTGGAAAATCTCTAGCAGTGGCGCCCGAACAGGGACCTGAAAGC) can be mixed with increasing concentrations of compound from 0 to 100 μM in 50 mM Tris-HCl (pH 8.7), 10 mM KCl, 10 mM MgCl₂, 0.4 mg/ml bovine serum albumin, 1 mM dithiothreitol, 15% (v/v) glycerol, and 0.05 mM of each dCTP, dGTP, dTTP, dATP in a final reaction volume of 20 μl for 5 min at 37° C. (all concentrations represent final concentrations after mixing). The reactions can be stopped by mixing with 0.3 M (final) EDTA. Products can be separated on a 20% polyacrylamide gel and quantitated on a Bio-Rad Molecular Imager FX. Results from the experiments can be fit to a dose response equation, (y min+((y max)−(y min)))/(1+(compound concentration)/IC₅₀)\hat{ }slope) to determine IC₅₀ values using Graphpad Prism or SynergySoftware Kaleidagraph. Data can be normalized to controls.

Human DNA Polymerase Gamma—Enzyme can be purchased from Chimerx (cat #1076) and assayed based on their recommendations with some modifications. A mixture of 0.625 units of Human DNA Polymerase Gamma and a 5′end radiolabeled 24 nt DNA primer (5′-TCAGGTCCCTGTTCGGGCGCCACT) anneal to a 36 nt DNA template (5′-TCTCTAGAAGTGGCGCCCGAACAGGGACCTGAAAGC) can be mixed with increasing concentrations of compound from 0 to 100 μM in 50 mM Tris-HCl (pH 7.8), 100 mM NaCl, 5 mM MgCl₂, and 0.05 mM of each dCTP, dGTP, dTTP, dATP in a final reaction volume of 20 μl for 200 min at 37° C. (all concentrations represent final concentrations after mixing). The reactions can be stopped by mixing with 0.3 M (final) EDTA. Products can be separated on a 20% polyacrylamide gel and quantitated on a Bio-Rad Molecular Imager FX. Results from the experiments can be fit to a dose response equation, (y min+((y max)−(y min)))/(1+(compound concentration)/IC₅₀)\hat{ }slope) to determine IC₅₀ values using Graphpad Prism or SynergySoftware Kaleidograph. Data can be normalized to controls.

### Example 10

### Cellular Pharmacology in HepG2 Cells

HepG2 cells are obtained from the American Type Culture Collection (Rockville, MD), and are grown in 225 cm² tissue culture flasks in minimal essential medium supplemented with non-essential amino acids, 1% penicillin-streptomycin. The medium is renewed every three days, and the cells are subcultured once a week. After detachment of the adherent monolayer with a 10 minute exposure to 30 mL of trypsin-EDTA and three consecutive washes with medium, confluent HepG2 cells are seeded at a density of 2.5×10⁶ cells per well in a 6-well plate and exposed to 10 μM of [3H] labeled active compound (500 dpm/pmol) for the specified time periods.

The cells are maintained at 37° C. under a 5% CO₂ atmosphere. At the selected time points, the cells are washed three times with ice-cold phosphate-buffered saline (PBS).

Intracellular active compound and its respective metabolites are extracted by incubating the cell pellet overnight at −20° C. with 60% methanol followed by extraction with an additional 20 pal of cold methanol for one hour in an ice bath. The extracts are then combined, dried under gentle filtered air flow and stored at −20° C. until HPLC analysis.

### Example 11

### Cellular Pharmacology in PBM Cells

Test compounds are incubated in PBM cells at 50 μM for 4 h at 37° C. Then the drug containing media is removed and the PBM cells are washed twice with PBS to remove extracellular drugs. The intracellular drugs are extracted from 10×10⁶ PBM cells using 1 mL 70% ice-cold methanol (containing 10 nM of the internal standard ddATP). Following precipitation, the samples are maintained at room temperature for 15 min followed by vortexing for 30 sec, and then stored 12 h at −20° C. The supernatant is then evaporated to dryness. Dry samples would be stored at −20° C. until LC-MS/MS analysis. Prior to analysis, each sample is reconstituted in 100 μL mobile phase A, and centrifuged at 20,000 g to remove insoluble particulates.

Gradient separation is performed on a Hypersil GOLD column (100×1.0 mm, 3 μm particle size; Thermo Scientific, Waltham, MA, USA). Mobile phase A consists of 2 mM ammonium phosphate and 3 mM hexylamine. Acetonitrile is increased from 10 to 80% in 15 min, and kept at 80% for 3 min. Equilibration at 10% acetonitrile lasts 15 min.

The total run time is 33 min. The flow rate is maintained at 50 μL/min and a 10 μL injection is used. The autosampler and the column compartment are typically maintained at 4.5 and 30° C., respectively.

The first 3.5 min of the analysis is diverted to waste. The mass spectrometer is operated in positive ionization mode with a spray voltage of 3.2 kV.

### Example 12

### MERS Assay

### Cells and Virus:

Human lung carcinoma cells (A-549) can be used for the primary antiviral assays and can be obtained from American Type Culture Collection (ATCC, Rockville, Md., USA). The cells can be passed in minimal essential medium (MEM with 0.15% NaCHO₃, Hyclone Laboratories, Logan, Utah, USA) supplemented with 10% fetal bovine serum. When evaluating compounds for efficacy, the serum can be reduced to a final concentration of 2% and the medium can contain gentamicin (Sigma-Aldrich, St. Louis, Mo.) at 50 μg/mL. Since the MERS-Co virus did not produce detectable virus cytopathic effects, virus replication in A549 cells can be detected by titering virus supernatant fluids from infected, compound-treated A549 cells in Vero 76 cells.

Vero 76 cells can be obtained from ATCC and can be routinely passed in MEM with 0.15% NaCHO₃ supplemented with 5% fetal bovine serum. When evaluating compounds, the serum can be reduced to a final concentration of 2% and supplemented with 50 μg/mL of gentamicin.

The Middle Eastern coronavirus strain EMC (MERS-CoV) was an original isolate from humans that was amplified in cell culture by Ron Fouchier (Erasmus Medical Center, Rotterdam, the Netherlands) and was obtained from the Centers for Disease Control (Atlanta, Ga.).

### Controls:

Infergen® (interferon alfacon-1, a recombinant non-naturally occurring type-I interferon (Blatt, L., et al., J. Interferon Cytokine Res. (1996) 16(7):489-499 and Alberti, A., BioDrugs (1999) 12(5):343-357) can be used as the positive control drug in all antiviral assays. Infergen=0.03 ng/mL.

### Antiviral Assay:

Virus can be diluted in MEM to a multiplicity of infection=0.001 and each compound can be diluted in MEM+2% FBS using a half-log 8 dilution series. Compound can be added first to 96 well plates of confluent A549 cells followed within 5 mins by virus. Each test compound dilution can be evaluated for inhibition in triplicate. After plating, the plates can be incubated at 37° C. for 4 d. The plates can then be frozen at −80° C.

### Virus Yield Reduction Assay:

Infectious virus yields from each well from the antiviral assay can be determined. Each plate from an antiviral assay can be thawed. Samples wells at each compound concentration tested can be pooled and titered for infectious virus by CPE assay in Vero 76 cells. The wells can be scored for CPE and virus titers calculated. A 90% reduction in virus yield can then be calculated by regression analysis. This represented a one log₁0 inhibition in titer when compared to untreated virus controls.

### Example 13

### Determining the Efficacy of the Compounds Against HCoV—OC43 and SARS-CoV-2 Infections

### Viruses

HCoV—OC43 was obtained from ATCC (Manasas, VA) and SARS-CoV-2 was provided by BEI Resources (NR-52281: USA-WA/2020). HCoV—OC43 and SARS-CoV-2 were propagated in appropriate cells, respectively and titrated by TCID₅₀ method followed by storage of aliquots at −80° C. until further use.

### Cells and Media:

For cytotoxicity and antiviral studies, the following immortalized/transformed cell lines were used: human colon epithelial cells (Caco-2; ATCC® HTB-37™, Manassas, VA, USA), human bronchial epithelial cells (Calu-3; ATCC® HTB-55™, Manassas, VA, USA), human small alveolar cells expressing the human ACE-2 receptor via lentivirus transduction (A549^{hACE2}; kind gift from Dr. Susan Weiss (Lei et al 2021)), and African Green Monkey kidney cells (Vero; ATCC® CCL-81™, Manassas, VA, USA). Media compositions were (1) Caco-2 and Calu-3: Eagle's minimum essential medium (EMEM), 10% fetal bovine serum (FBS), 100 U/mL penicillin-streptomycin (pen-strep), and 2 μM L-glutamine (L-glut), (2) A549^{hACE2} and Vero: Dulbecco's modified eagle medium (DMEM), 10% FBS, 100 U/mL pen-strep. Additional studies were performed in differentiated primary normal human bronchial/tracheal cells (NHBEs) derived from a single donor per culture (Lonza Biosciences CC-2540s, Basal, Switzerland) cultivated in 3D via standard air-liquid interface (ALI; StemCell Technologies 2021) or as custom apical-out lung organoids (HBO; Lee and LeCher et al unpublished). HBTECs were expanded in custom Pneuma-Cult™ Ex Plus medium (Stem Cell Technologies, Vancouver, B.C.) and differentiated in either custom Pneuma-Cult™ ALI medium (ALI; Stem Cell Technologies, Vancouver, B.C.) or Pneuma-Cult™ Organoid Apical-out medium (HBO; Stem Cell Technologies, Vancouver, B.C.) supplemented with hydrocortisone and heparin sulfate. For all experiments, cells were grown at 37° C. in a 95% O₂, 5% CO₂ incubator.

Antiviral Screening Assays: For standard antiviral screening, cells (Caco-2, Calu-3 monolayer, Ace-2^{h549}, and Vero) were grown to confluency (1×10⁵ cells) in 96-well plates. Dose-response curves were performed by treating cells with 2-fold serial dilutions (0-10 μM) of compounds of interest in respective base media containing 2% heat-inactivated FBS (AFBS) then infected with SARS-CoV2 at an MOI of 0.1 (Vero) or 1.0 (Caco2, Calu-3, A549^{hACE2}) for 48 (Vero) or 72 hr (Caco2, Calu-3, A549^{hACE2}). Cells/supernatants were collected in 150 μL RLT Buffer (Qiagen©, Hilden, Germany) for downstream RNA extraction (RNeasy 96 extraction kit; Qiagen©, Hilden, Germany) and subsequent qRT-PCR to detect viral load.

Advanced antiviral assays were also performed by dose-response assay with lead compounds in ALI-Calu-3s, ALI-NHBEs, and HBO—NHBEs with the following modifications: ALI-Calu3—1.8×10⁴ cells were seeded onto a 96-well 1.0 μm pore transwell insert (Corning, USA). After 3 days, media was removed from the apical chamber and cells were cultured for an additional week at ALI. ALI-NHBEs— 1.5×10⁵ cells were seeded onto a 24-well collagen-coated 0.4 μm pore transwell insert (Corning, USA) After 3 days, media was removed from the apical chamber and cells were cultured for an additional 3 weeks at ALI. For both ALI cultures, compounds were added at indicated dilutions to the basolateral chamber. Cells were washed 3× with HEPES-buffered salt solution (HBSS) on apical surface to remove excess mucus then infected by adding 50 μL of SARS-CoV2 virus (MOI 1.0) to the apical chamber for a 5 hr adsorption after which virus was removed and cells retained in ALI for an additional 3 days. For HBO cultures, 3×10³ cells were seeded in hanging-drop suspension with Matrigel® Basement Membrane Matrix (Corning, USA) to generate a single organoid per well and cultured for 21 days. Serially diluted compounds and virus (MOI 1.0) were added directly to the wells for a period of 3 days. Calu3-ALI and HBO infected cultures were collected in 150 μL RLT Buffer (Qiagen®, Hilden, Germany) while NHBE-ALI cultures were collected in 300 μL of Trizol™ Reagent and RNA extracted by phenyl-chloroform method according to manufactures' protocol (ThermoFisher Scientific, USA). All infections were conducted in a BSL-3 level laboratory at Emory University in accordance with the guidelines of the 5th edition of Biosafety in Microbiological and Biomedical Laboratories. All experiments were performed three independent times in duplicate or triplicate.

SARS-CoV2-Yield Inhibition Assay by qRT-PCR Assay:

Virus yield inhibition assays were performed as previously described (Zandi et al 2020). In brief, viral RNA was detected by real-time PCR using a 6-carboxyfluorescein (FAM)-labeled probe with primers against SARS-CoV2 non-structural protein 3 (nsp3). (SARS-CoV-2 FWD: AGA AGA TTG GTT AGA TGA TGA TAG T; SARS-CoV-2 REV:TTC CAT CTC TAA TTG AGG TTG AAC C; SARS-CoV-2 Probe: 56-FAM/TC CTC ACT GCC GTC TTG TTG ACC A/3BHQ_1) RNA isolated from uninfected cells was used as a negative control for virus detection. RNA was added to optimized 10 μM primer/probe mix in Mastermix (qScript™ XLT One-Step RT-qPCR ToughMix®; Quantabio, USA) and run on StepOne Plus real-time PCR (Roche, Germany) according to the manufacturer's protocol. CT values were calculated from replicate groups then virus yield quantified via standard curve. Median effective concentration of compounds (EC₅₀) and concentrations with a 90% inhibitory effect (EC₉₀) were calculated using GraphPad Prism, version 7 (GraphPad Software Inc., San Diego, CA) and reported as the mean±standard deviation.

### SARS-CoV2-Yield Inhibition by Neon-Green Reporter Assay:

Virus yield inhibition assays via neon-green reporter were performed as previously described (Tao et al 2021). In brief, cells were infected in the presence or absence of compounds as described above but with a neon green-expressing icSARS-CoV-2-mNG infectious clone (Xie et al 2020) at MOI 0.1 (Vero). Cultures were monitored daily for the neon green expression in control wells. After 48 (Vero), 72 (Caco-2, Calu-3, A549^{hACE2}, ALI-NHBE, & HBO-NHBE), or 96 (ALI-NHBE, & HBO-NHBE) hr post infection, all wells were imaged and antiviral activity of compounds was determined as percent reduction in mean relative fluorescence from controls.

One of two methods was employed to obtain images: (1) Living cells were imaged on Leica FC 7000 GT microscope with pE-300 fluorescent light housing using LAX software (Leica Biosystems), images processed with Image J software, and cells collected in RLT Buffer for downstream qRT-PCR, or (2) cells were directly fixed in 4% paraformaldehyde for 30 min for removal from the BSL3, permeabilized in 1% ND-40-PBS buffer, DAPI counterstained, and imaged on a Cytation 5 cell imaging multi-mode reader and quantified on Gen5 software (Biotek, Winooski, VT). Uninfected wells served as a negative control measure for background fluorescence.

The anti-SARS-CoV-2 activity is shown in Tables 1 and 2 below:

TABLE 1

| | Anti-SARS- | | | | Anti-SARS-CoV- | |
|---|---|---|---|---|---|---|
| | CoV-2 Activity | | Anti-SARS-CoV-2 | | 2 Activity | |
| | (Vero) (μM) | | Activity (Calu3) (μM) | | (Caco2) (μM) | |
| Compound | EC₅₀ | EC₉₀ | EC₅₀ | EC₉₀ | EC₅₀ | EC₉₀ |
| 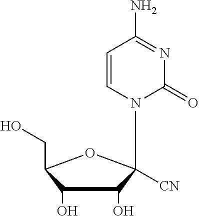 | 0.6 | 2.3 | 0.02 | 0.2 | 0.3 | 1.2 |
| Compound A | | | | | | |
| 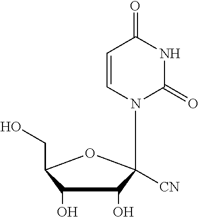 | 4.7 | 8.5 | 0.08 | 0.7 | 0.02 | 0.2 |
| 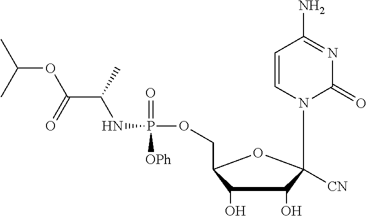 | >10 | >10 | >10 | >10 | 0.7 | 2.1 |
| 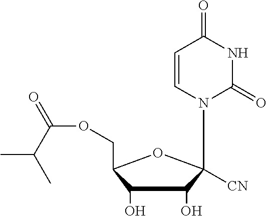 | >10 (−7.5) | ND | 0.2 | 0.7 | 2.1 | 5.7 |
| 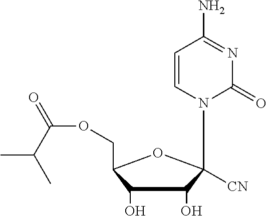 | 1.2 | 2.2 | 1.2 | 4.0 | 0.5 | 2.5 |
| 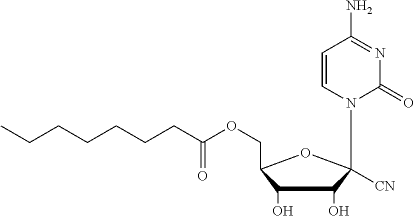 | 0.4 | 1.1 | 0.2 | 1.2 | 0.7 | 1.7 |
| 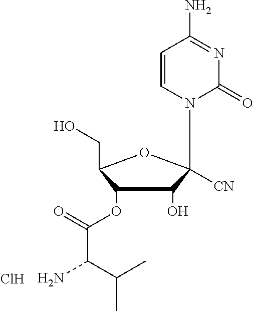 | 2.3 | 6.6 | 0.3 | 4.3 | 3.3 | 8.3 |
| NHC | 0.2 | 0.42 | 0.6 | 2.0 | 1 | >10 |
| Remdesivir | 1.0 | 3.5 | 0.1 | 0.6 | 0.004 | 0.04 |

ND: Not determined,
NHC = N⁴-hydroxycytidine

TABLE 2

| | Anti-SARS-CoV-2 | |
|---|---|---|
| | activity in Vero cells | |
| | (μM) | |
| Structures | EC₅₀ | EC₉₀ |
| 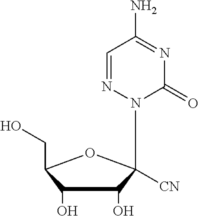 | 4.0 | 9.8 |
| 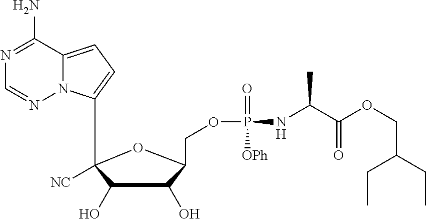 | <10 | |
| 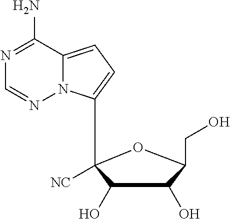 | >10 | |
| 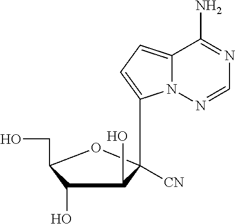 | <10 | |
| Remdesivir | 1.2 | 3.6 |

TABLE 3

| | Cytotoxicity | | | |
|---|---|---|---|---|
| | CC₅₀ (μM) | | | |
| Compound | PBM | CEM | Vero | Huh7 |
| 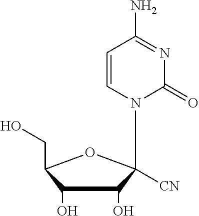 | 92 | 99 | >100 | >100 |
| Compound A | | | | |
| 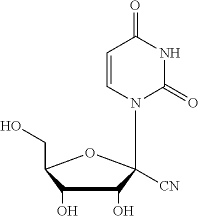 | >100 | 12.1 | >100 | >100 |
| 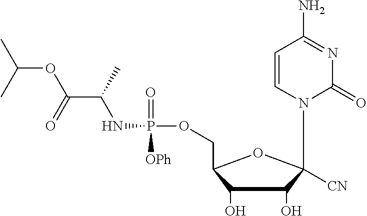 | >100 | >100 | >100 | >100 |
| 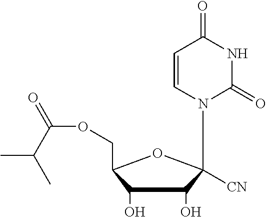 | >100 | 39.0 | >100 | 88.9 |
| 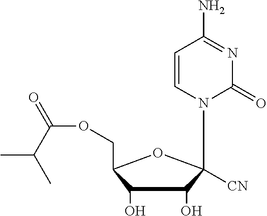 | 63.1 | 15.8 | 73.8 | >100 |
| 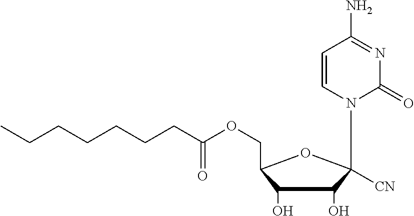 | >100 | 3.7 | 45.7 | 77.8 |
| 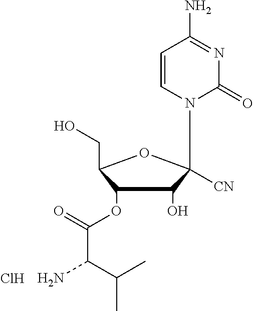 | >100 | — | 93.5 | — |
| NHC | 49.2 | 2.6 | 16.5 | 80.3 |
| Remdesivir | 6.4 | 12 | >100 | 1.5 |

TABLE 4

| | Anti-SARS-COV-2 Activity (μM) | | | | | |
|---|---|---|---|---|---|---|
| | Calu3 ALI | | HAE-ALI | | HAE-HBO | |
| Compound | EC₅₀ | EC₉₀ | EC₅₀ | EC₉₀ | EC₅₀ | EC₉₀ |
| 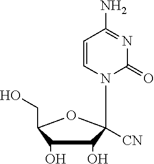 | 1.3 | 7.4 | 0.4 | 1.6 | 0.3 | 0.5 |
| Compound A | | | | | | |
| Remdesivir | 0.9 | 4.6 | 0.6 | 1.2 | 0.7 | 1.5 |

TABLE 5

Cytotoxicity CC₅₀ (μM)

| | Calu3 | Caco2 |
|---|---|---|
| Compound A | >100 | >100 |
| Cyclohexamide (+ve- | 10 | 10 |
| control) | | |

TABLE 6

| | Antiviral Activity | | | | Antiviral Activity | | | |
|---|---|---|---|---|---|---|---|---|
| | Against SARS-CoV2 | | | | Against SARS-CoV2 | | | |
| | Variants in Vero (μM) | | | | Variants in Calu3 (μM) | | | |
| | Compound A | | Remdesivir | | Compound A | | Remdesivir | |
| Alpha | | | | | | | | |
| Beta | 0.3 | 1.4 | 0.4 | 1.6 | 0.3 | 1.2 | 0.3 | 0.4 |
| Gamma | 0.8 | 1.0 | 1.1 | 2.2 | 1.1 | 1.2 | ND | ND |
| Delta | 0.2 | 0.3 | 0.3 | 2.1 | 0.04 | 0.1 | 0.26 | 0.3 |
| Lineage | ND | ND | ND | ND | 1.8 | 3.6 | 0.3 | 2.3 |
| A (Eng) | | | | | | | | |
| Lineage | 0.2 | 0.4 | 0.3 | 2.9 | 1.7 | 4.5 | 0.02 | 0.3 |
| A (Wa) | | | | | | | | |
| Alpha | 0.8 | 1.2 | 0.2 | 0.6 | 0.1 | 0.4 | 0.1 | 0.4 |

ND: Not determined

TABLE 7

| | Anti-Omicron | | Anti-Omicron | |
|---|---|---|---|---|
| | Activity | | Activity | |
| | (Calu-3) | | (Caco-2) | |
| | EC₅₀ | EC₉₀ | EC₅₀ | EC₉₀ |
| Compounds | (μM) | (μM) | (μM) | (μM) |
| remdesivir | 0.2-0.3 | 0.6-0.7 | 0.05 | 0.4 |
| NHC | 0.4 | 1.2 | >10 (13.3) | >10 |
| Compound A | 1.8-2.1 | 4.5-6.2 | 1.2 | 2.2 |
| 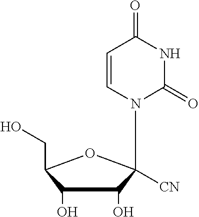 | 3.8 | >10 | >10 (11.9) | >10 |

### REFERENCES

Li, Y., Renner, D. M., Comar, C. E., Whelan, J. N., Reyes, H. M., Cardenas-Diaz, F. L., and Weiss, S. R. (2021). SARS-CoV-2 induces double-stranded RNA-mediated innate immune responses in respiratory epithelial-derived cells and cardiomyocytes. Proceedings of the National Academy of Sciences, 118(16).
Stem Cell Technologies. Model the human airway in vitro as ALI cultures or airway organoids.
Lee, J. H. and LeCher, J. C., et al (2021). Apical-out human bronchial organoid models for SARS-CoV-2 infection studies. Unpublished Study.
Zandi, K., Amblard, F., Musall, K., Downs-Bowen, J., Kleinbard, R., Oo, A., and Schinazi, R. F. (2020). Repurposing nucleoside analogs for human coronaviruses. Antimicrobial agents and chemotherapy, 65(1), e01652-20.
Tao, S., Zandi, K., Bassit, L., Ong, Y. T., Verma, K., Liu, P., and Schinazi, R. F. (2021). Comparison of anti-SARS-CoV-2 activity and intracellular metabolism of remdesivir and its parent nucleoside. Current Research in Pharmacology and Drug Discovery, 2, 100045.
Xie, X., Muruato, A., Lokugamage, K. G., Narayanan, K., Zhang, X., Zou, J., and Shi, P. Y. (2020). An infectious cDNA clone of SARS-CoV-2. Cell host & microbe, 27(5), 841-848.

### Evaluation in a Model of SARS-CoV-2 Infection of Human Lung Epithelium and Monocytes System:

In vitro transmigration experiments and infection with virus. The H441 Club cell line was grown on Alvetex scaffolds (ReproCELL, Glasgow, UK) coated with rat-tail collagen (Sigma) for 2 weeks at air liquid interface with 2% v/v Ultroser G (Crescent Chemical, Islandia, NY) in 50/50 DMEM/F12. The filters were then flipped and placed into fresh media in the bottom of the well. Virus (PR8: A/Puerto Rico/8/1934; OC43; or NR-52281, SARS-CoV-2 Isolate USA-WA1/2020) is added to the media such that that the multiplicity of infection (MOI) is 0.1 and incubated for 24 hours. This setup requires manual flipping of filters prior to transmigration, a delicate process to perform in BSL3 conditions. Thus, the epithelial cells must be infected while the cells are submerged and no longer at ALI, which may introduce artifacts reminiscent of pneumonia. The filters were transferred to RPMI media with LTB4 (100 nM) and CCL2 (250 pg/mL) with or without additional drugs. Drugs were used at a final concentration of 1 or 10 uM. The untreated condition contained 0.01% v/v DMSO as a vehicle control. Blood monocytes were purified using RosetteSep (StemCell). A total of around 10⁶ cells was loaded onto the Alvetex scaffold for transmigration, which was allowed to occur for 24 hours. After transmigration, TriPure (Roche) was added to epithelial cells and frozen at −80° C.

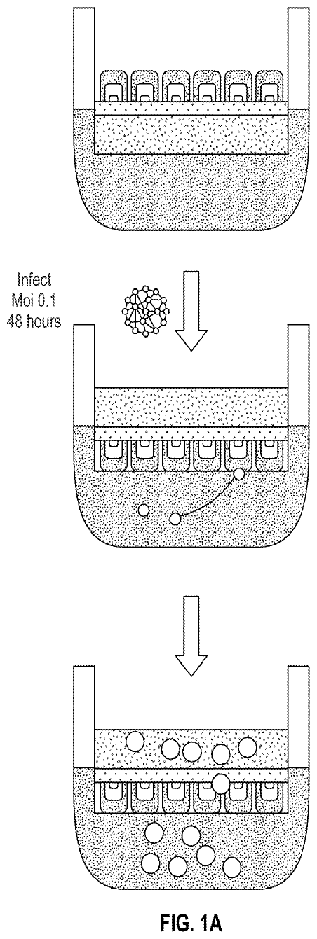

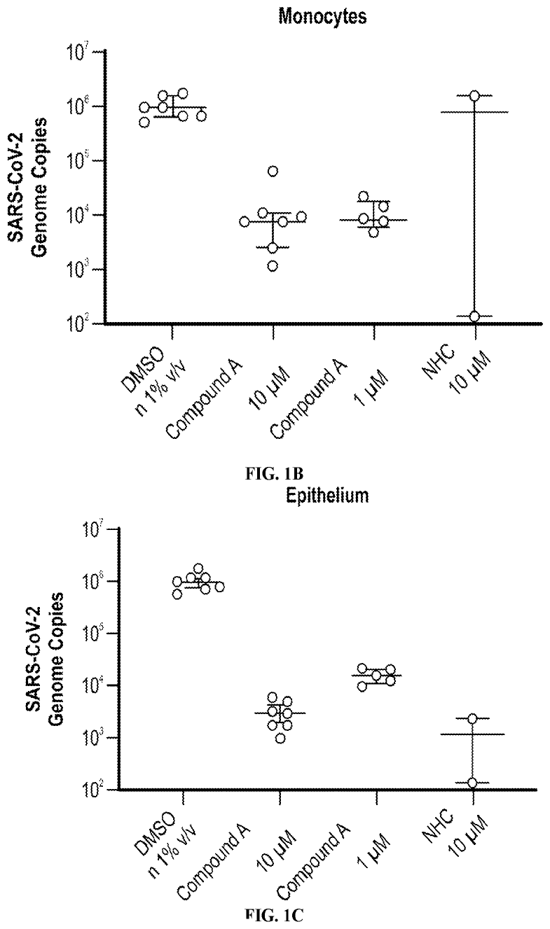

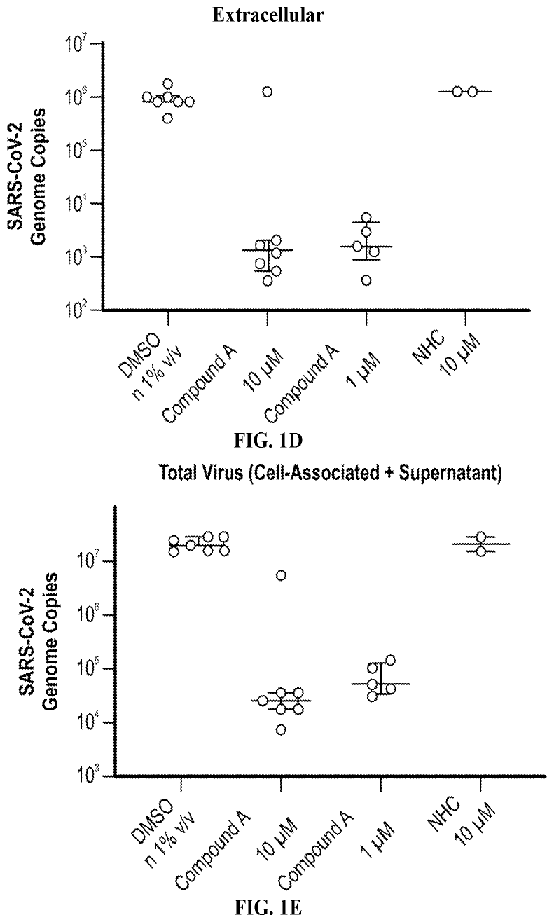

FIG. 1A is a schematic illustration of the model of SARS-CoV-2 infection of human lung epithelium and monocytes system. FIG. 1B is a chart showing the effect of Compound A at different concentrations on the number of copies of SARS-CoV-2 in infected monocytes in the model of SARS-CoV-2 infection of human lung epithelium and monocytes system. FIG. 1C is a chart showing the effect of Compound A at different concentrations on the number of copies of SARS-CoV-2 in infected epithelial cells in the model of SARS-CoV-2 infection of human lung epithelium and monocytes system. FIG. 1D is a chart showing the effect of Compound A at different concentrations on the number of copies of SARS-CoV-2 (extracellular) in the model of SARS-CoV-2 infection of human lung epithelium and monocytes system. FIG. 1E is a chart showing the effect of Compound A at different concentrations on the number of copies of SARS-CoV-2 (total virus; cell-associated and supernatant) in the model of SARS-CoV-2 infection of human lung epithelium and monocytes system. As shown in FIGS. 1A-E, compound A reduces viral loads by 2-3 log at 1 microM.

### Plasma Stability:

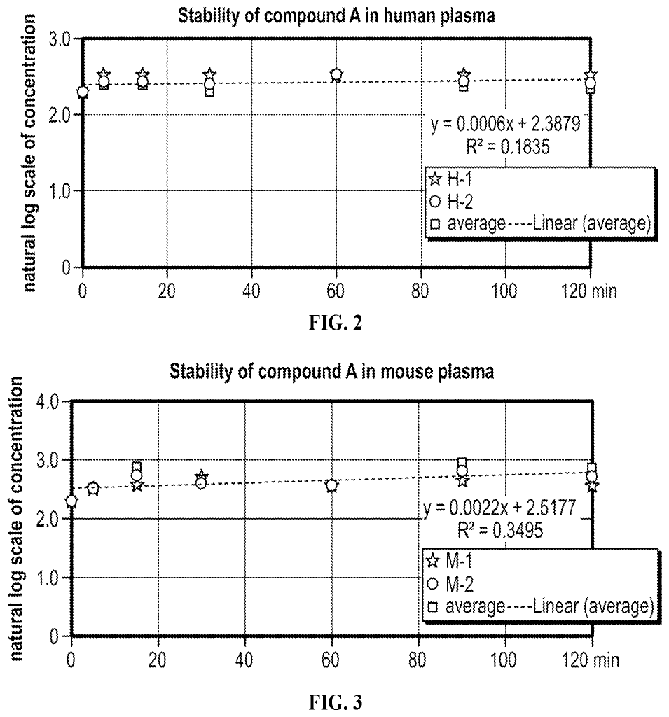

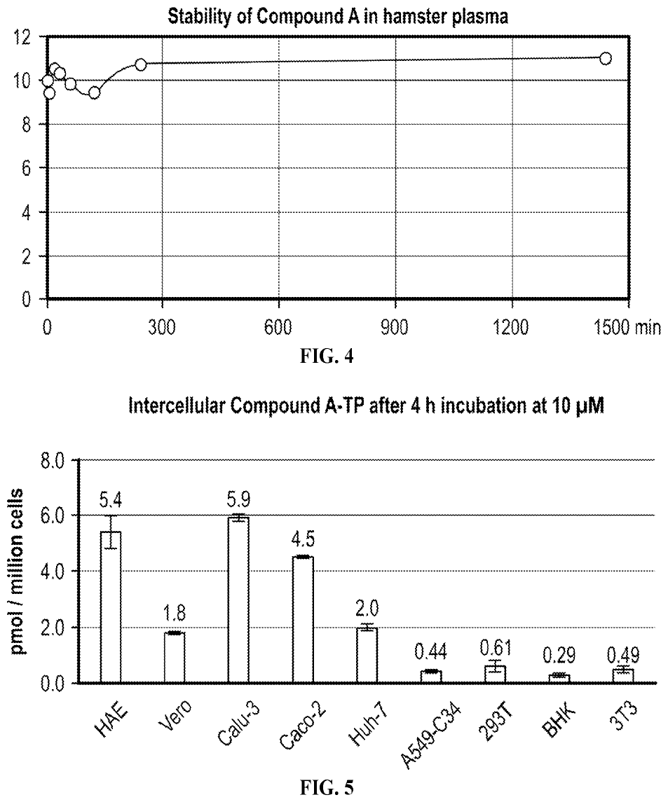

An aliquot of 450 μL of human, mouse or hamster plasma were exposed to 10 μM of compound and incubated at 37° C. At 0, 5, 15, 30, 60, 90 and 120 min, 50 μL of plasma sample was mixed with 200 μL of ice-cold methanol (70%). 50 μL supernatant was dried and reconstituted in 100 μL H₂O. Propantheline bromide was used as positive control. The supernatant was then subjected to LC-MS analysis (LC-MS condition: Instrument: Thermo TSQ Quantiva. Column: Kinetex C₈₈ (50×2.1 mm, 2.6 m). LC buffers: A): 0.1% formic acid, and B): acetonitrile. MRM methods: (269.2-112 positive), Indinavir (IS, 614.4-421.2 positive, IS)). The stability results are shown in FIGS. 2-4 (human plasma, mouse plasma and hamster plasma, respectively). Compound A is stable in human and mouse plasma up to 2 hours and stable up to 24 h in hamster plasma. Compound 23 was converted to compound A in hamster plasma in less than 5 min.

### Cellular Pharmacology:

The uptake and egress of Compound A was measured in cell culture in HAE cells, as well as a variety of other cells. The cell culture involved HAE cells seeded at a density of 0.15×10⁶/well, and other cells were seeded at a density of 1×10⁶/well. To measure uptake, the compound was incubated in cells for 4 hours at a concentration of 10 μM. To measure egress of the compound from the cells, the cells were pre-treated for 24 hr at a concentration of 10 μM, at which time the media was replaced, then cells were harvested at 0, 2, 4, 6, 8, 12, 24, and 32 hours.

LC-MS/MS: TSQ Quantiva. Buffer A: 2 mM NH₃H₂PO₄ with 3 mM Hexylamine; Buffer B: Acetonitrile; flow rate: 250 μL/min. HPLC column: Kinetex EVO C18 100×2.1 mm, 2.6 m. MS detection: SRM mode.

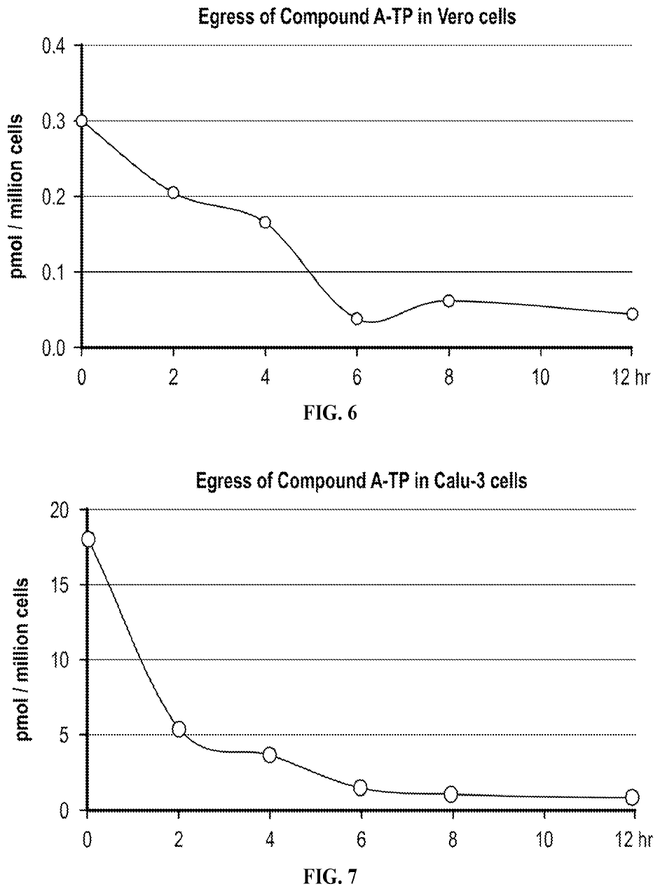

The data regarding the intracellular concentrations (pmol/million cells) in different types of cells (HAE, Vero, Calu-3, Caco-2, Huh-7, A549-C34, 293T, BHK, and 3T3 cells), measured at 4 hours, is shown in FIG. 5. Compound A triphosphate is found in a large variety of cell lines. The egress of Compound A-triphosphate was measured in Vero cells and Calu-3 cells (pmol/million cells) over time (0-12 hours), and the data is shown in FIGS. 6 and 7, respectively. The half life of Compound A triphosphate in Vero and Calu-3 cells is 4.1 and 1.7 hours, respectively.

### Non-Compartmental PK Analysis of IV and Oral Compound a in Mice

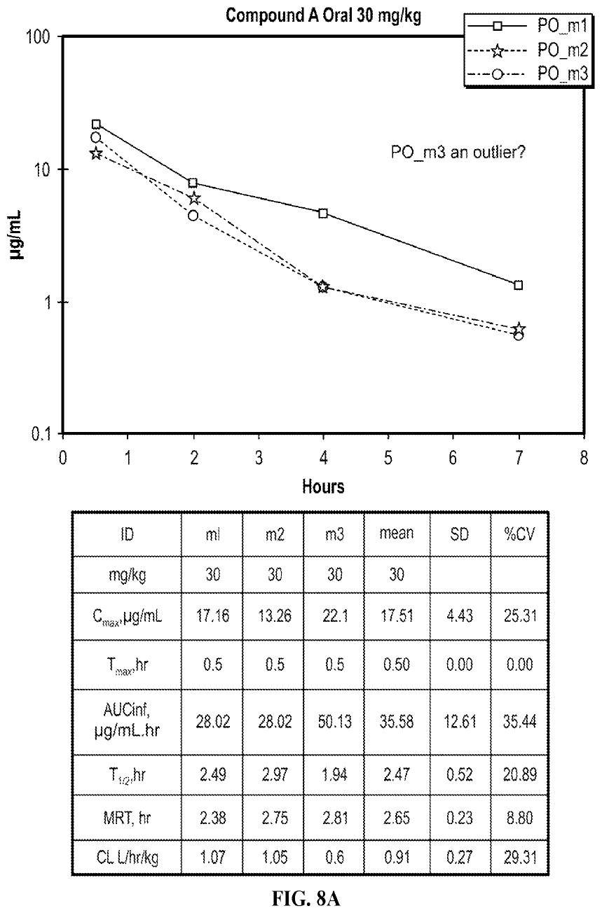

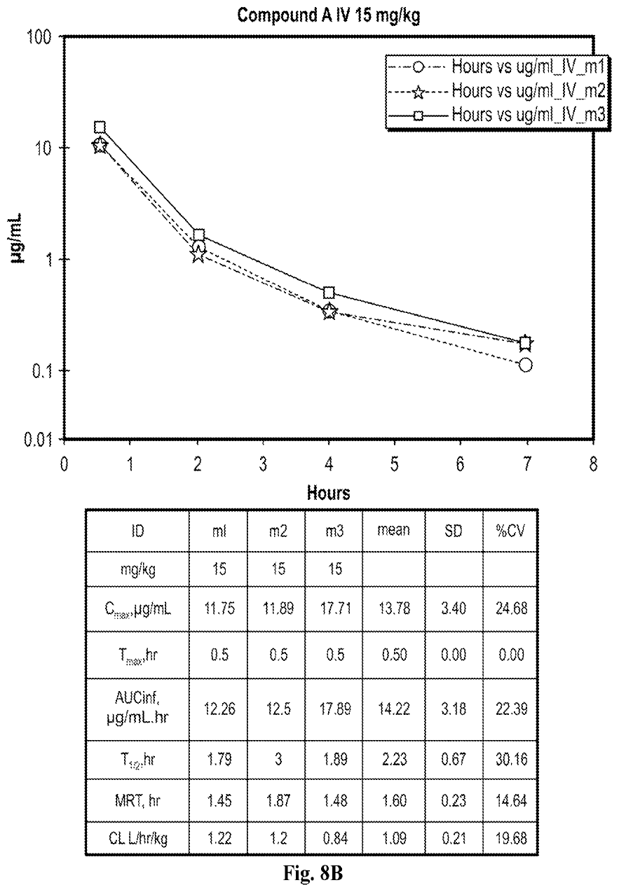

A study was performed to measure the concentration of Compound A (g/ml) over time (hours) when administered orally at a concentration of 30 mg/kg (FIG. 8A) and intravenously at a concentration of 15 mg/kg (FIG. 8B). Compound A displays excellent oral bioavailability in mice. As shown in FIG. 8B, the plasma concentration of Compound A, when administered to three mice, declined in a logarithmic manner over the course of 7 hours, from around 10 to around 0.1 μg/ml.” The following calculations/assumptions were made:

$$\% oral bioavailability = ( {AUC}_{PO} / {dose}_{PO} ) / ( {AUC}_{IV} / {dose}_{IV} ) \times 100 = 125 \%$$

Excluding PO_m3 (possible outlier), mean oral AUC_{PO}=28.3 μg/ml·hr and the % oral bioavailability=99.5%, and the mean oral CL=1.06 L/hr per kg.

The mean terminal t_{1/2}=2.47 and 2.23 hr for oral and IV doses, respectively.

Mean input time (MIT) into plasma of oral dose=MRT_{PO}−MRT_{1V}=2.65 hr−1.60 hr=1.05 hr.

Approximation, assuming 1-compartment PK: Kₐ is ˜1/MIT=0.95 hr⁻¹.

### Quantification of Compound A in Mouse Lung and Brain

CD-1 mice were treated with Compound A PO (30 mg/kg, 3 mice) or IV (15 mg/kg, 3 mice). Lung and brain samples were collected at 7 h.

### Sample Preparation:

Lung and brain samples were homogenized with 5× volume of 20% MeOH.

50 μL tissue homogenate was mixed with 250 μL MeOH (containing 40 nM indinavir as IS).

The supernatant was air-dried, then reconstituted in 150 μL of H₂O.

Subjected to LC-MS analysis.

Calibration curve range: 30 nM to 30 μM.

### LC-MS/MS Condition:

Instrument: TSQ Quantiva, Column: Kinetex XB—C8 (50×2.1 mm, 2.6 μm)

LC buffers: A): 0.1% formic acid, and B): Acetonitrile

LC gradients: 0-0.3 min, 2% B; 0.3-3 min, 2%-80% B; 3-3.2 min, 80% B; 3.2-3.5 min, 80%-2% B; 3.5-8 min, 2% B

MRM methods: Compound A (269.2→112 positive), Indinavir (IS, 614.4→421.2 positive, IS)

The data is summarized in Table 8:

TABLE 8
Quantification of Compound A in a Mouse Model

| | | Lung | | Brain | | Plasma | |
|---|---|---|---|---|---|---|---|
| | Mice | ng/g | μM* | ng/g | μM* | ng/g | μM* |
| PO | 1 | 881.8 | 3.29 | 73.9 | 0.28 | 560 | 2.09 |
| | 2 | 730.8 | 2.72 | 40.8 | 0.15 | 620 | 2.32 |
| | 3 | 1,615 | 6.02 | 44.7 | 0.17 | 1134 | 4.99 |
| | Average | 1,076 | 4.01 | 53.1 | 0.20 | 880 | 3.13 |
| IV | 1 | 223.3 | 0.83 | 39.3 | 0.15 | 100 | 0.37 |
| | 2 | 412.0 | 1.54 | 56.9 | 0.21 | 160 | 0.61 |
| | 3 | 327.1 | 1.22 | 117.8 | 0.44 | 160 | 0.59 |
| | Average | 320.8 | 1.20 | 71.3 | 0.27 | 140 | 0.52 |

*Assuming tissue density is 1 g/mL

### Single Dose Range-Finding Study in Golden Syrian Hamsters:

3 male Syrian hamsters were injected intraperitoneally with a Compound A starting at 5 mg/kg at time 0 with 25 mg/kg administered every 30 minutes upto a final 80 mg/kg. Clinical observations of toxicity were recorded. 12 and 24 hrs later: All animals were BAR (Bright Alert Responsive) and showed no signs of discomfort. There were no signs of toxicity in hamsters given compound A via IP injection and a total dose of 80 mg/kg. The data is summarized in Table 9.

TABLE 9
Clinical Observations of individual hamsters

| | | | |
|---|---|---|---|
| | Temp | Weight | |
| Dose: | (° C.) | (grams) | Notes |

Rodent 1

| | | | |
|---|---|---|---|
| First Dose | 35.5 | 115 | B.A.R.*Observed eating and |
| 5 mg/kg | | | drinking; No sign of discomfort |
| Second Dose | 36.3 | | B.A.R.*Observed eating and |
| 30 mg/kg (total) | | | drinking; No sign of discomfort |
| Third Dose | 35.9 | | B.A.R.* No sign of discomfort |
| 55 mg/kg (total) | | | |
| Fourth Dose | 35.3 | | B.A.R.* No sign of discomfort |
| 80 mg/kg (total) | | | |

Rodent 2

| | | | |
|---|---|---|---|
| First Dose | 36.6 | 148 | B.A.R.* Observed eating and |
| 5 mg/kg | | | drinking; No sign of discomfort |
| Second Dose | 37.6 | | B.A.R.* Observed eating and |
| 30 mg/kg (total) | | | drinking; No sign of discomfort |
| Third Dose | 36.8 | | B.A.R.* No sign of discomfort |
| 55 mg/kg (total) | | | |
| Fourth Dose | 36.2 | | B.A.R.* No sign of discomfort |
| 80 mg/kg (total) | | | |

Rodent 3

| | | | |
|---|---|---|---|
| First Dose | 36.5 | 132 | B.A.R.* Observed eating and |
| 5 mg/kg | | | drinking; No sign of discomfort |
| Second Dose | 36.8 | | B.A.R.* Observed eating and |
| 30 mg/kg (total) | | | drinking; No sign of discomfort |
| Third Dose | 36.8 | | B.A.R.* No sign of discomfort |
| 55 mg/kg (total) | | | |
| Fourth Dose | 36.2 | | B.A.R.* No sign of discomfort |
| 80 g/kg (total) | | | |

*B.A.R. = Bright Alert Responsive

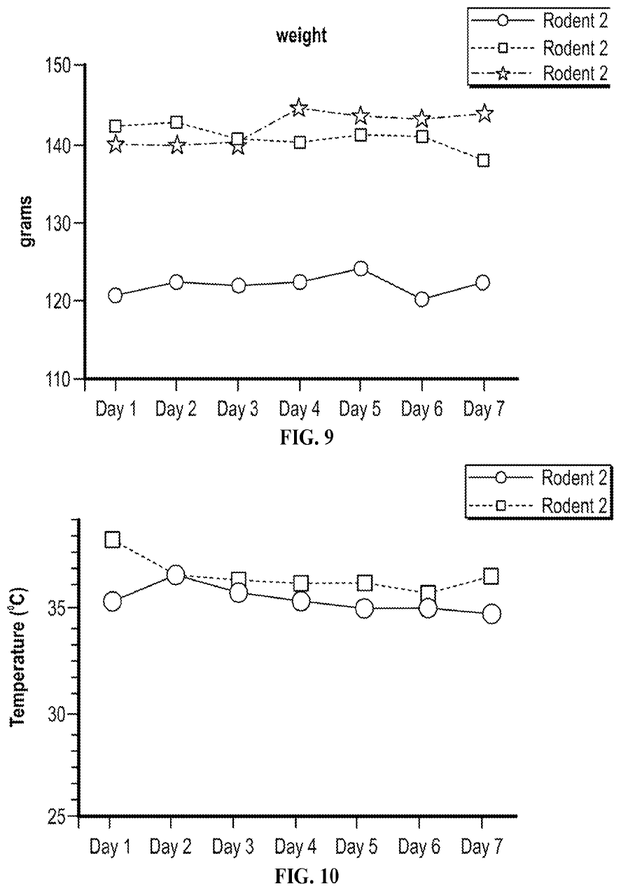

Multi-dose range-finding study in Golden Syrian Hamsters: 3 male Syrian hamsters were given 30 mg/kg of compound A by oral gavage daily for 7 days. The body weight of each of the individual hamsters during the nucleoside analog treatment was measured (grams per day) over a 7 day period. The data (shown in FIG. 9) show that the hamsters maintained their weight during the treatment. The body temperature of the hamsters was also measured over the course of the treatment in two of the hamsters (FIG. 9, Rodent 1 and Rodent 2). The third hamster lost the chip, and, despite re-chipping, temperatures were still not recording. The data is shown in FIG. 10. Compound A (30 mg/kg) does not affect weight and body temperature of male Syrian hamsters.

Daily observations of the hamsters were taken, particularly with respect to their ability to eat and drink normally, and other behavior and activity ratings (B.A.R.), and the observations are summarized below in Table 10. Briefly, all hamsters were able to eat and drink normally throughout their treatment with Compound A.

TABLE 10
Observations of hamsters following administration of Compound A.
Daily Observations:

| | Rodent 1 | Rodent 2 | Rodent 3 |
|---|---|---|---|
| Day 1 | B.A.R. Eating | B.A.R. Eating | B.A.R. Eating |
| | drinking normally | drinking normally | drinking normally |
| Day 2 | B.A.R. Eating | B.A.R. Eating | B.A.R. Eating |
| | drinking normally | drinking normally | drinking normally |
| Day 3 | B.A.R. Eating | B.A.R. Eating | B.A.R. Eating |
| | drinking normally | drinking normally | drinking normally |
| Day 4 | B.A.R. Eating | B.A.R. Eating | B.A.R. Eating |
| | drinking normally | drinking normally | drinking normally |
| Day 5 | B.A.R. Eating | B.A.R. Eating | B.A.R. Eating |
| | drinking normally | drinking normally | drinking normally |
| Day 6 | B.A.R. Eating | B.A.R. Eating | B.A.R. Eating |
| | drinking normally | drinking normally | drinking normally |
| Day 7 | B.A.R. Eating | B.A.R. Eating | B.A.R. Eating |
| | drinking normally | drinking normally | drinking normally |

No toxicity was observed with oral gavage of 30 mg/kg/day of nucleoside analog given daily for a period of 7 days.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

1. A method for treating or preventing a Coronaviridae, Flaviviridae, Picornaviridae, Bunyaviridae, or Togaviridae infection, comprising administering a treatment or preventative amount of a compound of Formula (A) to a patient in need of treatment or prevention thereof: 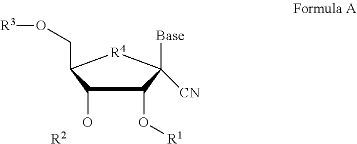
or a pharmaceutically acceptable salt or prodrug thereof, wherein:
R⁴ is O, CH₂, S, Se, or 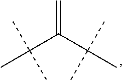
R¹ and R² are independently selected from the group consisting of H, an L-amino acid ester, a D-amino acid ester, an N-substituted L-amino acid ester, an N-substituted D-amino acid ester, an N,N-disubstituted L-amino acid ester, an N,N-disubstituted D-amino acid ester, (acyloxybenzyl)ester, (acyloxybenzyl)ether, optionally substituted bis-acyloxybenzyl)esters, optionally substituted (acyloxybenzyl)esters, an optionally substituted —C(O)—C₁₋₁₂R′, an optionally substituted —C(O)O—R′, an optionally substituted —C(O)S—R′, an optionally substituted —C(S)S—R′, an optionally substituted —C(NR′)OR′, an optionally substituted —C(NR′)SR′, an optionally substituted —C(NR′)N(R′)₂, and an optionally substituted —O—C(O)N(R′)₂, PEG ester, PEG carbonate, an optionally substituted —CH₂—O—C(O)—R′, an optionally substituted —CH₂—O—C(O)O—R′, an optionally substituted —CH₂—CH₂—S—C(O)—R′, a lipid ester, a lipid carbonate, (in which the lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy),
R′ is C₁₋₁₆ alkyl, C₂₋₁₆ alkenyl, C₂₋₁₆ alkynyl, or C₃₋₇ cycloalkyl,
wherein optional substituents are selected from the group consisting of halo, C₁₋₁₂ haloalkyl, C₁₋₁₆ alkyl, C₂₋₁₆ alkenyl, C₂₋₁₆ alkynyl, C₃₋₇ cycloalkyl, hydroxyl, carboxyl, C₁₋₁₂ acyl, aryl, heteroaryl, C₁₋₆ acyloxy, amino, amido, carboxyl derivatives, alkylamino, di-C₁₋₁₂-alkylamino, arylamino, C₁₋₁₂ alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, boronic acid and boronic ester;
R³ is H, an L-amino acid ester, a D-amino acid ester, an N-substituted L-amino acid ester, an N-substituted D-amino acid ester, an N,N-disubstituted L-amino acid ester, an N,N-disubstituted D-amino acid ester, (acyloxybenzyl) ester, (acyloxybenzyl) ether, optionally substituted bis-acyloxybenzyl) ester, optionally substituted (acyloxybenzyl) ester, an optionally substituted —C(O)—R′, an optionally substituted —C(O)O—R′, an optionally substituted —C(O) SR′, an optionally substituted —C(S)SR′, PEG ester, PEG carbonate, an optionally substituted —CH₂—O—C(O)—R′, an optionally substituted —CH₂—O—C(O)O—R′, an optionally substituted —CH₂—CH₂—S—C(O)—R′, an optionally substituted —C(NR′)OR′, an optionally substituted —C(NR′)SR′, an optionally substituted —C(NR′)N(R′)₂, an optionally substituted —O—C(O)N(R′)₂, a lipid ester, a lipid carbonate (in which a lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy), O—P(O)R⁶R⁷, or a mono-, di-, or triphosphate, wherein, when chirality exists at the phosphorous center, it may be wholly or partially Rₚ or Sₚ or any mixture thereof,
R⁶ and R⁷ are independently selected from the group consisting of:
(a) OR¹⁵ where R¹⁵ selected from the group consisting of H, 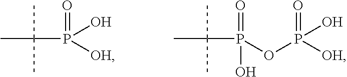
Li, Na, K, substituted or unsubstituted C₁₋₂₀alkyl, substituted or unsubstituted C₃₋₆cycloalkyl, optionally substituted —C(NR′)OR′, optionally substituted —C(NR′)SR′, optionally substituted —C(NR′)N(R′)₂, optionally substituted —O—C(O)N(R′)₂, C₁₋₄(alkyl)aryl, benzyl, C₁₋₆ haloalkyl, C₂₋₃(alkyl)OC₁₋₂₀(alkyl, C₂₋₃(alkyl)OC₁₋₂₀alkene, C₂₋₃(alkyl)OC₁₋₂₀alkyne, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₁)₀₋₆ CON(R′)₂;
where R¹⁶ is independently H, substituted or unsubstituted C₁₋₂₀ alkyl, substituted or unsubstituted C₁₋₂₀ alkene, substituted or unsubstituted C₁₋₂₀alkyne, the carbon chain derived from a fatty alcohol or C₁₋₂₀alkyl substituted with a C₁₋₆ alkyl, C₁₋₆ alkoxy, di(C₁₋₆ alkyl)-amino, fluoro, C₃₋₁₀cycloalkyl, cycloalkyl-C₁₋₆ alkyl, cycloheteroalkyl, aryl, heteroaryl, substituted aryl, or substituted heteroaryl: wherein the substituents are C₁₋₅ alkyl, C₁₋₅ alkene, C₁₋₅ alkyne, C₃₋₇ cycloalkyl or C₁₋₅ alkyl substituted with a C₁₋₆ alkyl, alkoxy, di(C₁₋₆ alkyl)-amino, fluoro, C₃₋₁₀ cycloalkyl, or cycloalkyl; and
(b) the ester of a D- or L-amino acid 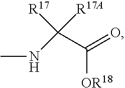
wherein R¹⁷ and R¹⁸ are, independently, H, C₁₋₂₀ (alkyl, C₁₋₂₀ alkene, C₁₋₂₀ alkyne, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and R^{17A} is H or C₁₋₂alkyl; 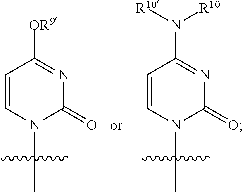
R^{9′} is H, an L-amino acid ester, a D-amino acid ester, an N-substituted L-amino acid ester, an N-substituted D-amino acid ester, an N,N-disubstituted L-amino acid ester, an N,N-disubstituted D-amino acid ester, an (acyloxybenzyl)ester, an (acyloxybenzyl)ether, an optionally substituted bis-acyloxybenzyl)ester, an optionally substituted (acyloxybenzyl)ester, an optionally substituted —C(O)—R′, an optionally substituted —C(O)O—R′, an optionally substituted —C(O)S—R′, an optionally substituted —C(S)S—R′, an optionally substituted C₁₋₁₂-alkyl, an optionally substituted C₂₋₁₂ alkenyl, an optionally substituted C₂₋₁₂ alkynyl, an optionally substituted C₃₋₆ cycloalkyl, an optionally substituted —C(NR′)OR′, an optionally substituted —C(NR′)SR′, an optionally substituted —C(NR′)N(R′)₂, an optionally substituted —O—C(O)N(R′)₂, a PEG ester, a PEG carbonate, an optionally substituted —CH₂—O—C(O)—R′, an optionally substituted —CH₂—O—C(O)O—R′, an optionally substituted —CH₂—CH₂—S—C(O)—R′, a lipid ester, or a lipid carbonate,
wherein a lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy),
R¹⁰ and R^{10′} are independently H, OH, an L-amino acid amide, a D-amino acid amide, (acyloxybenzyl)amide, (acyloxybenzyl)amine, optionally substituted (acyloxybenzyl)esters, an optionally substituted —C(O)—R′, an optionally substituted —C(O)O—R′, an optionally substituted —C(O)S—R′, an optionally substituted —C(S)S—R′, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, PEG amide, PEG carbamate, an optionally substituted —CH₂—O—C(O)—R′, an optionally substituted —CH₂—O—C(O)O—R′, an optionally substituted —CH₂—CH₂—S—C(O)—R′, a lipid amide, an optionally substituted —C(NR′)OR′, an optionally substituted —C(NR′)SR′, an optionally substituted —C(NR′)N(R′)₂, an optionally substituted —O—C(O)N(R′)₂, or a lipid carbamate, wherein a lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy), with the proviso that R¹⁰ and R^{10′} cannot both be OH.

2. The method of claim 1, wherein R⁴ is O.

3. The method of claim 1, wherein R⁴ is CH₂.

4. The method of claim 1, wherein R⁴ is S.

5. The method of claim 1, wherein R⁴ is O and R¹, R², R³, R⁹, R¹⁰ and R^{10′} are not all H.

6. The method of claim 1, wherein R¹ and R² are H, an L-amino acid ester, a D-amino acid ester or an optionally substituted —C(O—C₁₋₁₂ alkyl.

7. The method of claim 1, wherein R³ is H, an L-amino acid ester, a D-amino acid ester or an optionally substituted —C(O)—C₁₋₁₂ alkyl.

8. The method of claim 1, wherein R¹⁰ and R^{10′} are independently H, OH, an L-amino acid amide, a D-amino acid amide, an optionally substituted —C(O)—C₁₋₁₂ alkyl, with the proviso that R¹⁰ and R^{10′} cannot both be OH.

9. The method of claim 1, R⁹ is H, an L-amino acid ester with the oxygen to which it is attached, a D-amino acid ester with the oxygen to which it is attached, or an optionally substituted —C(O)—C₁₋₁₂ alkyl.

10. The method of claim 1, wherein R¹⁰ and R^{10′} are both H.

11. The method of claim 1, wherein the compound is one of the following compounds: 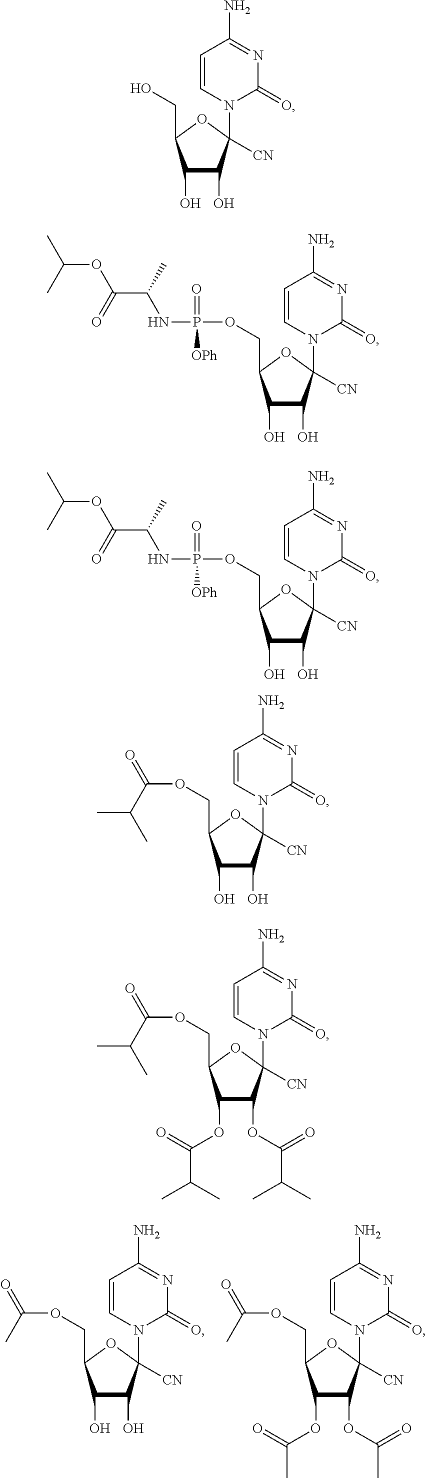 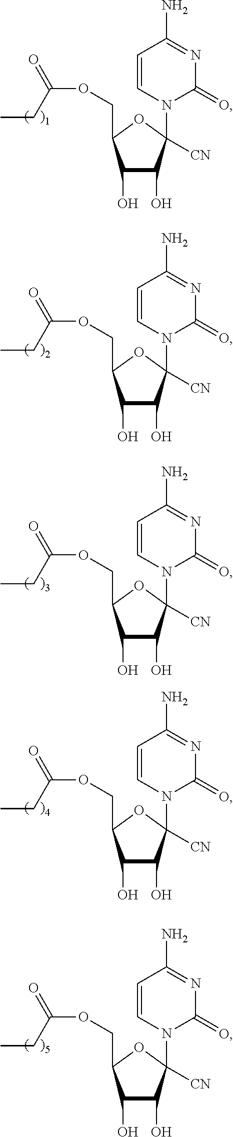 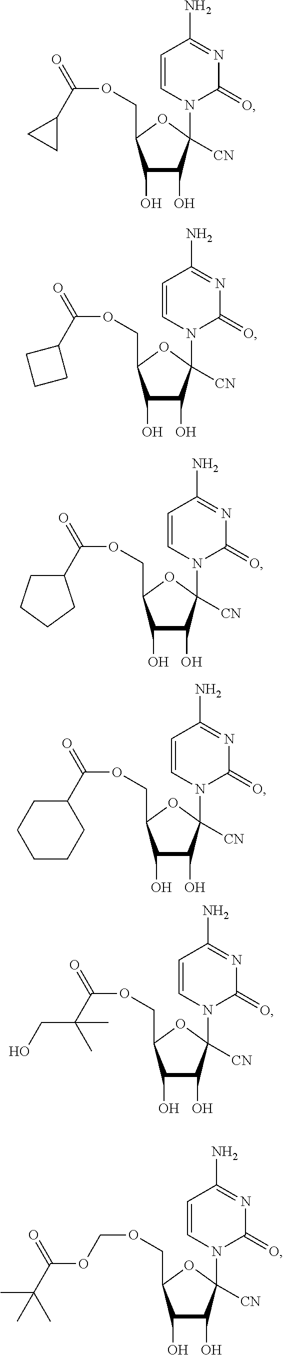 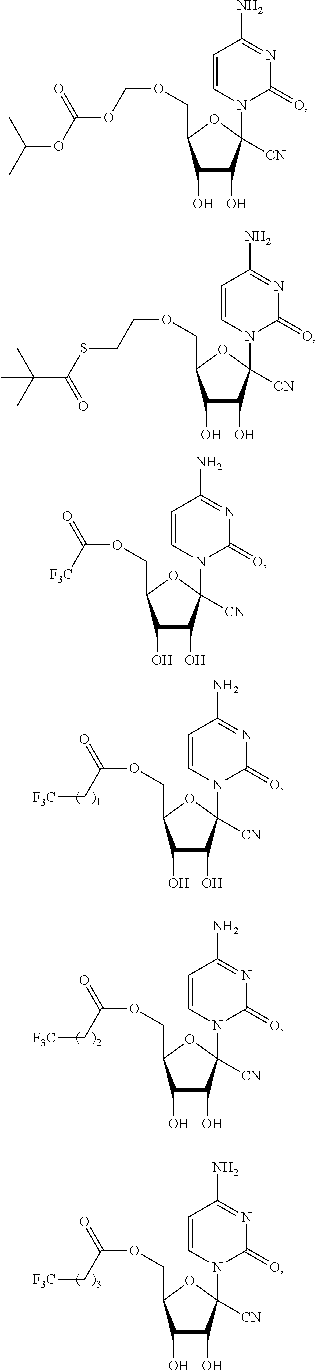 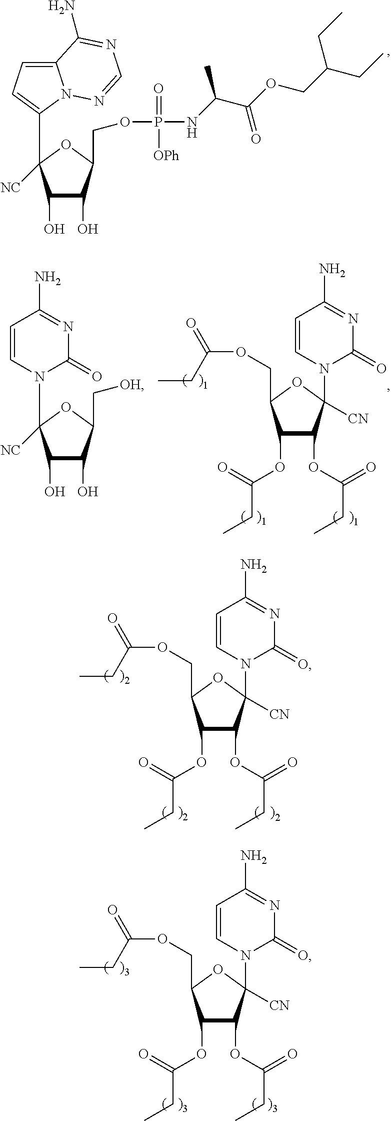 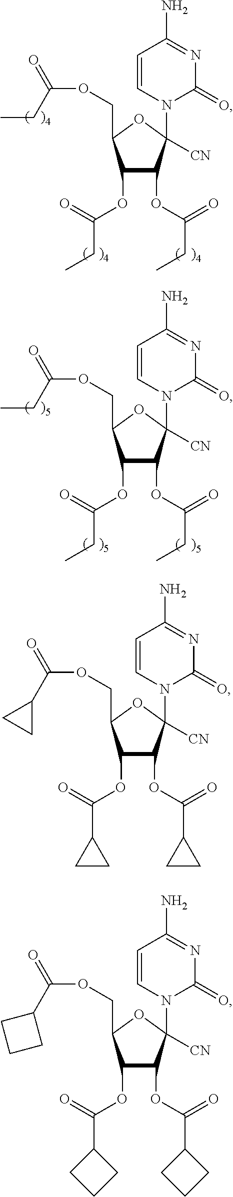 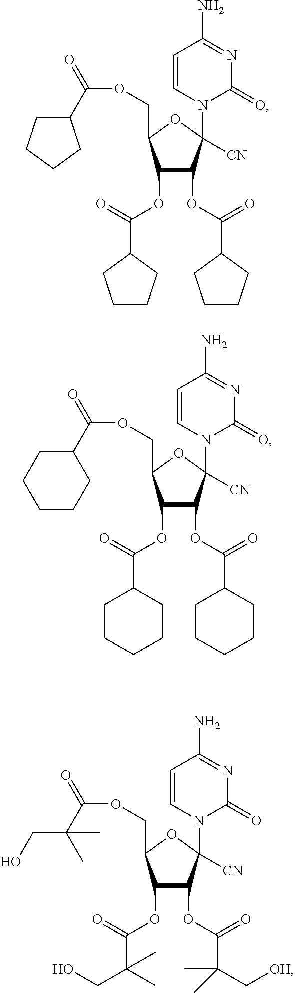 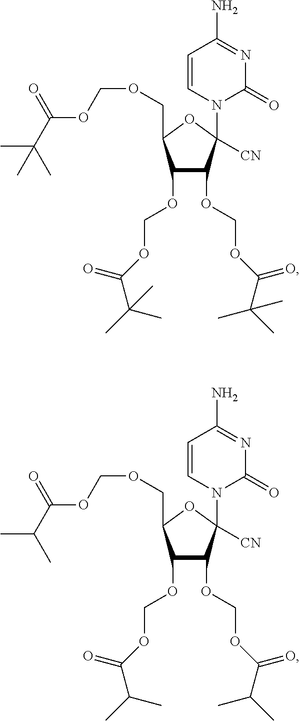 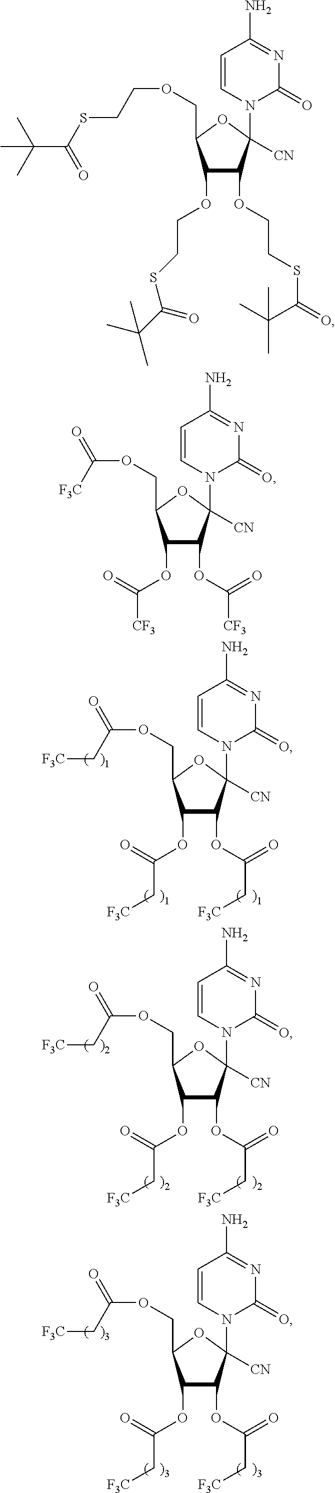 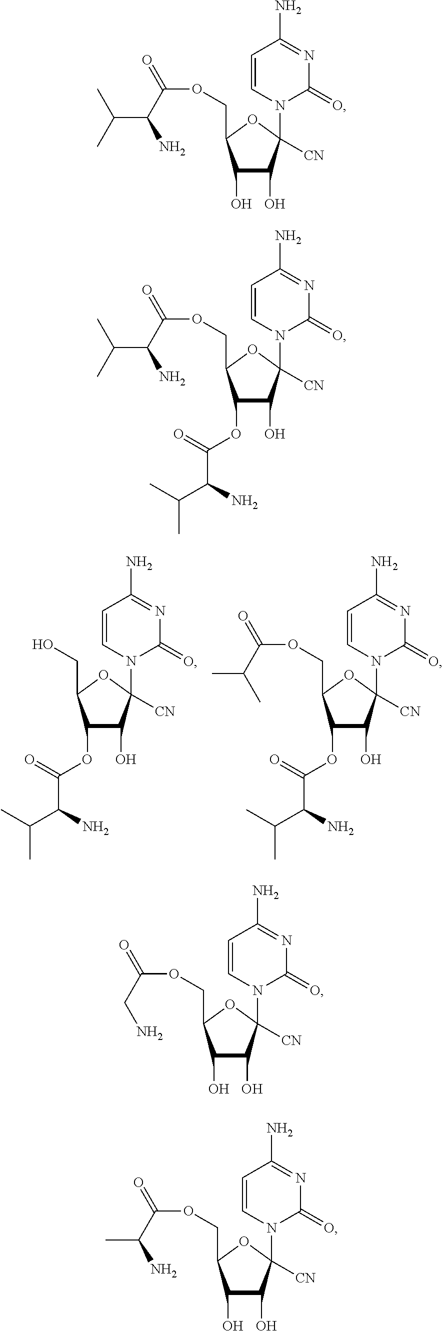 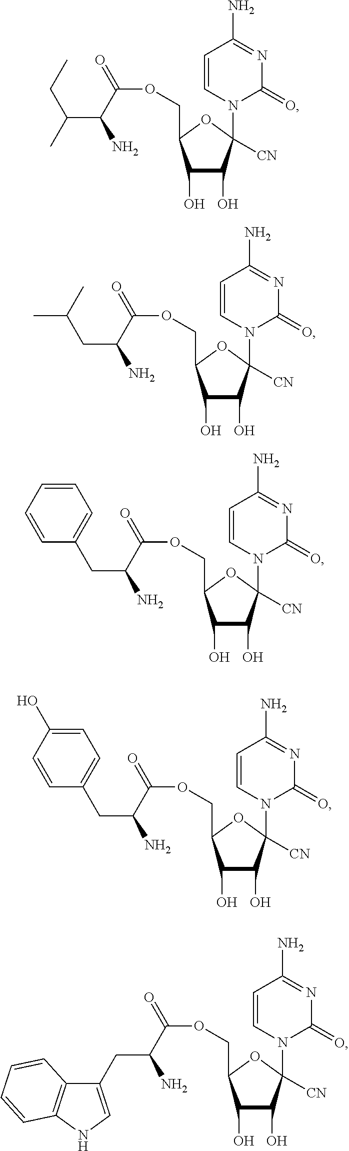 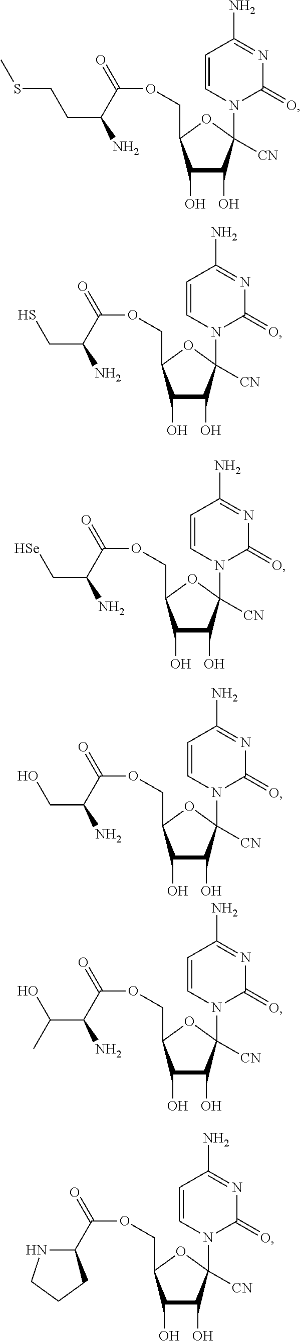 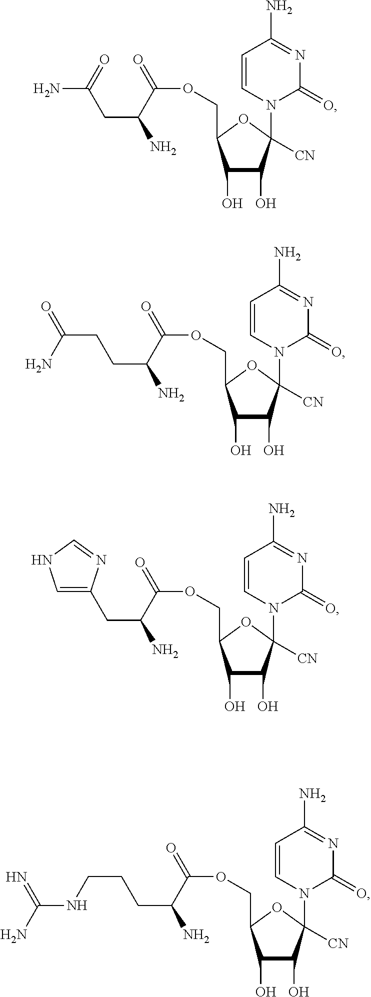 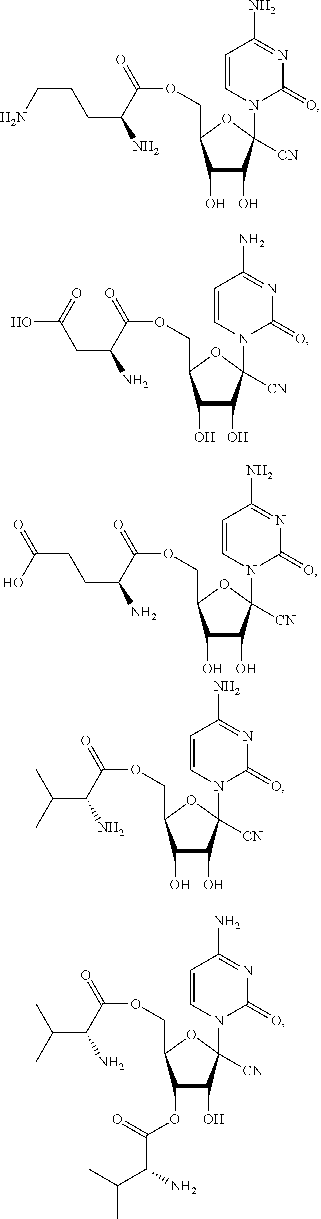 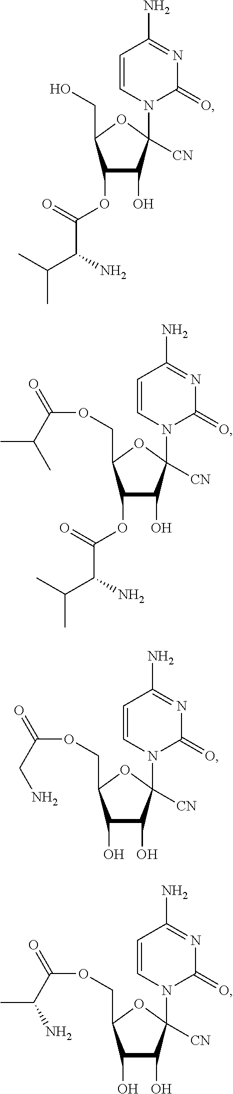 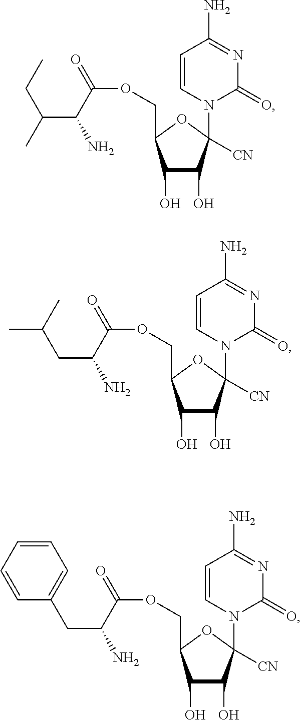 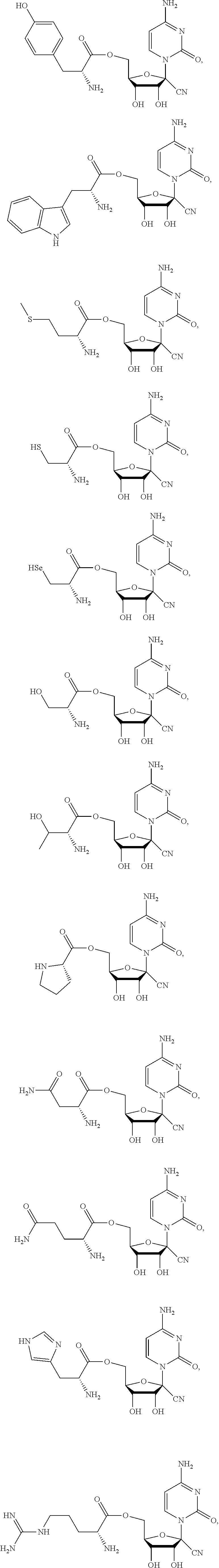 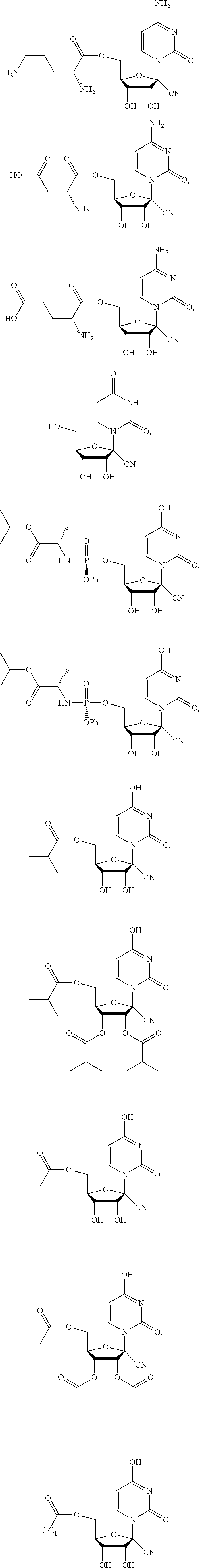 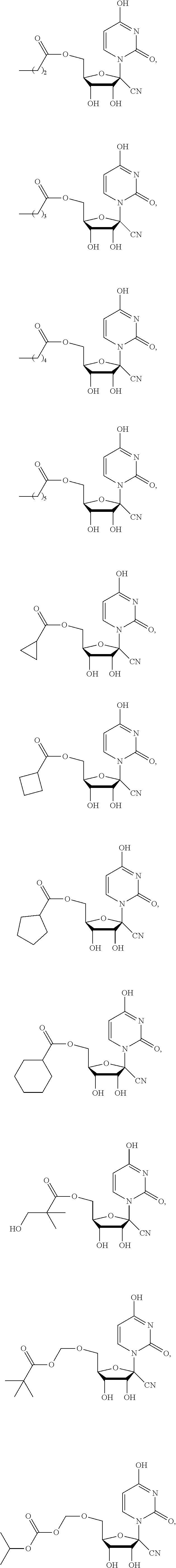 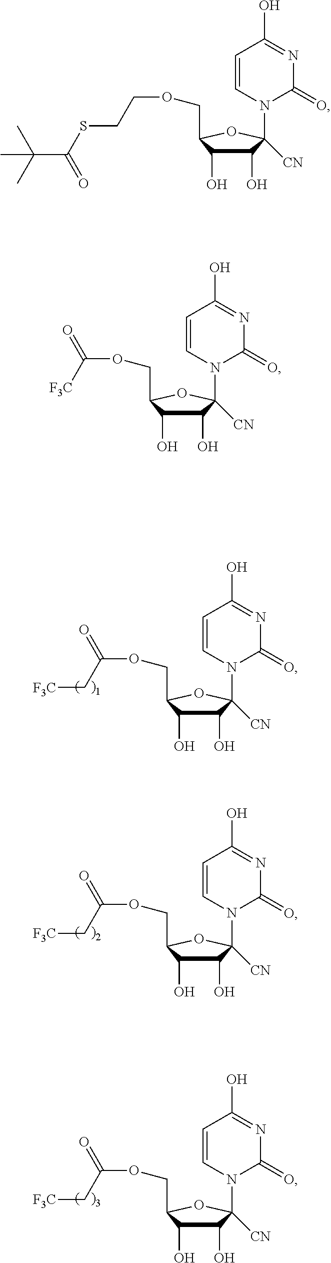 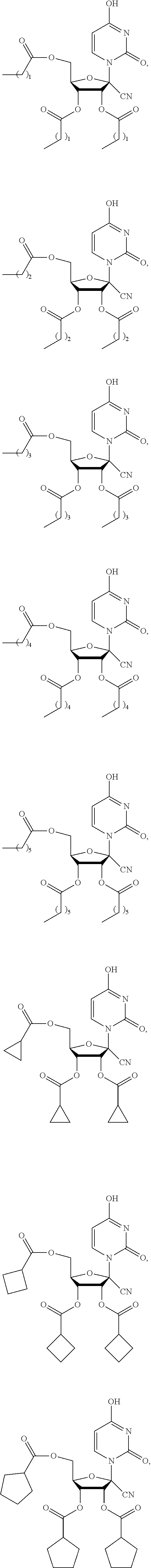 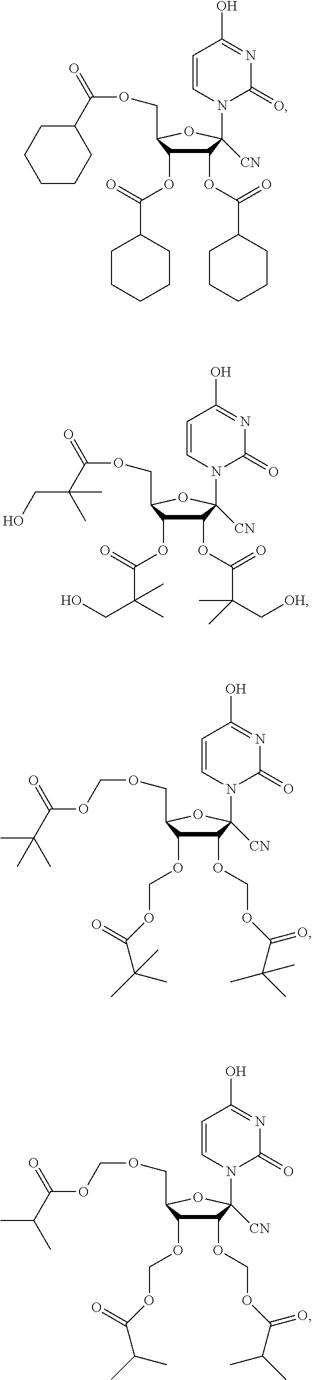 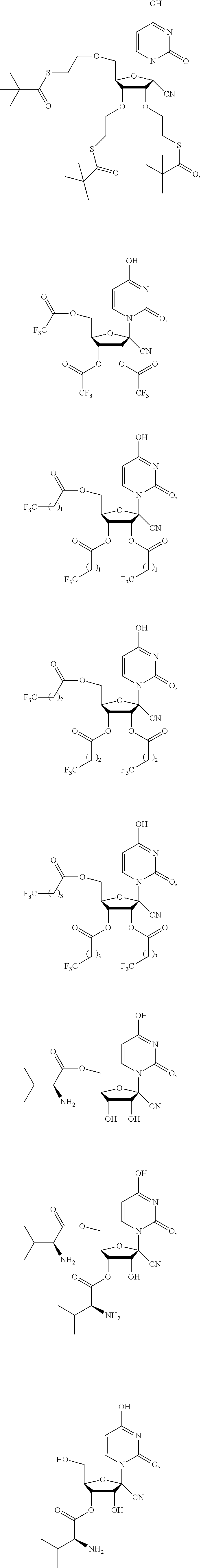 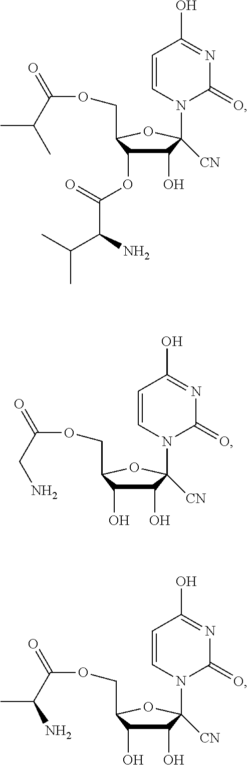 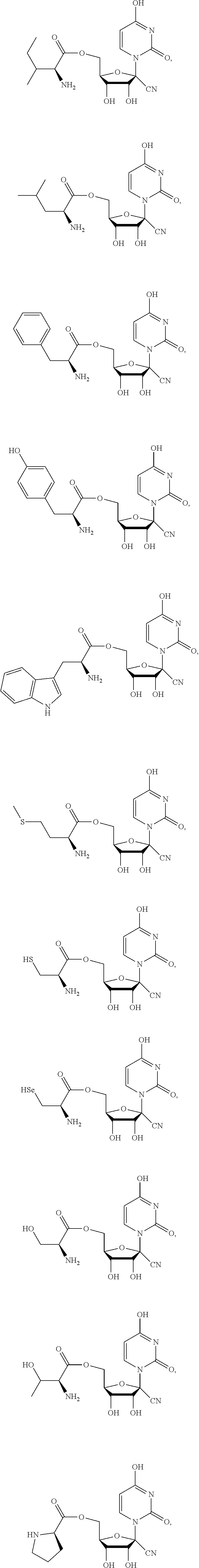 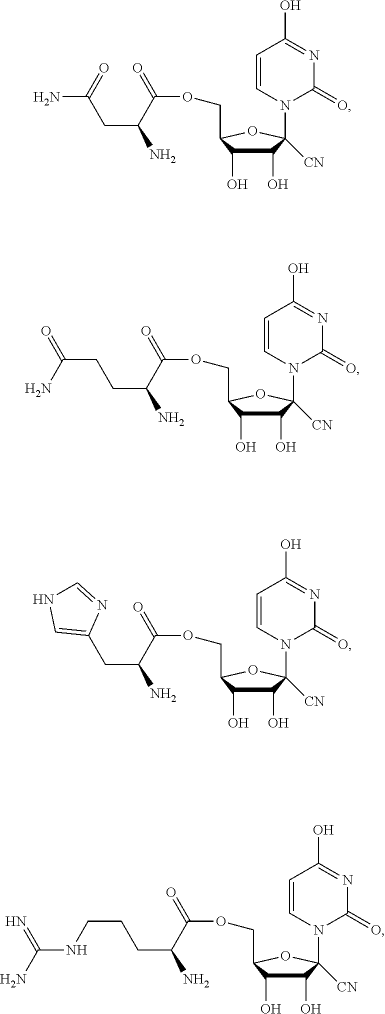 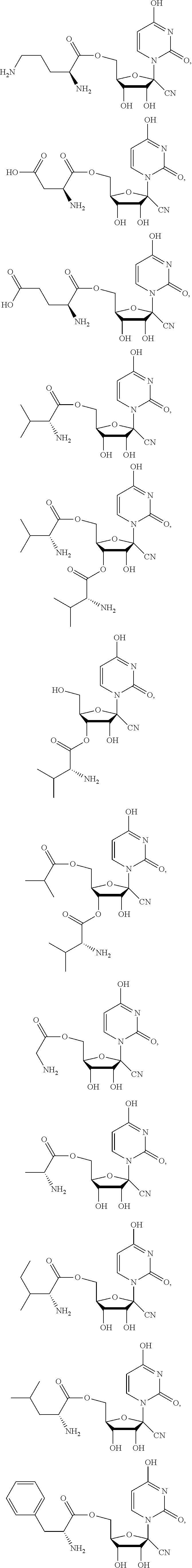 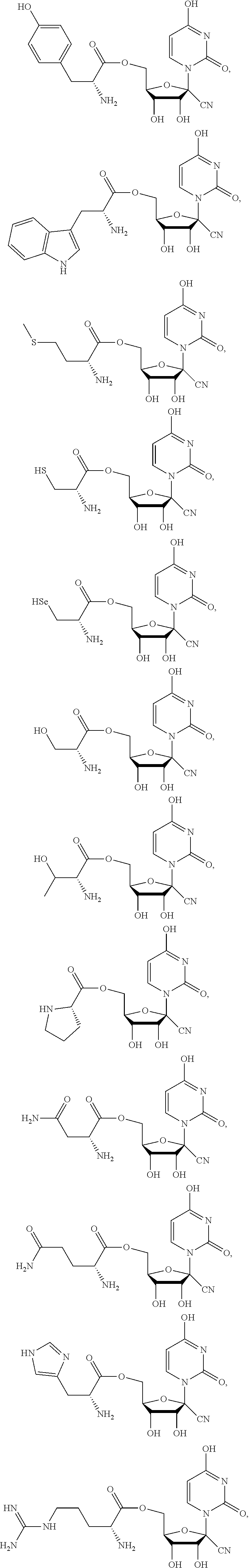 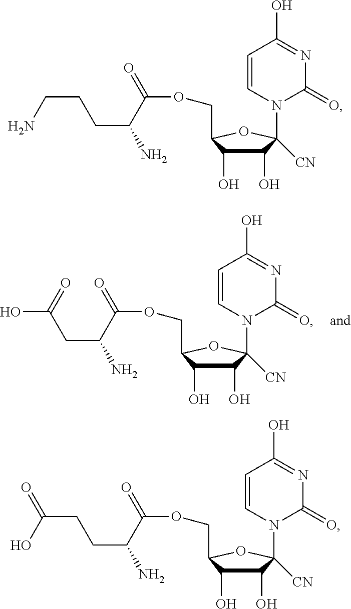
or a pharmaceutically acceptable salt or prodrug thereof.

12. The method of claim 1, wherein the compound has one of the following formulas: 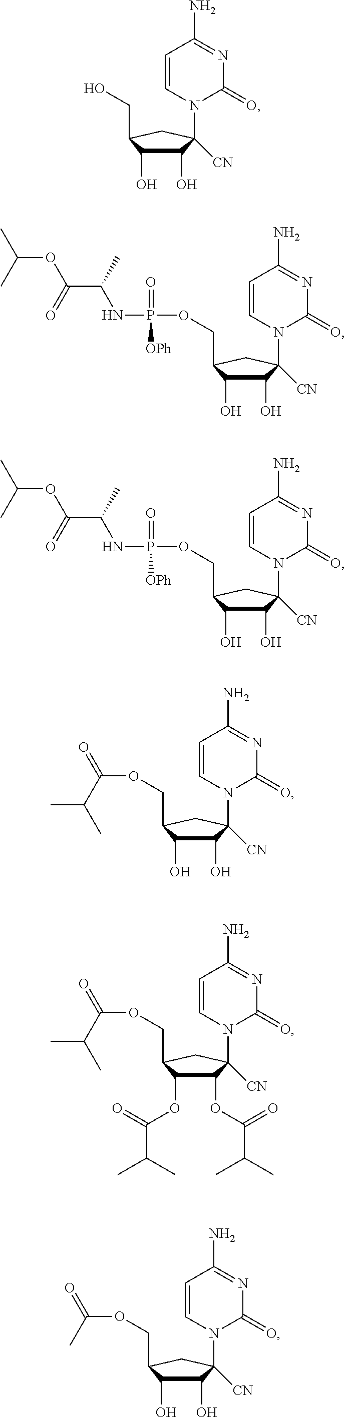 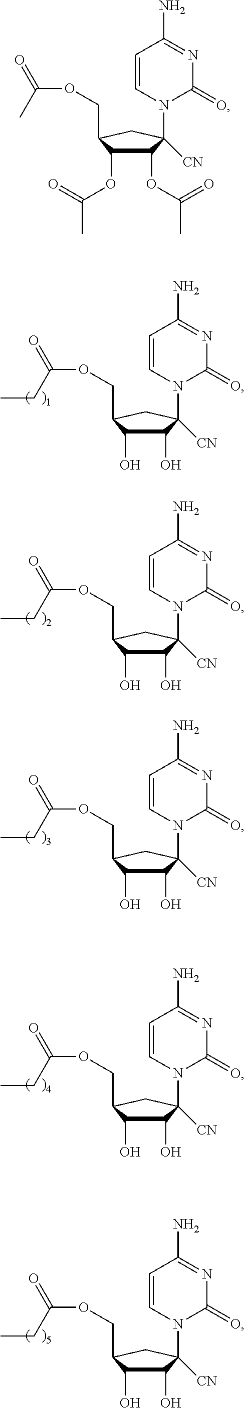 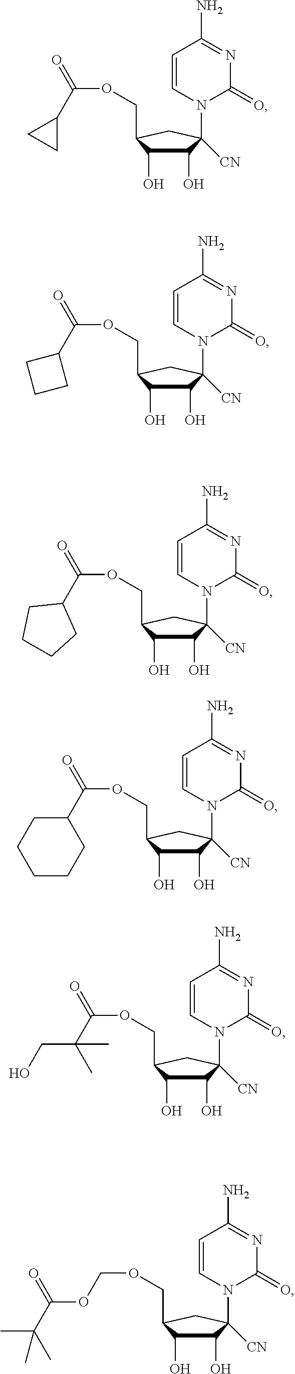 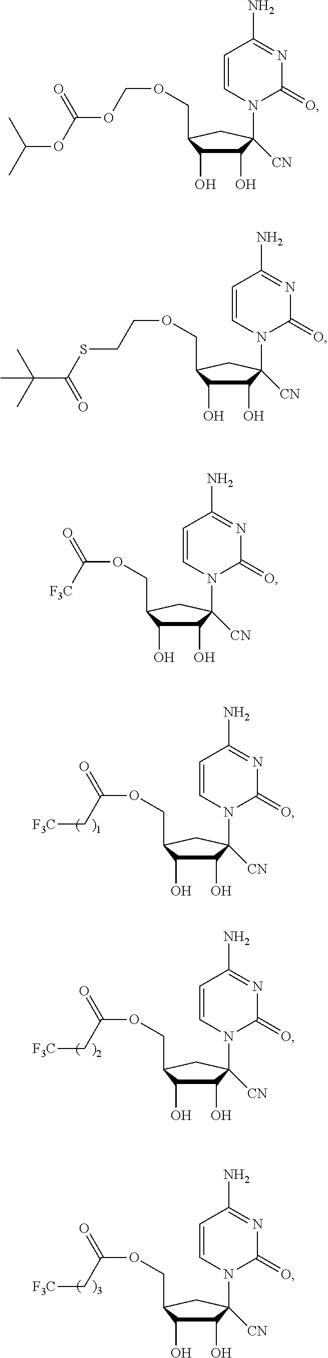 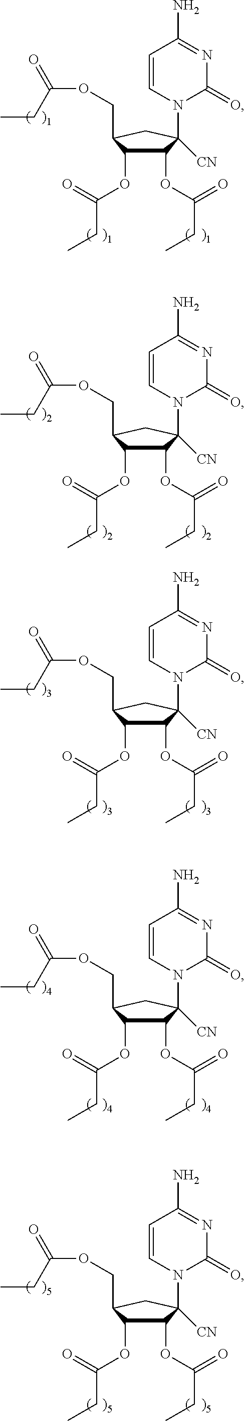 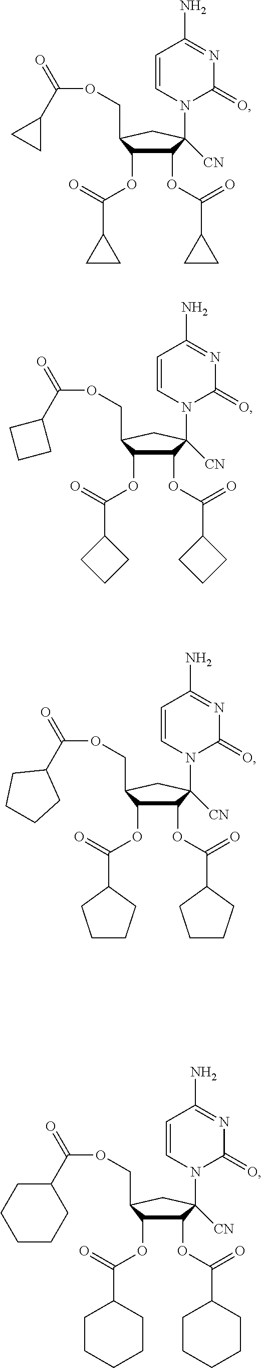 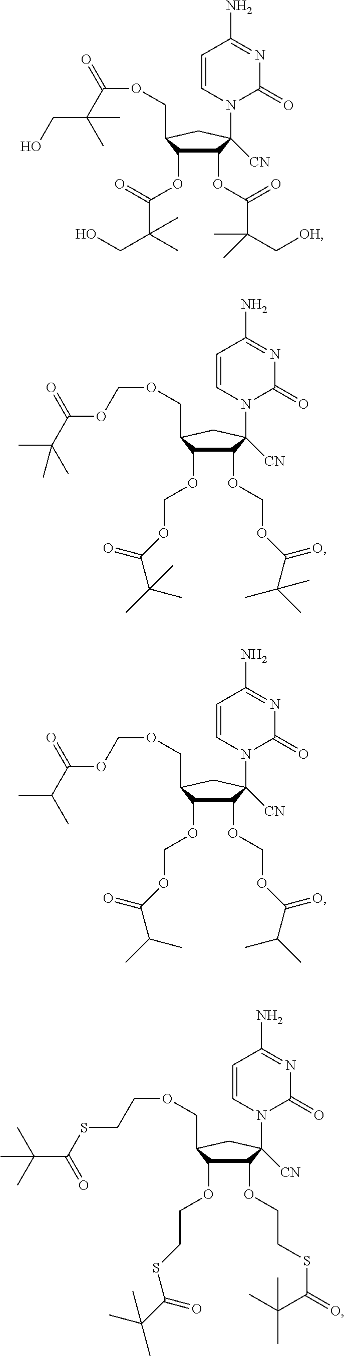 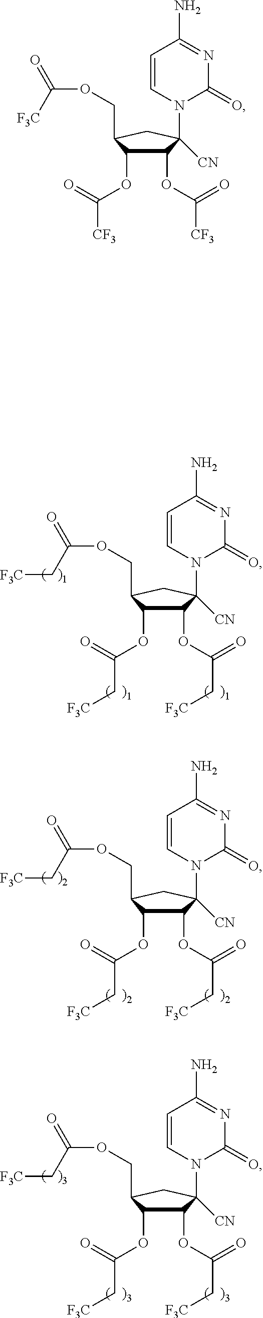 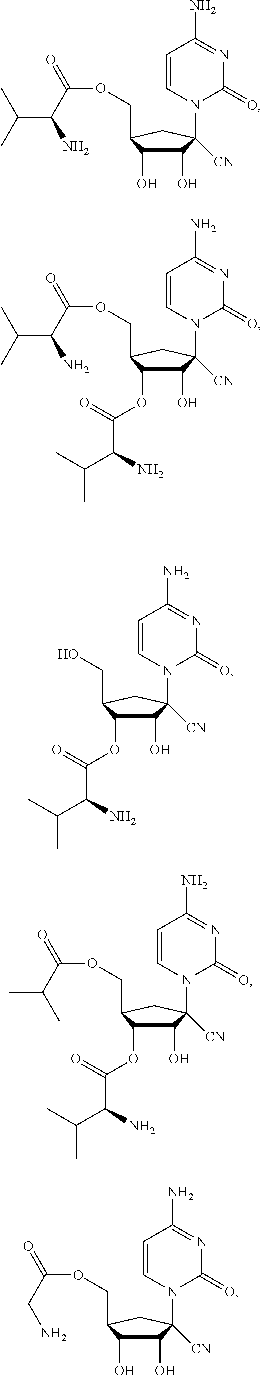 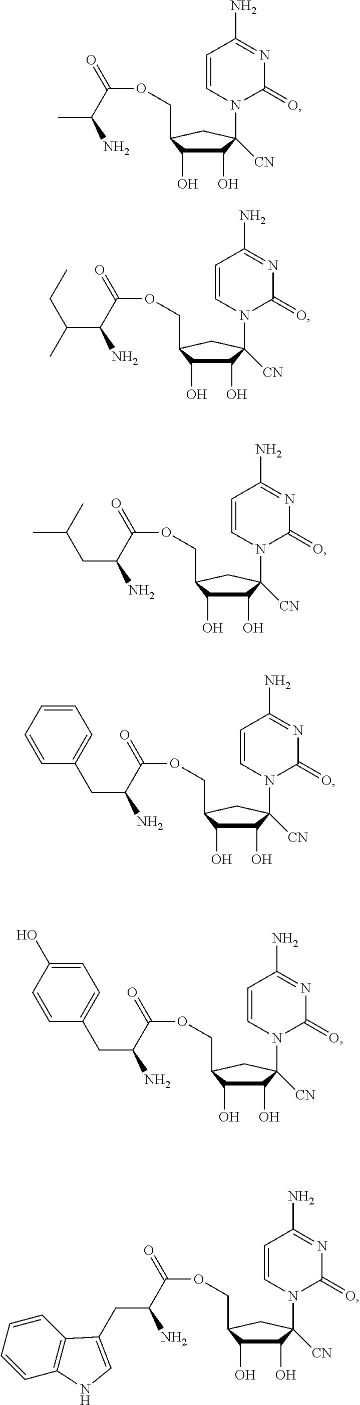 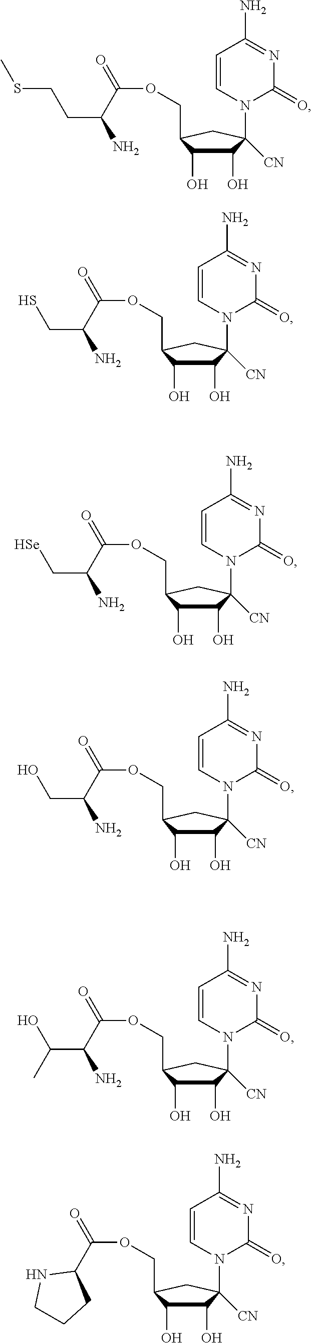 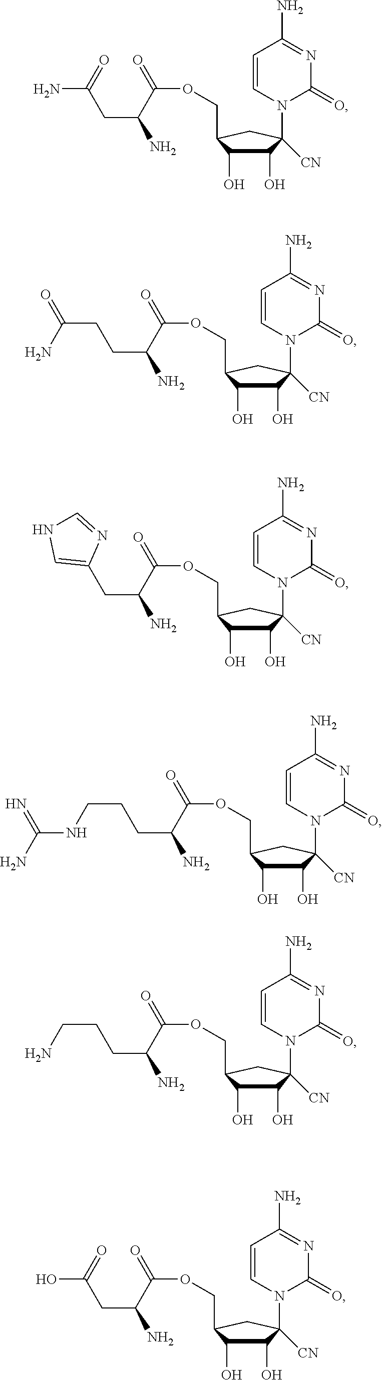 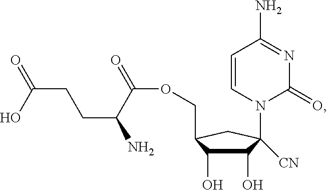 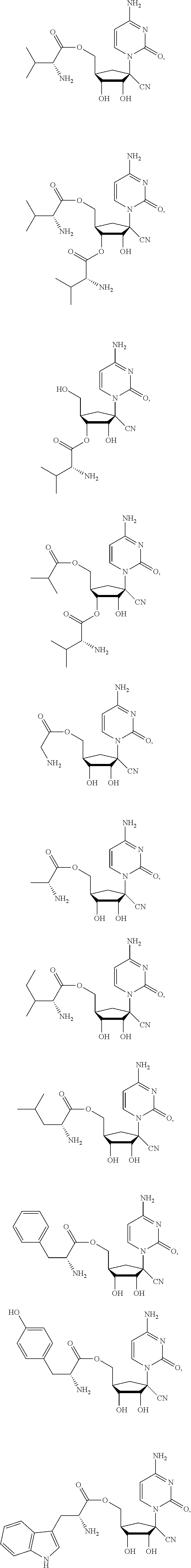 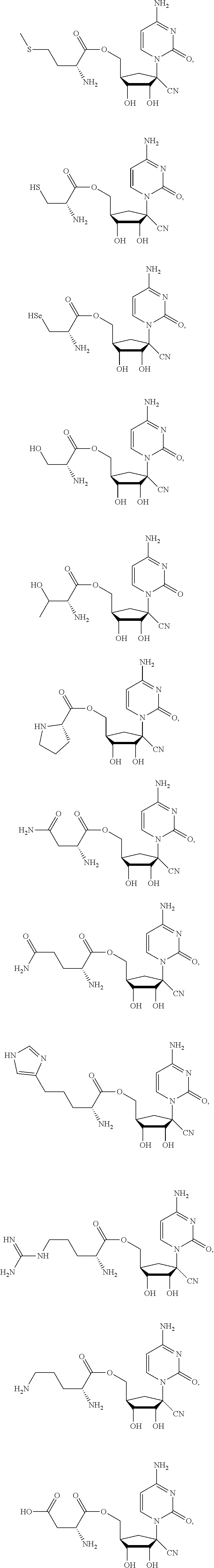 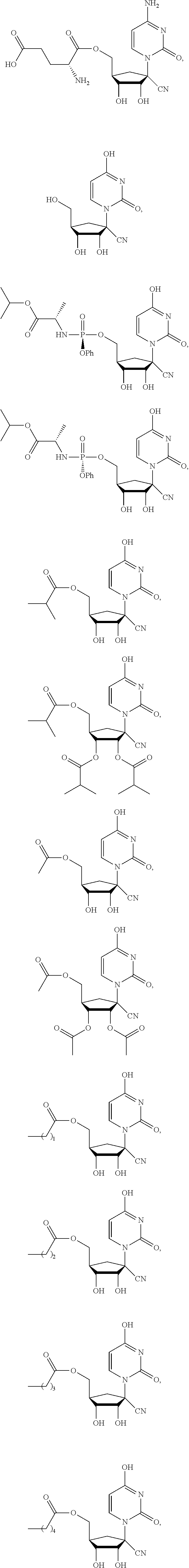 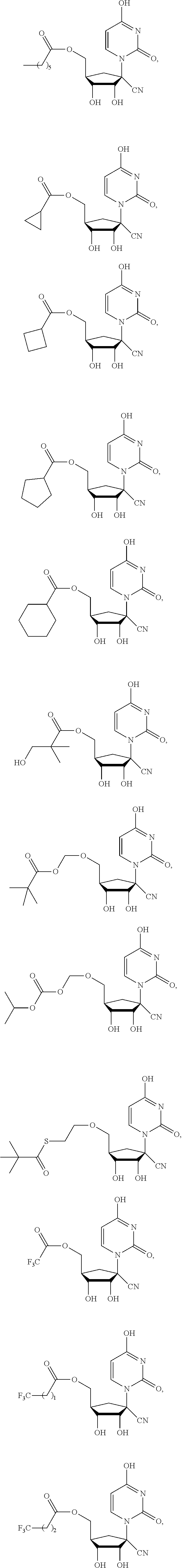 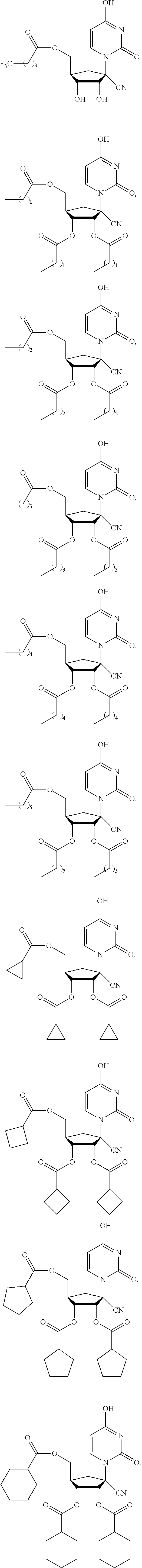 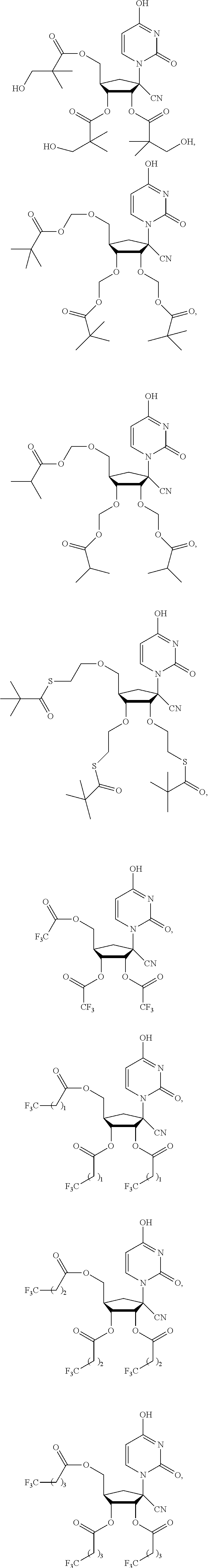 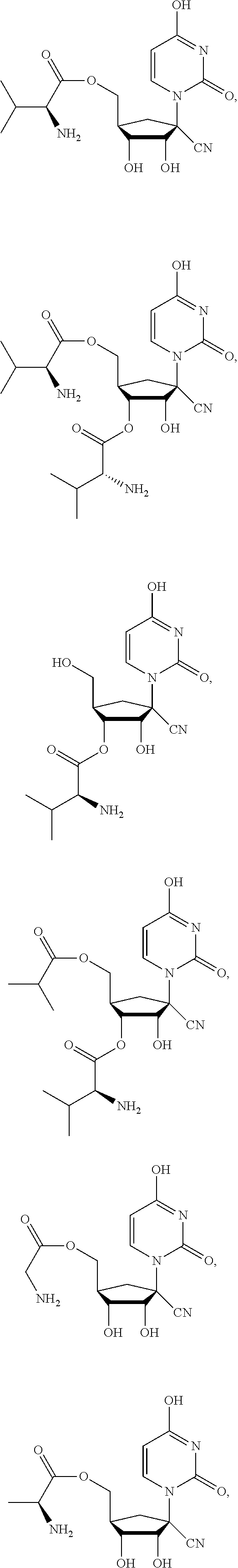 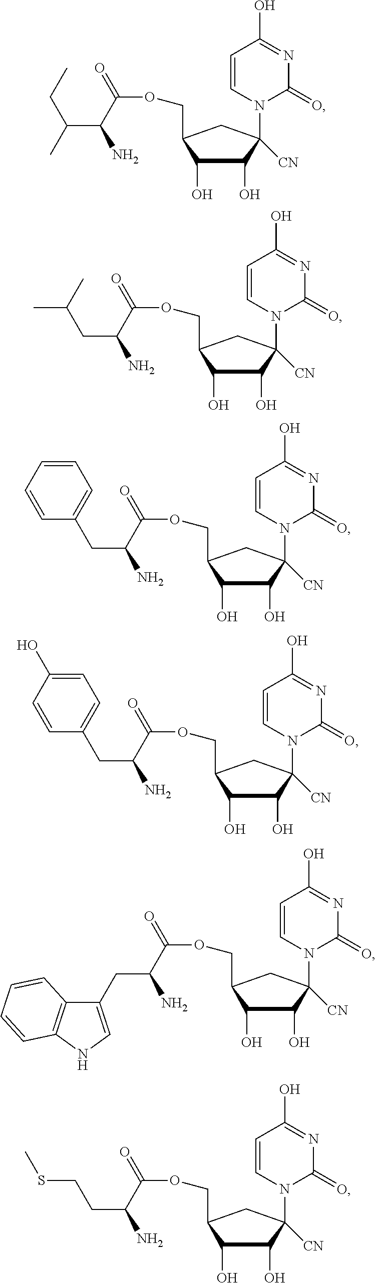 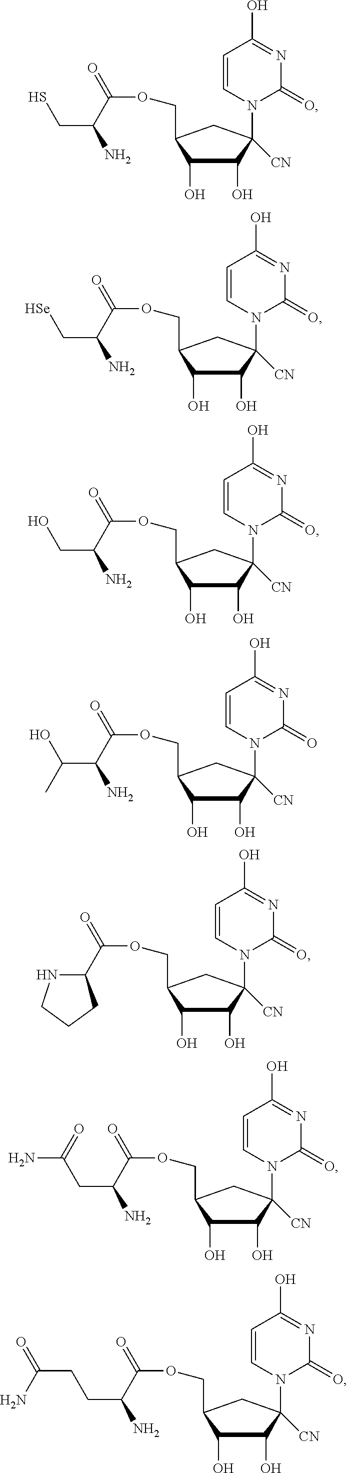 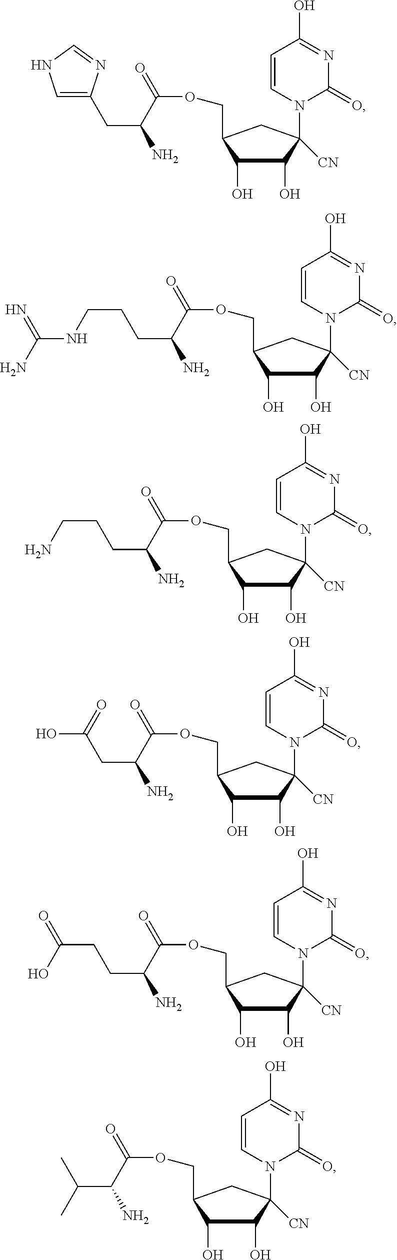 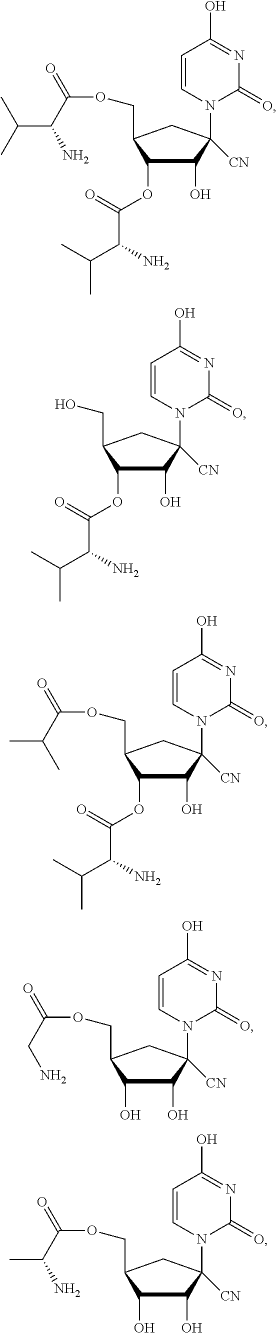 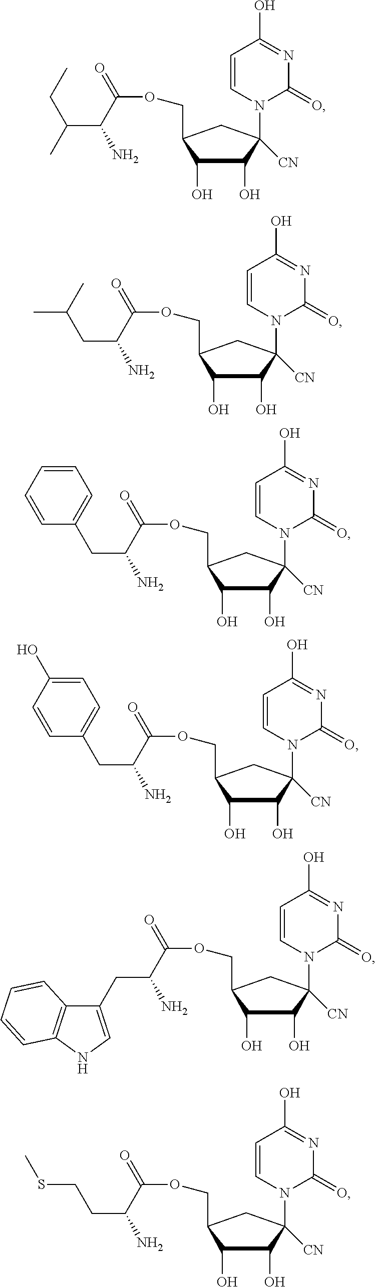 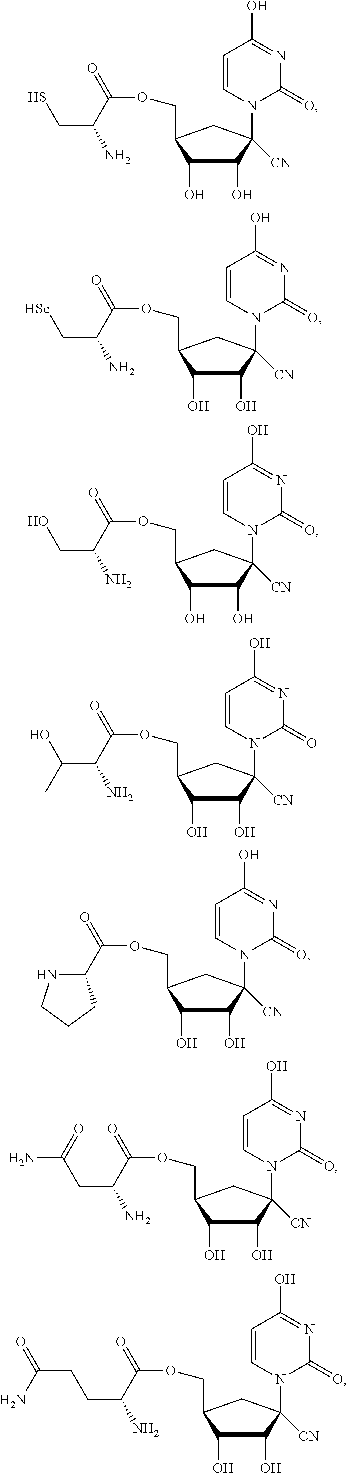 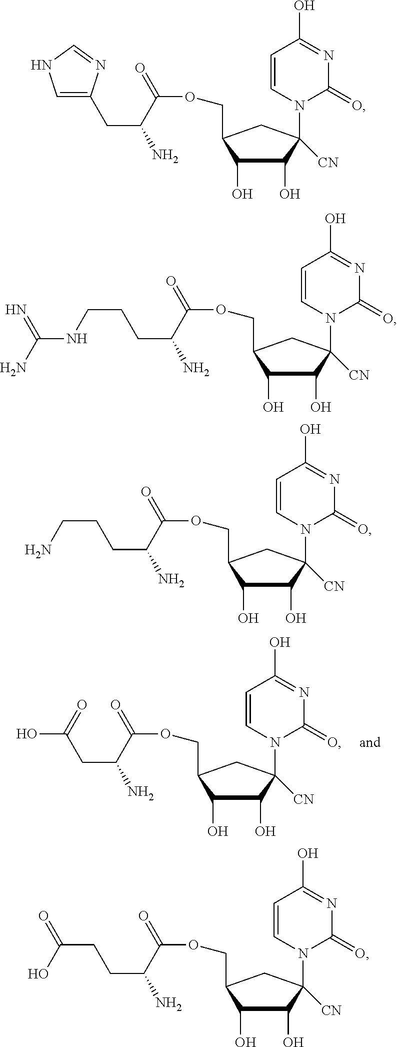
or a pharmaceutically acceptable salt or prodrug thereof.

13. The method of claim 10, wherein the compound is 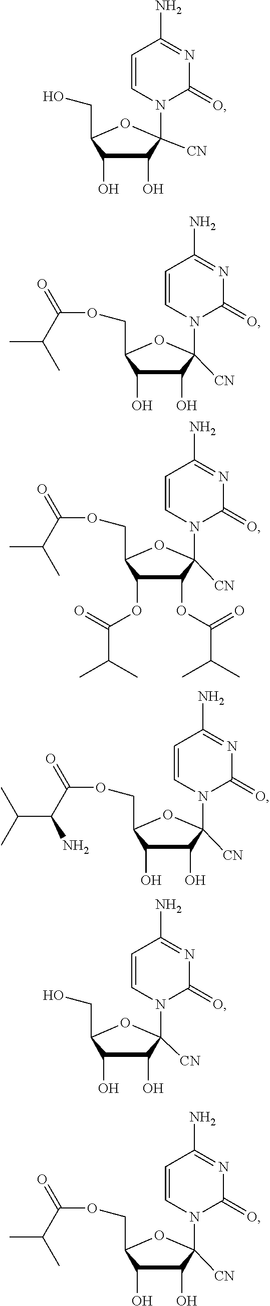 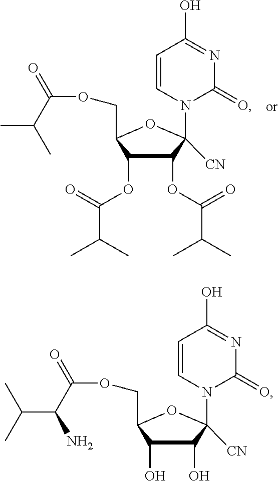
or a pharmaceutically-acceptable salt or prodrug thereof.

14. The method of claim 1, wherein the compound is 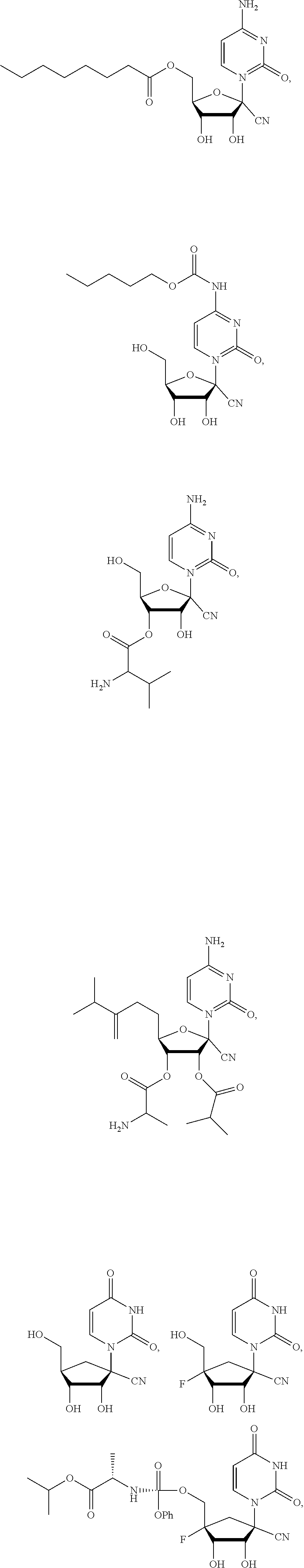
or a pharmaceutically-acceptable salt or prodrug thereof.

15-18. (canceled)

19. The method of claim 1, wherein the compound is co-administered with one or more additional active compounds selected from the group consisting of fusion inhibitors, entry inhibitors, protease inhibitors, polymerase inhibitors, antiviral nucleosides, viral entry inhibitors, viral maturation inhibitors, JAK inhibitors, angiotensin-converting enzyme 2 (ACE2) inhibitors, SARS-CoV-specific human monoclonal antibodies, including CR3022, and agents of distinct or unknown mechanism.

20-33. (canceled)

34. A method for treating or preventing a Coronaviridae, Flaviviridae, Picornaviridae, Bunyaviridae, or Togaviridae infection, comprising administering a treatment or preventative amount of a compound of Formula (A) to a patient in need of treatment or prevention thereof: 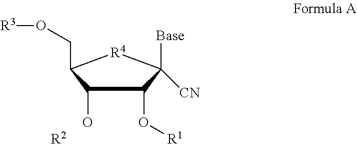
or a pharmaceutically acceptable salt or prodrug thereof, wherein:
R⁴ is O, CH₂, S, Se, or 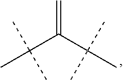
R¹ and R² are independently selected from the group consisting of H, an L-amino acid ester, a D-amino acid ester, an N-substituted L-amino acid ester, an N-substituted D-amino acid ester, an N,N-disubstituted L-amino acid ester, an N,N-disubstituted D-amino acid ester, (acyloxybenzyl)ester, (acyloxybenzyl)ether, optionally substituted bis-acyloxybenzyl)esters, optionally substituted (acyloxybenzyl)esters, an optionally substituted —C(O)—C₁₋₁₂R′, an optionally substituted —C(O)O—R′, an optionally substituted —C(O)S—R′, an optionally substituted —C(S)S—R′, an optionally substituted —C(NR′)OR′, an optionally substituted —C(NR′)SR′, an optionally substituted —C(NR′)N(R′)₂, and an optionally substituted —O—C(O)N(R′)₂, PEG ester, PEG carbonate, an optionally substituted —CH₂—O—C(O)—R′, an optionally substituted —CH₂—O—C(O)O—R′, an optionally substituted —CH₂—CH₂—S—C(O)—R′, a lipid ester, a lipid carbonate, (in which the lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy),
R′ is C₁₋₁₆ alkyl, C₂₋₁₆ alkenyl, C₂₋₁₂ alkynyl, or C₃₋₇ cycloalkyl, wherein optional substituents are selected from the group consisting of halo, C₁₋₁₂ haloalkyl, C₁₋₁₆ alkyl, C₂₋₁₆ alkenyl, C₂₋₁₆ alkynyl, C₃₋₇ cycloalkyl, hydroxyl, carboxyl, C₁₋₁₂ acyl, aryl, heteroaryl, C₁₋₆ acyloxy, amino, amido, carboxyl derivatives, alkylamino, di-C₁₋₁₂-alkylamino, arylamino, C₁₋₁₂ alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, boronic acid and boronic ester;
R³ is H, an L-amino acid ester, a D-amino acid ester, an N-substituted L-amino acid ester, an N-substituted D-amino acid ester, an N,N-disubstituted L-amino acid ester, an N,N-disubstituted D-amino acid ester, (acyloxybenzyl)ester, (acyloxybenzyl)ether, optionally substituted bis-acyloxybenzyl)ester, optionally substituted (acyloxybenzyl)ester, an optionally substituted —C(O)—R′, an optionally substituted —C(O)O—R′, an optionally substituted —C(O)SR′, an optionally substituted —C(S)SR′, PEG ester, PEG carbonate, an optionally substituted —CH₂—O—C(O)—R′, an optionally substituted —CH₂—O—C(O)O—R′, an optionally substituted —CH₂—CH₂—S—C(O)—R′, an optionally substituted —C(NR′)OR′, an optionally substituted —C(NR′)SR′, an optionally substituted —C(NR′)N(R′)₂, an optionally substituted —O—C(O)N(R′)₂, a lipid ester, a lipid carbonate (in which a lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy), O—P(O)R⁶R⁷, or a mono-, di-, or triphosphate, wherein, when chirality exists at the phosphorous center, it may be wholly or partially R, or S, or any mixture thereof,
R⁶ and R⁷ are independently selected from the group consisting of:
(a) OR¹⁵ where R¹⁵ selected from the group consisting of H, 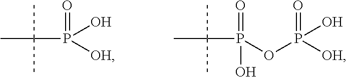
Li, Na, K, substituted or unsubstituted C₁₋₂₀alkyl, substituted or unsubstituted C₃₋₆cycloalkyl, optionally substituted —C(NR′)OR′, optionally substituted —C(NR′)SR′, optionally substituted —C(NR′)N(R′)₂, optionally substituted —O—C(O)N(R′)₂, C₁₋₄(alkyl)aryl, benzyl, C₁₋₆ haloalkyl, C₂₋₃(alkyl)OC₁₋₂₀alkyl, C₂₋₃(alkyl)OC₁₋₂₀alkene, C₂₋₃(alkyl)OC₁₋₂₀alkyne, aryl, and heteroaryl, such as phenyl and pyridinyl, wherein aryl and heteroaryl are optionally substituted with zero to three substituents independently selected from the group consisting of (CH₂)₀₋₆CO₂R⁶ and (CH₂)₀₋₆ CON(R¹⁶)₂;
where R¹⁶ is independently H, substituted or unsubstituted C₁₋₂₀ alkyl, substituted or unsubstituted C₁₋₂₀ alkene, substituted or unsubstituted C₁₋₂₀ alkyne, the carbon chain derived from a fatty alcohol or C₁₋₂₀ alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, C₁₋₅ alkene, C₁₋₅ alkyne, C₃₋₇ cycloalkyl or C₁₋₅ alkyl substituted with a C₁₋₆ alkyl, alkoxy, di(C₁₋₆ alkyl)-amino, fluoro, C₃₋₁₀ cycloalkyl, or cycloalkyl; and
(b) the ester of a D- or L-amino acid 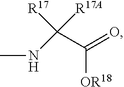
wherein R¹⁷ and R¹⁸ are, independently, H, C₁₋₂₀ alkyl, C₁₋₂₀ alkene, C₁₋₂₀ alkyne, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₃₋₁₀ cycloalkyl, or cycloalkyl; and R^{17A} is H or C₁₋₂alkyl;
Base is 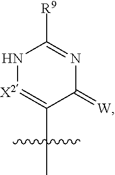
R^{9′} is H, an L-amino acid ester, a D-amino acid ester, an N-substituted L-amino acid ester, an N-substituted D-amino acid ester, an N,N-disubstituted L-amino acid ester, an N,N-disubstituted D-amino acid ester, an (acyloxybenzyl)ester, an (acyloxybenzyl)ether, an optionally substituted bis-acyloxybenzyl)ester, an optionally substituted (acyloxybenzyl)ester, an optionally substituted —C(O)—R′, an optionally substituted —C(O)O—R′, an optionally substituted —C(O)S—R′, an optionally substituted —C(S)S—R′, an optionally substituted C₁₋₁₂-alkyl, an optionally substituted C₂₋₁₂ alkenyl, an optionally substituted C₂₋₁₂ alkynyl, an optionally substituted C₃₋₆ cycloalkyl, an optionally substituted —C(NR)OR′, an optionally substituted —C(NR′)SR′, an optionally substituted —C(NR′)N(R′)₂, an optionally substituted —O—C(O)N(R′)₂, a PEG ester, a PEG carbonate, an optionally substituted —CH₂—O—C(O)—R¹ an optionally substituted —CH₂—O—C(O)O—R′, an optionally substituted —CH₂—CH₂—S—C(O)—R′, a lipid ester, or a lipid carbonate,
wherein a lipid is an optionally substituted C₁₂₋₂₂ alkyl, an optionally substituted C₁₂₋₂₂ alkenyl, an optionally substituted C₁₂₋₂₂ alkynyl or an optionally substituted C₁₂₋₂₂ alkoxy),
R¹⁰ and R^{10′} are independently H, OH, an L-amino acid amide, a D-amino acid amide, (acyloxybenzyl)amide, (acyloxybenzyl)amine, optionally substituted (acyloxybenzyl)esters, an optionally substituted —C(O)—R′, an optionally substituted —C(O)O—R′, an optionally substituted —C(O)S—R′, an optionally substituted —C(S)S—R′, an optionally substituted C₁₋₁₂ alkyl, an optionally substituted C₂₋₁₂ alkenyl, an optionally substituted C₂₋₁₂ alkynyl, an optionally substituted C₃₋₆ cycloalkyl, PEG amide, PEG carbamate, an optionally substituted —CH₂—O—C(O)—R′, an optionally substituted —CH₂—O—C(O)μO—R′, an optionally substituted —CH₂—CH₂—S—C(O)—R′, a lipid amide, an optionally substituted —C(NR′)OR′, an optionally substituted —C(NR′)SR′, an optionally substituted —C(NR′)N(R′)₂, an optionally substituted —O—C(O)N(R′)₂, or a lipid carbamate,
wherein a lipid is an optionally substituted C₁₂₋₂₂ alkyl, an optionally substituted C₁₂₋₂₂alkenyl, an optionally substituted C₁₂₋₂₂ alkynyl or an optionally substituted C₁₂₋₂₂ alkoxy), with the proviso that R¹⁰ and R^{10′} cannot both be OH.

35-62. (canceled)

63. A method for treating or preventing a Coronaviridae, Flaviviridae, Picornaviridae, Bunyaviridae, or Togaviridae infection, comprising administering a treatment or preventative amount of a compound of Formula (A) or Formula (A1) to a patient in need of treatment or prevention thereof: 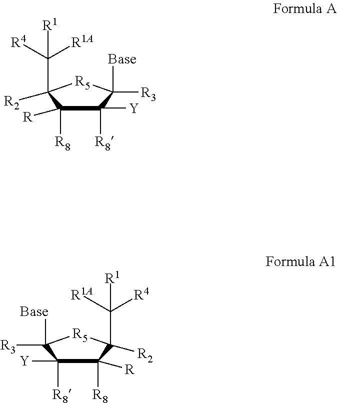
or a pharmaceutically acceptable salt or prodrug thereof, wherein:
Y and R are, independently, selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R¹ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆ alkenyl, C₂₋₆ alkynyl, and C₃₋₆ cycloalkyl, wherein the groups can be substituted with one or more substituents selected from the group consisting of halogen (fluoro, chloro, bromo or iodo), hydroxyl, amino, alkylamino, arylamino, alkoxy, nitro, and cyano,
R¹ is and R^{1A} are, independently, H, CH₃, CH₂F, CHF₂, or CF₃, wherein, when R¹ is Me, the carbon to which it is attached may be wholly or partially R or S or any mixture thereof, or R¹ and R^{1A} can combine to form a C₃₋₇ cycloalkyl ring;
R² is H, CN, N₃, F, CH₂-halogen, CH₂—N₃, O—CH₂—P—(OH)₃ substituted or unsubstituted C₁₋₈ alkyl, substituted or unsubstituted C₂₋₈ alkenyl or substituted or unsubstituted C₂₋₈ alkynyl;
R³ is CN,
R⁵ is O, S, Se, CH₂, CHF, CF₂, —C(CH₃)—, —C(cyclopropyl)-, C═CF₂ or C═CH₂,
R⁸ and R^{8′} are independently selected from the group consisting of H, OH, halo, an optionally substituted O-linked amino acid, substituted or unsubstituted C₁₋₆ alkyl, C₁₋₆ haloalkyl, C₁₋₆ alkoxy, substituted or unsubstituted C₂₋₆ alkenyl, substituted or unsubstituted C₂₋₆ alkynyl, substituted or unsubstituted C₃₋₆ cycloalkyl, cyano, cyanoalkyl, azido, azidoalkyl, OR′, SR′, wherein each R′ is independently a —C(O)—C₁₋₁₂ alkyl, —C(O)—C₂₋₁₂ alkenyl, —C(O)—C₂₋₁₂ alkynyl, —C(O)—C₃₋₆ cycloalkyl, —C(O)O—C₁₋₁₂ alkyl, —C(O)O—C₂₋₁₂ alkenyl, —C(O)O—C₂₋₁₂ alkynyl, —C(O)O—C₃₋₆ cycloalkyl, C₁₋₆ alkyl, C₁₋₆ haloalkyl, C₁₋₆ alkoxy, C₂₋₆alkenyl, C₂₋₆alkynyl, C₃₋₆cycloalkyl, wherein the groups can be substituted with one or more substituents selected from the group consisting of halogen (fluoro, chloro, bromo or iodo), hydroxyl, amino, alkylamino, arylamino, alkoxy, nitro, and cyano,
R⁴ is OH, an optionally substituted O-linked amino acid, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, OC₁₋₆ alkyl, OC₁₋₆haloalkyl, OC₁₋₆ alkoxy, OC₂₋₆ alkenyl, OC₂₋₆alkynyl, OC₃₋₆ cycloalkyl, O—P(O)R⁶R⁷, O—CH₂—P—(OH)₃, O—CH₂—P—(OH)₃, or a mono-, di-, or triphosphate, wherein, when chirality exists at the phosphorous center of R⁴, it may be wholly or partially R, or S, or any mixture thereof,
R⁶ and R⁷ are independently selected from the group consisting of:
(a) OR¹⁵ where R¹⁵ selected from the group consisting of H, 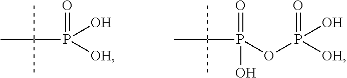
Li, Na, K, substituted or unsubstituted C₁₋₂₀ alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
where R¹⁶ is independently H, substituted or unsubstituted C₁₋₂₀ alkyl, the carbon chain derived from a fatty alcohol or C₁₋₂₀alkyl substituted with a C₁₋₆ alkyl, C₁₋₆ alkoxy, di(C₁₋₆ alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆ alkyl, alkoxy, di(C₁₋₆ alkyl)-amino, fluoro, C₃₋₁₀ cycloalkyl, or cycloalkyl;
(b) the ester of a D- or L-amino acid 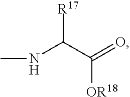
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₁₋₅ alkyl, or C₁₋₅ alkyl substituted with a C₁₋₆alkyl, alkoxy, di(C₁₋₆alkyl)-amino, fluoro, C₄₋₁₀ cycloalkyl, or cycloalkyl;
Base is selected from the group consisting of: 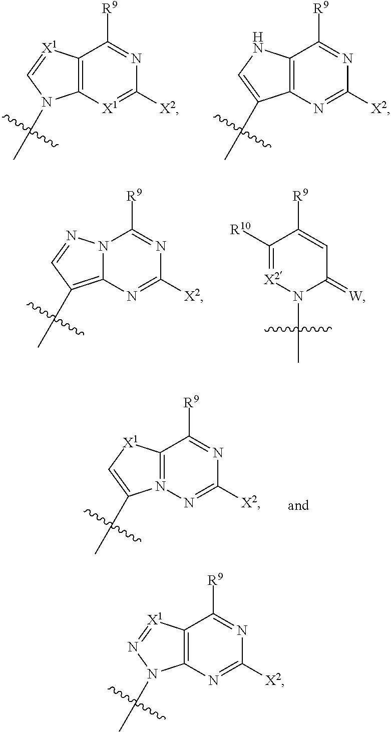
X¹ is CH, C—(C₁₋₆)alkyl, C—(C₂₋₆)alkenyl, C—(C₂₋₆)alkynyl, C—(C₃₋₇)cycloalkyl, C—(C₁₋₆) haloalkyl, C—(C₁₋₆)hydroxyalkyl, C—OR²², C—N(R²²)₂, C-halo, C—CN or N,
X^{1′} is CH, C—(C₁₋₆)alkyl, C—(C₂₋₆)alkenyl, C—(C₂₋₆)alkynyl, C-halo, C—CN or N
R⁹ and X² are independently H, OH, NH₂, halo (i.e., F, Cl, Br, or I), SH, NHOH, O(C₁₋₁₀)alkyl, O(C₂₋₁₀)alkene, O(C₂₋₁₀)alkyne, O(C₃₋₇)cycloalkyl, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, —O—C(O)O—C₃₋₆ cycloalkyl, S(C₁₋₁₀)alkyl, S(C₂₋₁₀)alkene, S(C₂₋₁₀)alkyne, S(C₃₋₇)cycloalkyl, an optionally unsaturated NH(C₁₋₁₀)alkyl, an optionally unsaturated N((C₁₋₁₀)alkyl)₂, NH(C₃₋₇)cycloalkyl, an optionally unsaturated NH(CO)(C₁₋₂₀)alkyl, an optionally unsaturated NH(CO)O(C₁₋₂₀)alkyl, NHOH, an optionally unsaturated NHO(CO)(C₁₋₂₀)alkyl, or an optionally unsaturated NHO(CO)NH(C₁₋₂₀)alkyl, (C₁₋₃)alkyl,
R^{9′} is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
R¹⁰ is H or F,
X^{2′} is N or CH, and
W is O or S.

64-72. (canceled)

73. A method for treating or preventing a Coronaviridae, Flaviviridae, Picornaviridae, Bunyaviridae, or Togaviridae infection, comprising administering a treatment or preventative amount of a compound of Formula (B) or (B1) to a patient in need of treatment or prevention thereof: 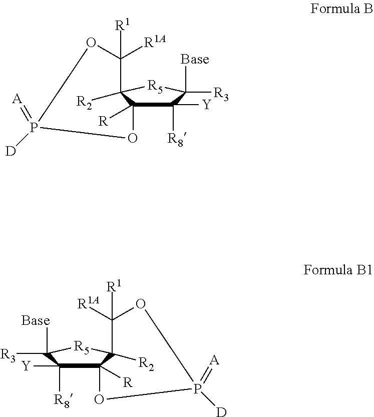
or a pharmaceutically acceptable salt or prodrug thereof, wherein:
Base, Y, R, R¹, R^{1A}, R², R³, R⁵, and R^{8′} are as defined in Formula A,
A is O or S, and
D is selected from the group consisting of:
(a) OR¹⁵ where R¹⁵ is selected from the group consisting of H, substituted or unsubstituted C₁₋₂₀alkyl, substituted or unsubstituted C₃₋₆cycloalkyl, C₁₋₄(alkyl)aryl, benzyl, C₁₋₆ haloalkyl, C₂₋₃(alkyl)OC₁₋₂₀alkyl, aryl, and heteroaryl, such as phenyl and pyridinyl, wherein aryl and heteroaryl are optionally substituted with zero to three substituents independently selected from the group consisting of (CH₂)₀₋₆CO₂R¹⁶ and (CH₂)₀₋₆ CON(R¹⁶)₂;
(b) the ester of a D- or L-amino acid 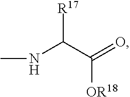
R¹⁷ and R¹⁸ are independently H, C₁₋₂₀ alkyl, the carbon chain derived from a fatty alcohol or C₁₋₂₀ alkyl optionally substituted with a C₁₋₆ alkyl, alkoxy, di(C₁₋₆alkyl)-amino, fluoro, C₃₋₁₀ cycloalkyl, cycloalkyl-C₁₋₆ alkyl, cycloheteroalkyl, aryl, heteroaryl, substituted aryl, or substituted heteroaryl; wherein the substituents are C₃₋₁₀ alkyl, or C₁₋₅ alkyl substituted with a C₁₋₆ alkyl, alkoxy, di(C₁₋₆alkyl)-amino, fluoro, C₃₋₁₀ cycloalkyl, or cycloalkyl; and 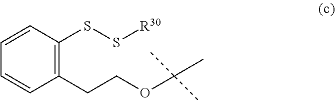
where R³⁰ is selected from the group consisting of substituted or unsubstituted C₁₋₂₀alkyl, substituted or unsubstituted C₃₋₆ cycloalkyl, substituted or unsubstituted (C₂₋₁₀)alkene, substituted or unsubstituted (C₂₋₁₀)alkyne, C₁₋₄(alkyl)aryl, aryl, heteroaryl, and C₁₋₆ haloalkyl.

74-79. (canceled)

80. A method for treating or preventing a Coronaviridae, Flaviviridae, Picornaviridae, Bunyaviridae, or Togaviridae infection, comprising administering a treatment or preventative amount of a compound of Formula (C) or (C1) to a patient in need of treatment or prevention thereof: 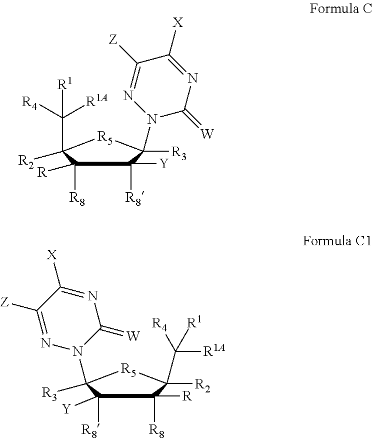
or a pharmaceutically acceptable salt or prodrug thereof, wherein:
R, R¹, R^{1A}, R², R³, R⁵, R⁸, R^{8′} and Y are as defined in Formula A,
X is OH, NH₂, SH, NHOH, —O—C(O)—C₁₋₁₂ alkyl, —O—C(O)—C₂₋₁₂ alkenyl, —O—C(O)—C₂₋₁₂ alkynyl, —O—C(O)—C₃₋₆ cycloalkyl, —O—C(O)O—C₁₋₁₂ alkyl, —O—C(O)O—C₂₋₁₂ alkenyl, —O—C(O)O—C₂₋₁₂ alkynyl, or —O—C(O)O—C₃₋₆ cycloalkyl,
Z is H or F, and
W is O or S.

81-90. (canceled)

91. A method for treating or preventing a Coronaviridae, Flaviviridae, Picornaviridae, Bunyaviridae, or Togaviridae infection, comprising administering a treatment or preventative amount of a compound of Formula (D) or (D1) to a patient in need of treatment or prevention thereof: 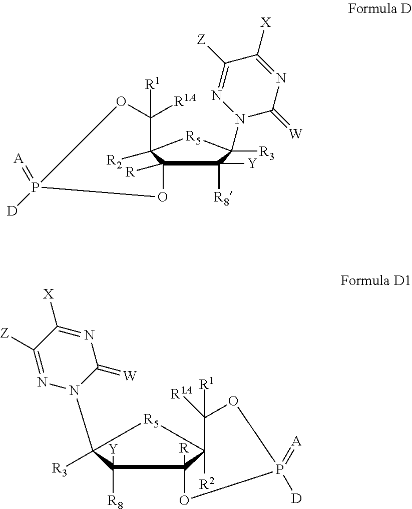
or a pharmaceutically acceptable salt or prodrug thereof, wherein R, R¹, R^{1A}, R², R³, R⁵, R^{8′} and Y are as defined in Formula A, and A and D are as defined in Formula C.

92-101. (canceled)

102. A method for treating or preventing a Coronaviridae, Flaviviridae, Picornaviridae, Bunyaviridae, or Togaviridae infection, comprising administering a treatment or preventative amount of a compound of Formula (E) or (E1) to a patient in need of treatment or prevention thereof: 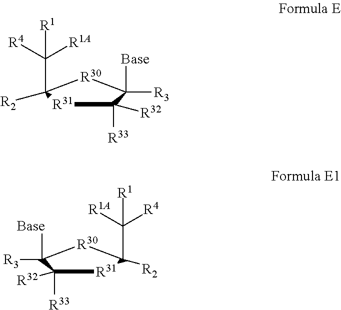
or a pharmaceutically acceptable salt or prodrug thereof, wherein:
Base, R¹, R^{1A}, R², R³, and R⁴ are as defined in Formula A,
R³⁰ is O, S, or CH₂,
R¹¹ is O or S,
R³¹ is O when R³⁰ is S, and
R³² and R³³ are independently H, F, C₁-C₃ alkyl, C₂-C₃ alkene, or C₂-C₃ alkyne.

103-110. (canceled)

111. A method for treating or preventing a Coronaviridae, Flaviviridae, Picornaviridae, Bunyaviridae, or Togaviridae infection, comprising administering a treatment or preventative amount of a compound of Formula (F) or (F1) to a patient in need of treatment or prevention thereof: 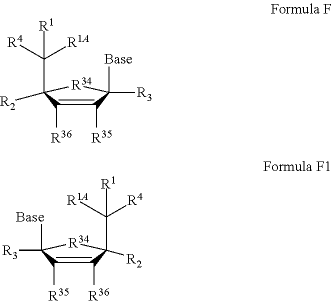
or a pharmaceutically acceptable salt or prodrug thereof, wherein:
Base, R¹, R^{1A}, R², R³, and R⁴ are as defined in Formula A,
R³⁴ is O, S, or CH₂, and
R³⁵ and R³⁶ are independently H, F or CH₃.

112-116. (canceled)

117. A method for treating or preventing a Coronaviridae, Flaviviridae, Picornaviridae, Bunyaviridae, or Togaviridae infection, comprising administering a treatment or preventative amount of a compound of one of the following formulas to a patient in need of treatment or prevention thereof: 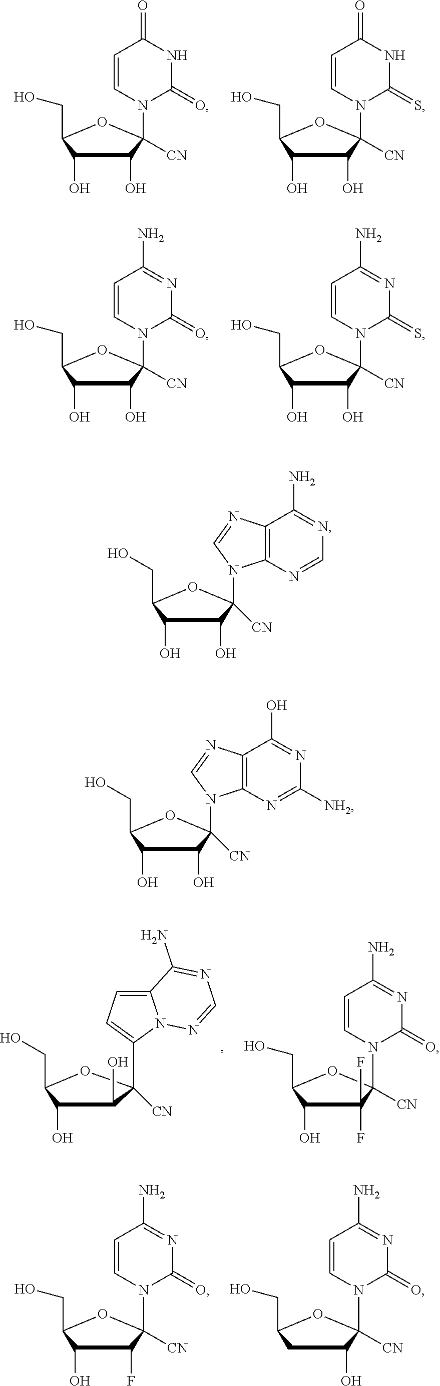 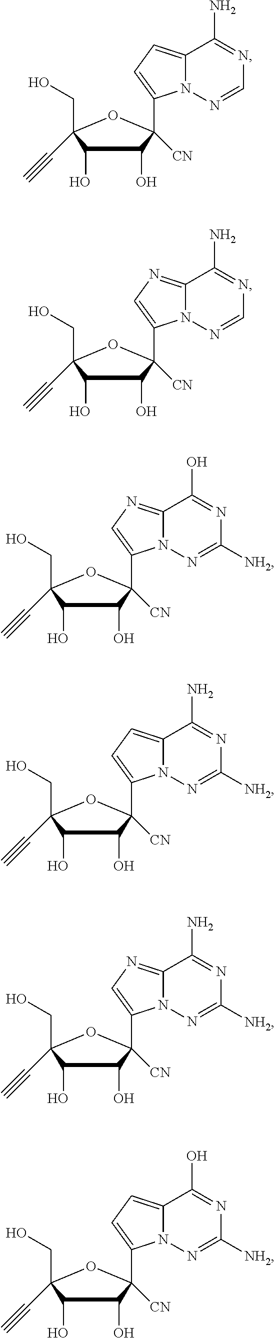
or a pharmaceutically-acceptable salt or prodrug thereof.

118-141. (canceled)
